(19) United States
(12) Patent Application Publication
(10) Pub. No.: US 2024/0239781 A1
(43) Pub. Date: Jul. 18, 2024

(54) 1H-PYRAZOLE DERIVATIVES AS SIGMA LIGANDS

(71) Applicant: ACONDICIONAMIENTO TARRASENSE, Barcelona (ES)

(72) Inventors: Ana Virginia LLORENTE-FERNÁNDEZ, Barcelona (ES); Carmen ALMANSA-ROSALES, Barcelona (ES); Mónica GARCÍA-LÓPEZ, Barcelona (ES); Ute CHRISTMANN, Barcelona (ES); Jose Luís DÍAZ-FERNÁNDEZ, Barcelona (ES)

(21) Appl. No.: 18/556,504

(22) PCT Filed: Apr. 19, 2022
(86) PCT No.: PCT/EP2022/060304

§ 371 Date: Oct. 20, 2023

(30) Foreign Application Priority Data
Apr. 20, 2021 (EP) 21382338.8

(51) Int. Cl. C07D 413/14; A61K 31/407; A61K 31/4155; A61K 31/422; A61K 31/438; A61K 31/444; A61K 31/553; C07D 401/14; C07D 403/14; C07D 471/10; C07D 487/10; C07D 498/04

(52) U.S. Cl. CPC C07D 413/14; A61K 31/407; A61K 31/4155; A61K 31/422; A61K 31/438; A61K 31/444; A61K 31/553; C07D 401/14; C07D 403/14; C07D 471/10; C07D 487/10; C07D 498/04

(57) ABSTRACT

The present invention relates to new 1H-pyrazole derivatives of formula (I) as sigma ligands having a great affinity for sigma receptors, especially the sigma-1 receptor (σ₁) and/or sigma-2 receptor (σ₂), as well as to the process for the preparation thereof, to compositions comprising them, and to their use as medicaments.

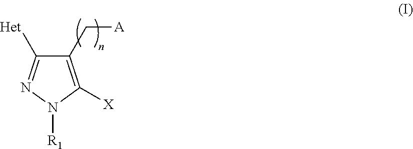

### FIELD OF THE INVENTION

The present invention relates to new 1H-pyrazole derivatives as sigma ligands having a great affinity for sigma receptors, especially the sigma-1 receptor (σ₁) and/or sigma-2 receptor (σ₂), as well as to the process for the preparation thereof, to compositions comprising them, and to their use as medicaments.

### BACKGROUND OF THE INVENTION

The search for new therapeutic agents has been greatly aided in recent years by better understanding of the structure of proteins and other biomolecules associated with target diseases. One important class of these proteins are the sigma (σ) receptors, originally discovered in the central nervous system (CNS) of mammals in 1976 and initially related to the dysphoric, hallucinogenic, and cardiac stimulant effects of opioids. Subsequent studies established a complete distinction between the a receptors binding sites and the classical opiate receptors. From studies of the biology and function of sigma receptors, evidence has been presented that sigma receptor ligands may be useful in the treatment of psychosis and movement disorders such as dystonia and tardive dyskinesia, and motor disturbances associated with Huntington's chorea or Tourette's syndrome and in Parkinson's disease [Walker, J. M. et al., Pharmacological Reviews, (1990), 42, 355]. It has been reported that the known sigma receptor ligand rimazole clinically shows effects in the treatment of psychosis [Snyder, S. H., Largent, B. L., J. Neuropsychiatry, (1989), 1, 7]. The sigma binding sites have preferential affinity for the dextrorotatory isomers of certain opiate benzomorphans, such as (+)-SKF-10047, (+)-cyclazocine, and (+)-pentazocine and also for some narcoleptics such as haloperidol. The sigma receptor has two subtypes that were initially discriminated by stereoselective isomers of these pharmacoactive drugs. (+)-SKF-10047 has nanomolar affinity for the sigma-1 (σ₁) site and has micromolar affinity for the sigma-2 (σ₂) site. Haloperidol has similar affinities for both subtypes.

The σ₁ receptor is expressed in numerous adult mammal tissues (e.g. central nervous system, ovary, testicle, placenta, adrenal gland, spleen, liver, kidney, gastrointestinal tract) as well as in embryo development from its earliest stages, and is apparently involved in a large number of physiological functions. Its high affinity for various pharmaceuticals has been described, such as for (+)-SKF-10047, (+)-pentazocine, haloperidol and rimazole, among others, known ligands with analgesic, anxiolytic, antidepressive, antiamnesic, antipsychotic and neuroprotective activity. Hence, the σ₁ receptor has possible physiological roles in processes related to analgesia, anxiety, addiction, amnesia, depression, schizophrenia, stress, neuroprotection and psychosis [Walker, J. M. et al., Pharmacological Reviews, (1990), 42, 355; Kaiser, C. et al., Neurotransmissions, (1991), 7 (1), 1-5; Bowen, W. D., Pharmaceutica Acta Helvetiae, (2000), 74, 211-218].

The σ₁ receptor is a ligand-regulated chaperone of 223 amino acids and 25 kDa cloned in 1996 and crystallized twenty years later [Hanner, M. et al., Proc. Natl. Acad. Sci. USA, (1996), 93, 8072-8077; Su, T. P. et al., Trends Pharmacol. Sci., (2010), 31, 557-566; Schmidt, H. R. et al., Nature, (2016), 532, 527-530]. Residing primarily in the interface between the endoplasmic reticulum (ER) and mitochondrion, referred to as the mitochondria-associated membrane (MAM), it can translocate to the plasma membrane or ER-membrane and regulate the activity of other proteins by modulating N-methyl-D-aspartic (NMDA) receptors and several ion channels [Monnet, F. P. et al., Eur. J. Pharmacol., (1990), 179, 441-445; Cheng, Z. X. et al., Exp. Neurol., (2010), 210, 128-136]. Owing to the role played by the σ₁R in modulating pain-related hypersensitivity and sensitization phenomena, σ₁R antagonists have been also proposed for the treatment of neuropathic pain [Drews, E. et al., Pain, 2009, 145, 269-270; De la Puente, B. et al., Pain (2009), 145, 294-303; Díaz, J. L. et al., J. Med. Chem., (2012), 55, 8211-8224; Romero et al., Brit. J. Pharm., (2012), 166, 2289-2306; Merlos, M. et al., Adv. Exp. Med. Biol., (2017), 964, 85-107]. Additionally, the σ₁ receptor has been known to modulate opioid analgesia, and the relationship between the μ-opioid and σ₁ receptors has been shown to involve direct physical interaction, which explains why σ₁ receptor antagonists enhance the antinociceptive effect of opioids without increasing their adverse effects [Chien, C. C. et al, J. Pharmacol. Exp. Ther., (1994), 271, 1583-1590; King, M. et al, Eur. J. Pharmacol., (1997), 331, R5-6; Kim, F. J. et al., Mol. Pharmacol., (2010), 77, 695-703; Zamanillo, D. et al., Eur. J. Pharmacol., (2013), 716, 78-93].

The σ₂ receptor was initially identified by radioligand binding as a site with high affinity for di-o-tolylguanidine (DTG) and haloperidol [Hellewell, S. B. et al., Brain Res., (1990), 527, 244-253]. Two decades later, progesterone receptor membrane component 1 (PGRMC1), a cytochrome-related protein that binds directly to heme and regulates lipid and drug metabolism and hormone signaling, was proposed as the complex where resides the σ₂R binding site [Xu, J. et al., Nat. Commun., (2011), 2, 380]. Finally, in 2017, the σ₂R subtype was purified and identified as transmembrane protein-97 (TMEM97), an endoplasmic-reticulum-resident molecule implicated in cholesterol homeostasis due to its association with the lysosomal Niemann-Pick cholesterol transporter type 1 (NPC1) [Alon, A. et al., Proc. Natl. Acad. Sci. USA, (2017), 114, 7160-7165; Ebrahimi-Fakhari, D. et al., Human Molecular Genetics, (2016), 25, 3588-3599]. The role of σ₂ receptor in cholesterol pathways was known since the 1990s and recent studies published by Mach et al. on modulation of trafficking and internalization of LDL by the formation of a ternary complex between LDLR, PGRMC1 and TMEM97, reinforces this association [Moebius, F. F. et al., Trends Pharmacol. Sci., (1997), 18, 67-70; Riad, A. et al., Sci. Rep., (2018), 8, 16845].

σ₂R/TMEM97, previously known also as meningioma-associated protein, MAC30, is expressed in various normal and diseased human tissues and up-regulation in certain tumors and down-regulation in other suggested that this protein played a distinct role in human malignancies. The cloning of σ₂ receptor confirmed its overexpression in epithelial, colorectal, ovarian lung and breast cancers [Moparthi, S. B. et al., Int. J. Oncol., (2007), 30, 91-95; Yan, B. Y. et al., Chemotherapy, (2010), 56, 424-428; Zhao, Z. R.; Chemotherapy, (2011), 57, 394-401; Ding, H. et al., Asian Pac. J. Cancer Prev., (2016), 17, 2705-2710]. σ₂R/TMEM97 has a molecular weight of 18-21.5 kDa and its sequence predicts a four transmembrane domain protein with cytosolic N and C terminal [Hellewell, S. B. et al., Eur. J. Pharmacol. Mol. Pharmacol. Sect., (1994), 268, 9-18]. The potential signal transduction of σ₂ receptor is not yet understood, but it seems to modulate Ca²⁺ and K⁺ channels, and to interact with caspases, epidermal growth factor receptor (EGFR), and with mammalian target of rapamycin, mTOR, signaling pathways [Vilner, B. J. et al., J. Pharmacol. Exp. Ther., (2000), 292, 900-911; Wilke, R. A. et al., J. Biol. Chem., (1999), 274, 18387-18392; Huang, Y.-S. et al., Med. Res. Rev., (2014), 34, 532-566]. These findings would explain the apoptotic effect of some σ₂ ligands by lysosome dysfunction, reactive oxygen species (ROS) production and caspase-dependent events [Ostenfeld, M. S. et al., Autophagy, (2008), 4, 487-499; Hornick, J. R. et al., J. Exp. Clin. Cancer Res., (2012), 31, 41; Zeng, C. et al., Br. J. Cancer, (2012), 106, 693-701; Pati, M. L. et al., BMC Cancer, (2017), 17, 51].

The σ₂ receptor is involved also in dopaminergic transmission, microglia activation, and neuroprotection [Guo, L. et al., Curr. Med. Chem. (2015), 22, 989-1003]. Terada et al. published in 2018 that σ₂ ligands enhance nerve growth factor (NGF)-induced neurite outgrowth in PC12 cells [Terada, K. et al., Plos One, (2018), 13, e0209250]. The σ₂ receptor plays a key role in amyloid β (Aβ)-induced synaptotoxicity, and σ₂ receptor ligands that block the interaction of Aβ oligomers with the σ₂ receptor have been shown to be neuroprotective [Izzo, N. J. et al., Plos One, (2014), 9, e111899]. σ₂ receptor modulators improve cognitive performance in a transgenic mouse model of Alzheimer's disease (AD), and in two mouse traumatic brain injury models, and could also reduce ischemic stroke injury by enhancing glial cell survival, blocking ischemia-induced glial cell activation, and decreasing nitrosative stress [Katnik, C. et al., J. Neurochem., (2016), 139, 497-509; Yi, B. et al., J. Neurochem., (2017), 140, 561-575; Vázquez-Rosa, E. et al., ACS Chem. Neurosci., (2019), 10, 1595-1602]. The 62 receptor has been implicated in other neurological disorders as schizophrenia [Harvey, P. D. et al., Schizophrenia Research (2020), 215, 352-356], alcohol abuse [Scott, L. L. et al., Neuropsychopharmacology, (2018), 43, 1867-1875] and pain [Sahn, J. J. et al., ACS Chem. Neurosci., (2017), 8, 1801-1811]. Norbenzomorphan UKH-1114, a σ₂ ligand, relieved mechanical hypersensitivity in the spared nerve injury (SNI) mice model of neuropathic pain, an effect explained by the preferential expression of σ₂R/TMEM97 gene in structures involved in pain such as the dorsal root ganglion (DRG).

The σ₂ receptor requires two acidic groups (Asp29, Asp56) for ligand binding, similar to σ₁R, which requires Asp126 and Glu172. σ₁R and σ₂R might have similarities in their binding sites but not necessarily other structural similarities if their amino acid sequences are compared. As σ₁R, σ₂ receptor interacts with a wide range of signaling proteins, receptors and channels, but the question if σ₂ receptor has a primarily structural or a modulatory activity remains to be answered. Several classes of σ₂ receptor ligands have been developed since Perregaard et al., synthesized Siramesine and indole analogues in 1995 [Perregaard, J. et al., J. Med. Chem., (1995), 38, 1998-2008]: tropanes [Bowen, W. D. et al., Eur. J. Pharmacol., (1995), 278, 257-260], norbenzomorphans [Sahn, J. J. et al., ACS Med. Chem. Lett., (2017), 8, 455-460], tetrahydroisoquinolines [Sun, Y.-T. et al., Eur. J. Med. Chem., (2018), 147, 227-237] or isoindolines [Grundmana, M. et al., Alzheimer's & Dementia: Translational Research & Clinical Interventions, (2019), 5, 20-26] amongst others [Berardi, F. et al., J. Med. Chem., (2004), 47, 2308-2317]. Many of these ligands present a lack of selectivity over serotoninergic receptors but mainly, there is a difficulty to reach a high selectivity over σ₁. Several σ₁-selective ligands are available, but ligands with high selectivity for σ₂ over 6, are relatively scarce. A significant challenge for the study of the σ₂ receptor is the paucity of highly σ₂-selective ligands.

In view of the potential therapeutic applications of agonists or antagonists of the sigma receptor, a great effort has been directed to find selective ligands. Thus, the prior art has disclosed different sigma receptor ligands, as outlined above.

Nevertheless, there is still a need to find compounds having pharmacological activity towards the sigma receptor, being both effective, selective, and/or having good “drugability” properties, i.e. good pharmaceutical properties related to administration, distribution, metabolism and excretion.

Surprisingly, it has been observed that the new 1H-pyrazole derivatives with general Formula (I) show a selective affinity for sigma receptors, in particular, for σ₁ and σ₂. These compounds are therefore particularly suitable as pharmacologically active agents in medicaments for the prophylaxis and/or treatment of disorders or diseases related to sigma receptors.

### SUMMARY OF THE INVENTION

The present invention discloses novel compounds with great affinity to sigma receptors which might be used for the treatment of sigma related disorders or diseases. In particular, the compounds of the invention can be useful for the treatment of pain and pain related disorders and/or CNS (Central Nervous System) disorders.

The invention is directed in a main aspect to a compound of Formula (I),

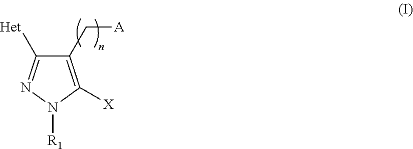

wherein Het, X, A, R₁ and n are as defined below in the detailed description.

A further aspect of the invention refers to the processes for preparation of compounds of formula (I).

It is also an aspect of the invention a pharmaceutical composition comprising a compound of formula (I).

Finally, it is an aspect of the invention a compound of formula (I) for use in therapy and more particularly for the treatment of pain and pain related conditions and/or CNS (Central Nervous System) disorders.

### DETAILED DESCRIPTION OF THE INVENTION

The invention is directed to a family of compounds, in particular to 1H-pyrazole derivatives which show a pharmacological activity towards the sigma receptors, thus solving the above problem of identifying alternative or improved pain and/or CNS treatments by offering such compounds.

The applicant has found that the problem of providing a new effective and alternative solution for treating pain and pain related disorders and/or CNS (Central Nervous System) disorders can surprisingly be solved by using compounds binding to the sigma receptors.

In a first aspect, the present invention is directed to a compound of formula (I):

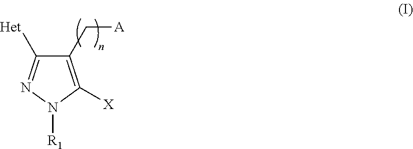

wherein:
X is a halogen atom;
Het is a 5 to 12-membered heterocyclic ring or ring system optionally substituted by one or more R₂ radicals;
A represents —CO—B or —CH₂—B
B is one of the following moieties:

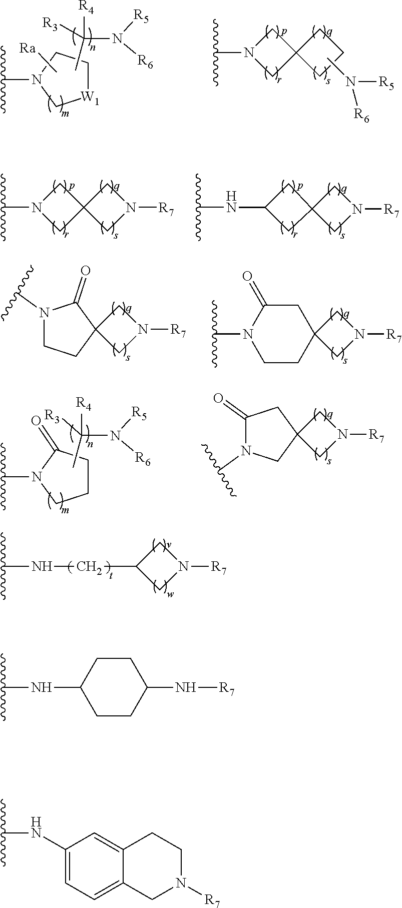

Rₐ is a hydrogen atom or a branched or unbranched C₁₋₆ alkyl radical;
R₁ is a branched or unbranched C₁₋₆ alkyl radical; an aryl radical or a 5 or 6-membered N-containing heteroaromatic ring;
R₂ is a hydrogen atom, a branched or unbranched C₁₋₆ alkyl radical; a C₃₋₉ cycloalkyl radical; a halogen atom; a C₁₋₆ haloalkyl radical or —OR₂, being R_{2′} a hydrogen atom or a C₁₋₆ alkyl radical;
R₃ and R₄ represent a hydrogen atom, a branched or unbranched C₁₋₆ alkyl radical; or alternatively R₃ and R₄ form together with the atom they are attached to a C₃₋₉ cycloalkyl group;
R₅ is a hydrogen atom or a branched or unbranched C₁₋₆ alkyl radical;
R₆ is a branched or unbranched C₁₋₆ alkyl radical; CH(R_{6′})—(CH₂)ₖ-aryl being R_{6′} a hydrogen atom or a C₁₋₆ alkyl radical; a —(CH₂)ⱼ-heterocyclyl; or a —CH₂—CO— heterocyclyl, the heterocyclyl being optionally mono- or bisubstituted;
R₇ is a branched or unbranched C₁₋₆ alkyl radical; —CH(R_{7′})—(CH₂)ₖ-aryl being R_{7′} a hydrogen atom or a C₁₋₆ alkyl radical, the aryl being optionally mono- or bisubstituted; a —(CH₂)ⱼ-heterocyclyl; or a —CH₂—CO-heterocyclyl, the heterocyclyl being optionally mono- or bisubstituted;
W₁ is —CH₂— or —O—
j is 1 or 2;
k is 0 or 1;
n is 0, 1 or 2;
m is 1, 2 or 3;
p is 1, 2 or 3;
q is 1, 2 or 3;
r is 1, 2 or 3;
s is 1, 2 or 3;
t is 0, 1 or 2;
v is 1, 2, 3 or 4;
w is 1, 2, 3 or 4;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof, or a corresponding solvate thereof.

Unless otherwise stated, the compounds of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by ¹³C- or ¹⁴C-enriched carbon, or the replacement of at least one nitrogen by ¹⁵N-enriched nitrogen are within the scope of this invention.

The compounds of general formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrugs.

For the sake of clarity the expression “a compound according to formula (I), wherein R₁, R₂, R₃ . . . are as defined below in the detailed description” would (just like the expression “a compound of formula (I) as defined in the claims) refer to “a compound according to formula (I)”, wherein the definitions of the respective substituents R₁ etc. (also from the cited claims) are applied.

For clarity purposes, all groups and definitions described in the present description and referring to compounds of formula (I), also apply to all intermediates of synthesis.

“Halogen” or “halo” as referred in the present invention represent fluorine, chlorine, bromine or iodine. When the term “halo” is combined with other substituents, such as for instance “C₁₋₆ haloalkyl” or “C₁₋₆ haloalkoxy” it means that the alkyl or alkoxy radical can respectively contain at least one halogen atom.

“C₁₋₆ alkyl”, as referred to in the present invention, are saturated aliphatic radicals. They may be unbranched (linear) or branched and are optionally substituted. C₁₋₆-alkyl as expressed in the present invention means an alkyl radical of 1, 2, 3, 4, 5 or 6 carbon atoms. Preferred alkyl radicals according to the present invention include but are not restricted to methyl, ethyl, propyl, n-propyl, isopropyl, butyl, n-butyl, tert-butyl, isobutyl, sec-butyl, 1-methylpropyl, 2-methylpropyl, 1,1-dimethylethyl, pentyl, n-pentyl, 1,1-dimethylpropyl, 1,2-dimethylpropyl, 2,2-dimethylpropyl, 3,3-dimethylbutyl, hexyl, 1-methylpentyl. The most preferred alkyl radical are C₁₋₆ alkyl, such as methyl, ethyl, propyl, n-propyl, isopropyl, butyl, n-butyl, tert-butyl, isobutyl, sec-butyl, isopentyl, 1-methylpropyl, 2-methylpropyl, 1,1-dimethylethyl or 3,3-dimethylbutyl. Alkyl radicals, as defined in the present invention, are optionally mono- or polysubstituted by substitutents independently selected from a halogen, branched or unbranched C₁₋₆-alkoxy, branched or unbranched C₁₋₆-alkyl, C₁₋₆-haloalcoxy, C₁₋₆-haloalkyl, trihaloalkyl or a hydroxyl group.

“C₁₋₆ alkoxy” as referred to in the present invention, is understood as meaning an alkyl radical as defined above attached via oxygen linkage to the rest of the molecule. Examples of alkoxy include, but are not limited to methoxy, ethoxy, propoxy, butoxy or tert-butoxy.

“C₃₋₉ Cycloalkyl” as referred to in the present invention, is understood as meaning saturated and unsaturated (but not aromatic), cyclic hydrocarbons having from 3 to 9 carbon atoms which can optionally be unsubstituted, mono- or polysubstituted. Examples for cycloalkyl radical preferably include but are not restricted to cyclopropyl, cyclobutyl, cyclopentyl, cyclohexyl. Cycloalkyl radicals, as defined in the present invention, are optionally mono- or polysubstituted by substitutents independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

A cycloalkylalkyl group/radical C₁₋₆, as defined in the present invention, comprises a branched or unbranched, optionally at least mono-substituted alkyl chain of 1 to 6 atoms which is bonded to a cycloalklyl group, as defined above. The cycloalkylalkyl radical is bonded to the molecule through the alkyl chain. A preferred cycloalkylalkyl group/radical is a cyclopropylmethyl group or a cyclopentylpropyl group, wherein the alkyl chain is optionally branched or substituted. Preferred substituents for cycloalkylalkyl group/radical, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

A heterocyclyl radical or group (also called heterocyclyl hereinafter) is understood as meaning 4 to 18 membered mono or fused polycyclic heterocyclic ring systems, with at least one saturated or unsaturated ring which contains one or more heteroatoms selected from the group consisting of nitrogen, oxygen and/or sulfur in the ring. A heterocyclic group can also be substituted once or several times.

Subgroups inside the heterocyclyls as understood herein include heteroaryls and non-aromatic heterocyclyls.
the heteroaryl (being equivalent to heteroaromatic radicals or aromatic heterocyclyls) is an aromatic 5 to 18 membered mono or fused polycyclic heterocyclic ring system, including spirofused ring systems, of one or more rings of which at least one aromatic ring contains one or more heteroatoms from the group consisting of nitrogen, oxygen and/or sulfur in the ring; preferably it is a 5 to 18 membered mono or fused polycyclic aromatic heterocyclic ring system of one or two rings of which at least one aromatic ring contains one or more heteroatoms selected from the group consisting of nitrogen, oxygen and/or sulfur in the ring; more preferably it is selected from furan, benzofuran, thiophene, benzothiophene, pyrrole, pyridine, pyrimidine, pyrazine, quinoline, isoquinoline, phthalazine, benzothiazole, indole, benzotriazole, carbazole, quinazoline, thiazole, imidazole, pyrazole, oxazole, isoxazole, dihydro-4H-pyrano[3,4-d]isoxazole, oxadiazole, thiophene, dihydro-4H-pyrano[3,4-d]isoxazole and benzimidazole;
the non-aromatic heterocyclyl is a 4 to 18 membered mono or fused polycyclic heterocyclic ring system, including spiro fused ring systems, of one or more rings of which at least one ring—with this (or these) ring(s) then not being aromatic—contains one or more heteroatoms from the group consisting of nitrogen, oxygen and/or sulfur in the ring; preferably it is a 4 to 18 membered mono or fused polycyclic heterocyclic ring system of one or two rings of which one or both rings
with this one or two rings then not being aromatic—contain/s one or more heteroatoms selected from the group consisting of nitrogen, oxygen and/or sulfur in the ring, more preferably it is selected from azetidine, oxetane, tetrahydrofuran, azepan, oxazepan, pyrrolidine, piperidine, piperazine, tetrahydropyran, morpholine, indoline, oxopyrrolidine, benzodioxane, 2,6-diazaspiro[3.4]octane 2,7-diazaspiro[3.5]nonane, 2,8-diazaspiro[4.5]decane, 3,9-diazaspiro[5.5]undecane, 2,9-diazaspiro[5.5]undecane, 6-azaspiro[3.4]octane, 7-azaspiro[3.5]nonane, 3-azaspiro[5.5]undecane, especially is azepan, oxazepan, pyrrolidine, piperidine, 2,6-diazaspiro[3.4]octane 2,7-diazaspiro[3.5]nonane, 2,8-diazaspiro[4.5]decane, 3,9-diazaspiro[5.5]undecane, 2,9-diazaspiro[5.5]undecane, 6-azaspiro[3.4]octane, 7-azaspiro[3.5]nonane, 3-azaspiro[5.5]undecane, benzodioxane, morpholine, tetrahydropyran, oxopyrrolidine and pyrrolidine.

Preferably, in the context of this invention heterocyclyl is defined as a 4 to 18 membered mono or fused polycyclic ring system, including spirofused ring systems, of one or more saturated or unsaturated rings of which at least one ring contains one or more heteroatoms selected from the group consisting of nitrogen, oxygen and/or sulfur in the ring. Preferably it is a 4 to 18 membered mono or fused polycyclic heterocyclic ring system of one or two saturated or unsaturated rings of which at least one ring contains one or more heteroatoms selected from the group consisting of nitrogen, oxygen, and sulfur in the ring. More preferably, it is a 4 to 12 membered mono or bicyclic heterocyclyl ring system containing one nitrogen atom and optionally a second heteroatom selected from nitrogen and oxygen. In another preferred embodiment of the invention, said heterocyclyl is a substituted mono or bicyclic heterocyclyl ring system.

Preferred examples of heterocyclyls include azetidine, azepane, oxazepan, pyrrolidine, piperidine, oxetane, tetrahydrofuran, imidazole, oxadiazole, tetrazole, pyridine, pyrimidine, piperazine, benzofuran, benzimidazole, indazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, tetrahydroisoquinoline, phthalazine, benzo-1,2,5-thiadiazole, indole, benzotriazole, benzoxazole oxopyrrolidine, pyrimidine, benzodioxolane, benzodioxane, carbazole, quinazoline, 2,6-diazaspiro[3.4]octane 2,7-diazaspiro[3.5]nonane, 2,8-diazaspiro[4.5]decane, 3,9-diazaspiro[5.5]undecane, 2,9-diazaspiro[5.5]undecane, 6-azaspiro[3.4]octane, 7-azaspiro[3.5]nonane, 3-azaspiro[5.5]undecane octahydropyrrolo[3,4-c]pyrrole, especially is pyridine, piperazine, pyrazine, indazole, benzodioxane, thiazole, benzothiazole, morpholine, tetrahydropyran, pyrazole, imidazole, piperidine, thiophene, indole, benzimidazole, pyrrolo[2,3-b]pyridine, benzoxazole, oxopyrrolidine, pyrimidine, oxazepane, pyrrolidine, azetidine, azepane, oxazepane, oxetane, tetrahydrofuran, 2,6-diazaspiro[3.4]octane 2,7-diazaspiro[3.5]nonane, 2,8-diazaspiro[4.5]decane, 3,9-diazaspiro[5.5]undecane, 2,9-diazaspiro[5.5]undecane, 6-azaspiro[3.4]octane, 7-azaspiro[3.5]nonane, 3-azaspiro[5.5]undecane.

An N-containing heterocyclyl is a heterocyclic ring system of one or more saturated or unsaturated rings of which at least one ring contains a nitrogen and optionally one or more further heteroatoms selected from the group consisting of nitrogen, oxygen and/or sulfur in the ring; preferably is a heterocyclic ring system of one or two saturated or unsaturated rings of which at least one ring contains a nitrogen and optionally one or more further heteroatoms selected from the group consisting of nitrogen, oxygen and/or sulfur in the ring, more preferably is selected from azetidine, azepane, oxazepam, pyrrolidine, imidazole, oxadiazole, tetrazole, azetidine, pyridine, pyrimidine, piperidine, piperazine, benzimidazole, indazole, benzothiazole, benzodiazole, morpholine, indoline, triazole, isoxazole, pyrazole, pyrrole, pyrazine, pyrrolo[2,3-b]pyridine, quinoline, quinolone, isoquinoline, tetrahydrothienopyridine, phthalazine, benzo-1,2,5-thiadiazole, indole, benzotriazole, benzoxazole oxopyrrolidine, carbazole, thiazole, 2,6-diazaspiro[3.4]octane 2,7-diazaspiro[3.5]nonane, 2,8-diazaspiro[4.5]decane, 3,9-diazaspiro[5.5]undecane, 2,9-diazaspiro[5.5]undecane, 6-azaspiro[3.4]octane, 7-azaspiro[3.5]nonane, 3-azaspiro[5.5]undecane or octahydropyrrolo[3,4-c]pyrrole.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

“Heterocycloalkyl” as referred to in the present invention, are understood as meaning saturated and unsaturated (but not aromatic), generally 5 or 6 membered cyclic hydrocarbons which can optionally be unsubstituted, mono- or polysubstituted and which have at least one heteroatom in their structure selected from N, O or S. Examples for heterocycloalkyl radical preferably include but are not restricted to pyrroline, pyrrolidine, pyrazoline, aziridine, azetidine, tetrahydropyrrole, oxirane, oxetane, dioxetane, tetrahydropyrane, tetrahydrofurane, dioxane, dioxolane, oxazolidine, piperidine, piperazine, morpholine, azepane or diazepane. Heterocycloalkyl radicals, as defined in the present invention, are optionally mono- or polysubstituted by substitutents independently selected from a halogen atom, branched or unbranched C₁₋₆-alkyl, branched or unbranched C₁₋₆-alkoxy, C₁₋₆-haloalkoxy, C₁₋₆-haloalkyl, trihaloalkyl or a hydroxyl group. More preferably heterocycloalkyl in the context of the present invention are 5 or 6-membered ring systems optionally at least monosubstituted.

A heterocycloalkylalkyl group/radical C₁₋₆, as defined in the present invention, comprises a linear or branched, optionally at least mono-substituted alkyl chain of 1 to 6 atoms which is bonded to a cycloalklyl group, as defined above. The heterocycloalkylalkyl radical is bonded to the molecule through the alkyl chain. A preferred heterocycloalkylalkyl group/radical is a piperidinethyl group or a piperazinylmethyl group, wherein the alkyl chain is optionally branched or substituted. Preferred substituents for cycloalkylalkyl group/radical, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

“Aryl” as referred to in the present invention, is understood as meaning ring systems with at least one aromatic ring but without heteroatoms even in only one of the rings. These aryl radicals may optionally be mono- or polysubstituted by substitutents independently selected from a halogen atom, —CN, branched or unbranched C₁₋₆-alkyl, branched or unbranched C₁₋₆-alkoxy, C₁₋₆-haloalcoxy, C₁₋₆-haloalkyl, a heterocyclyl group and a hydroxyl group. Preferred examples of aryl radicals include but are not restricted to phenyl, naphthyl, fluoranthenyl, fluorenyl, tetralinyl, indanyl or anthracenyl radicals, which may optionally be mono- or polysubstituted, if not defined otherwise. More preferably aryl in the context of the present invention is a 6-membered ring system optionally at least mono or polysubstituted.

An arylalkyl radical C₁₋₆, as defined in the present invention, comprises an unbranched or branched, optionally at least mono-substituted alkyl chain of 1 to 6 carbon atoms which is bonded to an aryl group, as defined above. The arylalkyl radical is bonded to the molecule through the alkyl chain. A preferred arylalkyl radical is a benzyl group or a phenetyl group, wherein the alkyl chain is optionally branched or substituted. Preferred substituents for arylalkyl radicals, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

Heteroarylalkyl group/radical C₁₋₆ as defined in the present invention, comprises a linear or branched, optionally at least mono-substituted alkyl chain of 1 to 6 carbon atoms which is bonded to an heteroaryl group, as defined above. The heteroarylalkyl radical is bonded to the molecule through the alkyl chain. A preferred heteroarylalkyl radical is a pyridinylmethyl group, wherein the alkyl chain is optionally branched or substituted. Preferred substituents for heteroarylalkyl radicals, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

The term “condensed” according to the present invention means that a ring or ring-system is attached to another ring or ring-system, whereby the terms “annulated” or “annelated” are also used by those skilled in the art to designate this kind of attachment.

The term “ring system” according to the present invention refers to a system consisting of at least one ring of connected atoms but including also systems in which two or more rings of connected atoms are joined with “joined” meaning that the respective rings are sharing one (like a spiro structure), two or more atoms being a member or members of both joined rings. The “ring system” thus defined comprises saturated, unsaturated or aromatic carbocyclic rings which contain optionally at least one heteroatom as ring member and which are optionally at least mono-substituted and may be joined to other carbocyclic ring systems such as aryl radicals, heteroaryl radicals, cycloalkyl radicals etc.

The terms “condensed”, “annulated” or “annelated” are also used by those skilled in the art to designate this kind of join.

A leaving group is a group that in a heterolytic bond cleavage keeps the electron pair of the bond. Suitable leaving groups are well known in the art and include Cl, Br, I and —O—SO₂R¹⁴, wherein R¹⁴ is F, C₁₋₄-alkyl, C₁₋₄-haloalkyl, or optionally substituted phenyl. The preferred leaving groups are Cl, Br, I, tosylate, mesylate, triflate, nonaflate and fluorosulphonate.

“Protecting group” is a group that is chemically introduced into a molecule to avoid that a certain functional group from that molecule undesirably reacts in a subsequent reaction. Protecting groups are used, among others, to obtain chemoselectivity in chemical reactions. The preferred protecting group in the context of the invention are Boc (tert-butoxycarbonyl) or Teoc (2-(trimethylsilyl)ethoxycarbonyl).

The term “salt” is to be understood as meaning any form of the active compound according to the invention in which this assumes an ionic form or is charged and is coupled with a counter-ion (a cation or anion). The definition particularly includes physiologically acceptable salts, this term must be understood as equivalent to “pharmaceutically acceptable salts”.

The term “pharmaceutically acceptable salts” in the context of this invention means any salt that is tolerated physiologically (normally meaning that it is not toxic, particularly as a result of the counter-ion) when used in an appropriate manner for a treatment, particularly applied or used in humans and/or mammals. This definition specifically includes in the context of this invention a salt formed by a physiologically tolerated acid, i.e. salts of a specific active compound with physiologically tolerated organic or inorganic acids—particularly when used on humans and/or mammals. Examples of this type of salts are those formed with: hydrochloric acid, hydrobromic acid, sulphuric acid, methanesulfonic acid, formic acid, acetic acid, oxalic acid, succinic acid, malic acid, tartaric acid, mandelic acid, fumaric acid, lactic acid or citric acid. In addition, the pharmaceutically acceptable salts may be formed with a physiologically tolerated cation, preferably inorganic, particularly when used on humans and/or mammals. Salts with alkali and alkali earth metals are particularly preferred, as well as those formed with ammonium cations (NH₄⁺). Preferred salts are those formed with (mono) or (di)sodium, (mono) or (di)potassium, magnesium or calcium. These physiologically acceptable salts may also be formed with anions or acids and, in the context of this invention, are understood as being salts formed by at least one compound used in accordance with the invention—normally protonated, for example in nitrogen—such as a cation and at least one physiologically tolerated anion, particularly when used on humans and/or mammals.

The compounds of the invention may be present in crystalline form or in amorphous form.

Any compound that is a solvate of a compound according to formula (I) defined above is understood to be also covered by the scope of the invention. Methods of solvation are generally known within the art. Suitable solvates are pharmaceutically acceptable solvates. The term “solvate” is to be understood as meaning any form of the active compound according to the invention in which this compound has attached to it via non-covalent binding another molecule (most likely a polar solvent) especially including hydrates and alcoholates, like methanolate or ethanolate.

The term “co-crystal” is to be understood as a crystalline material comprising a specific active compound with at least one additional component, usually a co-crystal former, and of which at least two of the constituents are held together by weak interactions. Weak interaction is being defined as an interaction which is neither ionic nor covalent and includes for example: hydrogen bonds, van der Waals forces, and π-π interactions.

The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the compounds of the invention: esters, amino acid esters, phosphate esters, metal salts sulfonate esters, carbamates, and amides. Examples of well-known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a prodrug of a compound of general formula (I) is within the scope of the invention. Particularly favored prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Any compound that is a N-oxide of a compound according to the invention like a compound according to formula (I) defined above is understood to be also covered by the scope of the invention.

The compounds of formula (I) as well as their salts or solvates are preferably in pharmaceutically acceptable or substantially pure form. By pharmaceutically acceptable pure form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

Unless otherwise defined, all the groups above mentioned that can be substituted or unsubstituted may be substituted at one or more available positions by one or more suitable groups such as a halogen, preferably Cl or F; OR′, ═O, SR′, SOR′, SO₂R′, OSO₂R′, OSO₃R′, NO₂, NHR′, NR′R″, ═N—R′, N(R′)COR′, N(COR′)₂, N(R′)SO₂R′, N(R′)C(═NR′)N(R′)R′, N₃, CN, halogen, COR′, COOR′, OCOR′, OCOOR′, OCONHR′, OCONR′R″, CONHR′, CONR′R″, CON(R′)OR′, CON(R′)SO₂R′, PO(OR′)₂, PO(OR′)R′, PO(OR′)(N(R′)R′), C₁₋₆ alkyl, C₃₋₁₀ cycloalkyl, C₂₋₆ alkenyl, C₂₋₆ alkynyl, aryl, and heterocyclic group, wherein each of the R′ and R″ groups is independently selected from the group consisting of hydrogen, C₁₋₆ alkyl, C₃₋₁₀ cycloalkyl, C₂₋₆ alkenyl, C₂₋₆ alkynyl, aryl and heterocyclic group. Where such groups are themselves substituted, the substituents may be chosen from the foregoing list.

In a particular and preferred embodiment of the invention, X is a halogen atom, preferably represented by a chlorine atom.

In a particular and preferred embodiment of the invention, Het is a group selected from:

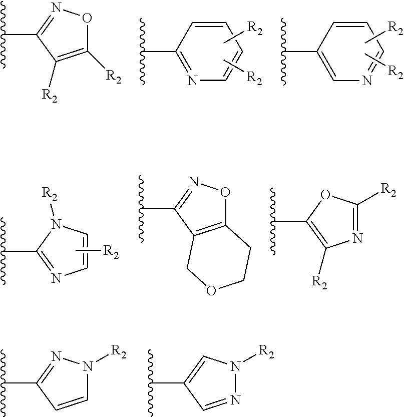

wherein R₂ is as defined along the detailed description and claims and, preferably, a hydrogen atom, methyl, ethyl, isopropyl, cyclopropyl, methoxy or hydroxyl.

In a particular embodiment A represents —CH₂—B.

In a further particular embodiment A represents —CO—B.

Within the two precedent embodiments, but within particular and preferred embodiments of the invention B is represented by:

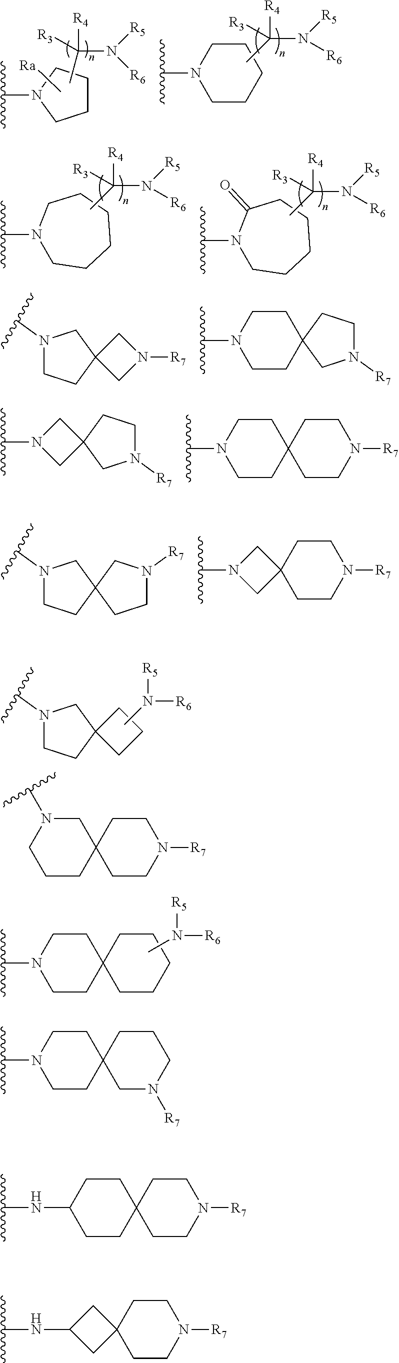

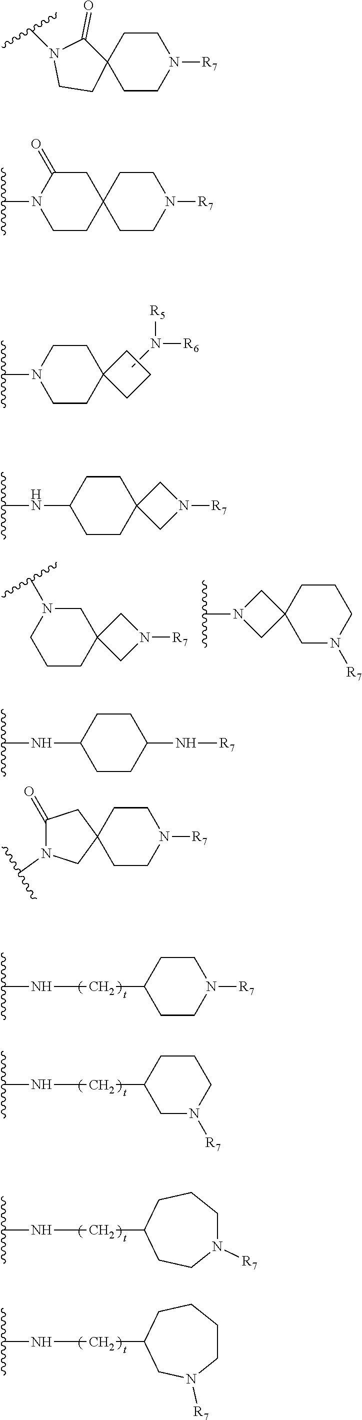

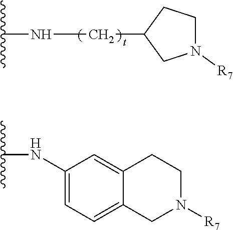

wherein Rₐ, R₂, R₃, R₄, R₅, R₆, R₇; t and n are as defined along the detailed description and claims.

In a further particular and preferred embodiment of the invention R₁ is methyl, ethyl propyl, isopropyl, pyridyl or phenyl that may optionally be substituted.

An additional particular and preferred embodiment of the invention is that where R₂ is a hydrogen atom, methyl, ethyl, isopropyl, trifluoromethyl, cyclopropyl, methoxy, ethoxy or hydroxyl.

Yet another particular and preferred embodiment of the invention is that wherein R₃ and R₄ represent a hydrogen atom; or alternatively that wherein R₃ and R₄ form together with the atom they are attached to a cycloalkyl, preferably a cyclopropane ring.

A further particular and preferred embodiment is that wherein R₅ is a hydrogen atom, methyl, ethyl, propyl, isobutyl, isopentyl or dimethylbutyl.

Similarly it is a preferred embodiment of the invention compounds where wherein R₆ is methyl, ethyl, propyl, isobutyl, isopentyl, dimethylbutyl, —(CH₂)ⱼ-phenyl, —(CH₂)ⱼ-tetrahydro-2H-pyranyl or —(CH₂)ⱼ-piperidinyl wherein j is 1 or 2.

Also preferred and particular embodiments of the invention are compounds of formula (I) wherein R₇ is methyl, ethyl, propyl, isobutyl, isopentyl, dimethylbutyl, —CH(R_{7′})(CH₂)ₖ-phenyl being R_{7′} a hydrogen atom or a C₁₋₆ alkyl radical and the phenyl being optionally mono- or bisubstituted with —CN or halogen, preferably F; or a —CH₂—CO-piperidine optionally substituted by one or two halogen atoms, preferably F.

In a particular embodiment of the invention W₁ is —CH₂—.

A further particular and preferred embodiment of the invention comprises a compound of formula (I):

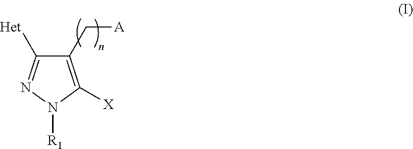

wherein:
X is a halogen atom, preferably a chlorine atom;
Het is a 5 to 12-membered heterocyclic ring or ring system optionally substituted by one or more R₂ radicals; preferably one of the following moieties:

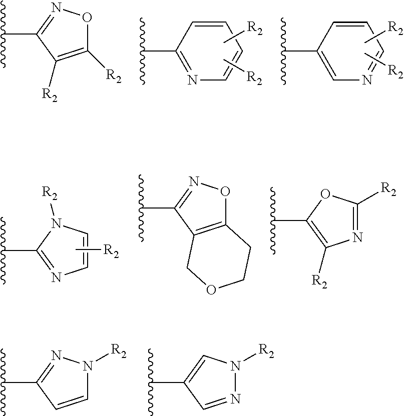

A represents —CO—B or —CH₂—B
B is one of the following moieties:

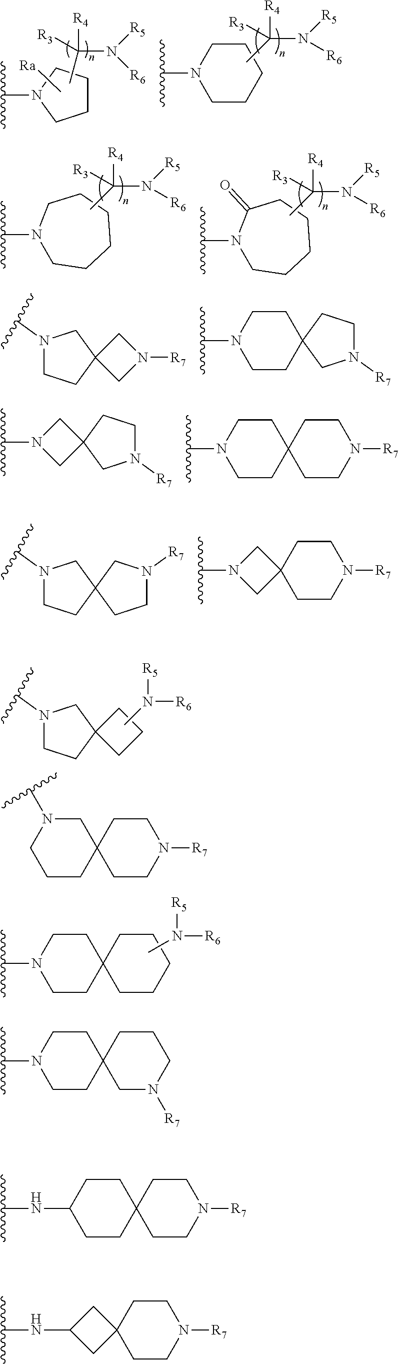

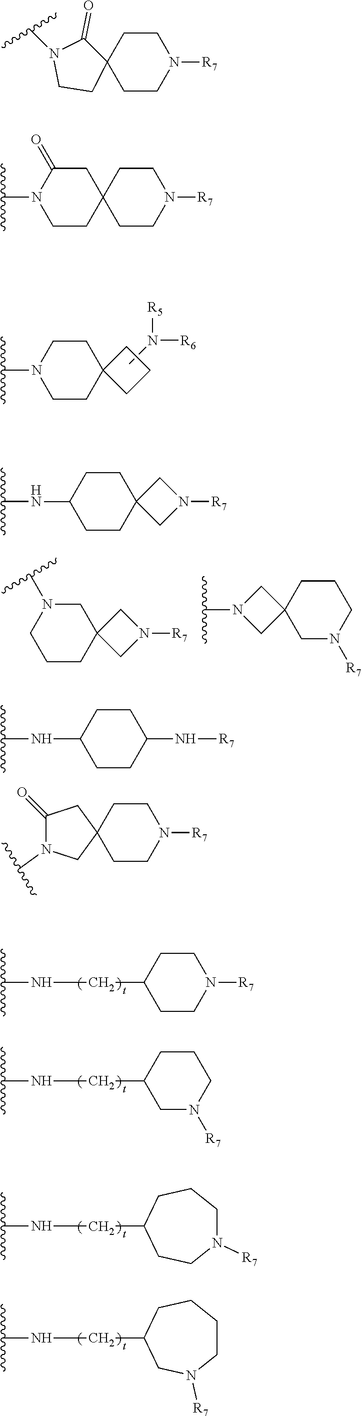

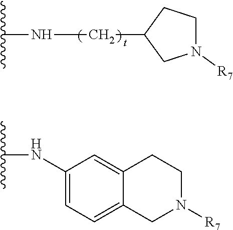

Rₐ is a hydrogen atom or a branched or unbranched C₁₋₆ alkyl radical, preferably Rₐ is a hydrogen atom or a methyl group;
R₁ is a branched or unbranched C₁₋₆ alkyl radical or an aryl radical, preferably R1 is methyl, ethyl, propyl, isopropyl, pyridyl or phenyl that may optionally be substituted;
R₂ is a hydrogen atom, a branched or unbranched C₁₋₆ alkyl radical; a C₃₋₉ cycloalkyl radical, a halogen atom or —OR₂, being R₂, a hydrogen atom or a C₁₋₆ alkyl radical; preferably R₂ is a hydrogen atom, methyl, ethyl, isopropyl, trifluoromethyl, cyclopropyl, methoxy, ethoxy or hydroxyl;
R₃ and R₄ represent a hydrogen atom; or alternatively R₃ and R₄ form together with the atom they are attached to a C₃₋₉ cycloalkyl group; preferably R₃ and R₄ represent a hydrogen atom, or alternatively R₃ and R₄ form together with the atom they are attached to a cyclopropane ring;
R₅ is a hydrogen atom or a branched or unbranched C₁₋₆ alkyl radical, preferably R₅ is a hydrogen atom, methyl, ethyl, propyl, isobutyl, isopentyl or dimethylbutyl;
R₆ is a branched or unbranched C₁₋₆ alkyl radical; a —(CH₂)ⱼ-aryl or a —(CH₂)ⱼ-heterocyclyl, preferably R₆ is methyl, ethyl, propyl, isobutyl, isopentyl, dimethylbutyl, —(CH₂)ⱼ-phenyl, —(CH₂)ⱼ-tetrahydro-2H-pyranyl or —(CH₂)ⱼ-piperidinyl;
R₇ is a branched or unbranched C₁₋₆ alkyl radical; —CH(R_{7′})—(CH₂)ₖ-aryl being R_{7′} a hydrogen atom or a C₁₋₆ alkyl radical and the aryl being optionally mono- or bisubstituted; or —CH₂—CO-heterocyclyl, the heterocyclyl being optionally mono- or bisubstituted; preferably R₇ is methyl, ethyl, propyl, isobutyl, isopentyl, dimethylbutyl, —CH(R_{7′})—(CH₂)ₖ-phenyl being R_{7′} a hydrogen atom or a C₁₋₆ alkyl radical and the phenyl being optionally mono- or bisubstituted with —CN or halogen, preferably F; or a —CH₂—CO-piperidine optionally substituted by one or two halogen atoms, preferably F
W₁ is —CH₂— or —O—; preferably —CH₂—
n is 0, 1 or 2;
t is 0, 1 or 2;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof, or a corresponding solvate thereof.

A further particular and preferred embodiment of the invention comprises a compound of formula (I):

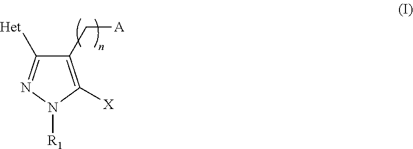

wherein:
X is a chlorine atom;
Het is one of the following moieties:

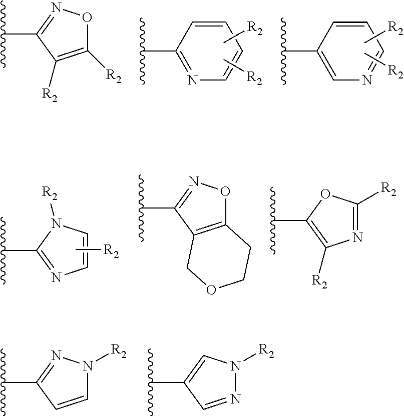

A represents —CO—B or —CH₂—B
B is one of the following moieties:

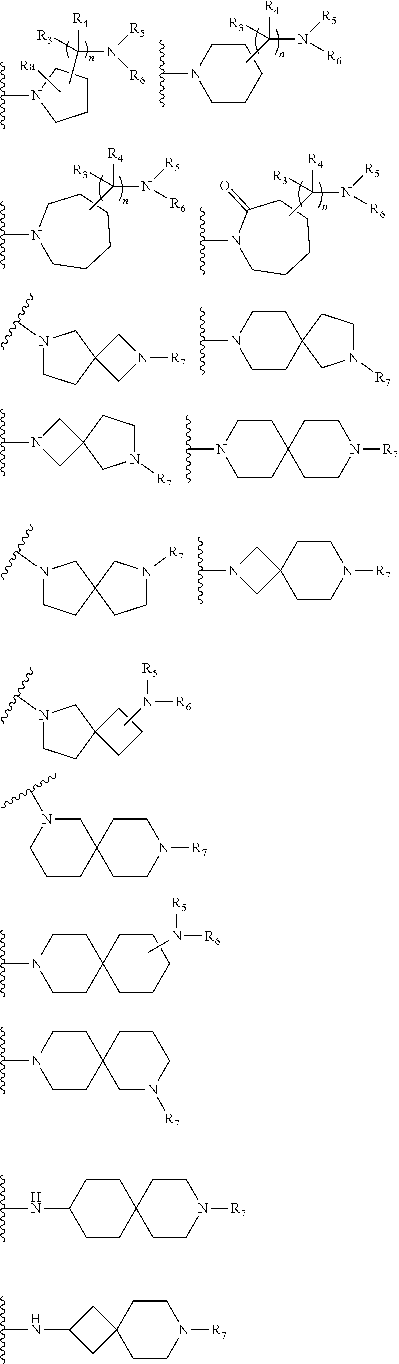

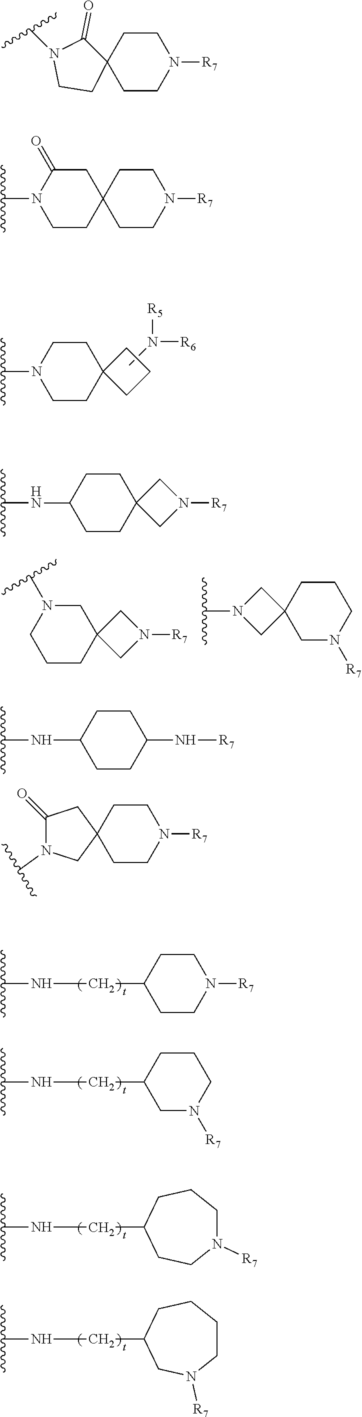

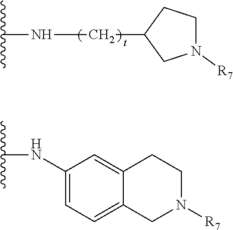

Rₐ is a hydrogen atom or a methyl group;
R₁ is methyl, ethyl propyl, isopropyl, pyridyl or phenyl that may optionally be substituted;
R₂ is a hydrogen atom, methyl, ethyl, isopropyl, trifluoromethyl, cyclopropyl, methoxy, ethoxy or hydroxyl;
R₃ and R₄ represent a hydrogen atom, or alternatively R₃ and R₄ form together with the atom they are attached a cyclopropane ring;
R₅ is a hydrogen atom, methyl, ethyl, propyl, isobutyl, isopentyl or dimethylbutyl;
R₆ is methyl, ethyl, propyl, isobutyl, isopentyl, dimethylbutyl, —(CH₂)ⱼ-phenyl, —(CH₂)ⱼ-tetrahydro-2H-pyranyl or —(CH₂)ⱼ-piperidinyl;
R₇ is methyl, ethyl, propyl, isobutyl, isopentyl, dimethylbutyl, —CH(R_{7′})—(CH₂)ₖ-phenyl being R_{7′} a hydrogen atom or a C₁₋₆ alkyl radical and the phenyl being optionally mono- or bisubstituted with —CN or halogen, preferably F; or a —CH₂—CO-piperidine optionally substituted by one or two halogen atoms, preferably F
W₁ is —CH₂—;
n is 0, 1 or 2;
t is 0, 1 or 2;
v is 1, 2, 3 or 4;
w is 1, 2, 3 or 4;
   wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof, or a corresponding solvate thereof.

A particularly preferred embodiment of the invention is represented by compounds of formula (I) having subformulas (Ia), (Ib), (Ic) or (Id):

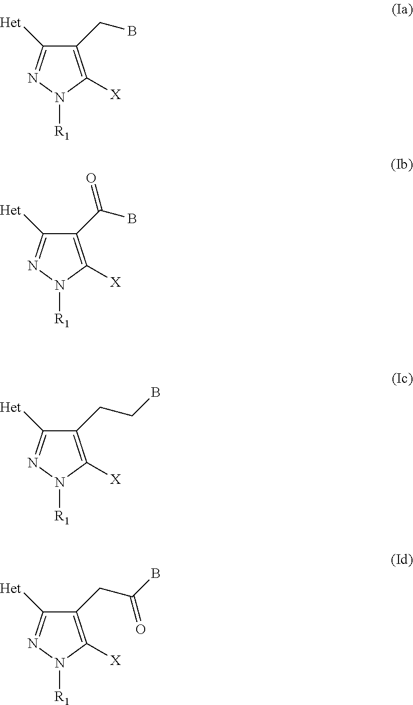

wherein Het, X, B and R₁ are as defined for formula (I) along the detailed description and claims.

A still more particular and preferred embodiment of the invention is represented by compounds of formula (I) having subformulas (Ie), (If), (Ig), (Ih), (Ii), (Ij), (Ik), (Im) or (In):

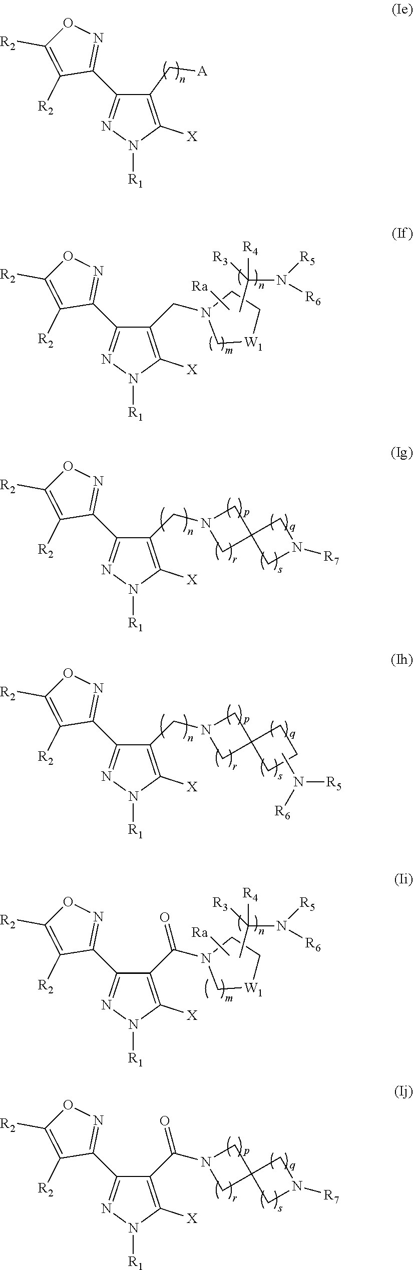

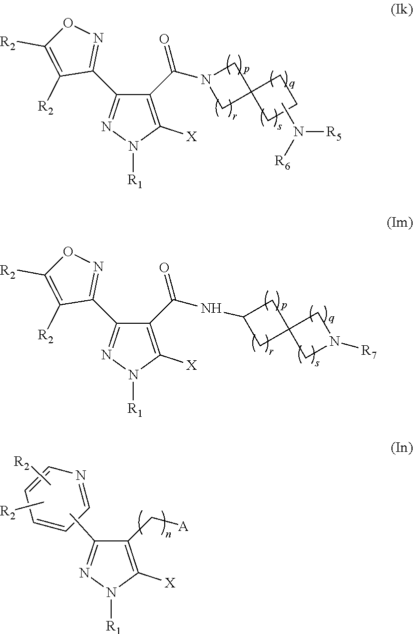

wherein A, X, W₁, Rₐ, R₁, R₂, R₃, R₄, R₅, Re, R₇, m, n, p, q, r and s are as defined along the detailed description and claims.

The compounds of the present invention represented by the above described formula (I), (Ia), (Ib), (Ic), (Id) (Ie), (If), (Ig), (Ih), (Ii), (Ij), (Ik), (Im) or (In) may include enantiomers depending on the presence of chiral centers or isomers depending on the presence of double bonds (e.g. Z, E). The single stereoisomers, enantiomers or diastereoisomers and mixtures thereof fall within the scope of the present invention.

The preferred compounds of the invention are selected from:
[1] N-((1-((5-Chloro-1-methyl-3-(5-methylisoxazol-3-yl)-1H-pyrazol-4-yl)methyl)pyrrolidin-3-yl)methyl)-3,3-dimethylbutan-1-amine;
[2] 1-((5-Chloro-1-methyl-3-(5-methylisoxazol-3-yl)-1H-pyrazol-4-yl)methyl)-N-isopentylazepan-3-amine;
[3] 1-((5-Chloro-1-methyl-3-(5-methylisoxazol-3-yl)-1H-pyrazol-4-yl)methyl)-N-phenethylazepan-3-amine;
[4] 1-((5-Chloro-1-methyl-3-(5-methylisoxazol-3-yl)-1H-pyrazol-4-yl)methyl)-N-((tetrahydro-2H-pyran-4-yl)methyl)azepan-3-amine;
[5] (R)-1-((5-Chloro-1-methyl-3-(5-methylisoxazol-3-yl)-1H-pyrazol-4-yl)methyl)-N-isopentylpiperidin-3-amine;
[6] (S)-1-((5-Chloro-1-methyl-3-(5-methylisoxazol-3-yl)-1H-pyrazol-4-yl)methyl)-N-isopentylpiperidin-3-amine;
[7] (S)-1-((5-Chloro-1-methyl-3-(5-methylisoxazol-3-yl)-1H-pyrazol-4-yl)methyl)-N-phenethylpiperidin-3-amine;
[8] 1-((5-Chloro-1-methyl-3-(5-methylisoxazol-3-yl)-1H-pyrazol-4-yl)methyl)-N-(3,3-dimethylbutyl)azepan-3-amine;
[9] N-Butyl-1-((5-chloro-1-methyl-3-(5-methylisoxazol-3-yl)-1H-pyrazol-4-yl)methyl)azepan-3-amine;
[10] (R)-1-((5-Chloro-1-methyl-3-(5-methylisoxazol-3-yl)-1H-pyrazol-4-yl)methyl)-N-isopentylazepan-3-amine;
[11] 1-((5-Chloro-1-methyl-3-(5-methylisoxazol-3-yl)-1H-pyrazol-4-yl)methyl)-N-isopentylpyrrolidin-3-amine;
[12] (S)-1-((5-Chloro-1-methyl-3-(5-methylisoxazol-3-yl)-1H-pyrazol-4-yl)methyl)-N-isopentylazepan-3-amine;
[13] 1-((5-Chloro-1-methyl-3-(5-methylisoxazol-3-yl)-1H-pyrazol-4-yl)methyl)-N-(3,3-dimethylbutyl)pyrrolidin-3-amine;
[14] 1-((5-Chloro-1-ethyl-3-(5-methylisoxazol-3-yl)-1H-pyrazol-4-yl)methyl)-N-isopentylazepan-3-amine;
[15] 1-((5-Chloro-3-(5-isopropylisoxazol-3-yl)-1-methyl-1H-pyrazol-4-yl)methyl)-N-(3,3-dimethylbutyl)azepan-3-amine;
[16] 1-((5-Chloro-3-(5-cyclopropylisoxazol-3-yl)-1-methyl-1H-pyrazol-4-yl)methyl)-N-(3,3-dimethylbutyl)azepan-3-amine;
[17] 1-((5-Chloro-3-(5-isopropylisoxazol-3-yl)-1-methyl-1H-pyrazol-4-yl)methyl)-N-isopentylazepan-3-amine;
[18] 1-((5-Chloro-3-(5-methylisoxazol-3-yl)-1-phenyl-1H-pyrazol-4-yl)methyl)-N-isopentylazepan-3-amine;
[19] 1-((5-Chloro-3-(5-cyclopropylisoxazol-3-yl)-1-methyl-1H-pyrazol-4-yl)methyl)-N-isopentylazepan-3-amine;
[20] 1-((5-Chloro-3-(5-ethylisoxazol-3-yl)-1-methyl-1H-pyrazol-4-yl)methyl)-N-(3,3-dimethylbutyl)azepan-3-amine;
[21] 1-((5-Chloro-3-(5-ethylisoxazol-3-yl)-1-methyl-1H-pyrazol-4-yl)methyl)-N-isopentylazepan-3-amine;
[22] 1-((5-Chloro-1-ethyl-3-(5-methylisoxazol-3-yl)-1H-pyrazol-4-yl)methyl)-N-(3,3-dimethylbutyl)azepan-3-amine;
[23] 1-((5-Chloro-1-methyl-3-(5-methylisoxazol-3-yl)-1H-pyrazol-4-yl)methyl)-N-(3,3-dimethylbutyl)azepan-4-amine;
[24] 1-((5-Chloro-1-methyl-3-(5-methylisoxazol-3-yl)-1H-pyrazol-4-yl)methyl)-N-isopentylazepan-4-amine;
[25] 1-((5-Chloro-1-methyl-3-(pyridin-2-yl)-1H-pyrazol-4-yl)methyl)-N-(3,3-dimethylbutyl)azepan-3-amine;
[26] 1-((5-Chloro-1-methyl-3-(pyridin-2-yl)-1H-pyrazol-4-yl)methyl)-N-isopentylazepan-3-amine;
[27] 1-((5-Chloro-1-methyl-3-(4-methylisoxazol-3-yl)-1H-pyrazol-4-yl)methyl)-N-(3,3-dimethylbutyl)azepan-3-amine;
[28] 1-((5-Chloro-1-methyl-3-(4-methylisoxazol-3-yl)-1H-pyrazol-4-yl)methyl)-N-isopentylazepan-3-amine;
[29] N-((1-((5-Chloro-1-methyl-3-(5-methylisoxazol-3-yl)-1H-pyrazol-4-yl)methyl)pyrrolidin-3-yl)methyl)-3-methylbutan-1-amine;
[30] 1-((5-Chloro-1-methyl-3-(pyridin-3-yl)-1H-pyrazol-4-yl)methyl)-N-(3,3-dimethylbutyl)azepan-3-amine;
[31] 1-((5-Chloro-1-methyl-3-(pyridin-3-yl)-1H-pyrazol-4-yl)methyl)-N-isopentylazepan-3-amine;
[32] 1-((5-Chloro-1-methyl-3-(1-methyl-1H-imidazol-2-yl)-1H-pyrazol-4-yl)methyl)-N-isopentylazepan-3-amine;
[33] 1-((5-Chloro-3-(isoxazol-3-yl)-1-methyl-1H-pyrazol-4-yl)methyl)-N-(3,3-dimethylbutyl)azepan-3-amine;
[34] 1-((5-Chloro-3-(isoxazol-3-yl)-1-methyl-1H-pyrazol-4-yl)methyl)-N-isopentylazepan-3-amine;
[35] 1-((5-Chloro-3-(5-methoxypyridin-2-yl)-1-methyl-1H-pyrazol-4-yl)methyl)-N-(3,3-dimethylbutyl)azepan-3-amine;
[36] 1-((5-Chloro-3-(5-methoxypyridin-2-yl)-1-methyl-1H-pyrazol-4-yl)methyl)-N-isopentylazepan-3-amine;
[37] 1-((5-Chloro-3-(4,5-dimethylisoxazol-3-yl)-1-methyl-1H-pyrazol-4-yl)methyl)-N-isopentylazepan-3-amine;
[38] 1-((5-Chloro-3-(4,5-dimethylisoxazol-3-yl)-1-methyl-1H-pyrazol-4-yl)methyl)-N-(3,3-dimethylbutyl)azepan-3-amine;
[39] 6-(5-Chloro-4-((3-(isopentylamino)azepan-1-yl)methyl)-1-methyl-1H-pyrazol-3-yl)pyridin-2-ol;
[40] (S)-1-((5-Chloro-3-(6,7-dihydro-4H-pyrano[3,4-d]isoxazol-3-yl)-1-methyl-1H-pyrazol-4-yl)methyl)-N-isopentylazepan-3-amine;
[41] N-((1-((5-Chloro-1-methyl-3-(5-methylisoxazol-3-yl)-1H-pyrazol-4-yl)methyl)piperidin-4-yl)methyl)-3-methylbutan-1-amine;
[42] N-((1-((5-Chloro-1-methyl-3-(5-methylisoxazol-3-yl)-1H-pyrazol-4-yl)methyl)pyrrolidin-3-yl)methyl)-N-isobutyl-2-methylpropan-1-amine;
[43] N-((1-((5-Chloro-1-methyl-3-(5-methylisoxazol-3-yl)-1H-pyrazol-4-yl)methyl)piperidin-4-yl)methyl)-2-methylpropan-1-amine;
[44] N-Benzyl-1-(1-((5-chloro-1-methyl-3-(5-methylisoxazol-3-yl)-1H-pyrazol-4-yl)methyl)pyrrolidin-3-yl)methanamine;
[45] N-((1-((5-Chloro-1-methyl-3-(5-methylisoxazol-3-yl)-1H-pyrazol-4-yl)methyl)pyrrolidin-3-yl)methyl)-2-phenylethanamine;
[46] N-((1-((5-Chloro-1-methyl-3-(5-methylisoxazol-3-yl)-1H-pyrazol-4-yl)methyl)pyrrolidin-3-yl)methyl)-N-propylpropan-1-amine;
[47] N-((1-((5-Chloro-1-methyl-3-(5-methylisoxazol-3-yl)-1H-pyrazol-4-yl)methyl)pyrrolidin-3-yl)methyl)propan-1-amine;
[48] N-((1-((5-Chloro-1-methyl-3-(5-methylisoxazol-3-yl)-1H-pyrazol-4-yl)methyl)piperidin-3-yl)methyl)-2-methylpropan-1-amine;
[49] N-((1-((5-Chloro-1-methyl-3-(5-methylisoxazol-3-yl)-1H-pyrazol-4-yl)methyl)pyrrolidin-3-yl)methyl)-2-methylpropan-1-amine;
[50] 3-(5-Chloro-4-((2-(3,3-dimethylbutyl)-2,8-diazaspiro[4.5]decan-8-yl)methyl)-1-methyl-1H-pyrazol-3-yl)-5-methylisoxazole;
[51] 3-(5-Chloro-4-((6-(3,3-dimethylbutyl)-2,6-diazaspiro[3.4]octan-2-yl)methyl)-1-methyl-1H-pyrazol-3-yl)-5-methylisoxazole;
[52] 3-(5-Chloro-4-((2-(3,3-dimethylbutyl)-2,6-diazaspiro[3.4]octan-6-yl)methyl)-1-methyl-1H-pyrazol-3-yl)-5-methylisoxazole;
[53] 3-(5-Chloro-4-((9-(3,3-dimethylbutyl)-3,9-diazaspiro[5.5]undecan-3-yl)methyl)-1-methyl-1H-pyrazol-3-yl)-5-methylisoxazole;
[54] 3-(5-Chloro-4-((7-(3,3-dimethylbutyl)-2,7-diazaspiro[4.4]nonan-2-yl)methyl)-1-methyl-1H-pyrazol-3-yl)-5-methylisoxazole;
[55] N-Benzyl-1-(1-((5-chloro-1-methyl-3-(5-methylisoxazol-3-yl)-1H-pyrazol-4-yl)methyl)piperidin-3-yl)methanamine;
[56] N-Benzyl-1-(1-((5-chloro-1-methyl-3-(5-methylisoxazol-3-yl)-1H-pyrazol-4-yl)methyl)piperidin-4-yl)methanamine;
[57] N-((1-((5-Chloro-1-methyl-3-(5-methylisoxazol-3-yl)-1H-pyrazol-4-yl)methyl)piperidin-3-yl)methyl)-2-phenylethanamine;
[58] N-((1-((5-Chloro-1-methyl-3-(5-methylisoxazol-3-yl)-1H-pyrazol-4-yl)methyl)piperidin-3-yl)methyl)-3-methylbutan-1-amine;
[59] N-((1-((5-Chloro-1-methyl-3-(5-methylisoxazol-3-yl)-1H-pyrazol-4-yl)methyl)azepan-3-yl)methyl)-3-methylbutan-1-amine;
[60] 3-(4-((2-Benzyl-2,6-diazaspiro[3.4]octan-6-yl)methyl)-5-chloro-1-methyl-1H-pyrazol-3-yl)-5-methylisoxazole;
[61] 3-(5-Chloro-1-methyl-4-((2-phenethyl-2,6-diazaspiro[3.4]octan-6-yl)methyl)-1H-pyrazol-3-yl)-5-methylisoxazole;
[62] 1-(1-((5-Chloro-1-methyl-3-(5-methylisoxazol-3-yl)-1H-pyrazol-4-yl)methyl)pyrrolidin-3-yl)-N-isopentylcyclopropanamine;
[63] 1-(1-((5-Chloro-1-methyl-3-(5-methylisoxazol-3-yl)-1H-pyrazol-4-yl)methyl)piperidin-3-yl)-N-isopentylcyclopropanamine;
[64] N-((4-((5-Chloro-1-methyl-3-(5-methylisoxazol-3-yl)-1H-pyrazol-4-yl)methyl)-1,4-oxazepan-2-yl)methyl)-3-methylbutan-1-amine;
[65] 3-(5-Chloro-1-methyl-4-((9-(1-phenylethyl)-3,9-diazaspiro[5.5]undecan-3-yl)methyl)-1H-pyrazol-3-yl)-5-methylisoxazole;
[66] 3-(4-((9-Benzyl-3,9-diazaspiro[5.5]undecan-3-yl)methyl)-5-chloro-1-methyl-1H-pyrazol-3-yl)-5-methylisoxazole;
[67] 3-(5-Chloro-4-((9-isopentyl-3,9-diazaspiro[5.5]undecan-3-yl)methyl)-1-methyl-1H-pyrazol-3-yl)-5-methylisoxazole;
[68] 3-(5-Chloro-1-methyl-4-((9-neopentyl-3,9-diazaspiro[5.5]undecan-3-yl)methyl)-1H-pyrazol-3-yl)-5-methylisoxazole;
[69] (S)-3-(5-Chloro-4-((7-(3,3-dimethylbutyl)-2,7-diazaspiro[4.4]nonan-2-yl)methyl)-1-methyl-1H-pyrazol-3-yl)-5-methylisoxazole;
[70] (R)-3-(5-Chloro-4-((7-(3,3-dimethylbutyl)-2,7-diazaspiro[4.4]nonan-2-yl)methyl)-1-methyl-1H-pyrazol-3-yl)-5-methylisoxazole;
[71] (R)-N-((1-((5-Chloro-1-methyl-3-(5-methylisoxazol-3-yl)-1H-pyrazol-4-yl)methyl)pyrrolidin-3-yl)methyl)-2-phenylethanamine;
[72] (S)-N-((1-((5-chloro-1-methyl-3-(5-methylisoxazol-3-yl)-1H-pyrazol-4-yl)methyl)pyrrolidin-3-yl)methyl)-2-phenylethanamine;
[73] (S)-N-((1-((5-Chloro-1-methyl-3-(5-methylisoxazol-3-yl)-1H-pyrazol-4-yl)methyl)pyrrolidin-3-yl)methyl)-3,3-dimethylbutan-1-amine;
[74] (R)-N-((1-((5-Chloro-1-methyl-3-(5-methylisoxazol-3-yl)-1H-pyrazol-4-yl)methyl)pyrrolidin-3-yl)methyl)-3,3-dimethylbutan-1-amine;
[75] (2s,4r)-6-((5-Chloro-1-methyl-3-(5-methylisoxazol-3-yl)-1H-pyrazol-4-yl)methyl)-N-(3,3-dimethylbutyl)-6-azaspiro[3.4]octan-2-amine;
[76] (2r,4s)-6-((5-Chloro-1-methyl-3-(5-methylisoxazol-3-yl)-1H-pyrazol-4-yl)methyl)-N-(3,3-dimethylbutyl)-6-azaspiro[3.4]octan-2-amine;
[77] N-(((3R,4S)-1-((5-Chloro-1-methyl-3-(5-methylisoxazol-3-yl)-1H-pyrazol-4-yl)methyl)-4-methylpyrrolidin-3-yl)methyl)-3,3-dimethylbutan-1-amine;
[78] N-(((3S,4R)-1-((5-Chloro-1-methyl-3-(5-methylisoxazol-3-yl)-1H-pyrazol-4-yl)methyl)-4-methylpyrrolidin-3-yl)methyl)-3,3-dimethylbutan-1-amine;
[79] 1-((5-Chloro-1-methyl-3-(5-methylisoxazol-3-yl)-1H-pyrazol-4-yl)methyl)-N-(2-(piperidin-1-yl)ethyl)azepan-3-amine;
[80] 1-((5-Chloro-1-methyl-3-(5-methylisoxazol-3-yl)-1H-pyrazol-4-yl)methyl)-N-isopentyl-N-methylazepan-3-amine;
[81] 1-((5-Chloro-1-methyl-3-(5-methylisoxazol-3-yl)-1H-pyrazol-4-yl)methyl)-N-methyl-N-((tetrahydro-2H-pyran-4-yl)methyl)azepan-3-amine;
[82] 1-((5-Chloro-1-methyl-3-(5-methylisoxazol-3-yl)-1H-pyrazol-4-yl)methyl)-N-methyl-N-(2-(piperidin-1-yl)ethyl)azepan-3-amine;
[83] (5-Chloro-1-methyl-3-(5-methylisoxazol-3-yl)-1H-pyrazol-4-yl)(3-(((3,3-dimethylbutyl)amino)methyl)piperidin-1-yl)methanone;
[84] (5-Chloro-1-methyl-3-(5-methylisoxazol-3-yl)-1H-pyrazol-4-yl)(3-(isopentylamino)azepan-1-yl)methanone;
[85] (5-Chloro-1-methyl-3-(5-methylisoxazol-3-yl)-1H-pyrazol-4-yl)(3-(phenethylamino)azepan-1-yl)methanone;
[86] (3-(Benzylamino)azepan-1-yl)(5-chloro-1-methyl-3-(5-methylisoxazol-3-yl)-1H-pyrazol-4-yl)methanone;
[87] (5-Chloro-1-methyl-3-(5-methylisoxazol-3-yl)-1H-pyrazol-4-yl)(2-(3,3-dimethylbutyl)-2,8-diazaspiro[4.5]decan-8-yl)methanone;
[88] (5-Chloro-1-methyl-3-(5-methylisoxazol-3-yl)-1H-pyrazol-4-yl)(9-(3,3-dimethylbutyl)-3,9-diazaspiro[5.5]undecan-3-yl)methanone;
[89] (5-Chloro-1-methyl-3-(5-methylisoxazol-3-yl)-1H-pyrazol-4-yl)(9-isobutyl-3,9-diazaspiro[5.5]undecan-3-yl)methanone;
[90] (5-Chloro-1-methyl-3-(5-methylisoxazol-3-yl)-1H-pyrazol-4-yl)(9-isopentyl-3,9-diazaspiro[5.5]undecan-3-yl)methanone;
[91] (5-Chloro-1-methyl-3-(5-methylisoxazol-3-yl)-1H-pyrazol-4-yl)(2-(3,3-dimethylbutyl)-2,9-diazaspiro[5.5]undecan-9-yl)methanone;
[92] (5-Chloro-1-methyl-3-(5-methylisoxazol-3-yl)-1H-pyrazol-4-yl)(9-(3,3-dimethylbutyl)-2,9-diazaspiro[5.5]undecan-2-yl)methanone;
[93] (5-Chloro-1-methyl-3-(5-methylisoxazol-3-yl)-1H-pyrazol-4-yl)(9-(isopentylamino)-3-azaspiro[5.5]undecan-3-yl)methanone;
[94] (5-Chloro-1-methyl-3-(5-methylisoxazol-3-yl)-1H-pyrazol-4-yl)(8-(3,3-dimethylbutyl)-2,8-diazaspiro[4.5]decan-2-yl)methanone;
[95] (5-Chloro-1-methyl-3-(5-methylisoxazol-3-yl)-1H-pyrazol-4-yl)(3-((isobutylamino)methyl)piperidin-1-yl)methanone;
[96] (5-Chloro-1-methyl-3-(5-methylisoxazol-3-yl)-1H-pyrazol-4-yl)(9-((3,3-dimethylbutyl)amino)-3-azaspiro[5.5]undecan-3-yl)methanone;
[97] (5-Chloro-3-(5-ethylisoxazol-3-yl)-1-methyl-1H-pyrazol-4-yl)(9-(3,3-dimethylbutyl)-3,9-diazaspiro[5.5]undecan-3-yl)methanone;
[98] (9-(3,3-Dimethylbutyl)-3,9-diazaspiro[5.5]undecan-3-yl)(5-fluoro-1-methyl-3-(5-methylisoxazol-3-yl)-1H-pyrazol-4-yl)methanone;
[99] (5-Chloro-1-ethyl-3-(5-methylisoxazol-3-yl)-1H-pyrazol-4-yl)(9-(3,3-dimethylbutyl)-3,9-diazaspiro[5.5]undecan-3-yl)methanone;
[100] (5-Chloro-1-methyl-3-(4-methylisoxazol-3-yl)-1H-pyrazol-4-yl)(9-(3,3-dimethylbutyl)-3,9-diazaspiro[5.5]undecan-3-yl)methanone;
[101] (5-Chloro-3-(5-methylisoxazol-3-yl)-1-phenyl-1H-pyrazol-4-yl)(9-(3,3-dimethylbutyl)-3,9-diazaspiro[5.5]undecan-3-yl)methanone;
[102] (5-Chloro-3-(6-methoxypyridin-2-yl)-1-methyl-1H-pyrazol-4-yl)(9-(3,3-dimethylbutyl)-3,9-diazaspiro[5.5]undecan-3-yl)methanone;
[103] (5-Chloro-1-methyl-3-(4-methylisoxazol-3-yl)-1H-pyrazol-4-yl)(9-(3,3-dimethylbutyl)-2,9-diazaspiro[5.5]undecan-2-yl)methanone;
[104] (5-Chloro-1-methyl-3-(5-methylisoxazol-3-yl)-1H-pyrazol-4-yl)(7-(3,3-dimethylbutyl)-2,7-diazaspiro[3.5]nonan-2-yl)methanone;
[105] (5-Chloro-1-methyl-3-(5-methylisoxazol-3-yl)-1H-pyrazol-4-yl)(2-(3,3-dimethylbutyl)-2,6-diazaspiro[3.4]octan-6-yl)methanone;
[106] 5-Chloro-N-(7-(3,3-dimethylbutyl)-7-azaspiro[3.5]nonan-2-yl)-1-methyl-3-(5-methylisoxazol-3-yl)-1H-pyrazole-4-carboxamide;
[107] 5-Chloro-N-(3-(3,3-dimethylbutyl)-3-azaspiro[5.5]undecan-9-yl)-1-methyl-3-(5-methylisoxazol-3-yl)-1H-pyrazole-4-carboxamide;
[108] (R)-(5-Chloro-1-methyl-3-(5-methylisoxazol-3-yl)-1H-pyrazol-4-yl)(3-(isopentylamino)piperidin-1-yl)methanone;
[109] (S)-(5-Chloro-1-methyl-3-(5-methylisoxazol-3-yl)-1H-pyrazol-4-yl)(3-(isopentylamino)piperidin-1-yl)methanone;
[110] (5-Chloro-1-methyl-3-(5-methylisoxazol-3-yl)-1H-pyrazol-4-yl)(3-(methyl(phenethyl)amino)azepan-1-yl)methanone;
[111] (5-Chloro-1-methyl-3-(5-methylisoxazol-3-yl)-1H-pyrazol-4-yl)(3-(isopentyl(methyl)amino)azepan-1-yl)methanone;
[112] 2-(9-(5-Chloro-1-methyl-3-(5-methylisoxazol-3-yl)-1H-pyrazole-4-carbonyl)-3,9-diazaspiro[5.5]undecan-3-yl)-1-(piperidin-1-yl)ethanone;
[113] 2-(9-(5-Chloro-1-methyl-3-(5-methylisoxazol-3-yl)-1H-pyrazole-4-carbonyl)-3,9-diazaspiro[5.5]undecan-3-yl)-1-(4,4-difluoropiperidin-1-yl)ethanone;
[114] 2-((5-Chloro-1-methyl-3-(5-methylisoxazol-3-yl)-1H-pyrazol-4-yl)methyl)-8-(3,3-dimethylbutyl)-2,8-diazaspiro[4.5]decan-1-one;
[115] 3-((5-Chloro-1-methyl-3-(5-methylisoxazol-3-yl)-1H-pyrazol-4-yl)methyl)-9-(3,3-dimethylbutyl)-3,9-diazaspiro[5.5]undecan-2-one;
[116] 2-((5-Chloro-1-methyl-3-(5-methylisoxazol-3-yl)-1H-pyrazol-4-yl)methyl)-8-(3,3-dimethylbutyl)-2,8-diazaspiro[4.5]decan-3-one;
[117] 1-((5-Chloro-1-methyl-3-(5-methylisoxazol-3-yl)-1H-pyrazol-4-yl)methyl)-3-(isopentylamino)azepan-2-one;
[118] 1-((5-Chloro-1-methyl-3-(5-methylisoxazol-3-yl)-1H-pyrazol-4-yl)methyl)-4-(isopentylamino)azepan-2-one;
[119] (S)-1-(2-(5-Chloro-1-methyl-3-(5-methylisoxazol-3-yl)-1H-pyrazol-4-yl)ethyl)-N-isopentylazepan-3-amine;
[120] (2-(5-Chloro-1-methyl-3-(5-methylisoxazol-3-yl)-1H-pyrazol-4-yl)-1-(9-isopentyl-3,9-diazaspiro[5.5]undecan-3-yl)ethenone;
[121] (5-Chloro-1-methyl-3-(5-methylisoxazol-3-yl)-1H-pyrazol-4-yl)(3-(((3,3-dimethylbutyl)(methyl)amino)methyl)piperidin-1-yl)methanone;
[122] (5-Chloro-1-methyl-3-(5-methylisoxazol-3-yl)-1H-pyrazol-4-yl)(3-((isobutyl(methyl)amino)methyl)piperidin-1-yl)methanone;
[123] (5-Chloro-1-methyl-3-(5-methylisoxazol-3-yl)-1H-pyrazol-4-yl)((2r,4s)-2-((3,3-dimethylbutyl)amino)-6-azaspiro[3.4]octan-6-yl)methanone;
[124] (5-Chloro-3-(6-hydroxypyridin-2-yl)-1-methyl-1H-pyrazol-4-yl)(9-(3,3-dimethylbutyl)-3,9-diazaspiro[5.5]undecan-3-yl)methanone;
[125] (R)-(5-Chloro-1-methyl-3-(5-methylisoxazol-3-yl)-1H-pyrazol-4-yl)(3-((isopentylamino)methyl)piperidin-1-yl)methanone; and
[126] (S)-(5-Chloro-1-methyl-3-(5-methylisoxazol-3-yl)-1H-pyrazol-4-yl)(3-((isopentylamino)methyl)piperidin-1-yl)methanone;
[127] 5-Chloro-N-(7-(3,3-dimethylbutyl)-7-azaspiro[3.5]nonan-2-yl)-1-ethyl-3-(5-methylisoxazol-3-yl)-1H-pyrazole-4-carboxamide;
[128] (5-Chloro-1-methyl-3-(5-methylisoxazol-3-yl)-1H-pyrazol-4-yl)(9-(3,4-difluorobenzyl)-2,9-diazaspiro[5.5]undecan-2-yl)methanone;
[129] (5-Chloro-1-methyl-3-(5-methylisoxazol-3-yl)-1H-pyrazol-4-yl)(9-(4-fluorobenzyl)-3,9-diazaspiro[5.5]undecan-3-yl)methanone;
[130] (5-Chloro-1-methyl-3-(5-methylisoxazol-3-yl)-1H-pyrazol-4-yl)(9-(3,4-difluorobenzyl)-3,9-diazaspiro[5.5]undecan-3-yl)methanone;
[131] 4-((9-(5-Chloro-1-methyl-3-(5-methylisoxazol-3-yl)-1H-pyrazole-4-carbonyl)-3,9-diazaspiro[5.5]undecan-3-yl)methyl)-2-fluorobenzonitrile;
[132] (5-Chloro-3-(5-isopropylisoxazol-3-yl)-1-methyl-1H-pyrazol-4-yl)(9-(3,3-dimethylbutyl)-2,9-diazaspiro[5.5]undecan-2-yl)methanone;
[133] (5-Chloro-3-(2-ethyl-4-methyloxazol-5-yl)-1-methyl-1H-pyrazol-4-yl)(9-(3,3-dimethylbutyl)-2,9-diazaspiro[5.5]undecan-2-yl)methanone;
[134] 5-Chloro-N-(2-(3,3-dimethylbutyl)-2-azaspiro[3.5]nonan-7-yl)-1-ethyl-3-(5-methylisoxazol-3-yl)-1H-pyrazole-4-carboxamide;
[135] (5-Chloro-1,1′-dimethyl-1H,1′H-[3,4′-bipyrazol]-4-yl)(9-(3,3-dimethylbutyl)-2,9-diazaspiro[5.5]undecan-2-yl)methanone;
[136] 5-Chloro-N-(7-(3,3-dimethylbutyl)-7-azaspiro[3.5]nonan-2-yl)-1-ethyl-3-(5-ethylisoxazol-3-yl)-1H-pyrazole-4-carboxamide;
[137] 5-Chloro-N-(2-(3,3-dimethylbutyl)-2-azaspiro[3.5]nonan-7-yl)-1-ethyl-3-(5-ethylisoxazol-3-yl)-1H-pyrazole-4-carboxamide;
[138] (5-Chloro-1′-ethyl-1-methyl-1H,1′H-[3,3′-bipyrazol]-4-yl)(9-(3,3-dimethylbutyl)-2,9-diazaspiro[5.5]undecan-2-yl)methanone;
[139] 5-Chloro-N-(7-(3,3-dimethylbutyl)-7-azaspiro[3.5]nonan-2-yl)-1-ethyl-3-(isoxazol-3-yl)-1H-pyrazole-4-carboxamide;
[140] 5-Chloro-N-(7-(3,3-dimethylbutyl)-7-azaspiro[3.5]nonan-2-yl)-1,1′-dimethyl-1H,1′H-[3,4′-bipyrazole]-4-carboxamide;
[141] 5-Chloro-N-(7-(3,3-dimethylbutyl)-7-azaspiro[3.5]nonan-2-yl)-1-methyl-3-(6-methylpyridin-2-yl)-1H-pyrazole-4-carboxamide;
[142] 5-Chloro-N-(3-(3,3-dimethylbutyl)-3-azaspiro[5.5]undecan-9-yl)-1-ethyl-3-(isoxazol-3-yl)-1H-pyrazole-4-carboxamide;
[143] 5-Chloro-N-(3-(3,3-dimethylbutyl)-3-azaspiro[5.5]undecan-9-yl)-1-ethyl-3-(5-methylisoxazol-3-yl)-1H-pyrazole-4-carboxamide;
[144] (5-Chloro-1-methyl-3-(5-methylisoxazol-3-yl)-1H-pyrazol-4-yl)(6-(3,3-dimethylbutyl)-2,6-diazaspiro[3.5]nonan-2-yl)methanone;
[145] 5-Chloro-N-(7-(3,3-dimethylbutyl)-7-azaspiro[3.5]nonan-2-yl)-1′-ethyl-1-methyl-1H,1′H-[3,4′-bipyrazole]-4-carboxamide;
[146] 5-Chloro-N-(3-(3,3-dimethylbutyl)-3-azaspiro[5.5]undecan-9-yl)-1′-ethyl-1-methyl-1H,1′H-[3,4′-bipyrazole]-4-carboxamide;
[147] 5-Chloro-N-(3-(3,3-dimethylbutyl)-3-azaspiro[5.5]undecan-9-yl)-1-methyl-3-(6-(trifluoromethyl)pyridin-2-yl)-1H-pyrazole-4-carboxamide;
[148] 5-Chloro-N-(3-(3,3-dimethylbutyl)-3-azaspiro[5.5]undecan-9-yl)-1-methyl-3-(6-methylpyridin-2-yl)-1H-pyrazole-4-carboxamide;
[149] 5-Chloro-N-(7-(3,3-dimethylbutyl)-7-azaspiro[3.5]nonan-2-yl)-1-ethyl-1′-isopropyl-1H,1′H-[3,4′-bipyrazole]-4-carboxamide;
[150] (5-Chloro-1-ethyl-3-(5-methylisoxazol-3-yl)-1H-pyrazol-4-yl)(9-(3,3-dimethylbutyl)-2,9-diazaspiro[5.5]undecan-2-yl)methanone;
[151] 5-Chloro-N-(3-(3,3-dimethylbutyl)-3-azaspiro[5.5]undecan-9-yl)-1-ethyl-3-(6-methylpyridin-2-yl)-1H-pyrazole-4-carboxamide;
[152] (5-Chloro-1-ethyl-3-(5-isopropylisoxazol-3-yl)-1H-pyrazol-4-yl)(9-(3,3-dimethylbutyl)-2,9-diazaspiro[5.5]undecan-2-yl)methanone;
[153] N-(7-Benzyl-7-azaspiro[3.5]nonan-2-yl)-5-chloro-1-methyl-3-(5-methylisoxazol-3-yl)-1H-pyrazole-4-carboxamide;
[154] 5-Chloro-1-methyl-3-(5-methylisoxazol-3-yl)-N-(7-neopentyl-7-azaspiro[3.5]nonan-2-yl)-1H-pyrazole-4-carboxamide;
[155] (5-Chloro-1-methyl-3-(6-(trifluoromethyl)pyridin-2-yl)-1H-pyrazol-4-yl)(9-(3,3-dimethylbutyl)-2,9-diazaspiro[5.5]undecan-2-yl)methanone;
[156] (9-Benzyl-2,9-diazaspiro[5.5]undecan-2-yl)(5-chloro-1-methyl-3-(5-methylisoxazol-3-yl)-1H-pyrazol-4-yl)methanone;
[157] (5-Chloro-1-methyl-3-(5-methylisoxazol-3-yl)-1H-pyrazol-4-yl)(9-neopentyl-2,9-diazaspiro[5.5]undecan-2-yl)methanone;
[158] 5-Chloro-N-(2-(1-(3,3-dimethylbutyl)piperidin-4-yl)ethyl)-1-ethyl-3-(5-methylisoxazol-3-yl)-1H-pyrazole-4-carboxamide;
[159] 5-Chloro-N-(1-(3,3-dimethylbutyl)piperidin-4-yl)-1-ethyl-3-(5-methylisoxazol-3-yl)-1H-pyrazole-4-carboxamide;
[160] 5-Chloro-N-(1-(3,3-dimethylbutyl)azepan-4-yl)-1-ethyl-3-(5-methylisoxazol-3-yl)-1H-pyrazole-4-carboxamide;
[161] 5-Chloro-N-(1-(3,3-dimethylbutyl)piperidin-4-yl)-1-ethyl-3-(6-(trifluoromethyl)pyridin-2-yl)-1H-pyrazole-4-carboxamide;
[162] 5-Chloro-N-(1-(3,3-dimethylbutyl)piperidin-4-yl)-1-ethyl-3-(5-isopropylisoxazol-3-yl)-1H-pyrazole-4-carboxamide;
[163] 5-Chloro-N-(1-(3,3-dimethylbutyl)piperidin-4-yl)-1-ethyl-3-(isoxazol-3-yl)-1H-pyrazole-4-carboxamide;
[164] (S)-5-Chloro-N-(1-(3,3-dimethylbutyl)pyrrolidin-3-yl)-1-ethyl-3-(5-methylisoxazol-3-yl)-1H-pyrazole-4-carboxamide;
[165] (R)-5-Chloro-N-(1-(3,3-dimethylbutyl)pyrrolidin-3-yl)-1-ethyl-3-(5-methylisoxazol-3-yl)-1H-pyrazole-4-carboxamide;
[166] 5-Chloro-N-(1-(3,3-dimethylbutyl)piperidin-4-yl)-1-methyl-3-(5-methylisoxazol-3-yl)-1H-pyrazole-4-carboxamide;
[167] 5-Chloro-N-(1-(3,3-dimethylbutyl)piperidin-4-yl)-1-methyl-3-(6-(trifluoromethyl)pyridin-2-yl)-1H-pyrazole-4-carboxamide;
[168] 5-Chloro-N-(1-(3,3-dimethylbutyl)piperidin-4-yl)-1-methyl-3-(6-methylpyridin-2-yl)-1H-pyrazole-4-carboxamide;
[169] (5-Chloro1-methyl-3-(5-methylisoxazol-3-yl)-1H-pyrazol-4-yl)((2s,4r)-2-((3,3-dimethylbutyl)amino)-6-azaspiro[3.4]octan-6-yl)methanone;
[170] (5-Chloro-3-(5-methylisoxazol-3-yl)-1-(pyridin-4-yl)-1H-pyrazol-4-yl)(9-(3,3-dimethylbutyl)-3,9-diazaspiro[5.5]undecan-3-yl)methanone;
[171] (5-Chloro-1-ethyl-3-(isoxazol-3-yl)-1H-pyrazol-4-yl)(9-(3,3-dimethylbutyl)-3,9-diazaspiro[5.5]undecan-3-yl)methanone;
[172] (5-Chloro-3-(5-methylisoxazol-3-yl)-1-(pyridin-2-yl)-1H-pyrazol-4-yl)(9-(3,3-dimethylbutyl)-3,9-diazaspiro[5.5]undecan-3-yl)methanone;
[173] (5-Chloro-1-methyl-3-(pyridin-2-yl)-1H-pyrazol-4-yl)(9-(3,3-dimethylbutyl)-3,9-diazaspiro[5.5]undecan-3-yl)methanone;
[174] (5-Chloro-1-methyl-3-(5-methylisoxazol-3-yl)-1H-pyrazol-4-yl)(2-((3,3-dimethylbutyl)amino)-7-azaspiro[3.5]nonan-7-yl)methanone;
[175] (9-Benzyl-3,9-diazaspiro[5.5]undecan-3-yl)(5-chloro-1-methyl-3-(5-methylisoxazol-3-yl)-1H-pyrazol-4-yl)methanone;
[176] (5-Chloro-3-(2-ethyl-4-methyloxazol-5-yl)-1-methyl-1H-pyrazol-4-yl)(9-(3,3-dimethylbutyl)-3,9-diazaspiro[5.5]undecan-3-yl)methanone;
[177] (5-Chloro-3-(5-isopropylisoxazol-3-yl)-1-methyl-1H-pyrazol-4-yl)(9-(3,3-dimethylbutyl)-3,9-diazaspiro[5.5]undecan-3-yl)methanone;
[178] (5-Chloro-1,1′-dimethyl-1H,1′H-[3,4′-bipyrazol]-4-yl)(9-(3,3-dimethylbutyl)-3,9-diazaspiro[5.5]undecan-3-yl)methanone;
[179] (5-Chloro-1′-ethyl-1-methyl-1H,1′H-[3,3′-bipyrazol]-4-yl)(9-(3,3-dimethylbutyl)-3,9-diazaspiro[5.5]undecan-3-yl)methanone;
[180] (5-Chloro-1-methyl-3-(6-(trifluoromethyl) undecane-2-yl)-1H-pyrazol-4-yl)(9-(3,3-dimethylbutyl)-3,9-diazaspiro[5.5] undecane-3-yl)methanone;
[181] (5-Chloro-1,1′-dimethyl-1H,1′H-[3,3′-bipyrazol]-4-yl)(9-(3,3-dimethylbutyl)-3,9-diazaspiro[5.5]undecan-3-yl)methanone;
[182] (5-Chloro-3-(5-methylisoxazol-3-yl)-1-propyl-1H-pyrazol-4-yl)(9-(3,3-dimethylbutyl)-3,9-diazaspiro[5.5]undecan-3-yl)methanone;
[183] (5-Chloro-1-isopropyl-3-(5-methylisoxazol-3-yl)-1H-pyrazol-4-yl)(9-(3,3-dimethylbutyl)-3,9-diazaspiro[5.5]undecan-3-yl)methanone;
[184] (5-Chloro-1-ethyl-3-(5-methylisoxazol-3-yl)-1H-pyrazol-4-yl)(7-(3,3-dimethylbutyl)-2,7-diazaspiro[3.5]nonan-2-yl)methanone;
[185] (5-Chloro-3-(5-methylisoxazol-3-yl)-1-propyl-1H-pyrazol-4-yl)(7-(3,3-dimethylbutyl)-2,7-diazaspiro[3.5]nonan-2-yl)methanone;
[186] (5-Chloro-3-(6-ethoxypyridin-2-yl)-1-methyl-1H-pyrazol-4-yl)(9-(3,3-dimethylbutyl)-3,9-diazaspiro[5.5]undecan-3-yl)methanone;
[187] (5-Chloro-1-ethyl-3-(isoxazol-3-yl)-1H-pyrazol-4-yl)(7-(3,3-dimethylbutyl)-2,7-diazaspiro[3.5]nonan-2-yl)methanone;
[188] (5-Chloro-1-isopropyl-3-(5-methylisoxazol-3-yl)-1H-pyrazol-4-yl)(7-(3,3-dimethylbutyl)-2,7-diazaspiro[3.5]nonan-2-yl)methanone;
[189] (5-Chloro-1-methyl-3-(6-methylpyridin-2-yl)-1H-pyrazol-4-yl)(9-(3,3-dimethylbutyl)-3,9-diazaspiro[5.5]undecan-3-yl)methanone;
[190] (5-Chloro-3-(5-cyclopropylisoxazol-3-yl)-1-ethyl-1H-pyrazol-4-yl)(9-(3,3-dimethylbutyl)-3,9-diazaspiro[5.5]undecan-3-yl)methanone;
[191] (5-Chloro-3-(5-cyclopropylisoxazol-3-yl)-1-methyl-1H-pyrazol-4-yl)(9-(3,3-dimethylbutyl)-3,9-diazaspiro[5.5]undecan-3-yl)methanone;
[192] (5-Chloro-1-ethyl-3-(5-isopropylisoxazol-3-yl)-1H-pyrazol-4-yl)(9-(3,3-dimethylbutyl)-3,9-diazaspiro[5.5]undecan-3-yl)methanone;
[193] (5-Chloro-1-methyl-3-(5-methylisoxazol-3-yl)-1H-pyrazol-4-yl)(2-(3,3-dimethylbutyl)-2,6-diazaspiro[3.5]nonan-6-yl)methanone;
[194] (5-Chloro-1-ethyl-3-(5-methylisoxazol-3-yl)-1H-pyrazol-4-yl)(2-(3,3-dimethylbutyl)-2,6-diazaspiro[3.5]nonan-6-yl)methanone;
[195] (5-Chloro-1-ethyl-3-(isoxazol-3-yl)-1H-pyrazol-4-yl)(2-(3,3-dimethylbutyl)-2,6-diazaspiro[3.5]nonan-6-yl)methanone;
[196] (5-Chloro-1′-ethyl-1-methyl-1H,1′H-[3,4′-bipyrazol]-4-yl)(9-(3,3-dimethylbutyl)-3,9-diazaspiro[5.5]undecan-3-yl)methanone;
[197] 5-Chloro-N-(2-(3,3-dimethylbutyl)-2-azaspiro[3.5]nonan-7-yl)-1-ethyl-3-(isoxazol-3-yl)-1H-pyrazole-4-carboxamide;
[198] (5-Chloro-1-ethyl-1′-isopropyl-1H,1′H-[3,4′-bipyrazol]-4-yl)(9-(3,3-dimethylbutyl)-3,9-diazaspiro[5.5]undecan-3-yl)methanone;
[199] (5-Chloro-1-ethyl-3-(6-(trifluoromethyl)pyridin-2-yl)-1H-pyrazol-4-yl)(9-(3,3-dimethylbutyl)-3,9-diazaspiro[5.5]undecan-3-yl)methanone;
[200] 5-Chloro-N-((1r,4r)-4-((3,3-dimethylbutyl)amino)cyclohexyl)-1-ethyl-3-(5-methylisoxazol-3-yl)-1H-pyrazole-4-carboxamide;
[201] 5-Chloro-N-((1s,4s)-4-((3,3-dimethylbutyl)amino)cyclohexyl)-1-ethyl-3-(5-methylisoxazol-3-yl)-1H-pyrazole-4-carboxamide;
[202] 5-Chloro-N-(1-(3,3-dimethylbutyl)piperidin-3-yl)-1-ethyl-3-(5-methylisoxazol-3-yl)-1H-pyrazole-4-carboxamide;
[203] 5-Chloro-N-((1-(3,3-dimethylbutyl)piperidin-4-yl)methyl)-1-ethyl-3-(5-methylisoxazol-3-yl)-1H-pyrazole-4-carboxamide;
[204] 5-Chloro-N-(2-(3,3-dimethylbutyl)-1,2,3,4-tetrahydroisoquinolin-6-yl)-1-ethyl-3-(5-methylisoxazol-3-yl)-1H-pyrazole-4-carboxamide;
[205] 5-Chloro-N-((1-(3,3-dimethylbutyl)piperidin-3-yl)methyl)-1-ethyl-3-(5-methylisoxazol-3-yl)-1H-pyrazole-4-carboxamide;
[206] 5-Chloro-N-(1-(3,3-dimethylbutyl)azepan-3-yl)-1-ethyl-3-(5-methylisoxazol-3-yl)-1H-pyrazole-4-carboxamide;
[207] (5-Chloro-1-ethyl-3-(2-methylpyridin-4-yl)-1H-pyrazol-4-yl)(9-(3,3-dimethylbutyl)-3,9-diazaspiro[5.5]undecan-3-yl)methanone;
[208] 2-(5-Chloro-1-methyl-3-(5-methylisoxazol-3-yl)-1H-pyrazol-4-yl)-1-(9-(3,3-dimethylbutyl)-3,9-diazaspiro[5.5]undecan-3-yl)ethan-1-one
[209] 2-(5-Chloro-1-ethyl-3-(isoxazol-3-yl)-1H-pyrazol-4-yl)-1-(9-(3,3-dimethylbutyl)-3,9-diazaspiro[5.5]undecan-3-yl)ethan-1-one;
[210] 2-(5-Chloro-1-ethyl-3-(5-methylisoxazol-3-yl)-1H-pyrazol-4-yl)-1-(9-(3,3-dimethylbutyl)-3,9-diazaspiro[5.5]undecan-3-yl)ethan-1-one;
[211] 2-(5-Chloro-1-ethyl-3-(isoxazol-3-yl)-1H-pyrazol-4-yl)-N-(2-(3,3-dimethylbutyl)-1,2,3,4-tetrahydroisoquinolin-6-yl)acetamide;
[212] 2-(5-Chloro-1-ethyl-3-(isoxazol-3-yl)-1H-pyrazol-4-yl)-N-((1-(3,3-dimethylbutyl)piperidin-3-yl)methyl)acetamide;
[213] 2-(5-Chloro-1-methyl-3-(6-(trifluoromethyl)pyridin-2-yl)-1H-pyrazol-4-yl)-1-(9-(3,3-dimethylbutyl)-3,9-diazaspiro[5.5]undecan-3-yl)ethan-1-one;
[214] 2-(5-Chloro-1-methyl-3-(5-methylisoxazol-3-yl)-1H-pyrazol-4-yl)-1-(9-(3,3-dimethylbutyl)-2,9-diazaspiro[5.5]undecan-2-yl)ethan-1-one;
[215] 2-(5-Chloro-1-methyl-3-(5-methylisoxazol-3-yl)-1H-pyrazol-4-yl)-N-(3-(3,3-dimethylbutyl)-3-azaspiro[5.5]undecan-9-yl)acetamide;
[216] 2-(5-Chloro-1-ethyl-3-(5-methylisoxazol-3-yl)-1H-pyrazol-4-yl)-N-(3-(3,3-dimethylbutyl)-3-azaspiro[5.5]undecan-9-yl)acetamide;
[217] 2-(5-Chloro-1-ethyl-3-(5-methylisoxazol-3-yl)-1H-pyrazol-4-yl)-1-(9-(3,3-dimethylbutyl)-2,9-diazaspiro[5.5]undecan-2-yl)ethan-1-one;
[218] 2-(5-Chloro-1-ethyl-3-(isoxazol-3-yl)-1H-pyrazol-4-yl)-1-(9-(3,3-dimethylbutyl)-2,9-diazaspiro[5.5]undecan-2-yl)ethan-1-one;
[219] 2-(5-Chloro-1-ethyl-3-(isoxazol-3-yl)-1H-pyrazol-4-yl)-N-(3-(3,3-dimethylbutyl)-3-azaspiro[5.5]undecan-9-yl)acetamide;
[220] 2-(5-Chloro-1-methyl-3-(5-methylisoxazol-3-yl)-1H-pyrazol-4-yl)-N-(1-(3,3-dimethylbutyl)piperidin-4-yl)acetamide;
[221] 2-(5-Chloro-1-ethyl-3-(isoxazol-3-yl)-1H-pyrazol-4-yl)-N-(1-(3,3-dimethylbutyl)piperidin-4-yl)acetamide;
[222] 2-(5-Chloro-1-methyl-3-(6-(trifluoromethyl)pyridin-2-yl)-1H-pyrazol-4-yl)-N-(1-(3,3-dimethylbutyl)piperidin-4-yl)acetamide;
[223] 2-(5-Chloro-1-ethyl-3-(6-(trifluoromethyl)pyridin-2-yl)-1H-pyrazol-4-yl)-N-(1-(3,3-dimethylbutyl)piperidin-4-yl)acetamide;
[224] 2-(5-Chloro-1-ethyl-3-(isoxazol-3-yl)-1H-pyrazol-4-yl)-N-(7-(3,3-dimethylbutyl)-7-azaspiro[3.5]nonan-2-yl)acetamide
[225] 2-((5-Chloro-1-ethyl-3-(5-methylisoxazol-3-yl)-1H-pyrazol-4-yl)methyl)-8-(3,3-dimethylbutyl)-2,8-diazaspiro[4.5]decan-3-one;
[226] 2-((5-Chloro-3-(5-methylisoxazol-3-yl)-1-propyl-1H-pyrazol-4-yl)methyl)-8-(3,3-dimethylbutyl)-2,8-diazaspiro[4.5]decan-3-one;
[227] 2-((5-Chloro-1-methyl-3-(6-(trifluoromethyl)pyridin-2-yl)-1H-pyrazol-4-yl)methyl)-8-(3,3-dimethylbutyl)-2,8-diazaspiro[4.5]decan-3-one;
[228] 2-((5-Chloro-1-methyl-3-(6-methylpyridin-2-yl)-1H-pyrazol-4-yl)methyl)-8-(3,3-dimethylbutyl)-2,8-diazaspiro[4.5]decan-3-one and
[229] 2-((5-Chloro-1-ethyl-3-(6-(trifluoromethyl)pyridin-2-yl)-1H-pyrazol-4-yl)methyl)-8-(3,3-dimethylbutyl)-2,8-diazaspiro[4.5]decan-3-one.
   or a pharmaceutical acceptable salt, stereoisomer, co-crystal, prodrug or solvate thereof.

In another aspect, the invention refers to the processes for obtaining the compounds of general formula (I). Several procedures have been developed for obtaining all the compounds of the invention, and the procedures will be explained below in methods A, B, C and D.

The obtained reaction products may, if desired, be purified by conventional methods, such as crystallization and chromatography. Where the processes described below for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

### Method A

Method A represents a first process for synthesizing compounds according to general formula (I). Method A allows the preparation of compounds of general formula (Ia), that is, compounds of general formula (I) where A represents —CH₂—B and n is 0.

Thus, a process is described for the preparation of a compound of general formula (Ia):

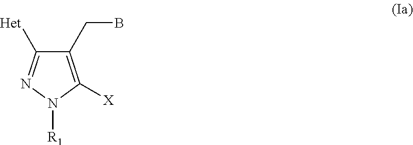

which comprises reacting a compound of formula (VI):

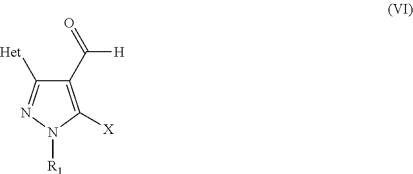

with a compound of formula (VII):

HB (VII)

wherein Het, X, B and R₁ are as defined along the detailed description and the claims. A compound of formula (Ia) can be obtained by reductive amination reaction of a compound of formula (VI) with an amine containing compound of formula (VII) in the presence of a reductive agent, preferably sodium triacetoxyborohydride, in an aprotic solvent, preferably tetrahydrofuran or dichloroethane, at a suitable temperature preferably reflux temperature. Alternatively, this reaction can be carried out under microwave heating.

### Method B

Method B represents a second process for synthesizing compounds according to general formula (I). Method B allows the preparation of compounds of general formula (Ib) that is compounds of formula (I) where A represents —(CH₂)ᵢ—CO—B and i being 0 and n being also 0.

Therefore, a process is described for the preparation of a compound of general formula (Ib):

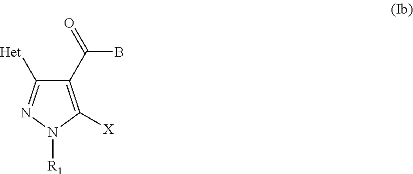

comprising the reaction between a compound of general formula (VIII):

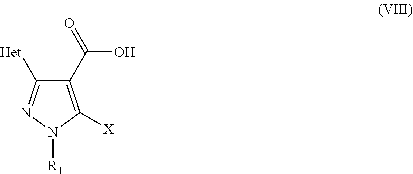

with a compound of formula (VII):

HB (VII)

wherein Het, X, B and R₁ are as defined along the detailed description and the claims.

A compound of formula I according to method B can be prepared acylation to an amide of formula (Ib) of a carboxylic acid of formula (VIII). The acylation reaction can be performed using amide coupling conditions, such as, EDC/HOBT/TEA in a suitable solvent, such as DMF at a suitable temperature, preferably room temperature.

### Method C

Method C is a further process for synthesizing compounds according to general formula (I). Method C allows the preparation of compounds of general formula (Ic), that is, compounds of general formula (I) where A represents —CH₂—B and n is 1.

Therefore, a process is described for the preparation of a compound of general formula (Ic):

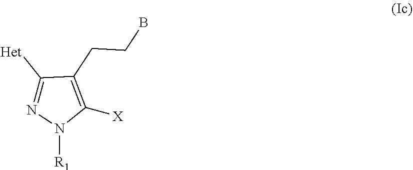

which comprises reacting a compound of formula (XII):

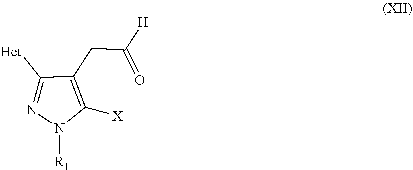

with a compound of formula (VII):

HB (VII)

wherein Het, X, B and R₁ are as defined as defined along the detailed description and the claims.

The carbaldehyde of formula (XII) can be converted into a compound of formula (Ic) through a reductive amination under conditions similar to those of method A. In this sense, reductive amination reaction of a compound of formula (XII) with an amine containing compound of formula (VII) can be carried out in the presence of a reductive agent, preferably sodium triacetoxyborohydride, in an aprotic solvent, preferably tetrahydrofuran or dichloroethane and at a suitable temperature, preferably reflux temperature. Alternatively, this reaction can be carried out under microwave heating.

### Method D

Method D is a further process for synthesizing compounds according to general formula (I). Method D allows the preparation of compounds of general formula (Id) that is compounds of formula (I) where A represents —(CH₂)ᵢ—CO—B i being 0 and n being 1.

Therefore, a process is described for the preparation of a compound of general formula (Id):

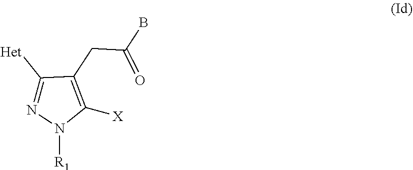

comprising the reaction between a compound of general formula (XIII):

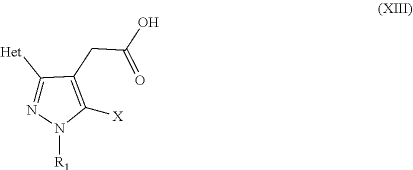

with a compound of formula (VII):

HB (VII)

wherein Het, X, B and R₁ are as defined along the detailed description and the claims.

Amide compounds of formula (Id) are obtained by treating a carboxylic acid compound of general formula (XIII) in the acylation conditions described in method B. Thus, the acylation reaction can be performed using amide coupling conditions, such as, EDC/HOBT/TEA in a suitable solvent, such as DMF at a suitable temperature, preferably room temperature.

In scheme 1 below, the synthetic routes of methods A, B, C and D are summarized wherein R₁, X, Het, and B have the meanings as defined in claim 1, A is alkyl and Y is a leaving group, such as chloro, iodo, mesylate or tosylate.

As shown in scheme 1, the compounds of formula (I) can be prepared in a multistep process comprising the following steps:

Step 1: A compound of formula (III) can be prepared by treating a carboxylic acid of formula (II) with 1,1′-carbonyldiimidazole in a suitable solvent, such as tetrahydrofuran or diethylether, at a suitable temperature, preferably at room temperature. The resulting 1-(1H-imidazol-1-yl)ketone can be reacted with an alkyl potassium malonate in the presence of a Lewis acid, such as MgCl₂ at a suitable temperature, preferably at room temperature followed by treatment with a solution of HCl at room temperature.

Step 2: A compound of formula (III) can be converted to a pyrazole derivative of formula (V) by treatment with a suitable substituted hydrazine of formula (IV), in a suitable solvent such as ethanol, in the presence of a suitable acid, such as hydrochloric acid, at a suitable temperature, preferably at reflux temperature.

Step 3: A carbaldehyde compound of formula (VI) wherein X is a chlorine atom can be prepared from a compound of formula (V) using phosphorous oxychloride and N,N-dimethylformamide in at a suitable temperature, between 0° C. and room temperature. Compounds where X is a halogen different than chlorine can be prepared from a compound of formula (VI) by a halide-exchange reaction, using an halide source such as KF, in a suitable solvent such as DMF, at a suitable temperature, preferably at reflux temperature using microwave heating.

Step 4: Which corresponds to the process described above in method A for producing a compound of formula (Ia) where a reductive amination reaction of a compound of formula (VI) with an amine containing compound of formula (VII) in the presence of a reductive agent, preferably sodium triacetoxyborohydride, in an aprotic solvent, preferably tetrahydrofuran or dichloroethane, at a suitable temperature preferably reflux temperature, is carried out. Alternatively, this reaction can be carried out under microwave heating.

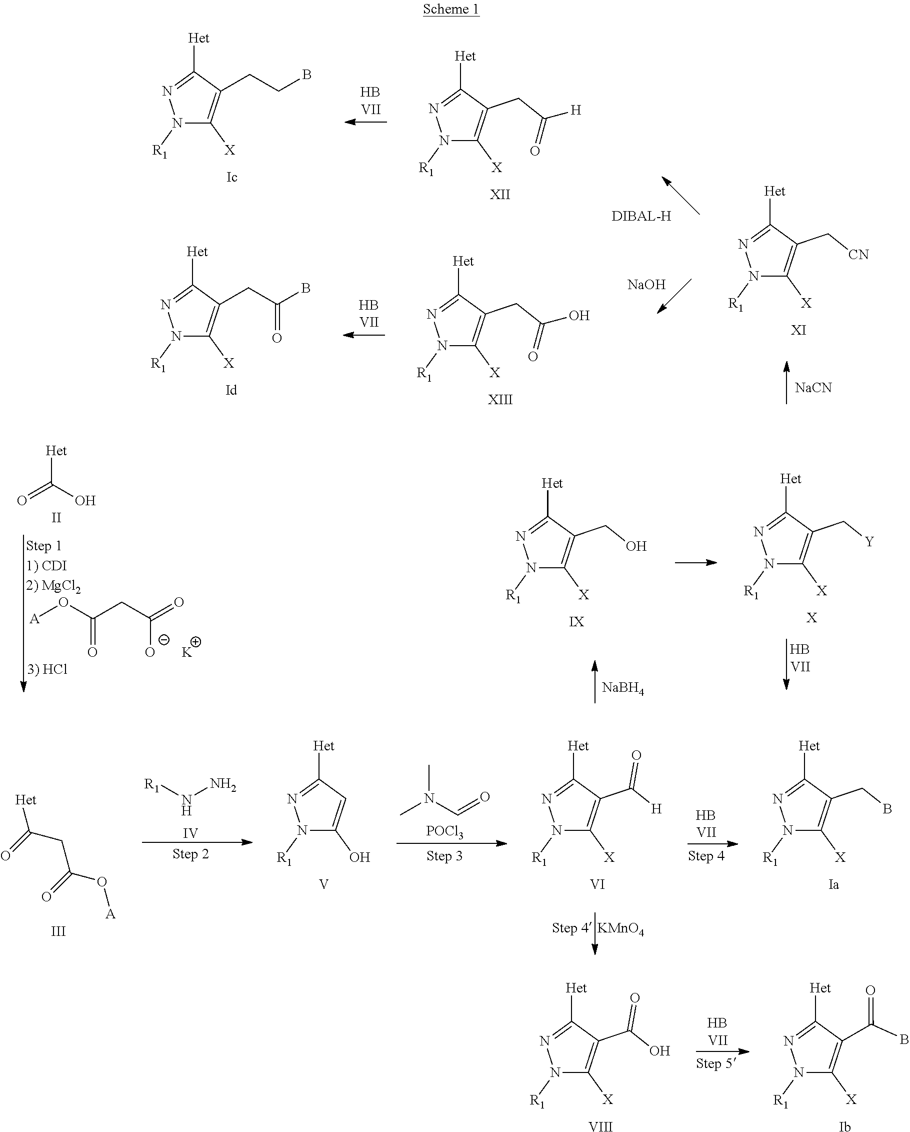

The process described by Steps 1 to 4 represents the most general route for the preparation of compounds of formula (Ia). Alternatively, they can be obtained by a three-step procedure from a carbaldehyde of formula (VI), involving reduction of the aldehyde with a suitable reducing agent, such as sodium borohydride, in a suitable solvent, such as methanol, at a suitable temperature, such as between 0° C. and room temperature. In a second step, a haloalkyl compound of formula (X) can be prepared by converting the hydroxyl group of a compound of formula (IX) into a leaving group by using for instance thionyl chloride in a suitable solvent, such as dichloromethane or toluene, preferably toluene, at a suitable temperature between room temperature and the reflux temperature, preferably at reflux temperature. Finally, a compound of formula (X) is used to alkylate a suitable amine containing compound of formula (VII), in a suitable solvent, such as acetonitrile, dichloromethane or DMF in the presence of a base, such NaH, at a suitable temperature comprised between 0° C. and room temperature.

The complete synthetic process leading to compounds of formula (Ib) is represented in step 4′ and step 5′.

Step 4′ and 5′. A compound of formula (Ib) can be obtained by a two-step procedure from a carbaldehyde of formula (VI), involving oxidation of compound (VI) to a carboxylic acid of formula (VIII) and then acylation to an amide of formula (Ib). The latter acylation step corresponds to method B described above. The oxidation reaction can be carried out using potassium permanganate, in a suitable solvent such as water, at a suitable temperature, preferably at the reflux temperature. Alternatively, the oxidation reaction can be carried out using Pinnick conditions with sulfamic acid and sodium chlorite in a suitable solvent such as a mixture of water and acetone, at a suitable temperature, preferably 0° C. The acylation reaction can be performed using amide coupling conditions, such as, EDC/HOBT/TEA in a suitable solvent, such as DMF at a suitable temperature, preferably room temperature.

Additionally, compounds of formula (Ic) or (Id) can be obtained from a haloalkyl compound of formula (X) following a three-step procedure. In a first step, an acetonitrile compound of formula (XI) can be prepared by treating compound of formula (X) with sodium cyanide in a suitable solvent such as dimethylsulfoxide, at a suitable temperature such as room temperature. The reaction of a compound of formula (XI) with a suitable reducing agent, such as DIBALH, in a suitable solvent such as tetrahydrofuran, at a suitable temperature comprised between 0° C. and room temperature, may give a carbaldehyde of formula (XII) which can be converted to a compound of formula (Ic) through a reductive amination method, such as that described in step 4.

Nitrile derivatives of formula (XI) are also intermediates in the preparation of compounds of formula (Id). In this case, a compound of formula (XIII) can be prepared by hydrolysis of a compound of formula (XI) in a basic medium such as sodium hydroxide solution, in a suitable solvent, such as methanol, at a suitable temperature, preferably the reflux temperature. Then, amide compounds of formula (Id) are obtained by treating a carboxylic acid compound of general formula (XIII) in the acylation conditions described above.

In addition, certain compounds of the present invention can also be obtained by functional group interconversion over compounds of formula (I) or any of the intermediates shown in Scheme 1. The following conversions are examples of transformations that may be carried out:
An amide group may be reduced to alkyl amine using a suitable reducing agent, such as AlH₃, in a suitable solvent, such as tetrahydrofuran, at a suitable temperature, preferably between −10° C. and room temperature.
An amino group can be alkylated using the reductive amination conditions described in step 4. In the specific case of methylation of an amino group in a compound of formula (I) it can be performed using formaldehyde in water in the presence of an acid catalyst such as formic acid, at a suitable temperature, preferably the reflux temperature.

In some of the processes described above it may be necessary to protect the reactive or labile groups present with suitable protecting groups, such as for example acetyl, allyl, Alloc (allyloxycarbonyl), Boc (tert-butoxycarbonyl), or benzyl for the protection of amino groups, and common silyl protecting groups for the protection of the hydroxyl group. The procedures for the introduction and removal of these protecting groups are well known in the art and can be found thoroughly described in the literature.

In addition, a compound of formula (I) can be obtained in enantiopure form by resolution of a racemic mixture either by chiral preparative HPLC or by crystallization of a diastereomeric salt or co-crystal. Alternatively, the resolution step can be carried out at a previous stage, using any suitable intermediate.

The compounds of formula (II), (IV) and (VII) used in the methods disclosed above are commercially available or can be synthesized following common procedures described in the literature and exemplified in the synthesis of the examples.

Turning to another aspect, the invention also relates to the therapeutic use of the compounds of general formula (I). As mentioned above, compounds of general formula (I) show a strong affinity to sigma receptors, especially to sigma-1 and/or sigma-2 receptors and can behave as agonists, antagonists, inverse agonists, partial antagonists or partial agonists thereof. Therefore, compounds of general formula (I) are useful as medicaments.

They are suitable for the treatment and/or prophylaxis of diseases and/or disorders mediated by sigma receptors and preferably by sigma-1 and/or sigma 2 receptors. In this sense, compounds of formula (I) are suitable for the treatment and/or prophylaxis of pain, especially neuropathic pain, inflammatory pain, and chronic pain or other pain conditions involving allodynia and/or hyperalgesia, or CNS disorder or diseases, selected from the group consisting of addiction to drugs and chemical substances including cocaine, amphetamine, ethanol and nicotine, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia or paranoia.

The compounds of general formula (I) are especially suited for the treatment of pain, especially neuropathic pain, inflammatory pain or other pain conditions involving allodynia and/or hyperalgesia or CNS disorder or diseases, selected from the group consisting of addiction to drugs and chemical substances including cocaine, amphetamine, ethanol and nicotine, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia or paranoia.

PAIN is defined by the International Association for the Study of Pain (IASP) as “an unpleasant sensory and emotional experience associated with actual or potential tissue damage, or described in terms of such damage (IASP, Classification of chronic pain, 2nd Edition, IASP Press (2002), 210). Even though pain is always subjective its causes or syndromes can be classified.

In a preferred embodiment compounds of the invention are used for the treatment and/or prophylaxis of allodynia and more specifically mechanical or thermal allodynia.

In another preferred embodiment compounds of the invention are used for the treatment and/or prophylaxis of hyperalgesia.

In yet another preferred embodiment the compounds of the invention are used for the treatment and/or prophylaxis of neuropathic pain and more specifically for the treatment and/or prophylaxis of hyperpathia.

A related aspect of the invention refers to the use of compounds of general formula (I) for the manufacture of a medicament for the treatment and/or prophylaxis of disorders and diseases mediated by sigma receptors and more preferably by sigma-1 receptors and/or sigma-2 receptors, as explained before.

Another related aspect of the invention refers to a method for the treatment and/or prophylaxis of disorders and diseases mediated by sigma receptors and more preferably by sigma-1 receptors and/or sigma-2 receptors, as explained before comprising the administration of a therapeutically effective amount of a compound of general formula (I) to a subject in need thereof.

Another aspect of the invention is a pharmaceutical composition, which comprises at least a compound of general formula (I) or a pharmaceutically acceptable salt, isomer, co-crystal, prodrug or solvate thereof, and at least a pharmaceutically acceptable carrier, additive, adjuvant or vehicle.

The pharmaceutical composition of the invention can be formulated as a medicament in different pharmaceutical forms comprising at least a compound binding to the sigma receptor and optionally at least one further active substance and/or optionally at least one auxiliary substance.

The auxiliary substances or additives can be selected among carriers, excipients, support materials, lubricants, fillers, solvents, diluents, colorants, flavour conditioners such as sugars, antioxidants and/or agglutinants. In the case of suppositories, this may imply waxes or fatty acid esters or preservatives, emulsifiers and/or carriers for parenteral application. The selection of these auxiliary materials and/or additives and the amounts to be used will depend on the form of application of the pharmaceutical composition.

The pharmaceutical composition in accordance with the invention can be adapted to any form of administration, be it orally or parenterally, for example pulmonarily, nasally, rectally and/or intravenously.

Preferably, the composition is suitable for oral or parenteral administration, more preferably for oral, intravenous, intraperitoneal, intramuscular, subcutaneous, intrathecal, rectal, transdermal, transmucosal or nasal administration.

The composition of the invention can be formulated for oral administration in any form preferably selected from the group consisting of tablets, dragees, capsules, pills, chewing gums, powders, drops, gels, juices, syrups, solutions and suspensions. The composition of the present invention for oral administration may also be in the form of multiparticulates, preferably microparticles, microtablets, pellets or granules, optionally compressed into a tablet, filled into a capsule or suspended in a suitable liquid. Suitable liquids are known to those skilled in the art.

Suitable preparations for parenteral applications are solutions, suspensions, reconstitutable dry preparations or sprays.

The compounds of the invention can be formulated as deposits in dissolved form or in patches, for percutaneous application.

Skin applications include ointments, gels, creams, lotions, suspensions or emulsions.

The preferred form of rectal application is by means of suppositories.

In a preferred embodiment, the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

The daily dosage for humans and animals may vary depending on factors that have their basis in the respective species or other factors, such as age, sex, weight or degree of illness and so forth. The daily dosage for humans may preferably be in the range from 1 to 2000, preferably 1 to 1500, more preferably 1 to 1000 milligrams of active substance to be administered during one or several intakes per day.

The following examples are merely illustrative of certain embodiments of the invention and cannot be considered as restricting it in any way.

The following abbreviations are used in the intermediates and examples:
AcOH: acetic acid
Aq: aqueous
Chx: cyclohexane
DIBALH diisobutylaluminium hydride
DMF: N,N-dimethylformamide
EDC·HCl: N-(3-dimethylaminopropyl)-N′-ethylcarbodiimide hydrochloride
Et₂O: diethyl ether
EtOAc: ethyl acetate
EtOH: ethanol
EX: example
h: hours
HOBt: 1-hydroxybenzotriazole
HPLC: High Performance Liquid Chromatography
MeCN: acetonitrile
MeOH: methanol
MS: mass spectrometry
Min.: minutes
Quant: quantitative
Rt.: retention time
rt: room temperature
Sat: saturated
Sol: solution
TFA: trifluoroacetic acid
THF: tetrahydrofuran
Wt: weight

The following methods were used to determine the HPLC-MS spectra:

### Method A

### “Agilent”

Column ZORBAX Extend-C18 RRHD 2.1×50 mm, 1.8 μm, temperature 35° C.; flow rate 0.61 mL/min; A: NH₄HCO₃ 10 mM, B: MeCN; gradient 0.3 min 98% A, 98% A to 100% B in 2.65 min; isocratic 2.05 min 100% B.

### Method B

### “Acquity”

Column ZORBAX Extend-C18 RRHD 2.1×50 mm, 1.8 μm, temperature 35° C.; flow rate 0.61 mL/min; A: NH₄HCO₃ 10 mM, B: MeCN, C: MeOH+0.1% formic acid; gradient 0.3 min 98% A, 98% A to 0:95:5 A:B:C in 2.7 min; 0:95:5 A:B:C to 100% B in 0.1 min; isocratic 2 min 100% B.

### Method C

### “Acquity”

Column UPLC BEH C18 2.1×50 mm, 1.7 μm; flow rate 0.60 mL/min; A: AcONH₄ 10 mM; B: MeCN; gradient: 0.2 min in 90% A, 90% A to 5% A in 3.3 min, isocratic 0.5 min 5% A.

The following method was used for reversed phase semipreparative HPLC purifications: Column XBridge C18 19×50 mm, 5 μm. rt, flow rate 20 mL/min; NH₄HCO₃ 10 mM/MeCN.

### Synthesis of Intermediates

### Intermediate 1. 5-Chloro-1-methyl-3-(5-methylisoxazol-3-yl)-1H-pyrazole-4-carbaldehyde

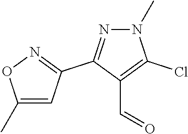

### Step 1. Ethyl 3-(5-methylisoxazol-3-yl)-3-oxopropanoate

To a solution of 5-methylisoxazole-3-carboxylic acid (17.3 g, 136 mmol) in THE (180 mL) CDI (26.5 g, 164 mmol) was gradually added at rt. To the white suspension, THE (180 mL) was added to facilitate strong stirring. After 1.5 h the gold solution was treated with MgCl₂ (13.6 g, 143 mmol) and ethyl potassium malonate (24.4 g, 143 mmol) and the suspension was stirred at rt overnight. The creamy orange suspension was slowly acidified to pH 4 with 6 M HCl solution. The volatiles were removed under reduced pressure and the residue extracted with CH₂Cl₂. The combined organic layers were dried over Na₂SO₄, filtered and concentrated to dryness to give the tittle compound (24.5 g, 92% yield) as a yellow liquid.

HPLC Rt (Method A): 1.66 min; ESI+−MS m/z: 198 (M+H)⁺.

¹H NMR (400 MHz, CD₃OD) δ (keto-form) 6.49 (q, J=0.9 Hz, 1H), 4.18 (q, J=7.1 Hz, 2H), 4.02 (s, 2H), 2.49 (d, J=0.9 Hz, 3H), 1.24 (t, J=7.1 Hz, 3H); (enol-form) 6.48 (q, J=0.9 Hz, 1H), 5.83 (s, 1H), 4.28 (q, J=7.1 Hz, 2H), 2.47 (d, J=0.9 Hz, 3H), 1.32 (t, J=7.1 Hz, 3H).

### Step 2. 1-Methyl-3-(5-methylisoxazol-3-yl)-1H-pyrazol-5-ol

To a solution of the compound obtained in Step 1 (27.5 g, 139 mmol) in EtOH (90 mL) methyl hydrazine (7.34 mL, 139 mmol) and conc. HCl (37%, 4.7 mL, 153 mmol) were added. The reaction mixture was heated at 80° C. for 2 h. The mixture was then cooled to rt and stirred overnight, after which a solid appeared. The suspension was cooled to 0° C. and the precipitate filtered. The solid was washed with cold EtOH and dried under reduced pressure to give the title compound (12.0 g, 48% yield).

HPLC Rt (Method A): 0.24, 0.47 min; ESI+−MS m/z: 180 (M+H)⁺.

¹H NMR (400 MHz, CDCl₃) δ 6.38 (q, J=0.9 Hz, 1H), 3.67 (s, 2H), 3.41 (s, 3H), 2.48 (d, J=0.9 Hz, 3H).

### Step 3. Title Compound

Phosphorous oxychloride (18.4 mL, 201 mmol) was added dropwise to DMF (5.19 mL, 67.0 mmol) at 5° C. with ice-cooling under argon. The pale pink solution was stirred at 0° C. for 10 min and then allowed to stir at rt for 10 min. Then, a solution of the compound obtained in Step 2 (6.0 g, 33.5 mmol) in DMF (80 mL) was added at rt under argon (exothermic). The resulting yellow solution was heated at 120° C. for 1 h. After cooling back to rt, the reaction was quenched with water and basified with Na₂CO₃ solution to pH 9-10. A solid appeared which was washed several times with water and finally dried under reduced pressure to give the title compound (3.89 g, 51% yield) as beige solid.

HPLC Rt (Method A): 1.62 min; ESI+−MS m/z: 226 (M+H)⁺.

¹H NMR (400 MHz, CD₃OD) δ 10.29 (s, 1H), 6.58 (q, J=0.9 Hz, 1H), 3.95 (s, 3H), 2.50 (d, J=1.0 Hz, 3H).

This method was used for the preparation of Intermediates 2-16 using suitable starting materials:

| INT | STRUCTURE | CHEMICAL NAME |
|---|---|---|
| 2 | 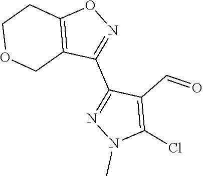 [SMILES: Cn1nc(-c2noc3c2COCC3)c(C=O)c1Cl] | 5-Chloro-3-(6,7-dihydro-4H- pyrano[3,4-d]isoxazol-3-yl)-1- methyl-1H-pyrazole-4- carbaldehyde |
| 3 | 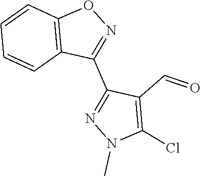 [SMILES: Cn1nc(-c2noc3ccccc23)c(C=O)c1Cl] | 3-(Benzo[d]isoxazol-3-yl)-5- chloro-1-methyl-1H-pyrazole-4- carbaldehyde |
| 4 | 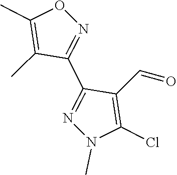 [SMILES: Cc1onc(-c2nn(C)c(Cl)c2C=O)c1C] | 5-Chloro-3-(4,5- dimethylisoxazol-3-yl)-1-methyl- 1H-pyrazole-4-carbaldehyde |
| 5 | 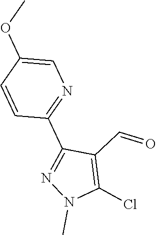 [SMILES: COc1ccc(-c2nn(C)c(Cl)c2C=O)nc1] | 5-Chloro-3-(5-methoxypyridin- 2-yl)-1-methyl-1H-pyrazole-4- carbaldehyde |
| 6 | 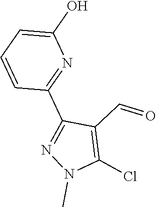 [SMILES: Cn1nc(-c2cccc(O)n2)c(C=O)c1Cl] | 5-Chloro-3-(6-hydroxypyridin-2- yl)-1-methyl-1H-pyrazole-4- carbaldehyde |
| 7 | 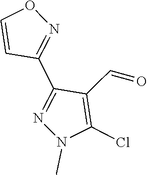 | 5-Chloro-3-(isoxazol-3-yl)-1- methyl-1H-pyrazole-4- carbaldehyde |
| 8 | 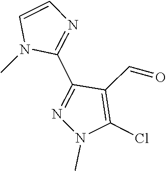 | 5-Chloro-1-methyl-3-(1-methyl- 1H-imidazol-2-yl)-1H-pyrazole- 4-carbaldehyde |
| 9 | 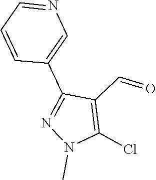 | 5-Chloro-1-methyl-3-(pyridin-3- yl)-1H-pyrazole-4-carbaldehyde |
| 10 | 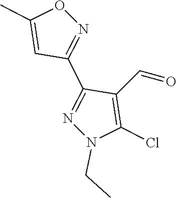 [SMILES: CCn1nc(-c2cc(C)on2)c(C=O)c1Cl] | 5-Chloro-1-ethyl-3-(5- methylisoxazol-3-yl)-1H- pyrazole-4-carbaldehyde |
| 11 | 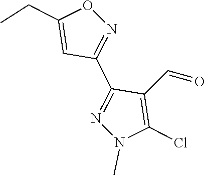 [SMILES: CCc1cc(-c2nn(C)c(Cl)c2C=O)no1] | 5-Chloro-3-(5-ethylisoxazol-3- yl)-1-methyl-1H-pyrazole-4- carbaldehyde |
| 12 | 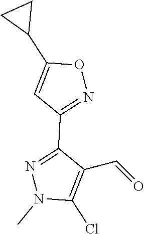 [SMILES: Cn1nc(-c2cc(C3CC3)on2)c(C=O)c1Cl] | 5-Chloro-3-(5- cyclopropylisoxazol-3-yl)-1- methyl-1H-pyrazole-4- carbaldehyde |
| 13 | 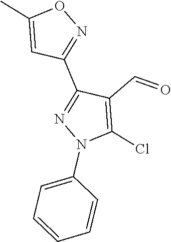 [SMILES: Cc1cc(-c2nn(-c3ccccc3)c(Cl)c2C=O)no1] | 5-Chloro-3-(5-methylisoxazol-3- yl)-1-phenyl-1H-pyrazole-4- carbaldehyde |
| 14 | 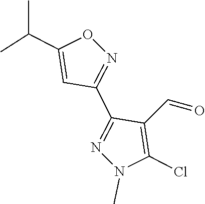 [SMILES: CC(C)c1cc(-c2nn(C)c(Cl)c2C=O)no1] | 5-Chloro-3-(5- isopropylisoxazol-3-yl)-1- methyl-1H-pyrazole-4- carbaldehyde |
| 15 | 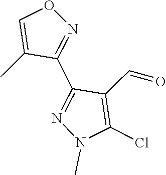 | 5-Chloro-1-methyl-3-(4- methylisoxazol-3-yl)-1H- pyrazole-4-carbaldehyde |
| 16 | 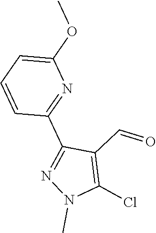 [SMILES: COc1cccc(-c2nn(C)c(Cl)c2C=O)n1] | 5-Chloro-3-(6-methoxypyridin- 2-yl)-1-methyl-1H-pyrazole-4- carbaldehyde |

### Intermediate 17. 5-Fluoro-1-methyl-3-(5-methylisoxazol-3-yl)-1H-pyrazole-4-carbaldehyde

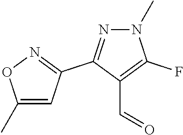

Intermediate 1 (300 mg, 1.33 mmol) was dissolved in DMF under argon in a MW tube and KF (232 mg, 3.99 mmol) was added. The reaction mixture was microwave irradiated at 1 h at 150° C. The reaction mixture was cooled to rt and the volatiles removed under reduced pressure. The residue was purified by flash chromatography (silica gel, Chx/EtOAc) to give the title compound (70 mg, 25% yield).

HPLC Rt (Method A): 1.52 min; ESI+−MS m/z: 210.0 (M+H)⁺.

### Intermediate 18. (S)—N-Isopentylazepan-3-amine

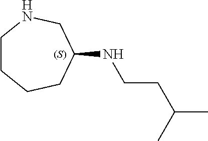

### Step 1. (S)-tert-Butyl 3-(isopentylamino)azepane-1-carboxylate

To a solution of (S)-tert-butyl 3-aminoazepane-1-carboxylate (300 mg, 1.40 mmol) in MeOH (15 mL), 3-methylbutanal (150 μL, 1.40 mmol) was added followed by NaBH₃CN (132 mg, 2.10 mmol). The reaction mixture was stirred at rt for 30 min. The solvent was removed under reduced pressure and the residue was partitioned between water and CH₂Cl₂. The aqueous phase was additionally extracted with CH₂Cl₂ and the combined organic layers were dried over Na₂SO₄, filtered and concentrated to dryness to give the title compound (679 mg, overweight), which was used in the next step without further purification.

HPLC Rt (Method B): 1.99 min; ESI+−MS m/z: 285.3 (M+H)⁺.

### Step 2. Title Compound

The compound obtained in Step 1 (400 mg, 1.41 mmol) was dissolved in CH₂Cl₂ (15 mL), TFA (2.15 mL, 28.1 mmol) was added and the mixture was stirred at rt overnight. All volatiles were removed under reduced pressure. The residue (TFA salt) was purified by SCX chromatography (SCX, Agilent bond column). The column was washed with MeOH and then the TFA salt, dissolved in minimal amount of MeOH, loaded onto the SCX column. The column was first eluted with methanol and then with 2 N NH₃ in MeOH to give the title compound (145 mg, 56% yield).

HPLC Rt (Method B): 1.06 min; ESI+−MS m/z: 185.2 (M+H)⁺.

This method was used for the preparation of Intermediate 19 using the opposite enantiomer

| INT | STRUCTURE | CHEMICAL NAME |
|---|---|---|
| 19 | 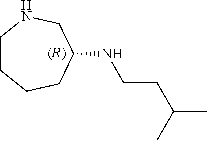 | (R)-N-Isopentylazepan-3-amine |

### Intermediate 20. 3-(Benzyl(isopentyl)amino)azepan-2-one

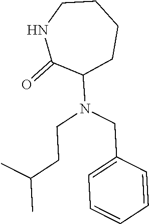 [SMILES: CC(C)CCN(Cc1ccccc1)C1CCCCNC1=O]

### Step 1. 3-(Isopentylamino)azepan-2-one

To a solution of 3-aminoazepan-2-one (300 mg, 2.34 mmol) in MeOH (15 mL), 3-methylbutanal (251 μL, 2.34 mmol) was added followed by NaBH₃CN (221 mg, 3.51 mmol). The reaction mixture was stirred at rt for 30 min. The solvent was removed under reduced pressure and the residue was partitioned between water and CH₂Cl₂. The aqueous phase was additionally extracted with CH₂Cl₂ and the combined organic layers were dried over Na₂SO₄, filtered and concentrated to dryness to give the title compound (433 mg, 93% yield).

HPLC Rt (Method B): 1.19 min; ESI+−MS m/z: 199.2 (M+H)⁺.

### Step 2. Title Compound

The compound obtained in Step 1 (433 mg, 2.18 mmol) was dissolved in MeCN (15 mL) and potassium carbonate (905 mg, 6.55 mmol) was added, followed by benzyl chloride (755 μL, 6.55 mmol). The reaction was heated at 70° C. overnight and then quenched with sat. aq. NaHCO₃ solution. The mixture was extracted with EtOAc and the combined organic layers were dried over Na₂SO₄, filtered and concentrated. The residue was purified by flash chromatography (silica gel, Chx/EtOAc) to give the title compound (293 mg, 47% yield).

HPLC Rt (Method B): 2.44 min; ESI+−MS m/z: 298.2 (M+H)⁺.

### Intermediate 21. 4-(Benzyl(isopentyl)amino)azepan-2-one

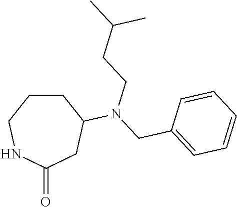 [SMILES: CC(C)CCN(Cc1ccccc1)C1CCCNC(=O)C1]

### Step 1. N-Benzyl-3-methylbutan-1-amine

To a solution of benzylamine (5.1 mL, 46.7 mmol) in MeOH (85 mL), 3-methylbutanal (5 mL, 46.7 mmol) was added followed by NaBH₃CN (4.4 g, 70 mmol). The reaction mixture was stirred at rt for 30 min. The solvent was removed under reduced pressure. The residue was partitioned between water and CH₂Cl₂. The aqueous phase was additionally extracted with CH₂Cl₂. The combined organic layers were dried over Na₂SO₄, filtered and concentrated. The residue was purified by flash chromatography (silica gel, Chx/EtOAc) to give the title compound (2.6 g, 31% yield).

HPLC Rt (Method A): 1.62 min; ESI+−MS m/z: 178.0 (M+H)⁺.

### Step 2. 3-(Benzyl(isopentyl)amino)cyclohexanone

To the compound obtained in Step 1 (710 mg, 4.01 mmol) and cyclohex-2-enone (351 μL, 3.64 mmol) in CH₂Cl₂ (5 mL), bismuth nitrate (350 mg, 3.64 mmol) was added. Then the CH₂Cl₂ was removed with a nitrogen current and the reaction mixture was stirred at rt overnight. CH₂Cl₂ (500 mL) was added and the mixture was filtered. The filtrate was washed with sat. aq. NaHCO₃ solution (2×), dried over Na₂SO₄, filtered and concentrated to dryness to give the title compound (873 mg, 88% yield).

HPLC Rt (Method A): 2.68 min; ESI+−MS m/z: 274.2 (M+H)⁺.

### Step 3. (Z)-3-(Benzyl(isopentyl)amino)cyclohexanone oxime

To the compound obtained in Step 2 (873 mg, 391 mmol) dissolved in ethanol (30 mL) hydroxylamine hydrochloride (488 mg, 7.03 mmol) and sodium acetate (655 mg, 7.98 mmol) were added. The resulting mixture was stirred for 1 h at rt and then quenched with water. The mixture was extracted with CH₂Cl₂ and the combined organic layers were dried over Na₂SO₄, filtered and concentrated to give the title compound (1.1 g, quant.).

HPLC Rt (Method A): 2.55 min; ESI+−MS m/z: 298.0 (M+H)⁺.

### Step 4. Title Compound

2,4,6-Trichloro-1,3,5-triazine (648 mg, 3.51 mmol) was stirred for 1 h in DMF (10 mL) and the compound obtained in Step 3 (921 mg, 3.19 mmol) was added at 0° C. The reaction mixture was stirred at rt overnight and then quenched with sat. aq. NaHCO₃ solution. The mixture was extracted with CH₂Cl₂ and the combined organic layers were dried over Na₂SO₄, filtered and concentrated. The residue was purified by flash chromatography (silica gel, Chx/EtOAc) to give the title compound (60 mg, 7% yield).

HPLC Rt (Method A): 2.31 min; ESI+−MS m/z: 289.0 (M+H)⁺.

### Intermediates 22-42

The following intermediates were synthesized following the method described in intermediate 1 using suitable starting materials:

| INT | STRUCTURE | CHEMICAL NAME |
|---|---|---|
| 22 | 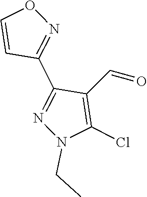 | 5-Chloro-1-ethyl-3- (isoxazol-3- yl)-1H-pyrazole- 4-carbaldehyde |
| 23 | 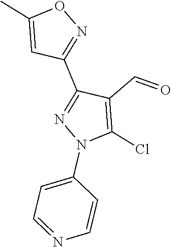 [SMILES: Cc1cc(-c2nn(-c3ccncc3)c(Cl)c2C=O)no1] | 5-Chloro-3- (5-methylisoxazol-3- yl)-1-(pyridin-4-yl)-1H- pyrazole- 4-carbaldehyde |
| 24 | 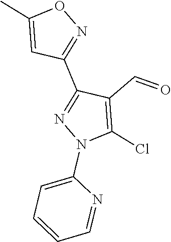 [SMILES: Cc1cc(-c2nn(-c3ccccn3)c(Cl)c2C=O)no1] | 5-Chloro-3-(5- methylisoxazol-3- yl)-1-(pyridin-2-yl)- 1H-pyrazole- 4-carbaldehyde |
| 25 | 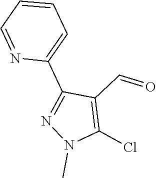 | 5-Chloro-1-methyl-3- (pyridin-2- yl)-1H-pyrazole-4- carbaldehyde |
| 26 | 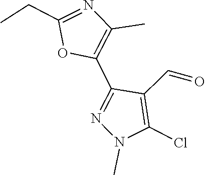 [SMILES: CCc1nc(C)c(-c2nn(C)c(Cl)c2C=O)o1] | 5-Chloro-3-(2-ethyl-4- methyloxazol-5-yl)-1- methyl-1H-pyrazole- 4-carbaldehyde |
| 27 | 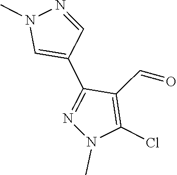 | 5-Chloro-1,1′-dimethyl- 1H,1′H- [3,4′-bipyrazole]-4- carbaldehyde |
| 28 | 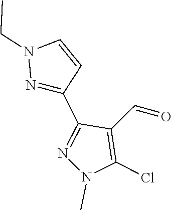 [SMILES: CCn1ccc(-c2nn(C)c(Cl)c2C=O)n1] | 5-Chloro-1′-ethyl-1- methyl-1H,1′H- [3,3′-bipyrazole]-4- carbaldehyde |
| 29 | 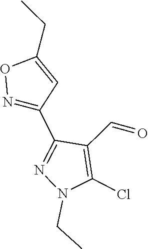 [SMILES: CCc1cc(-c2nn(CC)c(Cl)c2C=O)no1] | 5-Chloro-1-ethyl- 3-(5-ethylisoxazol- 3-yl)-1H-pyrazole- 4-carbaldehyde |
| 30 | 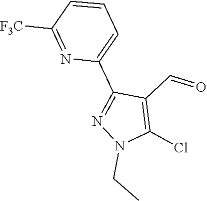 [SMILES: CCn1nc(-c2cccc(C(F)(F)F)n2)c(C=O)c1Cl] | 5-Chloro-1-ethyl- 3-(6-(trifluoromethyl) pyridin-2-yl)-1H- pyrazole-4-carbaldehyde |
| 31 | 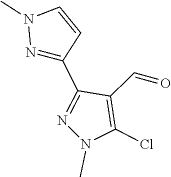 | 5-Chloro-1,1′-dimethyl- 1H,1′H- [3,3′-bipyrazole]-4- carbaldehyde |
| 32 | 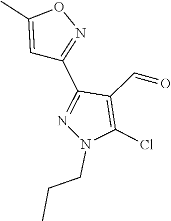 [SMILES: CCCn1nc(-c2cc(C)on2)c(C=O)c1Cl] | 5-Chloro-3- (5-methylisoxazol-3- yl)-1-propyl-1H- pyrazole-4- carbaldehyde |
| 33 | 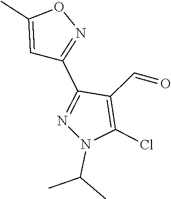 [SMILES: Cc1cc(-c2nn(C(C)C)c(Cl)c2C=O)no1] | 5-Chloro-1-isopropyl- 3-(5-methylisoxazol- 3-yl)-1H- pyrazole-4-carbaldehyde |
| 34 | 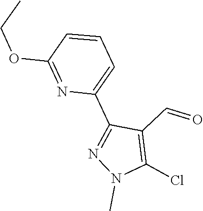 [SMILES: CCOc1cccc(-c2nn(C)c(Cl)c2C=O)n1] | 5-Chloro-3-(6- ethoxypyridin-2- yl)-1-methyl-1H- pyrazole-4- carbaldehyde |
| 35 | 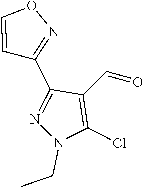 | 5-Chloro-1-ethyl-3- (isoxazol-3- yl)-1H-pyrazole-4- carbaldehyde |
| 36 | 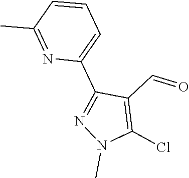 [SMILES: Cc1cccc(-c2nn(C)c(Cl)c2C=O)n1] | 5-Chloro-1-methyl- 3-(6-methylpyridin- 2-yl)-1H- pyrazole-4- carbaldehyde |
| 37 | 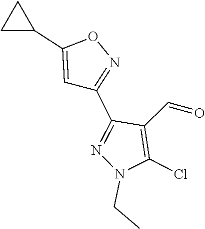 [SMILES: CCn1nc(-c2cc(C3CC3)on2)c(C=O)c1Cl] | 5-Chloro-3-(5- cyclopropylisoxazol- 3-yl)-1-ethyl-1H- pyrazole-4- carbaldehyde |
| 38 | 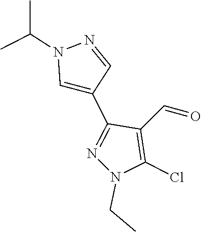 [SMILES: CCn1nc(-c2cnn(C(C)C)c2)c(C=O)c1Cl] | 5-Chloro-1-ethyl-1′- isopropyl-1H,1′H- [3,4′-bipyrazole]-4- carbaldehyde |
| 39 | 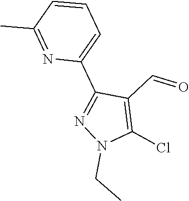 [SMILES: CCn1nc(-c2cccc(C)n2)c(C=O)c1Cl] | 5-Chloro-1-ethyl-3-(6- methylpyridin-2-yl)-1H- pyrazole-4-carbaldehyde |
| 40 | 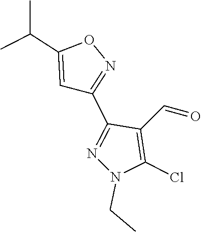 [SMILES: CCn1nc(-c2cc(C(C)C)on2)c(C=O)c1Cl] | 5-Chloro-1-ethyl-3-(5- isopropylisoxazol-3-yl)-1H- pyrazole-4-carbaldehyde |
| 41 | 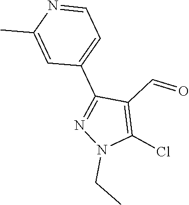 [SMILES: CCn1nc(-c2ccnc(C)c2)c(C=O)c1Cl] | 5-Chloro-1-ethyl-3-(2- methylpyridin-4-yl)-1H- pyrazole-4-carbaldehyde |
| 42 | 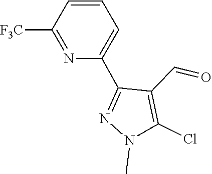 [SMILES: Cn1nc(-c2cccc(C(F)(F)F)n2)c(C=O)c1Cl] | 5-Chloro-1-methyl-3-(6- (trifluoromethyl) pyridin-2-yl)-1H- pyrazole-4-carbaldehyde |

### Intermediates 43-48

The following intermediates were synthesized following the method described in intermediate 18 using suitable starting materials:

| INT | STRUCTURE | CHEMICAL NAME |
|---|---|---|
| 43 | 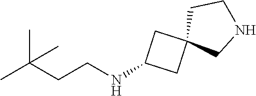 | (2s,4r)-N-(3,3-Dimethylbutyl)-6- azaspiro[3.4]octan-2-amine |
| 44 | 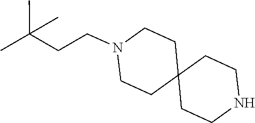 [SMILES: CC(C)(C)CCN1CCC2(CCNCC2)CC1] | 3-(3,3-Dimethylbutyl)-3,9- diazaspiro[5.5]undecane |
| 45 | 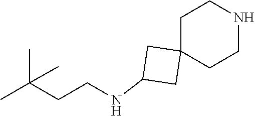 [SMILES: CC(C)(C)CCNC1CC2(CCNCC2)C1] | N-(3,3-Dimethylbutyl)-7- azaspiro[3.5]nonan-2-amine |
| 46 | 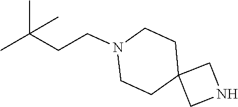 | 7-(3,3-Dimethylbutyl)-2,7- diazaspiro[3.5]nonane |
| 47 | 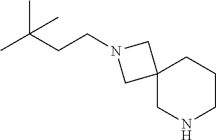 | 2-(3,3-Dimethylbutyl)-2,6- diazaspiro[3.5]nonane |
| 48 | 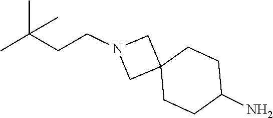 [SMILES: CC(C)(C)CCN1CC2(CCC(N)CC2)C1] | 2-(3,3-Dimethylbutyl)-2- azaspiro[3.5]nonan-7-amine |

### SYNTHESIS OF EXAMPLES

### Example 1. N-((1-((5-Chloro-1-methyl-3-(5-methylisoxazol-3-yl)-1H-pyrazol-4-yl)methyl)pyrrolidin-3-yl)methyl)-3,3-dimethylbutan-1-amine

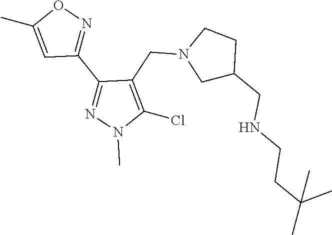 [SMILES: Cc1cc(-c2nn(C)c(Cl)c2CN2CCC(CNCCC(C)(C)C)C2)no1]

### Step 1. tert-Butyl ((1-((5-chloro-1-methyl-3-(5-methylisoxazol-3-yl)-1H-pyrazol-4-yl)methyl)pyrrolidin-3-yl)methyl)carbamate

5-Chloro-1-methyl-3-(5-methylisoxazol-3-yl)-1H-pyrazole-4-carbaldehyde (intermediate 1, 282 mg, 1.25 mmol) was dissolved in 1,2-dichloroethane (15 mL) and tert-butyl (pyrrolidin-3-ylmethyl)carbamate (300 mg, 1.50 mmol) was added, followed by sodium triacetoxyborohydride (526 mg, 2.50 mmol). The reaction mixture was stirred at 120° C. for 1.5 h. All volatiles were removed under reduced pressure. The residue was partitioned between EtOAc and sat. aq. NaHCO₃ solution and the aqueous layer was extracted with EtOAc. The combined organic layers were dried over Na₂SO₄, filtered and concentrated to dryness to give the title compound (512 mg, quant.). The crude product was used in the next step without further purification.

HPLC Rt (Method B): 1.97 min; ESI+−MS m/z: 410 (M+H)⁺.

### Step 2. (1-((5-Chloro-1-methyl-3-(5-methylisoxazol-3-yl)-1H-pyrazol-4-yl)methyl)pyrrolidin-3-yl)methanamine

The compound obtained in Step 1 (512 mg, 1.25 mmol) was dissolved in CH₂Cl₂ (12 mL) and the reaction solution cooled to 0° C. At this temperature TFA (1.91 mL, 25.0 mmol) was added. The resulting solution was slowly allowed to reach rt and stirred overnight. Water was added and the organic phase separated. Then the aqueous phase was basified and extracted with CH₂Cl₂ (2×). The combined organic layers were dried over Na₂SO₄, filtered and concentrated to dryness to give the title compound (266 mg, 69% yield).

HPLC Rt (Method B): 1.22 min; ESI+−MS m/z: 310 (M+H)⁺.

### Step 3. Title Compound

To a solution of the compound obtained in Step 2 (450 mg, 1.45 mmol) in MeOH (40 mL) 3,3-dimethylbutanal (182 μL, 1.45 mmol) was added followed by NaBH₃CN (183 mg, 91.8 mmol) and the resulting mixture was stirred at room temperature for 45 min. All volatiles were removed under reduced pressure. The residue was first purified by an acid work and the acidic phase extracted with EtOAc to remove impurities. Subsequently the aqueous phase was basified and extracted with EtOAc. The combined organic layers were dried over Na₂SO₄, filtered and concentrated to dryness to give the title compound (122 mg, 21% yield)

HPLC Rt (Method B): 1.83 min; ESI+−MS m/z: 394.3 (M+H)⁺.

This method was used for the preparation of examples 2-68 using suitable starting materials (and intermediates 1-19):

| | | | Rt | MS | HPLC |
|---|---|---|---|---|---|
| STRUCTURE | EX | CHEMICAL NAME | (min) | (M + H) | Method |
| 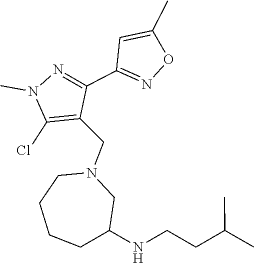 [SMILES: Cc1cc(-c2nn(C)c(Cl)c2CN2CCCCC(NCCC(C)C)C2)no1] | 2 | 1-((5-Chloro-1-methyl-3- (5-methylisoxazol-3-yl)- 1H-pyrazol-4-yl)methyl)-N- isopentylazepan-3-amine | 2.03 | 394.3 | B |
| 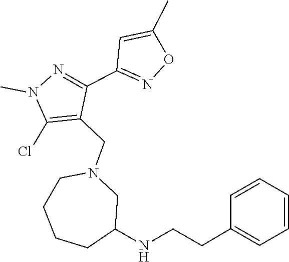 [SMILES: Cc1cc(-c2nn(C)c(Cl)c2CN2CCCCC(NCCc3ccccc3)C2)no1] | 3 | 1-((5-Chloro-1-methyl-3- (5-methylisoxazol-3-yl)- 1H-pyrazol-4-yl)methyl)-N- phenethylazepan-3-amine | 2.13 | 428.3 | B |
| 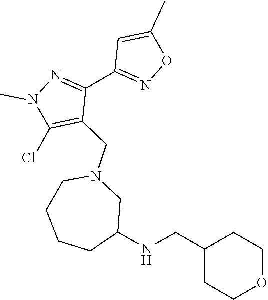 [SMILES: Cc1cc(-c2nn(C)c(Cl)c2CN2CCCCC(NCC3CCOCC3)C2)no1] | 4 | 1-((5-Chloro-1-methyl-3- (5-methylisoxazol-3-yl)- 1H-pyrazol-4-yl)methyl)-N- ((tetrahydro-2H-pyran-4- yl)methyl)azepan-3-amine | 1.77 | 422.3 | B |
| 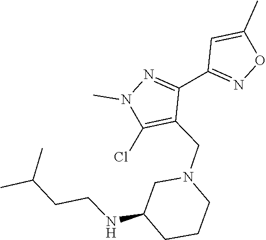 [SMILES: Cc1cc(-c2nn(C)c(Cl)c2CN2CCC[C@@H](NCCC(C)C)C2)no1] | 5 | (R)-1-((5-Chloro-1-methyl- 3-(5-methylisoxazol-3-yl)- 1H-pyrazol-4-yl)methyl)-N- isopentylpiperidin-3-amine | 1.90 | 380.2 | B |
| 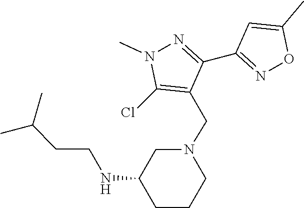 [SMILES: Cc1cc(-c2nn(C)c(Cl)c2CN2CCC[C@H](NCCC(C)C)C2)no1] | 6 | (S)-1-((5-Chloro-1-methyl- 3-(5-methylisoxazol-3-yl)- 1H-pyrazol-4-yl)methyl)-N- isopentylpiperidin-3-amine | 1.91 | 380.2 | B |
| 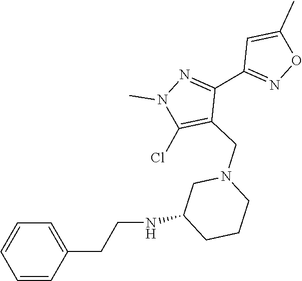 [SMILES: Cc1cc(-c2nn(C)c(Cl)c2CN2CCC[C@H](NCCc3ccccc3)C2)no1] | 7 | (S)-1-((5-Chloro-1-methyl- 3-(5-methylisoxazol-3-yl)- 1H-pyrazol-4-yl)methyl)-N- phenethylpiperidin-3- amine | 2.06 | 414.2 | B |
| 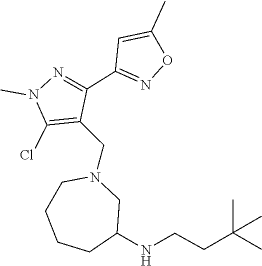 [SMILES: Cc1cc(-c2nn(C)c(Cl)c2CN2CCCCC(NCCC(C)(C)C)C2)no1] | 8 | 1-((5-Chloro-1-methyl-3- (5-methylisoxazol-3-yl)- 1H-pyrazol-4-yl)methyl)-N- (3,3-dimethylbutyl)azepan- 3-amine | 2.02 | 408.3 | B |
| 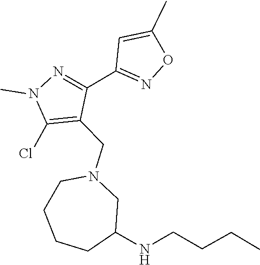 [SMILES: CCCCNC1CCCCN(Cc2c(-c3cc(C)on3)nn(C)c2Cl)C1] | 9 | N-Butyl-1-((5-chloro-1- methyl-3-(5- methylisoxazol-3-yl)-1H- pyrazol-4- yl)methyl)azepan-3-amine | 1.81 | 380.2 | B |
| 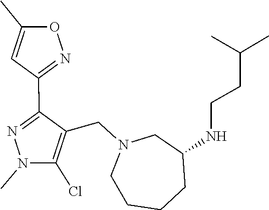 [SMILES: Cc1cc(-c2nn(C)c(Cl)c2CN2CCCC[C@@H](NCCC(C)C)C2)no1] | 10 | (R)-1-((5-Chloro-1-methyl- 3-(5-methylisoxazol-3-yl)- 1H-pyrazol-4-yl)methyl)-N- isopentylazepan-3-amine | 2.04 | 394.3 | B |
| 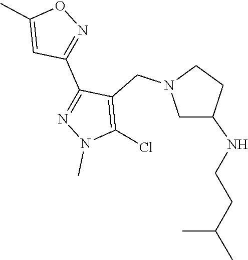 [SMILES: Cc1cc(-c2nn(C)c(Cl)c2CN2CCC(NCCC(C)C)C2)no1] | 11 | 1-((5-Chloro-1-methyl-3- (5-methylisoxazol-3-yl)- 1H-pyrazol-4-yl)methyl)-N- isopentylpyrrolidin-3- amine | 1.69 | 366.2 | B |
| 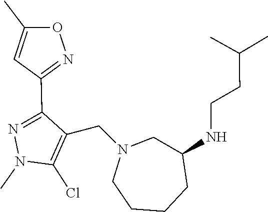 [SMILES: Cc1cc(-c2nn(C)c(Cl)c2CN2CCCC[C@H](NCCC(C)C)C2)no1] | 12 | (S)-1-((5-Chloro-1-methyl- 3-(5-methylisoxazol-3-yl)- 1H-pyrazol-4-yl)methyl)-N- isopentylazepan-3-amine | 1.99 | 394.3 | B |
| 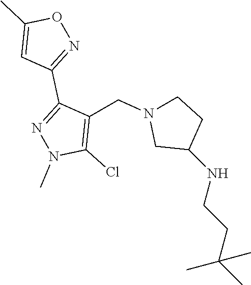 [SMILES: Cc1cc(-c2nn(C)c(Cl)c2CN2CCC(NCCC(C)(C)C)C2)no1] | 13 | 1-((5-Chloro-1-methyl-3- (5-methylisoxazol-3-yl)- 1H-pyrazol-4-yl)methyl)-N- (3,3- dimethylbutyl)pyrrolidin-3- amine | 1.80 | 380.2 | B |
| 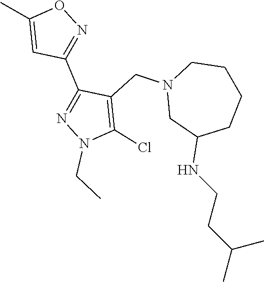 [SMILES: CCn1nc(-c2cc(C)on2)c(CN2CCCCC(NCCC(C)C)C2)c1Cl] | 14 | 1-((5-Chloro-1-ethyl-3-(5- methylisoxazol-3-yl)-1H- pyrazol-4-yl)methyl)-N- isopentylazepan-3-amine | 2.19 | 408.3 | B |
| 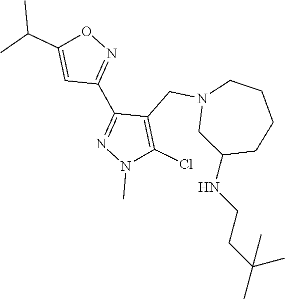 [SMILES: CC(C)c1cc(-c2nn(C)c(Cl)c2CN2CCCCC(NCCC(C)(C)C)C2)no1] | 15 | 1-((5-Chloro-3-(5- isopropylisoxazol-3-yl)-1- methyl-1H-pyrazol-4- yl)methyl)-N-(3,3- dimethylbutyl)azepan-3- amine | 2.41 | 436.3 | B |
| 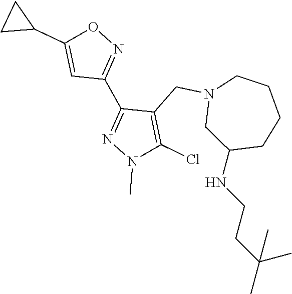 [SMILES: Cn1nc(-c2cc(C3CC3)on2)c(CN2CCCCC(NCCC(C)(C)C)C2)c1Cl] | 16 | 1-((5-Chloro-3-(5- cyclopropylisoxazol-3-yl)- 1-methyl-1H-pyrazol-4- yl)methyl)-N-(3,3- dimethylbutyl)azepan-3- amine | 2.32 | 434.3 | B |
| 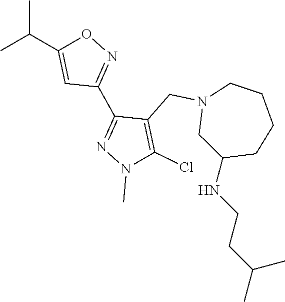 [SMILES: CC(C)CCNC1CCCCN(Cc2c(-c3cc(C(C)C)on3)nn(C)c2Cl)C1] | 17 | 1-((5-Chloro-3-(5- isopropylisoxazol-3-yl)-1- methyl-1H-pyrazol-4- yl)methyl)-N- isopentylazepan-3-amine | 2.34 | 422.3 | B |
| 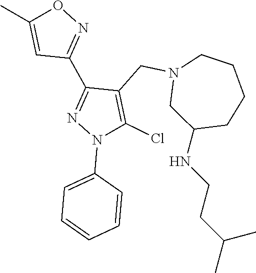 [SMILES: Cc1cc(-c2nn(-c3ccccc3)c(Cl)c2CN2CCCCC(NCCC(C)C)C2)no1] | 18 | 1-((5-Chloro-3-(5- methylisoxazol-3-yl)-1- phenyl-1H-pyrazol-4- yl)methyl)-N- isopentylazepan-3-amine | 2.43 | 456.3 | B |
| 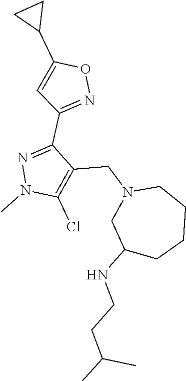 [SMILES: CC(C)CCNC1CCCCN(Cc2c(-c3cc(C4CC4)on3)nn(C)c2Cl)C1] | 19 | 1-((5-Chloro-3-(5- cyclopropylisoxazol-3-yl)- 1-methyl-1H-pyrazol-4- yl)methyl)-N- isopentylazepan-3-amine | 2.19 | 420.3 | B |
| 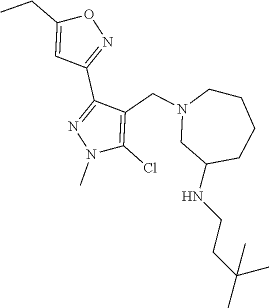 [SMILES: CCc1cc(-c2nn(C)c(Cl)c2CN2CCCCC(NCCC(C)(C)C)C2)no1] | 20 | 1-((5-Chloro-3-(5- ethylisoxazol-3-yl)-1- methyl-1H-pyrazol-4- yl)methyl)-N-(3,3- dimethylbutyl)azepan-3- amine | 2.32 | 422.3 | B |
| 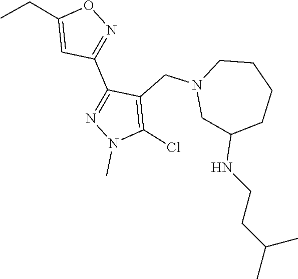 [SMILES: CCc1cc(-c2nn(C)c(Cl)c2CN2CCCCC(NCCC(C)C)C2)no1] | 21 | 1-((5-Chloro-3-(5- ethylisoxazol-3-yl)-1- methyl-1H-pyrazol-4- yl)methyl)-N- isopentylazepan-3-amine | 2.21 | 408.3 | B |
| 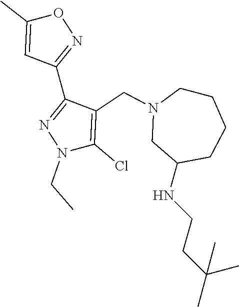 [SMILES: CCn1nc(-c2cc(C)on2)c(CN2CCCCC(NCCC(C)(C)C)C2)c1Cl] | 22 | 1-((5-Chloro-1-ethyl-3-(5- methylisoxazol-3-yl)-1H- pyrazol-4-yl)methyl)-N- (3,3-dimethylbutyl)azepan- 3-amine | 2.32 | 422.3 | B |
| 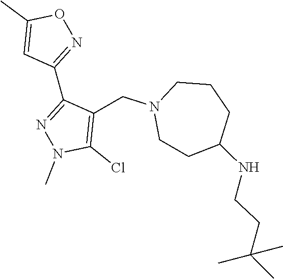 [SMILES: Cc1cc(-c2nn(C)c(Cl)c2CN2CCCC(NCCC(C)(C)C)CC2)no1] | 23 | 1-((5-Chloro-1-methyl-3- (5-methylisoxazol-3-yl)- 1H-pyrazol-4-yl)methyl)-N- (3,3-dimethylbutyl)azepan- 4-amine | 2.03 | 408.3 | B |
| 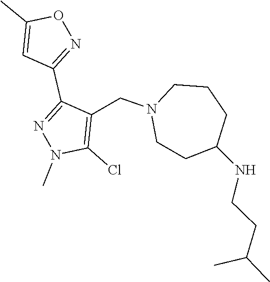 [SMILES: Cc1cc(-c2nn(C)c(Cl)c2CN2CCCC(NCCC(C)C)CC2)no1] | 24 | 1-((5-Chloro-1-methyl-3- (5-methylisoxazol-3-yl)- 1H-pyrazol-4-yl)methyl)-N- isopentylazepan-4-amine | 1.91 | 394.2 | B |
| 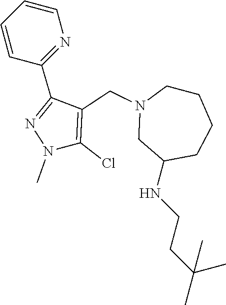 [SMILES: Cn1nc(-c2ccccn2)c(CN2CCCCC(NCCC(C)(C)C)C2)c1Cl] | 25 | 1-((5-Chloro-1-methyl-3- (pyridin-2-yl)-1H-pyrazol- 4-yl)methyl)-N-(3,3- dimethylbutyl)azepan-3- amine | 1.91 | 404.3 | B |
| 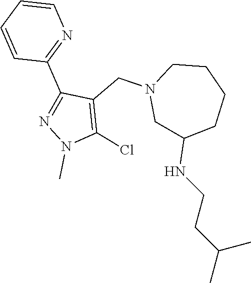 [SMILES: CC(C)CCNC1CCCCN(Cc2c(-c3ccccn3)nn(C)c2Cl)C1] | 26 | 1-((5-Chloro-1-methyl-3- (pyridin-2-yl)-1H-pyrazol- 4-yl)methyl)-N- isopentylazepan-3-amine | 1.81 | 390.3 | B |
| 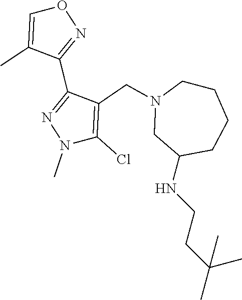 [SMILES: Cc1conc1-c1nn(C)c(Cl)c1CN1CCCCC(NCCC(C)(C)C)C1] | 27 | 1-((5-Chloro-1-methyl-3- (4-methylisoxazol-3-yl)- 1H-pyrazol-4-yl)methyl)-N- (3,3-dimethylbutyl)azepan- 3-amine | 2.21 | 408.2 | A |
| 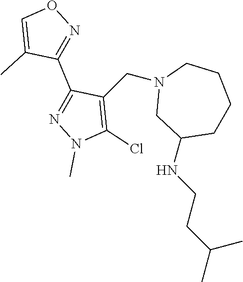 [SMILES: Cc1conc1-c1nn(C)c(Cl)c1CN1CCCCC(NCCC(C)C)C1] | 28 | 1-((5-Chloro-1-methyl-3- (4-methylisoxazol-3-yl)- 1H-pyrazol-4-yl)methyl)-N- isopentylazepan-3-amine | 2.81 | 394.2 | C |
| 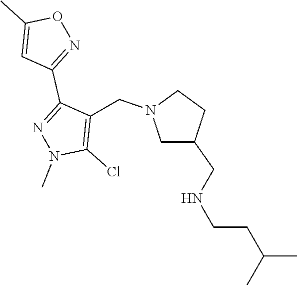 [SMILES: Cc1cc(-c2nn(C)c(Cl)c2CN2CCC(CNCCC(C)C)C2)no1] | 29 | N-((1-((5-Chloro-1-methyl- 3-(5-methylisoxazol-3-yl)- 1H-pyrazol-4- yl)methyl)pyrrolidin-3- yl)methyl)-3-methylbutan- 1-amine | 1.74 | 380.2 | B |
| 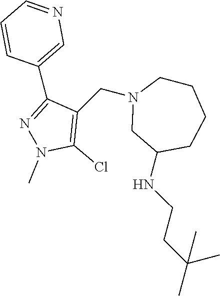 [SMILES: Cn1nc(-c2cccnc2)c(CN2CCCCC(NCCC(C)(C)C)C2)c1Cl] | 30 | 1-((5-Chloro-1-methyl-3- (pyridin-3-yl)-1H-pyrazol- 4-yl)methyl)-N-(3,3- dimethylbutyl)azepan-3- amine | 1.92 | 404.3 | B |
| 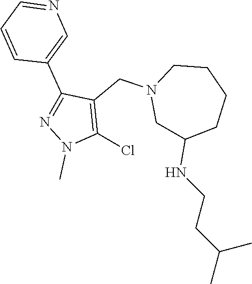 [SMILES: CC(C)CCNC1CCCCN(Cc2c(-c3cccnc3)nn(C)c2Cl)C1] | 31 | 1-((5-Chloro-1-methyl-3- (pyridin-3-yl)-1H-pyrazol- 4-yl)methyl)-N- isopentylazepan-3-amine | 1.82 | 390.3 | B |
| 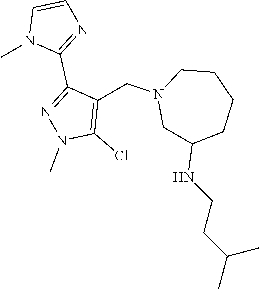 [SMILES: CC(C)CCNC1CCCCN(Cc2c(-c3nccn3C)nn(C)c2Cl)C1] | 32 | 1-((5-Chloro-1-methyl-3- (1-methyl-1H-imidazol-2- yl)-1H-pyrazol-4- yl)methyl)-N- isopentylazepan-3-amine | 2.01 | 393.3 | B |
| 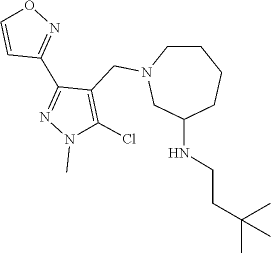 [SMILES: Cn1nc(-c2ccon2)c(CN2CCCCC(NCCC(C)(C)C)C2)c1Cl] | 33 | 1-((5-Chloro-3-(isoxazol-3- yl)-1-methyl-1H-pyrazol-4- yl)methyl)-N-(3,3- dimethylbutyl)azepan-3- amine | 2.00 | 394.1 | B |
| 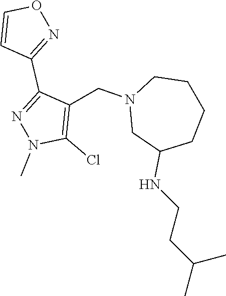 [SMILES: CC(C)CCNC1CCCCN(Cc2c(-c3ccon3)nn(C)c2Cl)C1] | 34 | 1-((5-Chloro-3-(isoxazol-3- yl)-1-methyl-1H-pyrazol-4- yl)methyl)-N- isopentylazepan-3-amine | 1.92 | 380.0 | B |
| 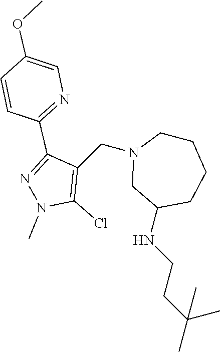 [SMILES: COc1ccc(-c2nn(C)c(Cl)c2CN2CCCCC(NCCC(C)(C)C)C2)nc1] | 35 | 1-((5-Chloro-3-(5- methoxypyridin-2-yl)-1- methyl-1H-pyrazol-4- yl)methyl)-N-(3,3- dimethylbutyl)azepan-3- amine | 2.16 | 434.1 | B |
| 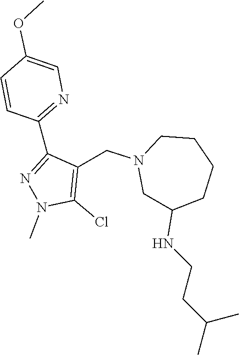 [SMILES: COc1ccc(-c2nn(C)c(Cl)c2CN2CCCCC(NCCC(C)C)C2)nc1] | 36 | 1-((5-Chloro-3-(5- methoxypyridin-2-yl)-1- methyl-1H-pyrazol-4- yl)methyl)-N- isopentylazepan-3-amine | 2.06 | 420.1 | B |
| 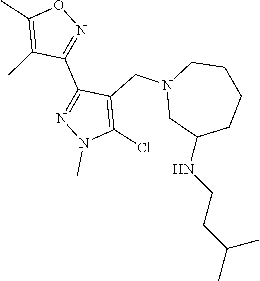 [SMILES: Cc1onc(-c2nn(C)c(Cl)c2CN2CCCCC(NCCC(C)C)C2)c1C] | 37 | 1-((5-Chloro-3-(4,5- dimethylisoxazol-3-yl)-1- methyl-1H-pyrazol-4- yl)methyl)-N- isopentylazepan-3-amine | 2.23 | 408.1 | B |
| 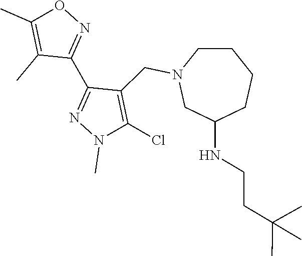 [SMILES: Cc1onc(-c2nn(C)c(Cl)c2CN2CCCCC(NCCC(C)(C)C)C2)c1C] | 38 | 1-((5-Chloro-3-(4,5- dimethylisoxazol-3-yl)-1- methyl-1H-pyrazol-4- yl)methyl)-N-(3,3- dimethylbutyl)azepan-3- amine | 2.37 | 422. | B |
| 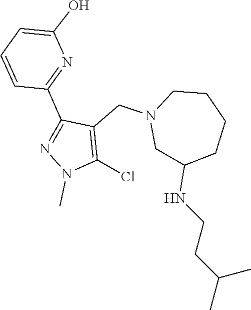 [SMILES: CC(C)CCNC1CCCCN(Cc2c(-c3cccc(O)n3)nn(C)c2Cl)C1] | 39 | 6-(5-Chloro-4-((3- (isopentylamino)azepan-1- yl)methyl)-1-methyl-1H- pyrazol-3-yl)pyridin-2-ol | 1.73 | 406.1 | B |
| 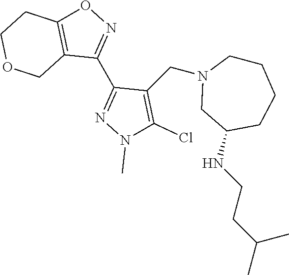 [SMILES: CC(C)CCN[C@H]1CCCCN(Cc2c(-c3noc4c3COCC4)nn(C)c2Cl)C1] | 40 | (S)-1-((5-Chloro-3-(6,7- dihydro-4H-pyrano[3,4- d]isoxazol-3-yl)-1-methyl- 1H-pyrazol-4-yl)methyl)-N- isopentylazepan-3-amine | 2.02 | 436.1 | B |
| 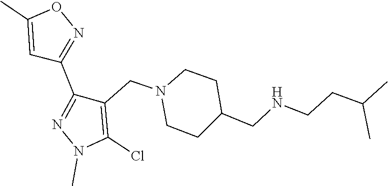 [SMILES: Cc1cc(-c2nn(C)c(Cl)c2CN2CCC(CNCCC(C)C)CC2)no1] | 41 | N-((1-((5-Chloro-1-methyl- 3-(5-methylisoxazol-3-yl)- 1H-pyrazol-4- yl)methyl)piperidin-4- yl)methyl)-3-methylbutan- 1-amine | 1.66 | 394.2 | A |
| 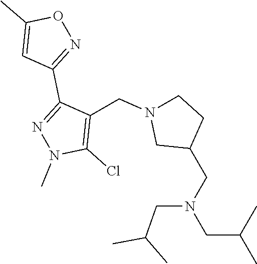 [SMILES: Cc1cc(-c2nn(C)c(Cl)c2CN2CCC(CN(CC(C)C)CC(C)C)C2)no1] | 42 | N-((1-((5-Chloro-1-methyl- 3-(5-methylisoxazol-3-yl)- 1H-pyrazol-4- yl)methyl)pyrrolidin-3- yl)methyl)-/-isobutyl-2- methylpropan-1-amine | 3.12 | 422.2 | A |
| 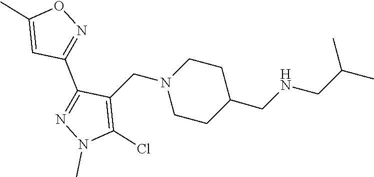 [SMILES: Cc1cc(-c2nn(C)c(Cl)c2CN2CCC(CNCC(C)C)CC2)no1] | 43 | N-((1-((5-Chloro-1-methyl- 3-(5-methylisoxazol-3-yl)- 1H-pyrazol-4- yl)methyl)piperidin-4- yl)methyl)-2- methylpropan-1-amine | 1.58 | 380.3 | B |
| 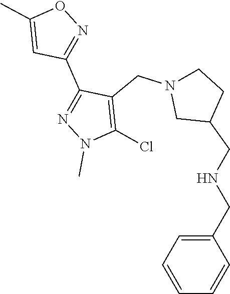 [SMILES: Cc1cc(-c2nn(C)c(Cl)c2CN2CCC(CNCc3ccccc3)C2)no1] | 44 | N-Benzyl-1-(1-((5-chloro- 1-methyl-3-(5- methylisoxazol-3-yl)-1H- pyrazol-4- yl)methyl)pyrrolidin-3- yl)methanamine | 1.86 | 400.3 | B |
| 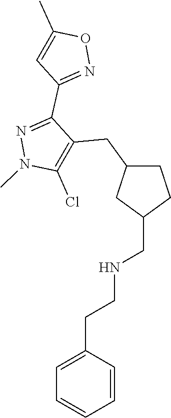 [SMILES: Cc1cc(-c2nn(C)c(Cl)c2CC2CCC(CNCCc3ccccc3)C2)no1] | 45 | N-((1-((5-Chloro-1-methyl- 3-(5-methylisoxazol-3-yl)- 1H-pyrazol-4- yl)methyl)pyrrolidin-3- yl)methyl)-2- phenylethanamine | 1.84 | 414.3 | B |
| 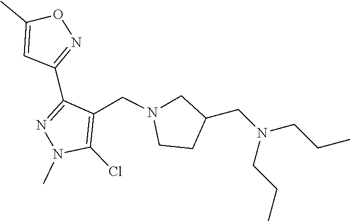 [SMILES: CCCN(CCC)CC1CCN(Cc2c(-c3cc(C)on3)nn(C)c2Cl)C1] | 46 | N-((1-((5-Chloro-1-methyl- 3-(5-methylisoxazol-3-yl)- 1H-pyrazol-4- yl)methyl)pyrrolidin-3- yl)methyl)-N-propylpropan- 1-amine | 2.13 | 394.3 | B |
| 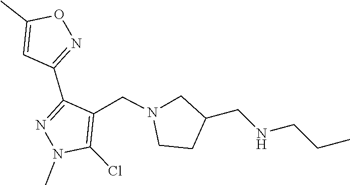 [SMILES: CCCNCC1CCN(Cc2c(-c3cc(C)on3)nn(C)c2Cl)C1] | 47 | N-((1-((5-Chloro-1-methyl- 3-(5-methylisoxazol-3-yl)- 1H-pyrazol-4- yl)methyl)pyrrolidin-3- yl)methyl)propan-1-amine | 1.44 | 352.2 | B |
| 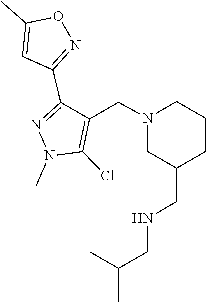 [SMILES: Cc1cc(-c2nn(C)c(Cl)c2CN2CCCC(CNCC(C)C)C2)no1] | 48 | N-((1-((5-Chloro-1-methyl- 3-(5-methylisoxazol-3-yl)- 1H-pyrazol-4- yl)methyl)piperidin-3- yl)methyl)-2- methylpropan-1-amine | 1.70 | 380.3 | B |
| 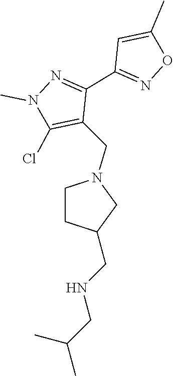 [SMILES: Cc1cc(-c2nn(C)c(Cl)c2CN2CCC(CNCC(C)C)C2)no1] | 49 | N-((1-((5-Chloro-1-methyl- 3-(5-methylisoxazol-3-yl)- 1H-pyrazol-4- yl)methyl)pyrrolidin-3- yl)methyl)-2- methylpropan-1-amine | 1.57 | 366.2 | B |
| 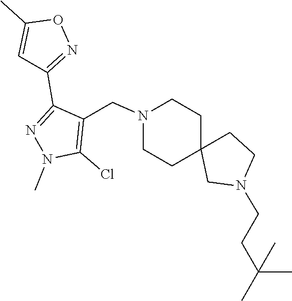 [SMILES: Cc1cc(-c2nn(C)c(Cl)c2CN2CCC3(CC2)CCN(CCC(C)(C)C)C3)no1] | 50 | 3-(5-Chloro-4-((2-(3,3- dimethylbutyl)-2,8- diazaspiro[4.5]decan-8- yl)methyl)-1-methyl-1H- pyrazol-3-yl)-5- methylisoxazole | 2.03 | 434.4 | B |
| 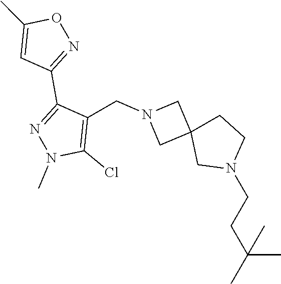 [SMILES: Cc1cc(-c2nn(C)c(Cl)c2CN2CC3(CCN(CCC(C)(C)C)C3)C2)no1] | 51 | 3-(5-Chloro-4-((6-(3,3- dimethylbutyl)-2,6- diazaspiro[3.4]octan-2- yl)methyl)-1-methyl-1H- pyrazol-3-yl)-5- methylisoxazole | 1.89 | 406.3 | B |
| 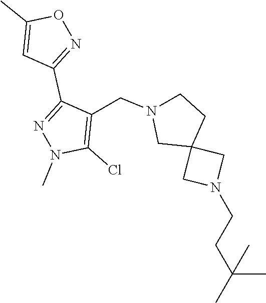 [SMILES: Cc1cc(-c2nn(C)c(Cl)c2CN2CCC3(C2)CN(CCC(C)(C)C)C3)no1] | 52 | 3-(5-Chloro-4-((2-(3,3- dimethylbutyl)-2,6- diazaspiro[3.4]octan-6- yl)methyl)-1-methyl-1H- pyrazol-3-yl)-5- methylisoxazole | 1.88 | 406.3 | B |
| 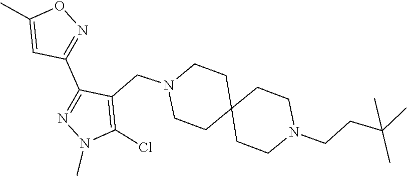 [SMILES: Cc1cc(-c2nn(C)c(Cl)c2CN2CCC3(CCN(CCC(C)(C)C)CC3)CC2)no1] | 53 | 3-(5-Chloro-4-((9-(3,3- dimethylbutyl)-3,9- diazaspiro[5.5]undecan-3- yl)methyl)-1-methyl-1H- pyrazol-3-yl)-5- methylisoxazole | 2.01 | 448.4 | B |
| 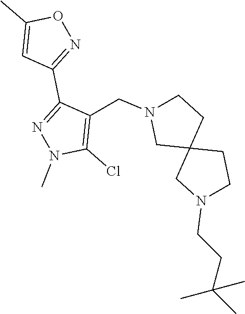 [SMILES: Cc1cc(-c2nn(C)c(Cl)c2CN2CCC3(CCN(CCC(C)(C)C)C3)C2)no1] | 54 | 3-(5-Chloro-4-((7-(3,3- dimethylbutyl)-2,7- diazaspiro[4.4]nonan-2- yl)methyl)-1-methyl-1H- pyrazol-3-yl)-5- methylisoxazole | 2.00 | 420.3 | B |
| 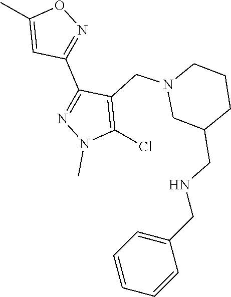 [SMILES: Cc1cc(-c2nn(C)c(Cl)c2CN2CCCC(CNCc3ccccc3)C2)no1] | 55 | N-Benzyl-1-(1-((5-chloro- 1-methyl-3-(5- methylisoxazol-3-yl)-1H- pyrazol-4- yl)methyl)piperidin-3- yl)methanamine | 2.05 | 414.3 | B |
| 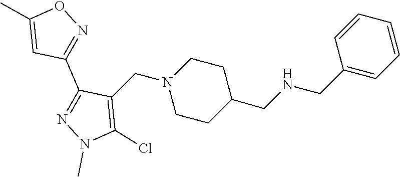 [SMILES: Cc1cc(-c2nn(C)c(Cl)c2CN2CCC(CNCc3ccccc3)CC2)no1] | 56 | N-Benzyl-1-(1-((5-chloro- 1-methyl-3-(5- methylisoxazol-3-yl)-1H- pyrazol-4- yl)methyl)piperidin-4- yl)methanamine | 1.88 | 414.3 | B |
| 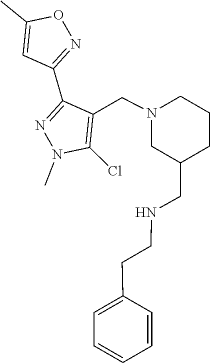 [SMILES: Cc1cc(-c2nn(C)c(Cl)c2CN2CCCC(CNCCc3ccccc3)C2)no1] | 57 | N-((1-((5-Chloro-1-methyl- 3-(5-methylisoxazol-3-yl)- 1H-pyrazol-4- yl)methyl)piperidin-3- yl)methyl)-2- phenylethanamine | 2.00 | 428.3 | B |
| 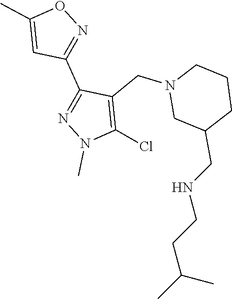 [SMILES: Cc1cc(-c2nn(C)c(Cl)c2CN2CCCC(CNCCC(C)C)C2)no1] | 58 | N-((1-((5-Chloro-1-methyl- 3-(5-methylisoxazol-3-yl)- 1H-pyrazol-4- yl)methyl)piperidin-3- yl)methyl)-3-methylbutan- 1-amine | 1.84 | 394.3 | B |
| 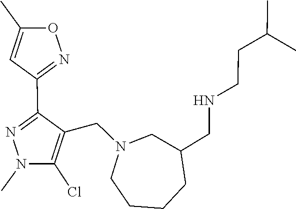 [SMILES: Cc1cc(-c2nn(C)c(Cl)c2CN2CCCCC(CNCCC(C)C)C2)no1] | 59 | N-((1-((5-Chloro-1-methyl- 3-(5-methylisoxazol-3-yl)- 1H-pyrazol-4- yl)methyl)azepan-3- yl)methyl)-3-methylbutan- 1-amine | 2.20 | 408.2 | A |
| 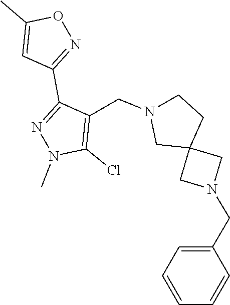 [SMILES: Cc1cc(-c2nn(C)c(Cl)c2CN2CCC3(C2)CN(Cc2ccccc2)C3)no1] | 60 | 3-(4-((2-Benzyl-2,6- diazaspiro[3.4]octan-6- yl)methyl)-5-chloro-1- methyl-1H-pyrazol-3-yl)-5- methylisoxazole | 2.02 | 412 | A |
| 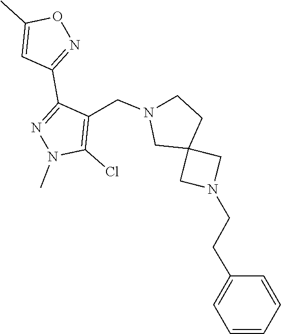 [SMILES: Cc1cc(-c2nn(C)c(Cl)c2CN2CCC3(C2)CN(CCc2ccccc2)C3)no1] | 61 | 3-(5-Chloro-1-methyl-4- ((2-phenethyl-2,6- diazaspiro[3.4]octan-6- yl)methyl)-1H-pyrazol-3- yl)-5-methylisoxazole | 1.98 | 426.2 | A |
| 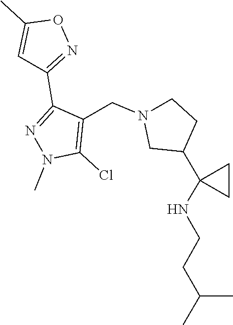 [SMILES: Cc1cc(-c2nn(C)c(Cl)c2CN2CCC(C3(NCCC(C)C)CC3)C2)no1] | 62 | 1-(1-((5-Chloro-1-methyl- 3-(5-methylisoxazol-3-yl)- 1H-pyrazol-4- yl)methyl)pyrrolidin-3-yl)- N- isopentylcyclopropanamine | 2.39 | 406.2 | A |
| 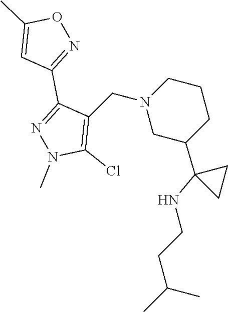 [SMILES: Cc1cc(-c2nn(C)c(Cl)c2CN2CCCC(C3(NCCC(C)C)CC3)C2)no1] | 63 | 1-(1-((5-Chloro-1-methyl- 3-(5-methylisoxazol-3-yl)- 1H-pyrazol-4- yl)methyl)piperidin-3-yl)-N- isopentylcyclopropanamine | 2.58 | 420.2 | A |
| 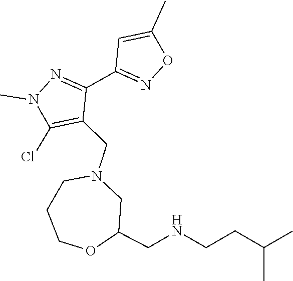 [SMILES: Cc1cc(-c2nn(C)c(Cl)c2CN2CCCOC(CNCCC(C)C)C2)no1] | 64 | N-((4-((5-Chloro-1-methyl- 3-(5-methylisoxazol-3-yl)- 1H-pyrazol-4-yl)methyl)- 1,4-oxazepan-2- yl)methyl)-3-methylbutan- 1-amine | 1.79 | 410.0 | B |
| 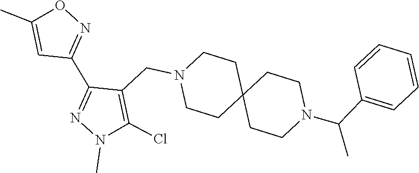 [SMILES: Cc1cc(-c2nn(C)c(Cl)c2CN2CCC3(CC2)CCN(C(C)c2ccccc2)CC3)no1] | 65 | 3-(5-Chloro-1-methyl-4- ((9-(1-phenylethyl)-3,9- diazaspiro[5.5]undecan-3- yl)methyl)-1H-pyrazol-3- yl)-5-methylisoxazole | 2.39 | 468.3 | B |
| 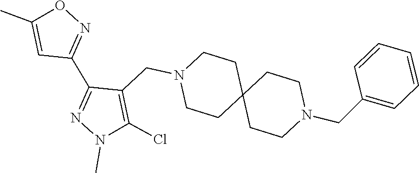 [SMILES: Cc1cc(-c2nn(C)c(Cl)c2CN2CCC3(CCN(Cc4ccccc4)CC3)CC2)no1] | 66 | 3-(4-((9-Benzyl-3,9- diazaspiro[5.5]undecan-3- yl)methyl)-5-chloro-1- methyl-1H-pyrazol-3-yl)-5- methylisoxazole | 2.32 | 454.3 | B |
| 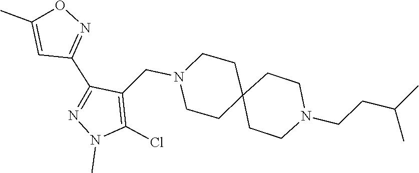 [SMILES: Cc1cc(-c2nn(C)c(Cl)c2CN2CCC3(CCN(CCC(C)C)CC3)CC2)no1] | 67 | 3-(5-Chloro-4-((9- isopentyl-3,9- diazaspiro[5.5]undecan-3- yl)methyl)-1-methyl-1H- pyrazol-3-yl)-5- methylisoxazole | 1.92 | 434.3 | B |
| 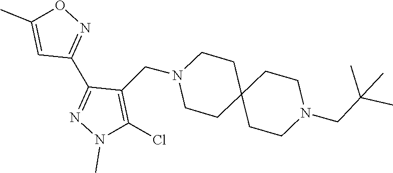 [SMILES: Cc1cc(-c2nn(C)c(Cl)c2CN2CCC3(CC2)CCN(CC(C)(C)C)CC3)no1] | 68 | 3-(5-Chloro-1-methyl-4- ((9-neopentyl-3,9- diazaspiro[5.5]undecan-3- yl)methyl)-1H-pyrazol-3- yl)-5-methylisoxazole | 2.94 | 434.3 | B |

### Examples 69 and 70. (S)-3-(5-Chloro-4-((7-(3,3-dimethylbutyl)-2,7-diazaspiro[4.4]nonan-2-yl)methyl)-1-methyl-1H-pyrazol-3-yl)-5-methylisoxazole and (R)-3-(5-chloro-4-((7-(3,3-dimethylbutyl)-2,7-diazaspiro[4.4]nonan-2-yl)methyl)-1-methyl-1H-pyrazol-3-yl)-5-methylisoxazole

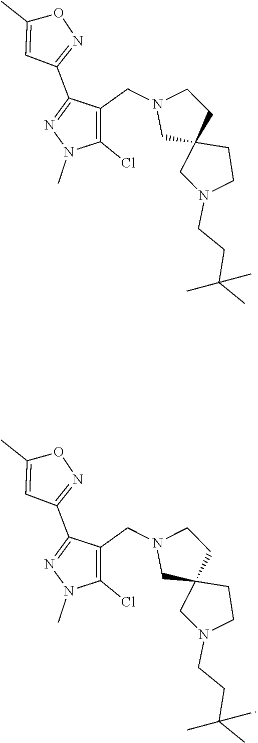

Starting from 3-(5-chloro-4-((7-(3,3-dimethylbutyl)-2,7-diazaspiro[4.4]nonan-2-yl)methyl)-1-methyl-1H-pyrazol-3-yl)-5-methylisoxazole, obtained following the procedure described in example 1, a chiral preparative HPLC separation (Column Chiralpak IC 20×250 mm, 5 μm; temperature: rt; eluent: n-Heptane/EtOH/Et₂NH 90/10/0.03 v/v/v; flow rate 16 mL/min; Rt1: 45.8 min; Rt2: 49.4 min) was carried out to give the title compounds.

HPLC Rt (Method A): 2.12 and 2.13 min; ESI+−MS m/z: 420.2 (M+H)⁺.

### Examples 71 and 72. (R)-N-((1-((5-Chloro-1-methyl-3-(5-methylisoxazol-3-yl)-1H-pyrazol-4-yl)methyl)pyrrolidin-3-yl)methyl)-2-phenylethanamine and (S)-N-((1-((5-chloro-1-methyl-3-(5-methylisoxazol-3-yl)-1H-pyrazol-4-yl)methyl)pyrrolidin-3-yl)methyl)-2-phenylethanamine

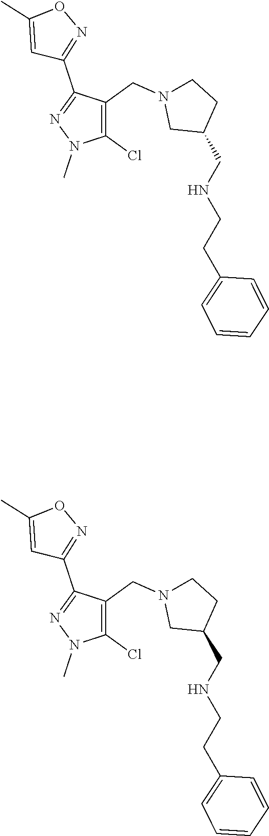 [SMILES: Cc1cc(-c2nn(C)c(Cl)c2CN2CC[C@H](CNCCc3ccccc3)C2)no1 ; Cc1cc(-c2nn(C)c(Cl)c2CN2CC[C@@H](CNCCc3ccccc3)C2)no1]

Starting from example 45, a chiral preparative HPLC separation (Column Chiralpak IB 20×250 mm, 5 μm; temperature: rt; eluent: n-Heptane/IPA/Et₂NH 80/20/0.1 v/v/v; flow rate 12 mL/min; Rt1: 10.3 min; Rt2: 13.8 min) was carried out to give the title compounds.

HPLC Rt (Method B): 1.88 min; ESI+−MS m/z: 414.3 (M+H)⁺.

HPLC Rt (Method A): 1.89 min; ESI+−MS m/z: 414.3 (M+H)⁺.

### Examples 73 and 74. (S)-N-((1-((5-Chloro-1-methyl-3-(5-methylisoxazol-3-yl)-1H-pyrazol-4-yl)methyl)pyrrolidin-3-yl)methyl)-3,3-dimethylbutan-1-amine and (R)-N-((1-((5-chloro-1-methyl-3-(5-methylisoxazol-3-yl)-1H-pyrazol-4-yl)methyl)pyrrolidin-3-yl)methyl)-3,3-dimethylbutan-1-amine

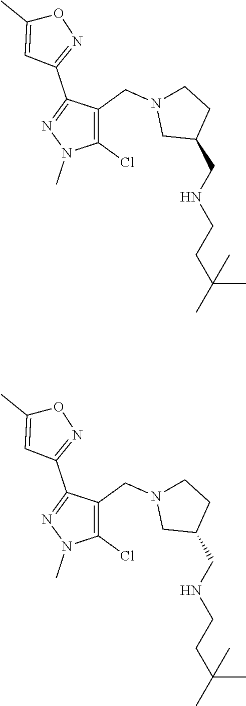

Starting from example 1, a chiral preparative HPLC separation [Column Chiralpak IG 20×250 mm, 5 μm; temperature: rt; eluent: n-Heptane/IPA/Et₂NH 95/5/0.1 v/v/v; flow rate 16 mL/min; Rt1: 29.9 min; Rt2: 33.0 min) was carried out to give the title compounds.

HPLC Rt (Method B): 1.85 and 1.87 min; ESI+−MS m/z: 394.3 (M+H)⁺.

### Examples 75 and 76. (2s,4r)-6-((5-Chloro-1-methyl-3-(5-methylisoxazol-3-yl)-1H-pyrazol-4-yl)methyl)-N-(3,3-dimethylbutyl)-6-azaspiro[3.4]octan-2-amine and (2r,4s)-6-((5-chloro-1-methyl-3-(5-methylisoxazol-3-yl)-1H-pyrazol-4-yl)methyl)-N-(3,3-dimethylbutyl-6-azaspiro[3.4]octan-2-amine

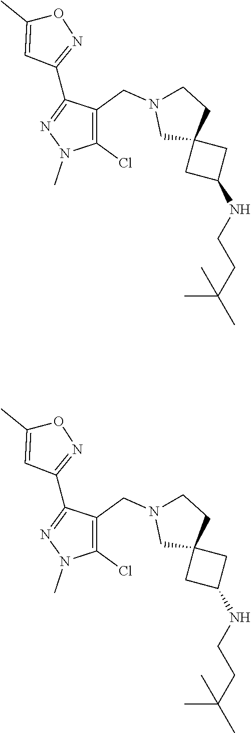 [SMILES: Cc1cc(-c2nn(C)c(Cl)c2CN2CC[C@]3(C2)C[C@H](NCCC(C)(C)C)C3)no1 ; Cc1cc(-c2nn(C)c(Cl)c2CN2CC[C@]3(C2)C[C@@H](NCCC(C)(C)C)C3)no1]

Starting from 6-((5-chloro-1-methyl-3-(5-methylisoxazol-3-yl)-1H-pyrazol-4-yl)methyl)-N-(3,3-dimethylbutyl)-6-azaspiro[3.4]octan-2-amine, obtained following the procedure described in example 1, a chiral preparative HPLC separation (Column Chiralpak IA 20×250 mm, 5 μm; temperature: rt; eluent: n-Heptane/EtOH/Et₂NH 95/5/0.02 v/v/v; flow rate 13 mL/min; Rt1: 12.9 min; Rt2: 13.8 min) was carried out to give the title compounds.

HPLC Rt (Method B): 1.89 min; ESI+−MS m/z: 420.3 (M+H)⁺.

HPLC Rt (Method B): 1.86 min; ESI+−MS m/z: 420.4 (M+H)⁺.

### Example 77. N-(((3R,4S)-1-((5-Chloro-1-methyl-3-(5-methylisoxazol-3-yl)-1H-pyrazol-4-yl)methyl)-4-methylpyrrolidin-3-yl)methyl)-3,3-dimethylbutan-1-amine

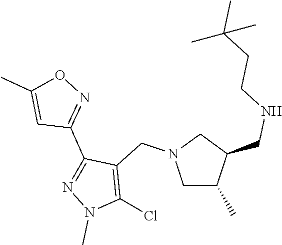 [SMILES: Cc1cc(-c2nn(C)c(Cl)c2CN2C[C@@H](CNCCC(C)(C)C)[C@H](C)C2)no1]

(3S,4S)-1-((5-Chloro-1-methyl-3-(5-methylisoxazol-3-yl)-1H-pyrazol-4-yl)methyl)-N-(3,3-dimethylbutyl)-4-methylpyrrolidine-3-carboxamide (216 mg, 0.51 mmol), obtained following a similar procedure to that described in example 1, dissolved in THE (10 mL) was added dropwise to a freshly prepared Alane solution (1 M in THF, 2.56 mL, 2.56 mmol) at 0° C. The reaction mixture was stirred at −10° C. for 1.5 h before it was allowed to reach rt and stirred overnight. The reaction mixture was quenched with water and the product extracted with EtOAc. The combined organic layers were dried over Na₂SO₄, filtered and concentrated to dryness. The residue was purified by reversed phase semipreparative HPLC to give the title compound (8 mg, 4% yield).

HPLC Rt (Method B): 2.00 min; ESI+−MS m/z: 408.3 (M+H)⁺.

This method was used for the preparation of example 78 using suitable starting materials:

| | | | Rt | MS | HPLC |
|---|---|---|---|---|---|
| STRUCTURE | EX | CHEMICAL NAME | (min) | (M + H) | Method |
| 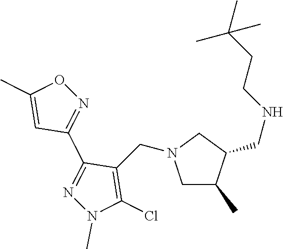 [SMILES: Cc1cc(-c2nn(C)c(Cl)c2CN2C[C@H](CNCCC(C)(C)C)[C@@H](C)C2)no1] | 78 | N-(((3S,4R)-1-((5-Chloro- 1-methyl-3-(5- methylisoxazol-3-yl)-1H- pyrazol-4-yl)methyl)-4- methylpyrrolidin-3- yl)methyl)-3,3- dimethylbutan-1-amine | 1.99 | 408.4 | B |

### Example 79. 1-((5-Chloro-1-methyl-3-(5-methylisoxazol-3-yl)-1H-pyrazol-4-yl)methyl)-N-(2-(piperidin-1-yl)ethyl)azepan-3-amine

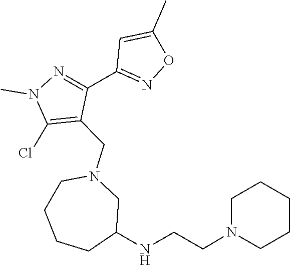 [SMILES: Cc1cc(-c2nn(C)c(Cl)c2CN2CCCCC(NCCN3CCCCC3)C2)no1]

### Step 1. N-(1-((5-Chloro-1-methyl-3-(5-methylisoxazol-3-yl)-1H-pyrazol-4-yl)methyl)azepan-3-yl)-2-(piperidin-1-yl)acetamide

2-(Piperidin-1-yl)acetic acid (44.2 mg, 0.31 mmol) was suspended in CH₂Cl₂ and EDC-HCl (118 mg, 0.62 mmol) was added followed by HOBt (94.6 mg, 0.62 mmol). Then 1-((5-chloro-1-methyl-3-(5-methylisoxazol-3-yl)-1H-pyrazol-4-yl)methyl)azepan-3-amine (100 mg, 0.31 mmol, obtained following a similar procedure to that described in example 1) and Et₃N (0.5 mL, 3.57 mmol) were added and the reaction mixture was stirred at rt overnight. Water was added and the mixture was extracted with CH₂Cl₂. The combined organic layers were dried over Na₂SO₄, filtered and concentrated to dryness to give the title compound (59.1 mg, 43% yield).

### Step 2. Title Compound

To a stirred solution of freshly prepared alane (1 M in THF, 658 μL, 0.66 mmol), the compound obtained in Step 1 (59.1 mg, 0.13 mmol) dissolved in THE (3 mL) was added dropwise at 0° C. The reaction mixture was stirred at −10° C. for 1.5 h before it was allowed to reach rt and stirred overnight. The reaction mixture was quenched with water and the product extracted with EtOAc. The combined organic layers were dried over Na₂SO₄, filtered and concentrated to dryness. The residue was purified by flash chromatography (silica gel, CH₂Cl₂/MeOH, then CH₂Cl₂/1M NH₃ in MeOH) to give the title compound (5 mg, 9% yield).

HPLC Rt (Method B): 2.01 min; ESI+−MS m/z: 435.3 (M+H)⁺.

### Example 80. 1-((5-Chloro-1-methyl-3-(5-methylisoxazol-3-yl)-1H-pyrazol-4-yl)methyl)-N-isopentyl-N-methylazepan-3-amine

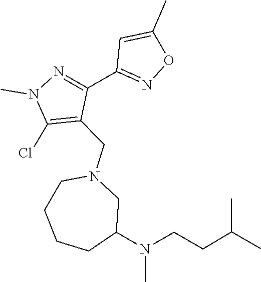 [SMILES: Cc1cc(-c2nn(C)c(Cl)c2CN2CCCCC(N(C)CCC(C)C)C2)no1]

A mixture of the compound obtained in example 2 (14.7 mg, 0.04 mmol), formic acid (70 μL, 0.002 mmol) and formaldehyde (37% in water, 231 μL, 3.10 mmol) were heated in a sealed tube at reflux temperature overnight. All volatiles were removed under reduced pressure to give the title compound (7.8 mg, 51% yield).

HPLC Rt (Method B): 2.27 min; ESI+−MS m/z: 408.3 (M+H)⁺.

This method was used for the preparation of examples 81 and 82 using suitable starting materials:

| | | | Rt | MS | HPLC |
|---|---|---|---|---|---|
| STRUCTURE | EX | CHEMICAL NAME | (min) | (M + H) | Method |
| 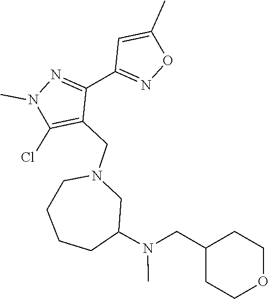 [SMILES: Cc1cc(-c2nn(C)c(Cl)c2CN2CCCCC(N(C)CC3CCOCC3)C2)no1] | 81 | 1-((5-Chloro-1-methyl-3- (5-methylisoxazol-3-yl)- 1H-pyrazol-4-yl)methyl)-N- methyl-N-((tetrahydro-2H- pyran-4-yl)methyl)azepan- 3-amine | 2.16 | 436.3 | B |
| 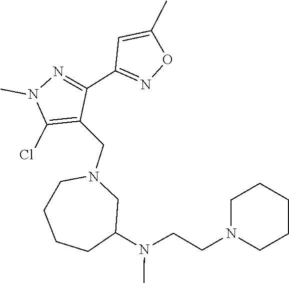 [SMILES: Cc1cc(-c2nn(C)c(Cl)c2CN2CCCCC(N(C)CCN3CCCCC3)C2)no1] | 82 | 1-((5-Chloro-1-methyl-3- (5-methylisoxazol-3-yl)- 1H-pyrazol-4-yl)methyl)-N- methyl-N-(2-(piperidin-1- yl)ethyl)azepan-3-amine | 1.90 | 449.3 | B |

### Example 83. (5-Chloro-1-methyl-3-(5-methylisoxazol-3-yl)-1H-pyrazol-4-yl)(3-(((3,3-dimethylbutyl)amino)methyl)piperidin-1-yl)methanone

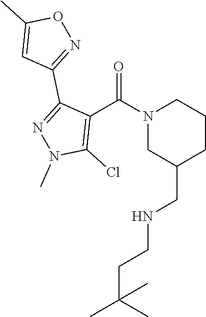 [SMILES: Cc1cc(-c2nn(C)c(Cl)c2C(=O)N2CCCC(CNCCC(C)(C)C)C2)no1]

### Step 1. 5-Chloro-1-methyl-3-(5-methylisoxazol-3-yl)-1H-pyrazole-4-carboxylic acid

### Method 1a

Potassium permanganate (11.2 g, 70.7 mmol) was added to a suspension of intermediate 1 (5.32 g, 23.6 mmol) in water (80 mL) and the reaction mixture was heated at 95° C. for 3 h. The suspension formed was filtered through a Millipore filter. The pale yellow solution was acidified to pH 8 with 2N HCl solution at 0° C. The resulting white precipitate was filtered, washed several times with water and dried under vacuum at 60° C. to give the title compound (2.81 g, 49%).

### Method 1b

Intermediate 1 (145 mg, 0.64 mmol) was dissolved in acetone and cooled down to 0° C. At this temperature sulfamic acid (69 mg, 0.71 mmol), dissolved in water (0.6 mL) was added. After 5 min sodium chlorite (87 mg, 0.96 mmol) was added and the reaction was stirred at 0° C. for 30 min. The volatiles were removed under reduced pressure and the solid residue was washed two times with a small amount of water. The solid obtained was dried under vacuum to give the title compound (153 mg, 98%).

HPLC Rt (Method A): 0.24 min; ESI+−MS m/z: 242 (M+H)⁺.

¹H NMR (400 MHz, CD₃OD) δ 6.53 (q, J=0.9 Hz, 1H), 3.94 (s, 3H), 2.49 (d, J=0.9 Hz, 3H).

### Step 2. tert-Butyl ((1-(5-chloro-1-methyl-3-(5-methylisoxazol-3-yl)-1H-pyrazole-4-carbonyl)piperidin-3-yl)methyl)carbamate

EDC-HCl (625 mg, 3.31 mmol) was suspended in DMF (20 mL) under argon atmosphere at rt. HOBt (507 mg, 3.31 mmol) was added followed by the compound obtained in Step 1 (400 mg, 1.65 mmol). Then, tert-butyl (piperidin-3-ylmethyl)carbamate (532 mg, 2.48 mmol) in DMF (25 mL) and Et₃N (578 μL, 4.14 mmol) were added. The reaction mixture was stirred at rt overnight and the residue was partitioned between water and EtOAc/Et₂O (2:1). The aqueous phase was extracted with EtOAc/Et₂O (2:1) and the combined organic layers were dried over Na₂SO₄, filtered and concentrated to dryness to give the title compound (681 mg, 94% yield).

HPLC Rt (Method A): 1.86 min; ESI+−MS m/z: 438 (M+H)⁺.

### Step 3. (3-(Aminomethyl)piperidin-1-yl)(5-chloro-1-methyl-3-(5-methylisoxazol-3-yl)-1H-pyrazol-4-yl)methanone, 2,2,2-trifluoroacetate salt

To a solution of the compound obtained in Step 2 (182 mg, 0.42 mmol) in CH₂Cl₂ (20 mL) TFA (254 μL, 3.33 mmol) was added at 0° C. The reaction mixture was stirred for 4 h at rt. The solvent was removed under reduced pressure to give the title compound, which was used in the next step without further purification (187 mg, quant.).

HPLC Rt (Method A): 1.17 min; ESI+−MS m/z: 338.2 (M+H)⁺.

### Step 4. Title Compound

To a solution of the compound obtained in Step 3 (187 mg, 0.41 mmol) in MeOH (5 mL) Et₃N (116 μL, 0.83 mmol) was added and the mixture stirred for 5 min (to liberate free base). Then, a solution of 3,3-dimethylbutanal (56.9 μL, 0.46 mmol) in MeOH was added followed by NaBH₃CN (52.1 mg, 0.83 mmol) and the reaction mixture stirred at rt for 1 h. The reaction was quenched with NaOH 10% and volatiles were removed under reduced pressure. The residue was partitioned between EtOAc and 10% NaOH solution. The combined organic layers were dried over Na₂SO₄, filtered and concentrated to dryness. The residue was purified by flash chromatography (silica gel, CH₂Cl₂/MeOH, then CH₂Cl₂/(1M NH₃ in MeOH) to give the title compound (65.3 mg, 37% yield).

HPLC Rt (Method A): 1.88 min; ESI+−MS m/z: 422.2 (M+H)⁺.

This method was used for the preparation of examples 84-105 using suitable starting materials (and intermediates 1-19):

| | | | Rt | MS | HPLC |
|---|---|---|---|---|---|
| STRUCTURE | EX | CHEMICAL NAME | (min) | (M + H) | Method |
| 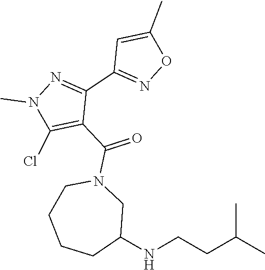 [SMILES: Cc1cc(-c2nn(C)c(Cl)c2C(=O)N2CCCCC(NCCC(C)C)C2)no1] | 84 | (5-Chloro-1-methyl-3-(5- methylisoxazol-3-yl)-1H- pyrazol-4-yl)(3- (isopentylamino)azepan-1- yl)methanone | 1.73 | 408.2 | B |
| 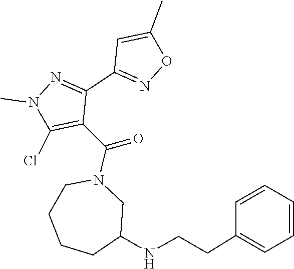 [SMILES: Cc1cc(-c2nn(C)c(Cl)c2C(=O)N2CCCCC(NCCc3ccccc3)C2)no1] | 85 | (5-Chloro-1-methyl-3-(5- methylisoxazol-3-yl)-1H- pyrazol-4-yl)(3- (phenethylamino)azepan- 1-yl)methanone | 1.90 | 442.2 | B |
| 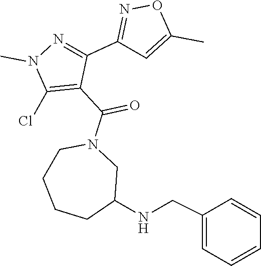 [SMILES: Cc1cc(-c2nn(C)c(Cl)c2C(=O)N2CCCCC(NCc3ccccc3)C2)no1] | 86 | (3-(Benzylamino)azepan- 1-yl)(5-chloro-1-methyl-3- (5-methylisoxazol-3-yl)- 1H-pyrazol-4- yl)methanone | 1.90 | 428.2 | B |
| 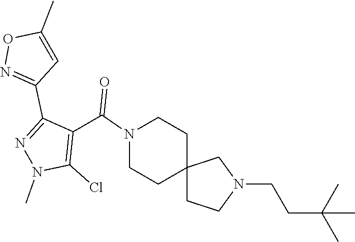 [SMILES: Cc1cc(-c2nn(C)c(Cl)c2C(=O)N2CCC3(CCN(CCC(C)(C)C)C3)CC2)no1] | 87 | (5-Chloro-1-methyl-3-(5- methylisoxazol-3-yl)-1H- pyrazol-4-yl)(2-(3,3- dimethylbutyl)-2,8- diazaspiro[4.5]decan-8- yl)methanone | 1.86 | 448.2 | A |
| 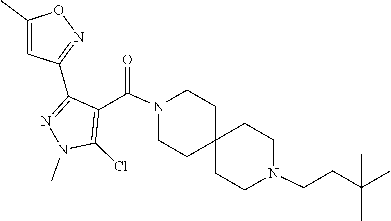 [SMILES: Cc1cc(-c2nn(C)c(Cl)c2C(=O)N2CCC3(CCN(CCC(C)(C)C)CC3)CC2)no1] | 88 | (5-Chloro-1-methyl-3-(5- methylisoxazol-3-yl)-1H- pyrazol-4-yl)(9-(3,3- dimethylbutyl)-3,9- diazaspiro[5.5]undecan-3- yl)methanone | 1.79 | 462.2 | A |
| 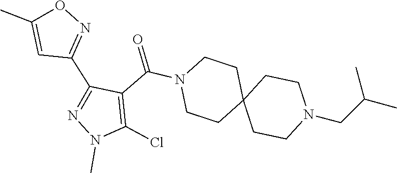 [SMILES: Cc1cc(-c2nn(C)c(Cl)c2C(=O)N2CCC3(CCN(CC(C)C)CC3)CC2)no1] | 89 | (5-Chloro-1-methyl-3-(5- methylisoxazol-3-yl)-1H- pyrazol-4-yl)(9-isobutyl- 3,9- diazaspiro[5.5]undecan-3- yl)methanone | 1.71 | 434.2 | A |
| 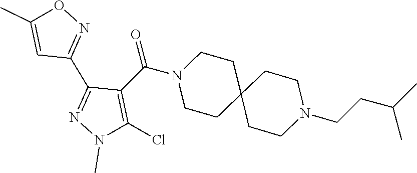 [SMILES: Cc1cc(-c2nn(C)c(Cl)c2C(=O)N2CCC3(CCN(CCC(C)C)CC3)CC2)no1] | 90 | (5-Chloro-1-methyl-3-(5- methylisoxazol-3-yl)-1H- pyrazol-4-yl)(9-isopentyl- 3,9- diazaspiro[5.5]undecan-3- yl)methanone | 1.66 | 448.0 | B |
| 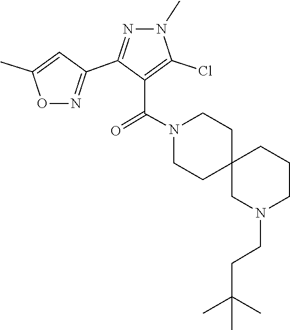 [SMILES: Cc1cc(-c2nn(C)c(Cl)c2C(=O)N2CCC3(CCCN(CCC(C)(C)C)C3)CC2)no1] | 91 | (5-Chloro-1-methyl-3-(5- methylisoxazol-3-yl)-1H- pyrazol-4-yl)(2-(3,3- dimethylbutyl)-2,9- diazaspiro[5.5]undecan-9- yl)methanone | 1.83 | 462.2 | A |
| 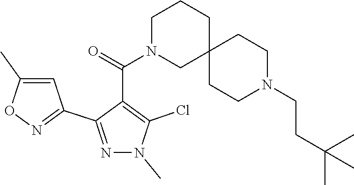 [SMILES: Cc1cc(-c2nn(C)c(Cl)c2C(=O)N2CCCC3(CCN(CCC(C)(C)C)CC3)C2)no1] | 92 | (5-Chloro-1-methyl-3-(5- methylisoxazol-3-yl)-1H- pyrazol-4-yl)(9-(3,3- dimethylbutyl)-2,9- diazaspiro[5.5]undecan-2- yl)methanone | 1.93 | 462.2 | A |
| 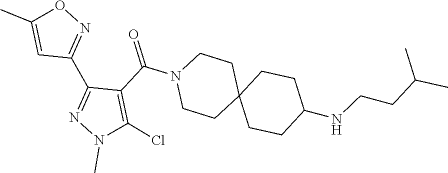 [SMILES: Cc1cc(-c2nn(C)c(Cl)c2C(=O)N2CCC3(CCC(NCCC(C)C)CC3)CC2)no1] | 93 | (5-Chloro-1-methyl-3-(5- methylisoxazol-3-yl)-1H- pyrazol-4-yl)(9- (isopentylamino)-3- azaspiro[5.5]undecan-3- yl)methanone | 1.66 | 462.2 | A |
| 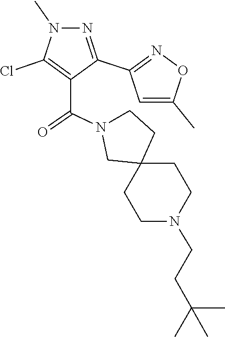 [SMILES: Cc1cc(-c2nn(C)c(Cl)c2C(=O)N2CCC3(CCN(CCC(C)(C)C)CC3)C2)no1] | 94 | (5-Chloro-1-methyl-3-(5- methylisoxazol-3-yl)-1H- pyrazol-4-yl)(8-(3,3- dimethylbutyl)-2,8- diazaspiro[4.5]decan-2- yl)methanone | 1.88 | 448.2 | A |
| 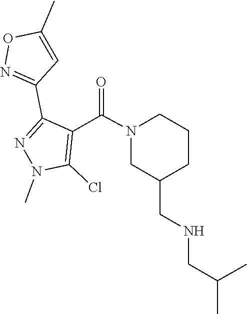 [SMILES: Cc1cc(-c2nn(C)c(Cl)c2C(=O)N2CCCC(CNCC(C)C)C2)no1] | 95 | (5-Chloro-1-methyl-3-(5- methylisoxazol-3-yl)-1H- pyrazol-4-yl)(3- ((isobutylamino)methyl) piperidin-1-yl)methanone | 1.609 | 394.2 | A |
| 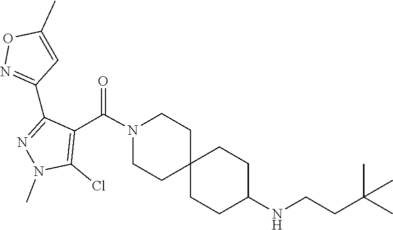 [SMILES: Cc1cc(-c2nn(C)c(Cl)c2C(=O)N2CCC3(CCC(NCCC(C)(C)C)CC3)CC2)no1] | 96 | (5-Chloro-1-methyl-3-(5- methylisoxazol-3-yl)-1H- pyrazol-4-yl)(9-((3,3- dimethylbutyl)amino)-3- azaspiro[5.5]undecan-3- yl)methanone | 1.90 | 476.2 | A |
| 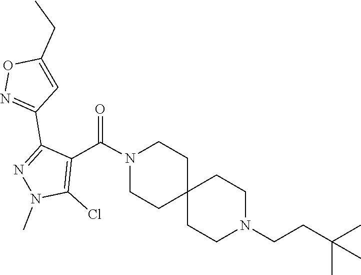 [SMILES: CCc1cc(-c2nn(C)c(Cl)c2C(=O)N2CCC3(CCN(CCC(C)(C)C)CC3)CC2)no1] | 97 | (5-Chloro-3-(5- ethylisoxazol-3-yl)-1- methyl-1H-pyrazol-4-yl)(9- (3,3-dimethylbutyl)-3,9- diazaspiro[5.5]undecan-3- yl)methanone | 2.07 | 476.2 | A |
| 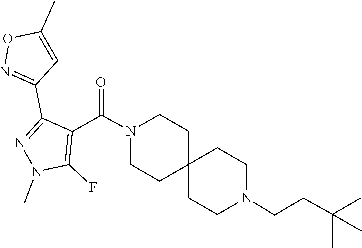 [SMILES: Cc1cc(-c2nn(C)c(F)c2C(=O)N2CCC3(CCN(CCC(C)(C)C)CC3)CC2)no1] | 98 | (9-(3,3-Dimethylbutyl)-3,9- diazaspiro[5.5]undecan-3- yl)(5-fluoro-1-methyl-3-(5- methylisoxazol-3-yl)-1H- pyrazol-4-yl)methanone | 1.73 | 446.4 | A |
| 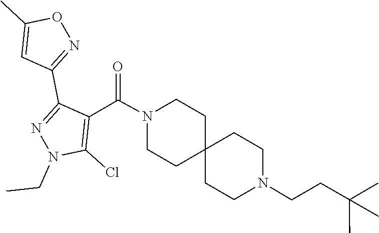 [SMILES: CCn1nc(-c2cc(C)on2)c(C(=O)N2CCC3(CCN(CCC(C)(C)C)CC3)CC2)c1Cl] | 99 | (5-Chloro-1-ethyl-3-(5- methylisoxazol-3-yl)-1H- pyrazol-4-yl)(9-(3,3- dimethylbutyl)-3,9- diazaspiro[5.5]undecan-3- yl)methanone | 1.94 | 476.2 | A |
| 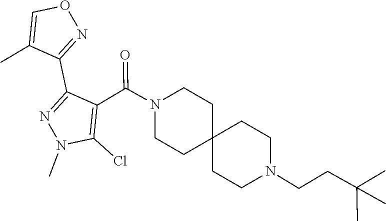 [SMILES: Cc1conc1-c1nn(C)c(Cl)c1C(=O)N1CCC2(CCN(CCC(C)(C)C)CC2)CC1] | 100 | (5-Chloro-1-methyl-3-(4- methylisoxazol-3-yl)-1H- pyrazol-4-yl)(9-(3,3- dimethylbutyl)-3,9- diazaspiro[5.5]undecan-3- yl)methanone | 1.92 | 462.2 | A |
| 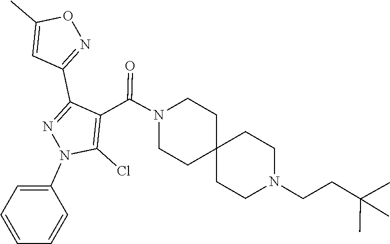 [SMILES: Cc1cc(-c2nn(-c3ccccc3)c(Cl)c2C(=O)N2CCC3(CCN(CCC(C)(C)C)CC3)CC2)no1] | 101 | (5-Chloro-3-(5- methylisoxazol-3-yl)-1- phenyl-1H-pyrazol-4-yl)(9- (3,3-dimethylbutyl)-3,9- diazaspiro[5.5]undecan-3- yl)methanone | 2.29 | 524.2 | A |
| 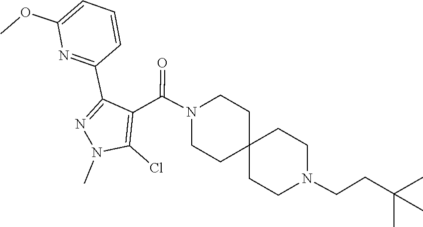 [SMILES: COc1cccc(-c2nn(C)c(Cl)c2C(=O)N2CCC3(CCN(CCC(C)(C)C)CC3)CC2)n1] | 102 | (5-Chloro-3-(6- methoxypyridin-2-yl)-1- methyl-1H-pyrazol-4-yl)(9- (3,3-dimethylbutyl)-3,9- diazaspiro[5.5]undecan-3- yl)methanone | 2.02 | 488.2 | A |
| 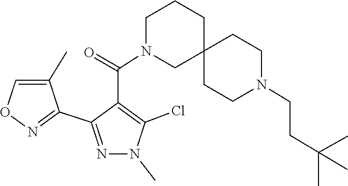 [SMILES: Cc1conc1-c1nn(C)c(Cl)c1C(=O)N1CCCC2(CCN(CCC(C)(C)C)CC2)C1] | 103 | (5-Chloro-1-methyl-3-(4- methylisoxazol-3-yl)-1H- pyrazol-4-yl)(9-(3,3- dimethylbutyl)-2,9- diazaspiro[5.5]undecan-2- yl)methanone | 2.06 | 462.2 | A |
| 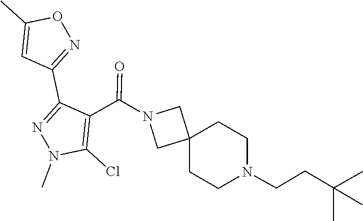 [SMILES: Cc1cc(-c2nn(C)c(Cl)c2C(=O)N2CC3(CCN(CCC(C)(C)C)CC3)C2)no1] | 104 | (5-Chloro-1-methyl-3-(5- methylisoxazol-3-yl)-1H- pyrazol-4-yl)(7-(3,3- dimethylbutyl)-2,7- diazaspiro[3.5]nonan-2- yl)methanone | 1.89 | 434.2 | A |
| 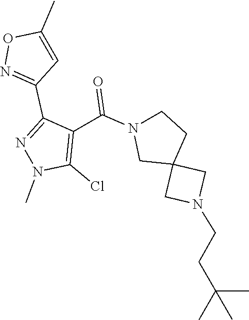 [SMILES: Cc1cc(-c2nn(C)c(Cl)c2C(=O)N2CCC3(CN(CCC(C)(C)C)C3)C2)no1] | 105 | (5-Chloro-1-methyl-3-(5- methylisoxazol-3-yl)-1H- pyrazol-4-yl)(2-(3,3- dimethylbutyl)-2,6- diazaspiro[3.4]octan-6- yl)methanone | 1.84 | 420.2 | A |
| 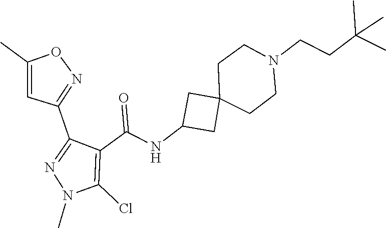 [SMILES: Cc1cc(-c2nn(C)c(Cl)c2C(=O)NC2CC3(CCN(CCC(C)(C)C)CC3)C2)no1] | 106 | 5-Chloro-N-(7-(3,3- dimethylbutyl)-7- azaspiro[3.5]nonan-2-yl)- 1-methyl-3-(5- methylisoxazol-3-yl)-1H- pyrazole-4-carboxamide | 1.90 | 448.2 | A |
| 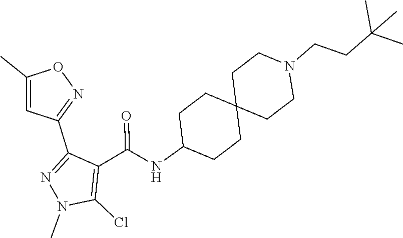 [SMILES: Cc1cc(-c2nn(C)c(Cl)c2C(=O)NC2CCC3(CC2)CCN(CCC(C)(C)C)CC3)no1] | 107 | 5-Chloro-N-(3-(3,3- dimethylbutyl)-3- azaspiro[5.5]undecan-9- yl)-1-methyl-3-(5- methylisoxazol-3-yl)-1H- pyrazole-4-carboxamide | 2.09 | 476.2 | A |

### Examples 108 and 109. (R)-(5-Chloro-1-methyl-3-(5-methylisoxazol-3-yl)-1H-pyrazol-4-yl)(3-(isopentylamino)piperidin-1-yl)methanone and (S)-(5-Chloro-1-methyl-3-(5-methylisoxazol-3-yl)-1H-pyrazol-4-yl)(3-(isopentylamino)piperidin-1-yl)methanone

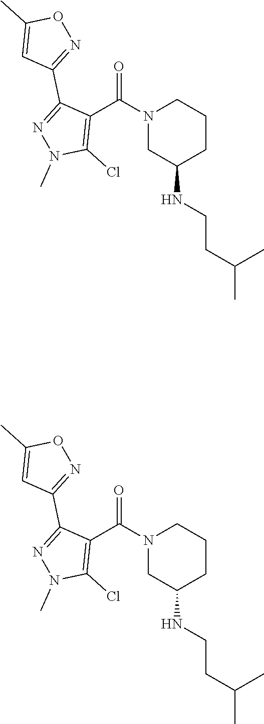

Starting from (5-chloro-1-methyl-3-(5-methylisoxazol-3-yl)-1H-pyrazol-4-yl)(3-(isopentylamino)piperidin-1-yl)methanone, obtained following the procedure described in example 83, a chiral preparative HPLC separation (Column AD-H 20×250 mm, 5 μm; temperature: r.t.; eluent n-Heptane/EtOH/Et₂NH 80/20/0.1 v/v/v; flow rate 13 mL/min; Rt1: 9.1 min; Rt2: 11.4 min) was carried out to give the title compounds.

HPLC Rt (Method B): 1.68 min; ESI+−MS m/z: 394.3 (M+H)⁺.

HPLC Rt (Method B): 1.68 min; ESI+−MS m/z: 394.3 (M+H)⁺.

### Example 110. (5-Chloro-1-methyl-3-(5-methylisoxazol-3-yl)-1H-pyrazol-4-yl)(3-(methyl(phenethyl)amino)azepan-1-yl)methanone

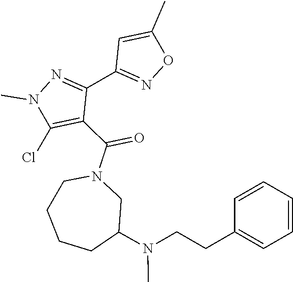 [SMILES: Cc1cc(-c2nn(C)c(Cl)c2C(=O)N2CCCCC(N(C)CCc3ccccc3)C2)no1]

Starting from the compound obtained in example 85 following the procedure described in example 80, the title compound was obtained.

HPLC Rt (Method B): 2.17 min; ESI+−MS m/z: 456.2 (M+H)⁺.

This method was used for the preparation of example 111 using suitable starting materials:

| | | | Rt | MS | HPLC |
|---|---|---|---|---|---|
| STRUCTURE | EX | CHEMICAL NAME | (min) | (M + H) | Method |
| 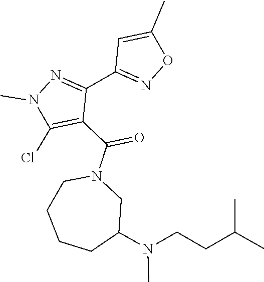 [SMILES: Cc1cc(-c2nn(C)c(Cl)c2C(=O)N2CCCCC(N(C)CCC(C)C)C2)no1] | 111 | (5-Chloro-1-methyl-3-(5- methylisoxazol-3-yl)-1H- pyrazol-4-yl)(3- (isopentyl(methyl)amino) azepan-1-yl)methanone | 2.11 | 422.2 | B |

### Example 112. 2-(9-(5-Chloro-1-methyl-3-(5-methylisoxazol-3-yl)-1H-pyrazole-4-carbonyl)-3,9-diazaspiro[5.5]undecan-3-yl)-1-(piperidin-1-yl)ethanone

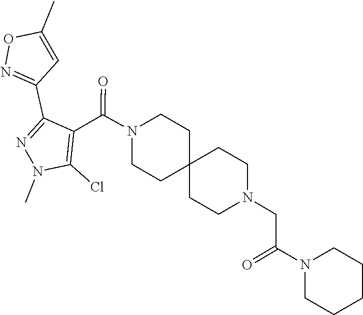 [SMILES: Cc1cc(-c2nn(C)c(Cl)c2C(=O)N2CCC3(CCN(CC(=O)N4CCCCC4)CC3)CC2)no1]

### Step 1. 9-(5-Chloro-1-methyl-3-(5-methylisoxazol-3-yl)-1H-pyrazole-4-carbonyl)-3,9-diazaspiro[5.5]undecan-3-ium 2,2,2-trifluoroacetate

tert-Butyl 9-(5-chloro-1-methyl-3-(5-methylisoxazol-3-yl)-1H-pyrazole-4-carbonyl)-3,9-diazaspiro[5.5]undecane-3-carboxylate (420 mg, 0.88 mmol), obtained following the procedure described in example 83 step 2, was dissolved in CH₂Cl₂ (15 mL) and the solution cooled to 0° C. At this temperature TFA (336 μL, 4.39 mmol) was added and the resulting solution was slowly allowed to reach rt and stirred overnight. The volatiles were removed under reduced pressure to give the title compound (454 mg, quant. overweight, TFA salt) as white solid.

HPLC Rt (Method A): 1.07 min; ESI+−MS m/z: 378.2 (M+H)⁺.

### Step 2. Title Compound

In a Radley tube 1-(chloroacetyl)piperidine (43.9 mg, 0.23 mmol) was dissolved in MeCN (5 mL). The compound obtained in Step 1 (110 mg, 0.22 mmol) in MeCN (3 mL) was added, followed by potassium carbonate (155 mg, 1.12 mmol) and KI (catalytic). The reaction mixture was heated at 70° C. overnight and was concentrated. The residue was taken up with EtOAc and water and the aqueous phase was extracted with EtOAc. The combined organic layers were dried over Na₂SO₄, filtered and concentrated to dryness. The residue was purified by flash chromatography (silica gel, CH₂Cl₂/MeOH) to give the title compound (67 mg, 57%) as oil.

HPLC Rt (Method A): 1.7 min; ESI+−MS m/z: 503.2 (M+H)⁺.

This method was used for the preparation of example 113 using suitable starting materials.

| | | | Rt | MS | HPLC |
|---|---|---|---|---|---|
| STRUCTURE | EX | CHEMICAL NAME | (min) | (M + H) | Method |
| 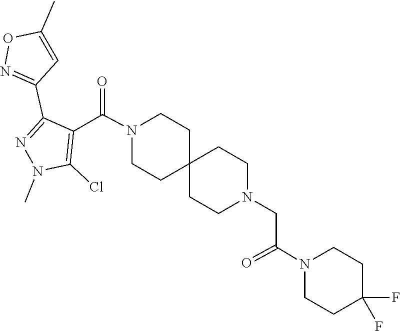 [SMILES: Cc1cc(-c2nn(C)c(Cl)c2C(=O)N2CCC3(CCN(CC(=O)N4CCC(F)(F)CC4)CC3)CC2)no1] | 113 | 2-(9-(5-Chloro-1-methyl-3- (5-methylisoxazol-3-yl)- 1H-pyrazole-4-carbonyl)- 3,9- diazaspiro[5.5]undecan-3- yl)-1-(4,4-difluoropiperidin- 1-yl)ethanone | 1.75 | 539.2 | A |

### Example 114. 2-((5-Chloro-1-methyl-3-(5-methylisoxazol-3-yl)-1H-pyrazol-4-yl)methyl)-8-(3,3-dimethylbutyl)-2,8-diazaspiro[4.5]decan-1-one

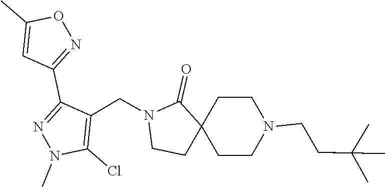 [SMILES: Cc1cc(-c2nn(C)c(Cl)c2CN2CCC3(CCN(CCC(C)(C)C)CC3)C2=O)no1]

### Step 1. (5-Chloro-1-methyl-3-(5-methylisoxazol-3-yl)-1H-pyrazol-4-yl)methanol

Intermediate 1 (500 mg, 2.22 mmol) was dissolved in MeOH (10 mL) and cooled to 0° C. Then sodium borohydride (92.2 mg, 2.44 mmol) was added gradually in two portions and the reaction mixture was stirred at rt for 3 h. The reaction was quenched with water at 0° C. and the volatiles were removed under reduced pressure. The residue was diluted with EtOAc and washed with water. The organic layer was dried over Na₂SO₄, filtered and concentrated to dryness to give the title compound (480 mg, 95% yield).

HPLC Rt (Method A): 1.39 min; ESI+−MS m/z: fragment 210.

### Step 2. 3-(5-Chloro-4-(chloromethyl)-1-methyl-1H-pyrazol-3-yl)-5-methylisoxazole

The compound obtained in Step 1 (505 mg, 2.22 mmol) was dissolved in toluene and thionyl chloride (345 μL, 4.73 mmol) was added dropwise at rt. The reaction mixture was heated at 100° C. for 4 h. All volatiles were removed under reduced pressure to give the title compound (544 mg, quant.).

HPLC Rt (Method A): 2.02 min; ESI+−MS m/z: 246 (M+H)⁺.

### Step 3. tert-Butyl 2-((5-chloro-1-methyl-3-(5-methylisoxazol-3-yl)-1H-pyrazol-4-yl)methyl)-1-oxo-2,8-diazaspiro[4.5]decane-8-carboxylate

To a solution of tert-butyl 1-oxo-2,8-diazaspiro[4.5]decane-8-carboxylate in DMF (2 mL) at 0° C. was added NaH (60% dispersion in mineral oil, 104 mg, 2.60 mmol). After stirring for 1 h at rt, the compound obtained in Step 2 (138 mg, 0.56 mmol) was added. The resulting reaction mixture was stirred for 1 h at rt. The reaction was then quenched by the addition of water and extracted with EtOAc/Et₂O (2:1). The combined organic layers were dried over Na₂SO₄, filtered and concentrated to dryness. The residue was purified by flash chromatography (silica gel, Chx/EtOAc) to give the title compound (148 mg, 74% yield).

HPLC Rt (Method A): 2.09 min; ESI+−MS m/z: 464.2 (M+H)⁺.

### Step 4. 2-((5-Chloro-1-methyl-3-(5-methylisoxazol-3-yl)-1H-pyrazol-4-yl)methyl)-2,8-diazaspiro[4.5]decan-1-one 2,2,2-trifluoroacetate

Starting from the compound obtained in step 3 (148 mg, 0.32 mmol) and following the procedure described in step 3 of example 83, the title compound was obtained (159 mg, quant.).

HPLC Rt (Method A): 1.30 min; ESI+−MS m/z: 364.2 (M+H)⁺.

### Step 5. Title Compound

Starting from the compound obtained in step 3 (152 mg, 0.32 mmol) and following the procedure described in step 4 of example 83, the title compound was obtained (51 mg, 36% yield).

HPLC Rt (Method A): 2.10 min; ESI+−MS m/z: 448.2 (M+H)⁺.

This method was used for the preparation of examples 115 and 116 using suitable starting materials:

| | | | Rt | MS | HPLC |
|---|---|---|---|---|---|
| STRUCTURE | EX | CHEMICAL NAME | (min) | (M + H) | Method |
| 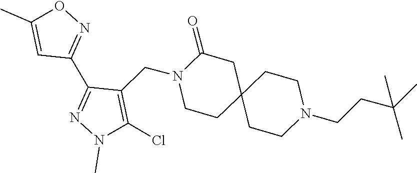 [SMILES: Cc1cc(-c2nn(C)c(Cl)c2CN2CCC3(CCN(CCC(C)(C)C)CC3)CC2=O)no1] | 115 | 3-((5-Chloro-1-methyl-3- (5-methylisoxazol-3-yl)- 1H-pyrazol-4-yl)methyl)-9- (3,3-dimethylbutyl)-3,9- diazaspiro[5.5]undecan-2- one | 1.86 | 462.2 | A |
| 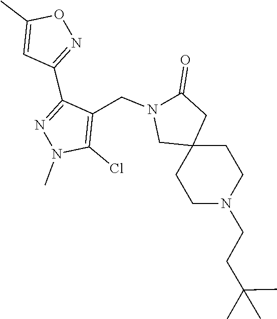 [SMILES: Cc1cc(-c2nn(C)c(Cl)c2CN2CC3(CCN(CCC(C)(C)C)CC3)CC2=O)no1] | 116 | 2-((5-Chloro-1-methyl-3- (5-methylisoxazol-3-yl)- 1H-pyrazol-4-yl)methyl)-8- (3,3-dimethylbutyl)-2,8- diazaspiro[4.5]decan-3- one | 1.96 | 448.2 | A |

### Example 117. 1-((5-Chloro-1-methyl-3-(5-methylisoxazol-3-yl)-1H-pyrazol-4-yl)methyl)-3-(isopentylamino)azepan-2-one

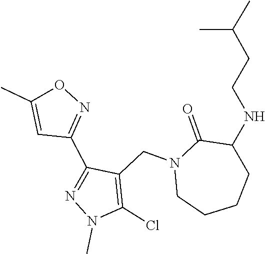 [SMILES: Cc1cc(-c2nn(C)c(Cl)c2CN2CCCCC(NCCC(C)C)C2=O)no1]

In a Radley tube 3-(benzyl(isopentyl)amino)-1-((5-chloro-1-methyl-3-(5-methylisoxazol-3-yl)-1H-pyrazol-4-yl)methyl)azepan-2-one (57 mg, 0.11 mmol), obtained following the procedure for example 114 step 3 with intermediate 20, was dissolved in MeOH (3 mL). Then ammonium formate (47.5 mg, 0.75 mmol) was added followed by 10% Pd/C (22.2 mg, 0.22 mmol) under argon atmosphere. The reaction mixture was heated at 65° C. overnight. After cooling back to rt, the reaction mixture was filtered through Millipore filter paper. The volatiles were removed under reduced pressure. The residue was taken up in water and EtOAc and the pH adjusted to basic. The product was extracted with EtOAc. The combined organic layers were dried over Na₂SO₄, filtered and concentrated to dryness. The residue was purified by reversed phase semipreparative HPLC to obtain the title compound (7 mg, 15% yield).

HPLC Rt (Method B): 1.96 min; ESI+−MS m/z: 408.3 (M+H)⁺.

### Example 118. 1-((5-Chloro-1-methyl-3-(5-methylisoxazol-3-yl)-1H-pyrazol-4-yl)methyl)-4-(isopentylamino)azepan-2-one

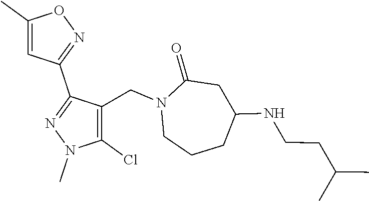 [SMILES: Cc1cc(-c2nn(C)c(Cl)c2CN2CCCC(NCCC(C)C)CC2=O)no1]

Starting from 4-(benzyl(isopentyl)amino)-1-((5-chloro-1-methyl-3-(5-methylisoxazol-3-yl)-1H-pyrazol-4-yl)methyl)azepan-2-one, obtained following the procedure described for example 114 step 3, with intermediate 21, the title compound was obtained following the procedure described for example 117 (4 mg, 6% yield).

HPLC Rt (Method B): 1.72 min; ESI+−MS m/z: 408.3 (M+H)⁺.

### Example 119. (S)-1-(2-(5-Chloro-1-methyl-3-(5-methylisoxazol-3-yl)-1H-pyrazol-4-yl)ethyl)-N-isopentylazepan-3-amine

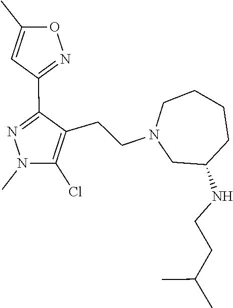 [SMILES: Cc1cc(-c2nn(C)c(Cl)c2CCN2CCCC[C@H](NCCC(C)C)C2)no1]

### Step 1. 2-(5-Chloro-1-methyl-3-(5-methylisoxazol-3-yl)-1H-pyrazol-4-yl)acetonitrile

3-(5-Chloro-4-(chloromethyl)-1-methyl-1H-pyrazol-3-yl)-5-methylisoxazole (43 mg, 0.18 mmol), obtained as described in step 2 of example 114, was dissolved in DMSO (2 mL) and sodium cyanide (34 mg, 0.70 mmol) was added. The reaction mixture was stirred at rt overnight. The reaction mixture was diluted with water and the product extracted with EtOAc. The combined organic layers were dried over Na₂SO₄, filtered and concentrated to dryness to give the title compound (35 mg, 85% yield).

HPLC Rt (Method B): 1.69 min; ESI+−MS m/z 237.1 (M+H)⁺.

### Step 2. 2-(5-Chloro-1-methyl-3-(5-methylisoxazol-3-yl)-1H-pyrazol-4-yl)acetaldehyde

To a solution of the compound obtained in Step 1 (204 mg, 0.86 mmol) in CH₂Cl₂/toluene (6 mL, 1:1) at 0° C., DIBALH (1 M in THF, 3.45 mL, 3.45 mmol), was added and the mixture was slowly allowed to warm up to rt and stirred for 2 h. The reaction mixture was quenched with sat aq. potassium sodium tartrate solution and stirred at rt for 1 h, before the product was extracted with CH₂Cl₂ followed by extraction with EtOAc. The combined organic layers were dried over Na₂SO₄, filtered and concentrated to dryness to give the title compound (90 mg, 44% yield), which was used in the next step without further purification.

HPLC Rt (Method B): 1.70 min; ESI+−MS m/z 240 (M+H)⁺.

### Step 3. Title Compound

To a solution of the compound obtained in Step 2 (70 mg, 0.29 mmol) in MeOH (5 mL) (S)-N-isopentylazepan-3-amine (intermediate 18, 53.8 mg, 0.29 mmol) and sodium cyanoborohydride (27.5 mg, 0.44 mmol) were added. The reaction mixture was stirred at rt for 30 min and then quenched with water and acidified. The aqueous phase was extracted with CH₂Cl₂ to remove impurities and then adjusted to basic pH and extracted with EtOAc. The EtOAc extracts were dried over Na₂SO₄, filtered and concentrated to dryness. The residue was purified by flash chromatography (alumina, Chx/EtOAc, then MeOH) to give the title compound (5 mg, 4% yield).

HPLC Rt (Method A): 2.14 min; ESI+−MS m/z 408.2 (M+H)⁺.

### Example 120. (2-(5-Chloro-1-methyl-3-(5-methylisoxazol-3-yl)-1H-pyrazol-4-yl)-1-(9-isopentyl-3,9-diazaspiro[5.5]undecan-3-yl)ethanone

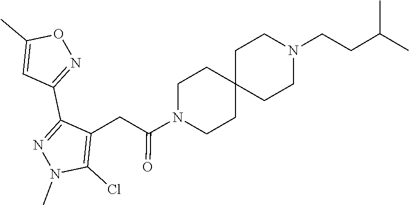 [SMILES: Cc1cc(-c2nn(C)c(Cl)c2CC(=O)N2CCC3(CCN(CCC(C)C)CC3)CC2)no1]

### Step 1. 2-(5-Chloro-1-methyl-3-(5-methylisoxazol-3-yl)-1H-pyrazol-4-yl)acetic acid

To 2-(5-chloro-1-methyl-3-(5-methylisoxazol-3-yl)-1H-pyrazol-4-yl)acetonitrile (150 mg, 0.63 mmol), obtained as described in example 119 step 1, in MeOH (5 mL), sodium hydroxide solution (20%, 5 mL) was added and the mixture was heated to reflux for 2 h. After cooling back to rt, the solvent was removed under reduced pressure. Water was then added and it was acidified with 6 N HCl, followed by extraction with EtOAc. The combined organic layers were dried over Na₂SO₄, filtered and concentrated to dryness to give the title compound (100 mg, 62% yield).

HPLC Rt (Method A): 0.96 min; ESI+−MS m/z 256 (M+H)⁺.

### Step 2. tert-Butyl 9-(2-(5-chloro-1-methyl-3-(5-methylisoxazol-3-yl)-1H-pyrazol-4-yl)acetyl)-3,9-diazaspiro[5.5]undecane-3-carboxylate

Starting from the compound obtained in step 1 (100 mg, 0.39 mmol) and following the procedure described in step 2 of example 83, the title compound was obtained (79 mg, 41% yield). The crude compound was used without further purification in the next step.

HPLC Rt (Method A): 2.23 min; ESI+−MS m/z 492.0 (M+H)⁺.

### Step 3. 2-(5-Chloro-1-methyl-3-(5-methylisoxazol-3-yl)-1H-pyrazol-4-yl)-1-(3,9-diazaspiro[5.5]undecan-3-yl)ethanone

Starting from the compound obtained in step 2 (79 mg, 0.16 mmol) and following the procedure described in step 2 of example 1, the title compound was obtained (69 mg, quant.).

HPLC Rt (Method A): 1.19 min; ESI+−MS m/z 392.0 (M+H)⁺.

### Step 4. Title Compound

To a solution of the compound obtained in Step 3 (69 mg, 0.18 mmol) in MeCN (4 mL) 1-bromo-3-methylbutane (31.4 μL, 0.26 mmol) and potassium carbonate (36.2 mg, 0.26 mmol) were added. The reaction mixture was stirred at rt for 3 hand concentrated. Water was added and the mixture was extracted with CH₂Cl₂ and then with EtOAc. The combined organic layers were dried over Na₂SO₄, filtered and concentrated to dryness. The residue was purified by flash chromatography (silica gel, Chx/EtOAc) to give the title compound (2 mg, 2% yield).

HPLC Rt (Method B): 1.80 min; ESI+−MS m/z 462.1 (M+H)⁺.

### Example 121. (5-Chloro-1-methyl-3-(5-methylisoxazol-3-yl)-1H-pyrazol-4-yl)(3-(((3,3-dimethylbutyl)(methyl)amino)methyl)piperidin-1-yl)methanone

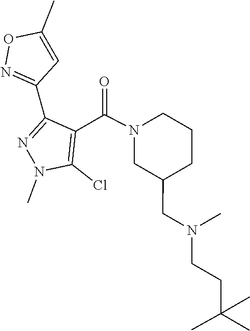 [SMILES: Cc1cc(-c2nn(C)c(Cl)c2C(=O)N2CCCC(CN(C)CCC(C)(C)C)C2)no1]

Starting from the compound obtained in example 83 following the procedure described in example 80, the title compound was obtained.

HPLC Rt (Method A): 2.26 min; ESI+−MS m/z: 436.2 (M+H)⁺.

This method was used for the preparation of example 122 using suitable starting materials.

| | | | Rt | MS | HPLC |
|---|---|---|---|---|---|
| STRUCTURE | EX | CHEMICAL NAME | (min) | (M + H) | Method |
| 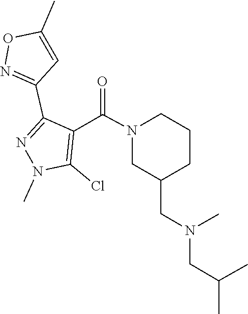 [SMILES: Cc1cc(-c2nn(C)c(Cl)c2C(=O)N2CCCC(CN(C)CC(C)C)C2)no1] | 122 | (5-Chloro-1-methyl-3-(5- methylisoxazol-3-yl)-1H- pyrazol-4-yl)(3- ((isobutyl(methyl)amino) methyl)piperidin-1- yl)methanone | 2.34 | 408.2 | A |

### Example 123. (5-Chloro-1-methyl-3-(5-methylisoxazol-3-yl)-1H-pyrazol-4-yl)((2r,4s)-2-((3,3-dimethylbutyl)amino)-6-azaspiro[3.4]octan-6-yl)methanone

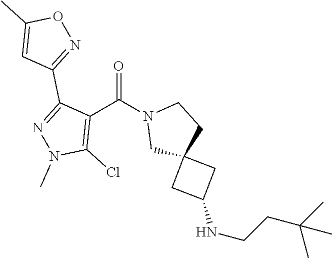 [SMILES: Cc1cc(-c2nn(C)c(Cl)c2C(=O)N2CC[C@]3(C2)C[C@@H](NCCC(C)(C)C)C3)no1]

Starting from (5-chloro-1-methyl-3-(5-methylisoxazol-3-yl)-1H-pyrazol-4-yl)(2-((3,3-dimethylbutyl)amino)-6-azaspiro[3.4]octan-6-yl)methanone, obtained following the procedure described in example 83, a chiral preparative HPLC separation (Column Chiralpak IB 20×250 mm, 5 μm; temperature: r.t.; eluent n-Heptane/IPA/Et₂NH 85/15/0.05 v/v/v; flow rate: 12 mL/min; Rt: 30.1 min) was carried out to give the title compound.

HPLC Rt (Method A): 1.70 min; ESI+−MS m/z: 434.2 (M+H)⁺.

### Example 124. (5-Chloro-3-(6-hydroxypyridin-2-yl)-1-methyl-1H-pyrazol-4-yl)(9-(3,3-dimethylbutyl)-3,9-diazaspiro[5.5]undecan-3-yl)methanone

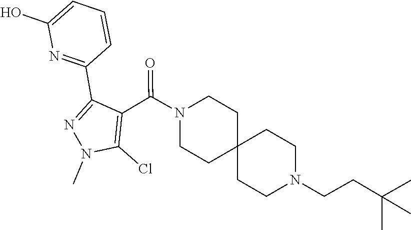 [SMILES: Cn1nc(-c2cccc(O)n2)c(C(=O)N2CCC3(CCN(CCC(C)(C)C)CC3)CC2)c1Cl]

A solution of 5-chloro-3-(6-methoxypyridin-2-yl)-1-methyl-1H-pyrazol-4-yl)(9-(3,3-dimethylbutyl)-3,9-diazaspiro[5.5]undecan-3-yl)methanone (example 102, 45 mg, 0.09 mmol), HBr (48% w/w, 10 μL) and AcOH (0.4 mL) was heated in a sealed tube at 100° C. for 2 h. The reaction was cooled down to rt, quenched at 0° C. with water and basified with sat. sodium carbonate solution. The product was extracted with EtOAc and the combined organic fractions dried over Na₂SO₄. The residue was purified by flash chromatography (silica gel, CH₂Cl₂/MeOH) to give the title compound (27 mg, 62% yield).

HPLC Rt (Method A): 1.52 min; ESI+−MS m/z: 474.2 (M+H)⁺.

### Examples 125 and 126. (R)-(5-Chloro-1-methyl-3-(5-methylisoxazol-3-yl)-1H-pyrazol-4-yl)(3-((isopentylamino)methyl)piperidin-1-yl)methanone and (S)-(5-Chloro-1-methyl-3-(5-methylisoxazol-3-yl)-1H-pyrazol-4-yl)(3-((isopentylamino)methyl)piperidin-1-yl)methanone

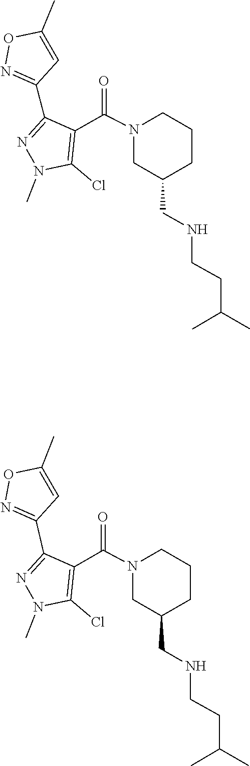 [SMILES: Cc1cc(-c2nn(C)c(Cl)c2C(=O)N2CCC[C@H](CNCCC(C)C)C2)no1 ; Cc1cc(-c2nn(C)c(Cl)c2C(=O)N2CCC[C@@H](CNCCC(C)C)C2)no1]

Starting from (5-chloro-1-methyl-3-(5-methylisoxazol-3-yl)-1H-pyrazol-4-yl)(3-((isopentylamino)methyl)piperidin-1-yl)methanone, obtained following the procedure described in example 83, a chiral preparative HPLC separation (Column Chiralpak AD-H 20×250 mm, 5 μm; temperature: r.t.; eluent n-Heptane/EtOH/Et₂NH 80/20/0.07 v/v/v; flow rate: 132 mL/min; Rt1: 10.6 min; Rt2: 12.1 min) was carried out to give the title compounds.

HPLC Rt (Method B): 1.55 and 1.56 min; ESI+−MS m/z: 408.1 (M+H)⁺.

### Examples 127-168

The following examples were synthesized following the method described in example 83 and using suitable starting materials:

| | | | Rt | MS | HPLC |
|---|---|---|---|---|---|
| STRUCTURE | EX | CHEMICAL NAME | (min) | (M + H) | Method |
| 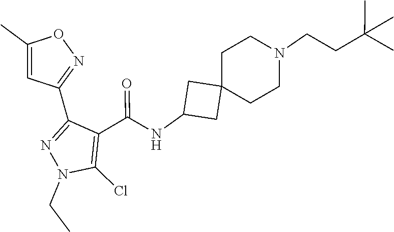 [SMILES: CCn1nc(-c2cc(C)on2)c(C(=O)NC2CC3(CCN(CCC(C)(C)C)CC3)C2)c1Cl] | 127 | 5-Chloro-N-(7-(3,3- dimethylbutyl)-7- azaspiro[3.5]nonan-2-yl)- 1-ethyl-3-(5- methylisoxazol-3-yl)-1H- pyrazole-4-carboxamide | 2.09 | 462.2 | A |
| 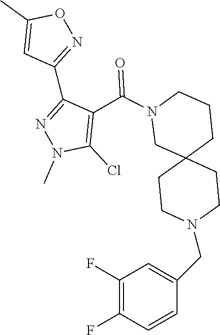 [SMILES: Cc1cc(-c2nn(C)c(Cl)c2C(=O)N2CCCC3(CCN(Cc4ccc(F)c(F)c4)CC3)C2)no1] | 128 | (5-Chloro-1-methyl-3-(5- methylisoxazol-3-yl)-1H- pyrazol-4-yl)(9-(3,4- difluorobenzyl)-2,9- diazaspiro[5.5]undecan-2- yl)methanone | 2.29 | 504.0 | A |
| 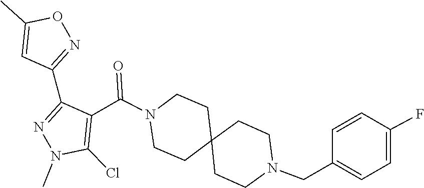 [SMILES: Cc1cc(-c2nn(C)c(Cl)c2C(=O)N2CCC3(CCN(Cc4ccc(F)cc4)CC3)CC2)no1] | 129 | (5-Chloro-1-methyl-3-(5- methylisoxazol-3-yl)-1H- pyrazol-4-yl)(9-(4- fluorobenzyl)-3,9- diazaspiro[5.5]undecan-3- yl)methanone | 2.07 | 486.0 | A |
| 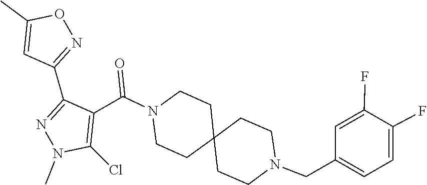 [SMILES: Cc1cc(-c2nn(C)c(Cl)c2C(=O)N2CCC3(CCN(Cc4ccc(F)c(F)c4)CC3)CC2)no1] | 130 | (5-Chloro-1-methyl-3-(5- methylisoxazol-3-yl)-1H- pyrazol-4-yl)(9-(3,4- difluorobenzyl)-3,9- diazaspiro[5.5]undecan-3- yl)methanone | 2.22 | 504.0 | A |
| 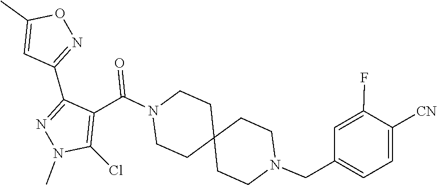 [SMILES: Cc1cc(-c2nn(C)c(Cl)c2C(=O)N2CCC3(CCN(Cc4ccc(C#N)c(F)c4)CC3)CC2)no1] | 131 | 4-((9-(5-Chloro-1-methyl- 3-(5-methylisoxazol-3-yl)- 1H-pyrazole-4-carbonyl)- 3,9- diazaspiro[5.5]undecan-3- yl)methyl)-2- fluorobenzonitrile | 2.10 | 511.0 | A |
| 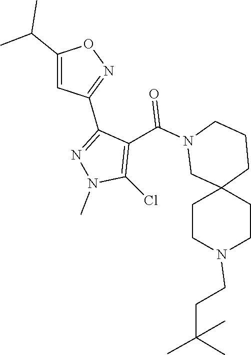 [SMILES: CC(C)c1cc(-c2nn(C)c(Cl)c2C(=O)N2CCCC3(CCN(CCC(C)(C)C)CC3)C2)no1] | 132 | (5-Chloro-3-(5- isopropylisoxazol-3-yl)-1- methyl-1H-pyrazol-4-yl)(9- (3,3-dimethylbutyl)-2,9- diazaspiro[5.5]undecan-2- yl)methanone | 2.21 | 490.2 | A |
| 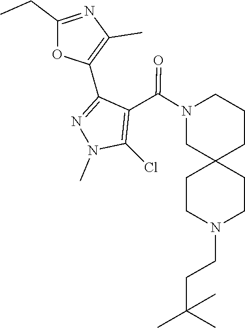 [SMILES: CCc1nc(C)c(-c2nn(C)c(Cl)c2C(=O)N2CCCC3(CCN(CCC(C)(C)C)CC3)C2)o1] | 133 | (5-Chloro-3-(2-ethyl-4- methyloxazol-5-yl)-1- methyl-1H-pyrazol-4-yl)(9- (3,3-dimethylbutyl)-2,9- diazaspiro[5.5]undecan-2- yl)methanone | 1.90 | 490.2 | A |
| 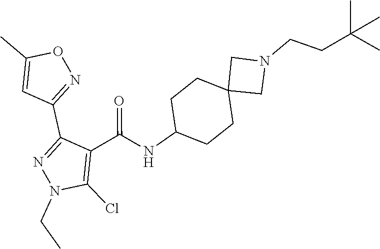 [SMILES: CCn1nc(-c2cc(C)on2)c(C(=O)NC2CCC3(CC2)CN(CCC(C)(C)C)C3)c1Cl] | 134 | 5-Chloro-N-(2-(3,3- dimethylbutyl)-2- azaspiro[3.5]nonan-7-yl)- 1-ethyl-3-(5- methylisoxazol-3-yl)-1H- pyrazole-4-carboxamide | 1.98 | 462.2 | A |
| 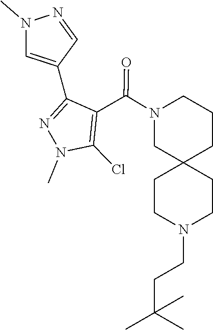 [SMILES: Cn1cc(-c2nn(C)c(Cl)c2C(=O)N2CCCC3(CCN(CCC(C)(C)C)CC3)C2)cn1] | 135 | (5-Chloro-1,1′-dimethyl- 1H,1′H-[3,4′-bipyrazol]-4- yl)(9-(3,3-dimethylbutyl)- 2,9- diazaspiro[5.5]undecan-2- yl)methanone | 1.68 | 461.2 | A |
| 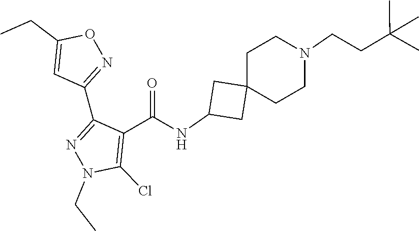 [SMILES: CCc1cc(-c2nn(CC)c(Cl)c2C(=O)NC2CC3(CCN(CCC(C)(C)C)CC3)C2)no1] | 136 | 5-Chloro-N-(7-(3,3- dimethylbutyl)-7- azaspiro[3.5]nonan-2-yl)- 1-ethyl-3-(5-ethylisoxazol- 3-yl)-1H-pyrazole-4- carboxamide | 2.26 | 476.2 | A |
| 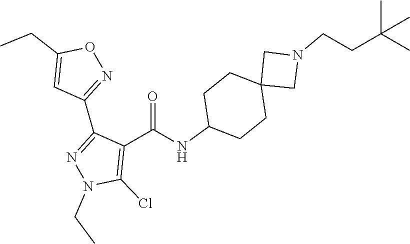 [SMILES: CCc1cc(-c2nn(CC)c(Cl)c2C(=O)NC2CCC3(CC2)CN(CCC(C)(C)C)C3)no1] | 137 | 5-Chloro-N-(2-(3,3- dimethylbutyl)-2- azaspiro[3.5]nonan-7-yl)- 1-ethyl-3-(5-ethylisoxazol- 3-yl)-1H-pyrazole-4- carboxamide | 2.15 | 476.2 | A |
| 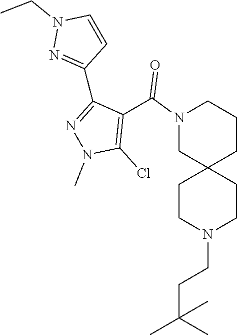 [SMILES: CCn1ccc(-c2nn(C)c(Cl)c2C(=O)N2CCCC3(CCN(CCC(C)(C)C)CC3)C2)n1] | 138 | (5-Chloro-1′-ethyl-1- methyl-1H,1′H-[3,3′- bipyrazol]-4-yl)(9-(3,3- dimethylbutyl)-2,9- diazaspiro[5.5]undecan-2- yl)methanone | 1.85 | 475.2 | A |
| 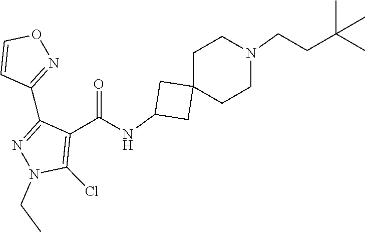 [SMILES: CCn1nc(-c2ccon2)c(C(=O)NC2CC3(CCN(CCC(C)(C)C)CC3)C2)c1Cl] | 139 | 5-Chloro-N-(7-(3,3- dimethylbutyl)-7- azaspiro[3.5]nonan-2-yl)- 1-ethyl-3-(isoxazol-3-yl)- 1H-pyrazole-4- carboxamide | 1.88 | 448.0 | A |
| 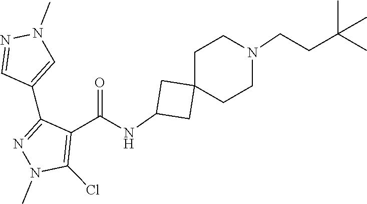 [SMILES: Cn1cc(-c2nn(C)c(Cl)c2C(=O)NC2CC3(CCN(CCC(C)(C)C)CC3)C2)cn1] | 140 | 5-Chloro-N-(7-(3,3- dimethylbutyl)-7- azaspiro[3.5]nonan-2-yl)- 1,1′-dimethyl-1H,1′H-[3,4′- bipyrazole]-4-carboxamide | 1.52 | 447.3 | B |
| 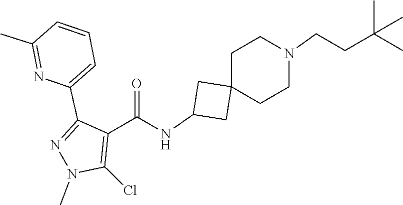 [SMILES: Cc1cccc(-c2nn(C)c(Cl)c2C(=O)NC2CC3(CCN(CCC(C)(C)C)CC3)C2)n1] | 141 | 5-Chloro-N-(7-(3,3- dimethylbutyl)-7- azaspiro[3.5]nonan-2-yl)- 1-methyl-3-(6- methylpyridin-2-yl)-1H- pyrazole-4-carboxamide | 2.12 | 548.3 | B |
| 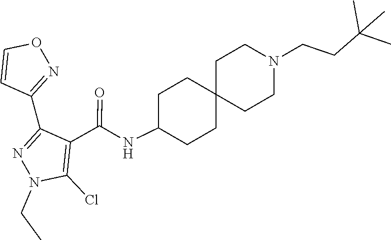 [SMILES: CCn1nc(-c2ccon2)c(C(=O)NC2CCC3(CC2)CCN(CCC(C)(C)C)CC3)c1Cl] | 142 | 5-Chloro-N-(3-(3,3- dimethylbutyl)-3- azaspiro[5.5]undecan-9- yl)-1-ethyl-3-(isoxazol-3- yl)-1H-pyrazole-4- carboxamide | 2.06 | 476.3 | B |
| 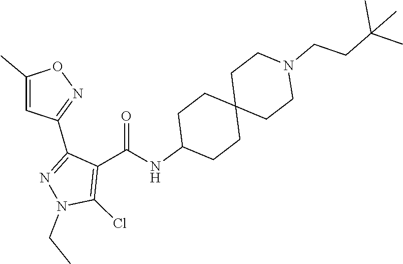 [SMILES: CCn1nc(-c2cc(C)on2)c(C(=O)NC2CCC3(CC2)CCN(CCC(C)(C)C)CC3)c1Cl] | 143 | 5-Chloro-N-(3-(3,3- dimethylbutyl)-3- azaspiro[5.5]undecan-9- yl)-1-ethyl-3-(5- methylisoxazol-3-yl)-1H- pyrazole-4-carboxamide | 2.25 | 490.3 | B |
| 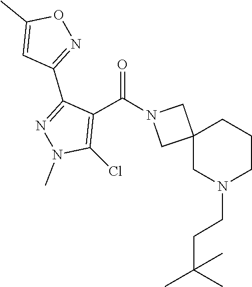 [SMILES: Cc1cc(-c2nn(C)c(Cl)c2C(=O)N2CC3(CCCN(CCC(C)(C)C)C3)C2)no1] | 144 | (5-Chloro-1-methyl-3-(5- methylisoxazol-3-yl)-1H- pyrazol-4-yl)(6-(3,3- dimethylbutyl)-2,6- diazaspiro[3.5]nonan-2- yl)methanone | 2.26 | 434.2 | B |
| 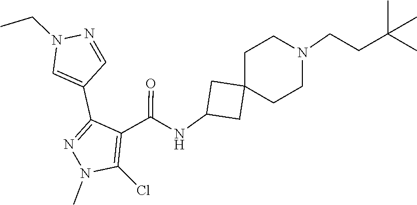 [SMILES: CCn1cc(-c2nn(C)c(Cl)c2C(=O)NC2CC3(CCN(CCC(C)(C)C)CC3)C2)cn1] | 145 | 5-Chloro-N-(7-(3,3- dimethylbutyl)-7- azaspiro[3.5]nonan-2-yl)- 1′-ethyl-1-methyl-1H,1′H- [3,4′-bipyrazole]-4- carboxamide | 1.65 | 461.3 | B |
| 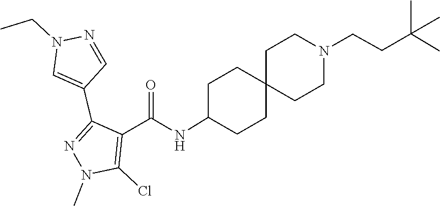 [SMILES: CCn1cc(-c2nn(C)c(Cl)c2C(=O)NC2CCC3(CC2)CCN(CCC(C)(C)C)CC3)cn1] | 146 | 5-Chloro-N-(3-(3,3- dimethylbutyl)-3- azaspiro[5.5]undecan-9- yl)-1′-ethyl-1-methyl- 1H, 1′H-[3,4′-bipyrazole]-4- carboxamide | 1.71 | 489.3 | B |
| 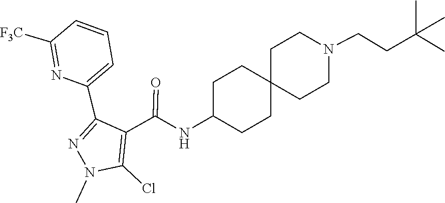 [SMILES: Cn1nc(-c2cccc(C(F)(F)F)n2)c(C(=O)NC2CCC3(CC2)CCN(CCC(C)(C)C)CC3)c1Cl] | 147 | 5-Chloro-N-(3-(3,3- dimethylbutyl)-3- azaspiro[5.5]undecan-9- yl)-1-methyl-3-(6- (trifluoromethyl)pyridin-2- yl)-1H-pyrazole-4- carboxamide | 2.27 | 540.3 | B |
| 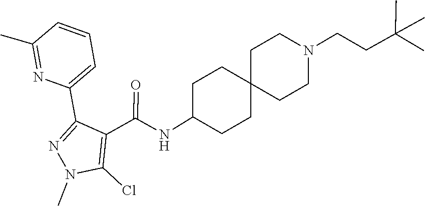 [SMILES: Cc1cccc(-c2nn(C)c(Cl)c2C(=O)NC2CCC3(CC2)CCN(CCC(C)(C)C)CC3)n1] | 148 | 5-Chloro-N-(3-(3,3- dimethylbutyl)-3- azaspiro[5.5]undecan-9- yl)-1-methyl-3-(6- methylpyridin-2-yl)-1H- pyrazole-4-carboxamide | 2.24 | 486.3 | B |
| 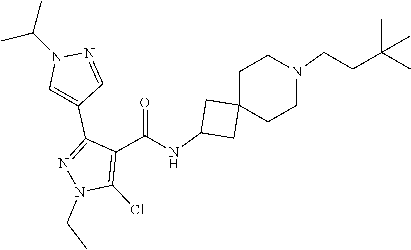 [SMILES: CCn1nc(-c2cnn(C(C)C)c2)c(C(=O)NC2CC3(CCN(CCC(C)(C)C)CC3)C2)c1Cl] | 149 | 5-Chloro-N-(7-(3,3- dimethylbutyl)-7- azaspiro[3.5]nonan-2-yl)- 1-ethyl-1′-isopropyl- 1H,1′H-[3,4′-bipyrazole]-4- carboxamide | 1.91 | 489.3 | B |
| 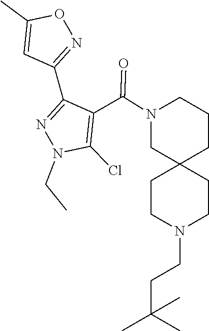 [SMILES: CCn1nc(-c2cc(C)on2)c(C(=O)N2CCCC3(CCN(CCC(C)(C)C)CC3)C2)c1Cl] | 150 | (5-Chloro-1-ethyl-3-(5- methylisoxazol-3-yl)-1H- pyrazol-4-yl)(9-(3,3- dimethylbutyl)-2,9- diazaspiro[5.5]undecan-2- yl)methanone | 2.19 | 476.3 | B |
| 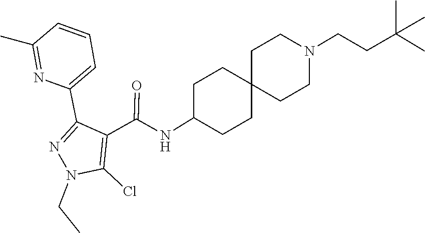 [SMILES: CCn1nc(-c2cccc(C)n2)c(C(=O)NC2CCC3(CC2)CCN(CCC(C)(C)C)CC3)c1Cl] | 151 | 5-Chloro-N-(3-(3,3- dimethylbutyl)-3- azaspiro[5.5]undecan-9- yl)-1-ethyl-3-(6- methylpyridin-2-yl)-1H- pyrazole-4-carboxamide | 2.39 | 500.3 | B |
| 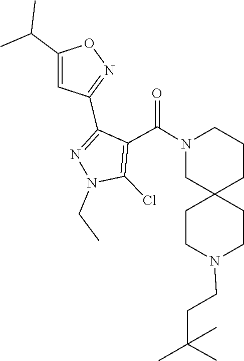 [SMILES: CCn1nc(-c2cc(C(C)C)on2)c(C(=O)N2CCCC3(CCN(CCC(C)(C)C)CC3)C2)c1Cl] | 152 | (5-Chloro-1-ethyl-3-(5- isopropylisoxazol-3-yl)-1H- pyrazol-4-yl)(9-(3,3- dimethylbutyl)-2,9- diazaspiro[5.5]undecan-2- yl)methanone | 2.47 | 504.3 | B |
| 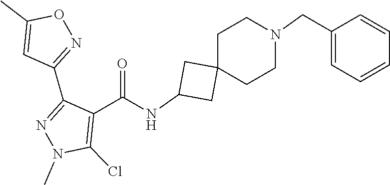 [SMILES: Cc1cc(-c2nn(C)c(Cl)c2C(=O)NC2CC3(CCN(Cc4ccccc4)CC3)C2)no1] | 153 | N-(7-Benzyl-7- azaspiro[3.5]nonan-2-yl)- 5-chloro-1-methyl-3-(5- methylisoxazol-3-yl)-1H- pyrazole-4-carboxamide | 2.10 | 454.2 | B |
| 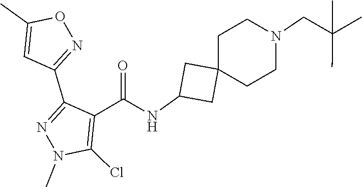 [SMILES: Cc1cc(-c2nn(C)c(Cl)c2C(=O)NC2CC3(CCN(CC(C)(C)C)CC3)C2)no1] | 154 | 5-Chloro-1-methyl-3-(5- methylisoxazol-3-yl)-N-(7- neopentyl-7- azaspiro[3.5]nonan-2-yl)- 1H-pyrazole-4- carboxamide | 2.60 | 434.3 | B |
| 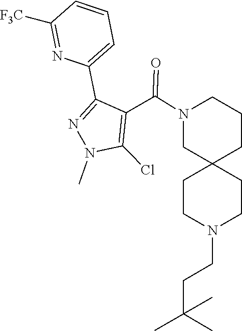 [SMILES: Cn1nc(-c2cccc(C(F)(F)F)n2)c(C(=O)N2CCCC3(CCN(CCC(C)(C)C)CC3)C2)c1Cl] | 155 | (5-Chloro-1-methyl-3-(6- (trifluoromethyl)pyridin-2- yl)-1H-pyrazol-4-yl)(9-(3,3- dimethylbutyl)-2,9- diazaspiro[5.5]undecan-2- yl)methanone | 2.32 | 526.2 | B |
| 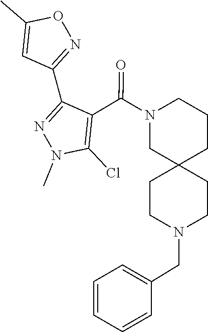 [SMILES: Cc1cc(-c2nn(C)c(Cl)c2C(=O)N2CCCC3(CCN(Cc4ccccc4)CC3)C2)no1] | 156 | (9-Benzyl-2,9- diazaspiro[5.5]undecan-2- yl)(5-chloro-1-methyl-3-(5- methylisoxazol-3-yl)-1H- pyrazol-4-yl)methanone | 2.10 | 468.2 | B |
| 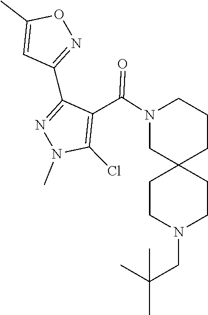 [SMILES: Cc1cc(-c2nn(C)c(Cl)c2C(=O)N2CCCC3(CCN(CC(C)(C)C)CC3)C2)no1] | 157 | (5-Chloro-1-methyl-3-(5- methylisoxazol-3-yl)-1H- pyrazol-4-yl)(9-neopentyl- 2,9- diazaspiro[5.5]undecan-2- yl)methanone | 2.45 | 448.3 | B |
| 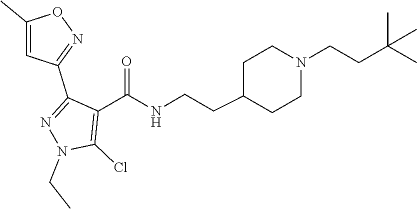 [SMILES: CCn1nc(-c2cc(C)on2)c(C(=O)NCCC2CCN(CCC(C)(C)C)CC2)c1Cl] | 158 | 5-Chloro-N-(2-(1-(3,3- dimethylbutyl)piperidin-4- yl)ethyl)-1-ethyl-3-(5- methylisoxazol-3-yl)-1H- pyrazole-4-carboxamide | 2.08 | 450.3 | B |
| 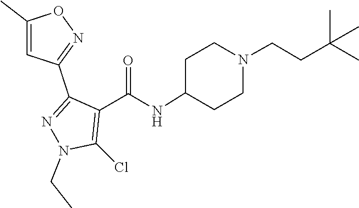 [SMILES: CCn1nc(-c2cc(C)on2)c(C(=O)NC2CCN(CCC(C)(C)C)CC2)c1Cl] | 159 | 5-Chloro-N-(1-(3,3- dimethylbutyl)piperidin-4- yl)-1-ethyl-3-(5- methylisoxazol-3-yl)-1H- pyrazole-4-carboxamide | 2.17 | 422.3 | B |
| 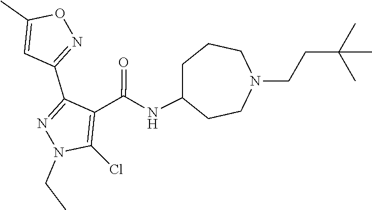 [SMILES: CCn1nc(-c2cc(C)on2)c(C(=O)NC2CCCN(CCC(C)(C)C)CC2)c1Cl] | 160 | 5-Chloro-N-(1-(3,3- dimethylbutyl)azepan-4- yl)-1-ethyl-3-(5- methylisoxazol-3-yl)-1H- pyrazole-4-carboxamide | 2.12 | 436.2 | B |
| 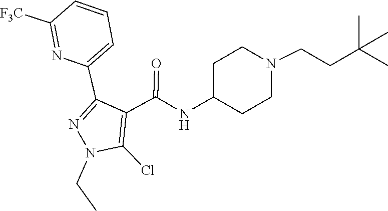 [SMILES: CCn1nc(-c2cccc(C(F)(F)F)n2)c(C(=O)NC2CCN(CCC(C)(C)C)CC2)c1Cl] | 161 | 5-Chloro-N-(1-(3,3- dimethylbutyl)piperidin-4- yl)-1-ethyl-3-(6- (trifluoromethyl)pyridin-2- yl)-1H-pyrazole-4- carboxamide | 2.36 | 486.2 | B |
| 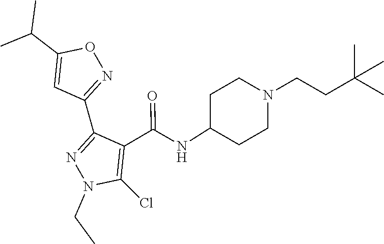 [SMILES: CCn1nc(-c2cc(C(C)C)on2)c(C(=O)NC2CCN(CCC(C)(C)C)CC2)c1Cl] | 162 | 5-Chloro-N-(1-(3,3- dimethylbutyl)piperidin-4- yl)-1-ethyl-3-(5- isopropylisoxazol-3-yl)-1H- pyrazole-4-carboxamide | 2.54 | 450.2 | B |
| 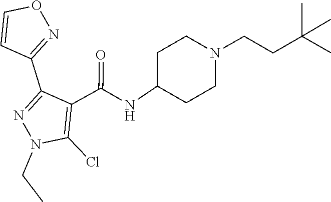 [SMILES: CCn1nc(-c2ccon2)c(C(=O)NC2CCN(CCC(C)(C)C)CC2)c1Cl] | 163 | 5-Chloro-N-(1-(3,3- dimethylbutyl)piperidin-4- yl)-1-ethyl-3-(isoxazol-3- yl)-1H-pyrazole-4- carboxamide | 1.96 | 408.2 | B |
| 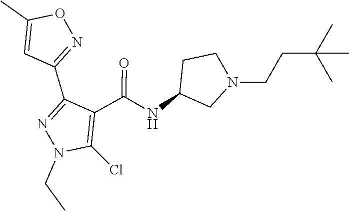 [SMILES: CCn1nc(-c2cc(C)on2)c(C(=O)N[C@H]2CCN(CCC(C)(C)C)C2)c1Cl] | 164 | (S)-5-Chloro-N-(1-(3,3- dimethylbutyl)pyrrolidin-3- yl)-1-ethyl-3-(5- methylisoxazol-3-yl)-1H- pyrazole-4-carboxamide | 2.25 | 408.2 | A |
| 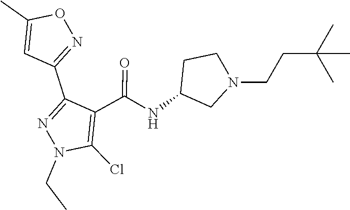 [SMILES: CCn1nc(-c2cc(C)on2)c(C(=O)N[C@@H]2CCN(CCC(C)(C)C)C2)c1Cl] | 165 | (R)-5-Chloro-N-(1-(3,3- dimethylbutyl)pyrrolidin-3- yl)-1-ethyl-3-(5- methylisoxazol-3-yl)-1H- pyrazole-4-carboxamide | 2.22 | 408.2 | B |
| 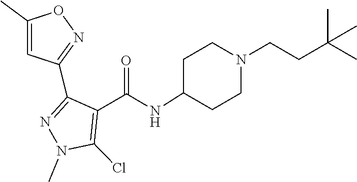 [SMILES: Cc1cc(-c2nn(C)c(Cl)c2C(=O)NC2CCN(CCC(C)(C)C)CC2)no1] | 166 | 5-Chloro-N-(1-(3,3- dimethylbutyl)piperidin-4- yl)-1-methyl-3-(5- methylisoxazol-3-yl)-1H- pyrazole-4-carboxamide | 1.92 | 408.2 | B |
| 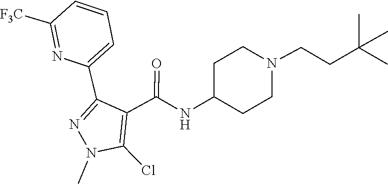 [SMILES: Cn1nc(-c2cccc(C(F)(F)F)n2)c(C(=O)NC2CCN(CCC(C)(C)C)CC2)c1Cl] | 167 | 5-Chloro-N-(1-(3,3- dimethylbutyl)piperidin-4- yl)-1-methyl-3-(6- (trifluoromethyl)pyridin-2- yl)-1H-pyrazole-4- carboxamide | 2.17 | 472.2 | B |
| 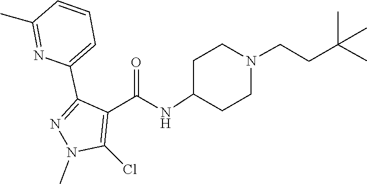 [SMILES: Cc1cccc(-c2nn(C)c(Cl)c2C(=O)NC2CCN(CCC(C)(C)C)CC2)n1] | 168 | 5-Chloro-N-(1-(3,3- dimethylbutyl)piperidin-4- yl)-1-methyl-3-(6- methylpyridin-2-yl)-1H- pyrazole-4-carboxamide | 2.10 | 418.2 | B |

### Example 169. (5-Chloro-1-methyl-3-(5-methylisoxazol-3-yl)-1H-pyrazol-4-yl)((2s,4r)-2-((3,3-dimethylbutyl)amino)-6-azaspiro[3.4]octan-6-yl)methanone

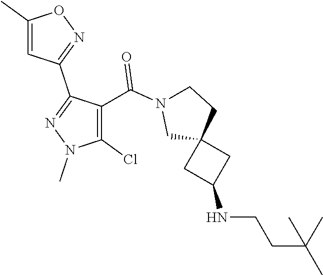 [SMILES: Cc1cc(-c2nn(C)c(Cl)c2C(=O)N2CC[C@]3(C2)C[C@H](NCCC(C)(C)C)C3)no1]

Starting from the product obtained in step 1 of example 83 and intermediate 43 and following the procedure described in step 2 of example 83, the title compound was obtained (72 mg, 50% yield).

HPLC Rt (Method A): 1.64 min; ESI+−MS m/z: 434.2 (M+H)⁺.

This method was used for the preparation of examples 170-207 using suitable starting materials.

| | | | Rt | MS | HPLC |
|---|---|---|---|---|---|
| STRUCTURE | EX | CHEMICAL NAME | (min) | (M + H) | Method |
| 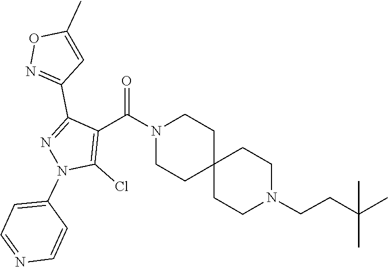 [SMILES: Cc1cc(-c2nn(-c3ccncc3)c(Cl)c2C(=O)N2CCC3(CCN(CCC(C)(C)C)CC3)CC2)no1] | 170 | (5-Chloro-3-(5- methylisoxazol-3-yl)-1- (pyridin-4-yl)-1H-pyrazol- 4-yl)(9-(3,3-dimethylbutyl)- 3,9- diazaspiro[5.5]undecan-3- yl)methanone | 1.95 | 525.2 | A |
| 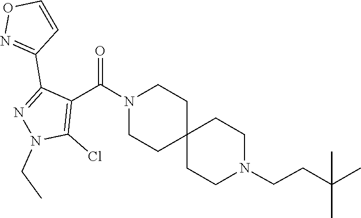 [SMILES: CCn1nc(-c2ccon2)c(C(=O)N2CCC3(CCN(CCC(C)(C)C)CC3)CC2)c1Cl] | 171 | (5-Chloro-1-ethyl-3- (isoxazol-3-yl)-1H-pyrazol- 4-yl)(9-(3,3-dimethylbutyl)- 3,9- diazaspiro[5.5]undecan-3- yl)methanone | 1.87 | 462.2 | A |
| 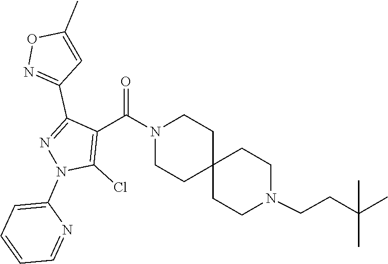 [SMILES: Cc1cc(-c2nn(-c3ccccn3)c(Cl)c2C(=O)N2CCC3(CCN(CCC(C)(C)C)CC3)CC2)no1] | 172 | (5-Chloro-3-(5- methylisoxazol-3-yl)-1- (pyridin-2-yl)-1H-pyrazol- 4-yl)(9-(3,3-dimethylbutyl)- 3,9- diazaspiro[5.5]undecan-3- yl)methanone | 2.02 | 525.2 | A |
| 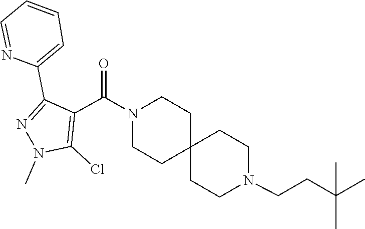 [SMILES: Cn1nc(-c2ccccn2)c(C(=O)N2CCC3(CCN(CCC(C)(C)C)CC3)CC2)c1Cl] | 173 | (5-Chloro-1-methyl-3- (pyridin-2-yl)-1H-pyrazol- 4-yl)(9-(3,3-dimethylbutyl)- 3,9- diazaspiro[5.5]undecan-3- yl)methanone | 1.80 | 458.2 | A |
| 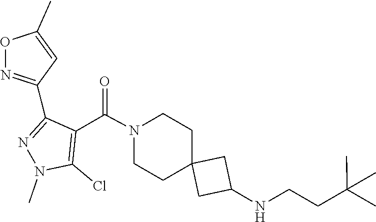 [SMILES: Cc1cc(-c2nn(C)c(Cl)c2C(=O)N2CCC3(CC2)CC(NCCC(C)(C)C)C3)no1] | 174 | (5-Chloro-1-methyl-3-(5- methylisoxazol-3-yl)-1H- pyrazol-4-yl)(2-((3,3- dimethylbutyl)amino)-7- azaspiro[3.5]nonan-7- yl)methanone | 1.72 | 448.2 | A |
| 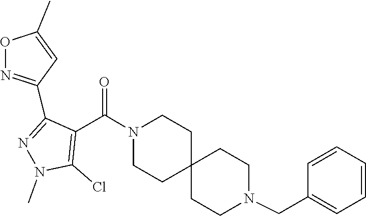 [SMILES: Cc1cc(-c2nn(C)c(Cl)c2C(=O)N2CCC3(CCN(Cc4ccccc4)CC3)CC2)no1] | 175 | (9-Benzyl-3,9- diazaspiro[5.5]undecan-3- yl)(5-chloro-1-methyl-3-(5- methylisoxazol-3-yl)-1H- pyrazol-4-yl)methanone | 2.00 | 468.2 | A |
| 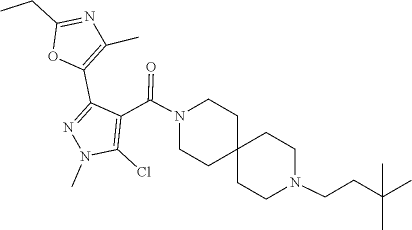 [SMILES: CCc1nc(C)c(-c2nn(C)c(Cl)c2C(=O)N2CCC3(CCN(CCC(C)(C)C)CC3)CC2)o1] | 176 | (5-Chloro-3-(2-ethyl-4- methyloxazol-5-yl)-1- methyl-1H-pyrazol-4-yl)(9- (3,3-dimethylbutyl)-3,9- diazaspiro[5.5]undecan-3- yl)methanone | 1.79 | 490.2 | A |
| 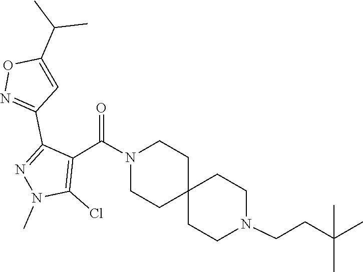 [SMILES: CC(C)c1cc(-c2nn(C)c(Cl)c2C(=O)N2CCC3(CCN(CCC(C)(C)C)CC3)CC2)no1] | 177 | (5-Chloro-3-(5- isopropylisoxazol-3-yl)-1- methyl-1H-pyrazol-4-yl)(9- (3,3-dimethylbutyl)-3,9- diazaspiro[5.5]undecan-3- yl)methanone | 2.18 | 490.3 | B |
| 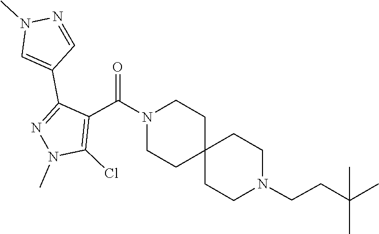 [SMILES: Cn1cc(-c2nn(C)c(Cl)c2C(=O)N2CCC3(CCN(CCC(C)(C)C)CC3)CC2)cn1] | 178 | (5-Chloro-1,1′-dimethyl- 1H,1′H-[3,4′-bipyrazol]-4- yl)(9-(3,3-dimethylbutyl)- 3,9- diazaspiro[5.5]undecan-3- yl)methanone | 1.56 | 461.2 | A |
| 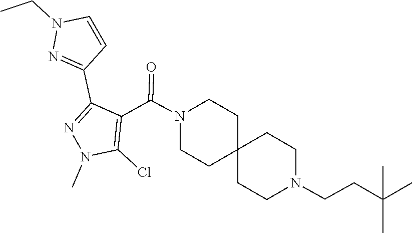 [SMILES: CCn1ccc(-c2nn(C)c(Cl)c2C(=O)N2CCC3(CCN(CCC(C)(C)C)CC3)CC2)n1] | 179 | (5-Chloro-1′-ethyl-1- methyl-1H,1′H-[3,3′- bipyrazol]-4-yl)(9-(3,3- dimethylbutyl)-3,9- diazaspiro[5.5]undecan-3- yl)methanone | 1.70 | 475.2 | A |
| 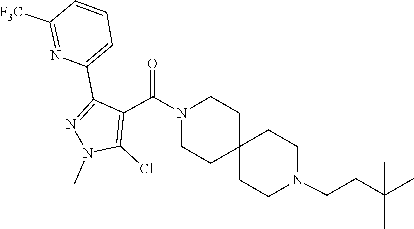 [SMILES: Cn1nc(-c2cccc(C(F)(F)F)n2)c(C(=O)N2CCC3(CCN(CCC(C)(C)C)CC3)CC2)c1Cl] | 180 | (5-Chloro-1-methyl-3-(6- (trifluoromethyl)111ndecane- 2-yl)-1H-pyrazol-4-yl)(9- (3,3-dimethylbutyl)-3,9- diazaspiro[5.5]111ndecane- 3-yl)methanone | 2.13 | 526.2 | A |
| 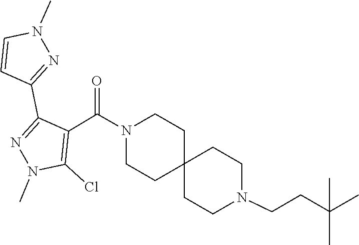 [SMILES: Cn1ccc(-c2nn(C)c(Cl)c2C(=O)N2CCC3(CCN(CCC(C)(C)C)CC3)CC2)n1] | 181 | (5-Chloro-1,1′-dimethyl- 1H,1′H-[3,3′-bipyrazol]-4- yl)(9-(3,3-dimethylbutyl)- 3,9- diazaspiro[5.5]undecan-3- yl)methanone | 1.62 | 461.2 | A |
| 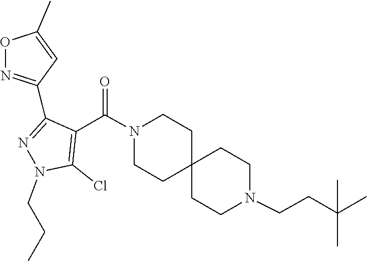 [SMILES: CCCn1nc(-c2cc(C)on2)c(C(=O)N2CCC3(CCN(CCC(C)(C)C)CC3)CC2)c1Cl] | 182 | (5-Chloro-3-(5- methylisoxazol-3-yl)-1- propyl-1H-pyrazol-4-yl)(9- (3,3-dimethylbutyl)-3,9- diazaspiro[5.5]undecan-3- yl)methanone | 2.10 | 490.2 | A |
| 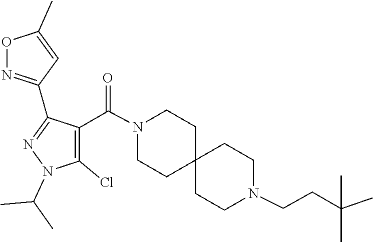 [SMILES: Cc1cc(-c2nn(C(C)C)c(Cl)c2C(=O)N2CCC3(CCN(CCC(C)(C)C)CC3)CC2)no1] | 183 | (5-Chloro-1-isopropyl-3-(5- methylisoxazol-3-yl)-1H- pyrazol-4-yl)(9-(3,3- dimethylbutyl)-3,9- diazaspiro[5.5]undecan-3- yl)methanone | 2.12 | 490.2 | A |
| 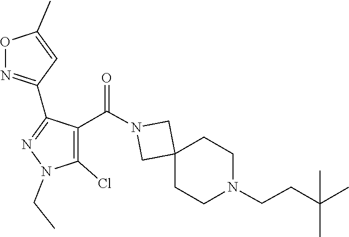 [SMILES: CCn1nc(-c2cc(C)on2)c(C(=O)N2CC3(CCN(CCC(C)(C)C)CC3)C2)c1Cl] | 184 | (5-Chloro-1-ethyl-3-(5- methylisoxazol-3-yl)-1H- pyrazol-4-yl)(7-(3,3- dimethylbutyl)-2,7- diazaspiro[3.5]nonan-2- yl)methanone | 2.00 | 448.2 | A |
| 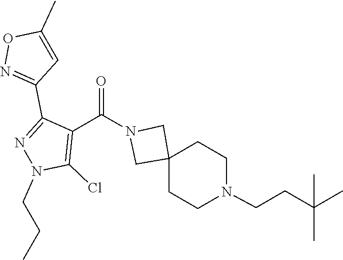 [SMILES: CCCn1nc(-c2cc(C)on2)c(C(=O)N2CC3(CCN(CCC(C)(C)C)CC3)C2)c1Cl] | 185 | (5-Chloro-3-(5- methylisoxazol-3-yl)-1- propyl-1H-pyrazol-4-yl)(7- (3,3-dimethylbutyl)-2,7- diazaspiro[3.5]nonan-2- yl)methanone | 2.16 | 462.2 | A |
| 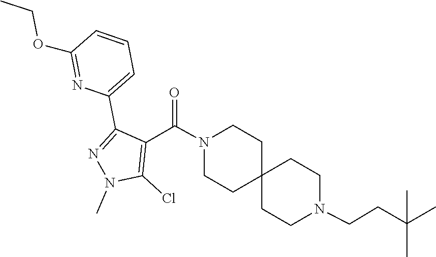 [SMILES: CCOc1cccc(-c2nn(C)c(Cl)c2C(=O)N2CCC3(CCN(CCC(C)(C)C)CC3)CC2)n1] | 186 | (5-Chloro-3-(6- ethoxypyridin-2-yl)-1- methyl-1H-pyrazol-4-yl)(9- (3,3-dimethylbutyl)-3,9- diazaspiro[5.5]undecan-3- yl)methanone | 2.10 | 502.2 | A |
| 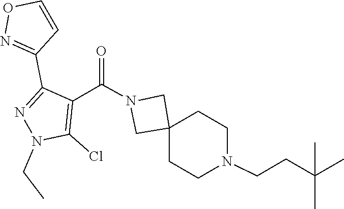 [SMILES: CCn1nc(-c2ccon2)c(C(=O)N2CC3(CCN(CCC(C)(C)C)CC3)C2)c1Cl] | 187 | (5-Chloro-1-ethyl-3- (isoxazol-3-yl)-1H-pyrazol- 4-yl)(7-(3,3-dimethylbutyl)- 2,7-diazaspiro[3.5]nonan- 2-yl)methanone | 1.91 | 434.2 | A |
| 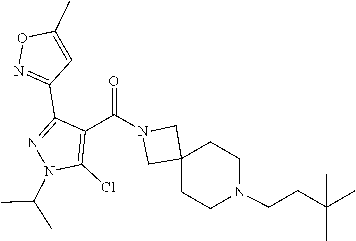 [SMILES: Cc1cc(-c2nn(C(C)C)c(Cl)c2C(=O)N2CC3(CCN(CCC(C)(C)C)CC3)C2)no1] | 188 | (5-Chloro-1-isopropyl-3-(5- methylisoxazol-3-yl)-1H- pyrazol-4-yl)(7-(3,3- dimethylbutyl)-2,7- diazaspiro[3.5]nonan-2- yl)methanone | 2.20 | 462.2 | A |
| 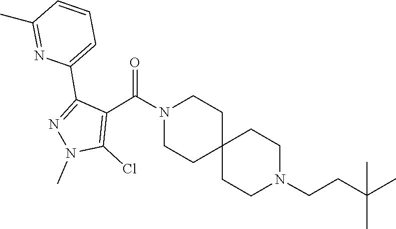 [SMILES: Cc1cccc(-c2nn(C)c(Cl)c2C(=O)N2CCC3(CCN(CCC(C)(C)C)CC3)CC2)n1] | 189 | (5-Chloro-1-methyl-3-(6- methylpyridin-2-yl)-1H- pyrazol-4-yl)(9-(3,3- dimethylbutyl)-3,9- diazaspiro[5.5]undecan-3- yl)methanone | 1.91 | 472.2 | A |
| 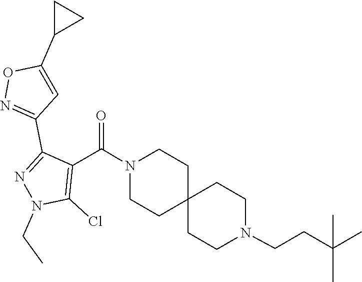 [SMILES: CCn1nc(-c2cc(C3CC3)on2)c(C(=O)N2CCC3(CCN(CCC(C)(C)C)CC3)CC2)c1Cl] | 190 | (5-Chloro-3-(5- cyclopropylisoxazol-3-yl)- 1-ethyl-1H-pyrazol-4-yl)(9- (3,3-dimethylbutyl)-3,9- diazaspiro[5.5]undecan-3- yl)methanone | 2.19 | 502.3 | B |
| 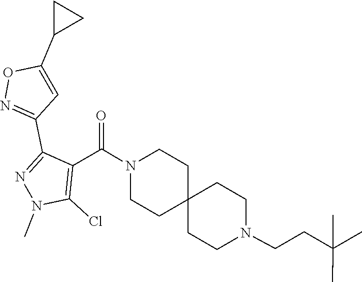 [SMILES: Cn1nc(-c2cc(C3CC3)on2)c(C(=O)N2CCC3(CCN(CCC(C)(C)C)CC3)CC2)c1Cl] | 191 | (5-Chloro-3-(5- cyclopropylisoxazol-3-yl)- 1-methyl-1H-pyrazol-4- yl)(9-(3,3-dimethylbutyl)- 3,9- diazaspiro[5.5]undecan-3- yl)methanone | 2.03 | 488.3 | B |
| 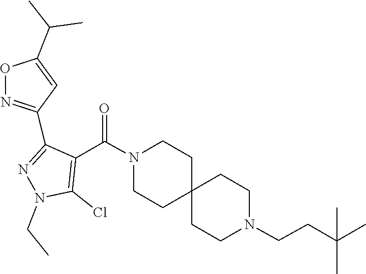 [SMILES: CCn1nc(-c2cc(C(C)C)on2)c(C(=O)N2CCC3(CCN(CCC(C)(C)C)CC3)CC2)c1Cl] | 192 | (5-Chloro-1-ethyl-3-(5- isopropylisoxazol-3-yl)-1H- pyrazol-4-yl)(9-(3,3- dimethylbutyl)-3,9- diazaspiro[5.5]undecan-3- yl)methanone | 2.36 | 504.3 | B |
| 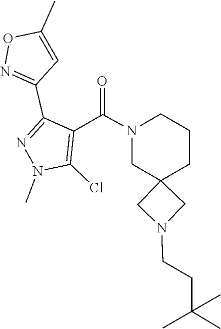 [SMILES: Cc1cc(-c2nn(C)c(Cl)c2C(=O)N2CCCC3(CN(CCC(C)(C)C)C3)C2)no1] | 193 | (5-Chloro-1-methyl-3-(5- methylisoxazol-3-yl)-1H- pyrazol-4-yl)(2-(3,3- dimethylbutyl)-2,6- diazaspiro[3.5]nonan-6- yl)methanone | 1.91 | 434.2 | B |
| 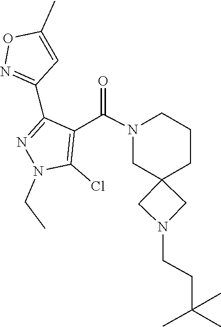 [SMILES: CCn1nc(-c2cc(C)on2)c(C(=O)N2CCCC3(CN(CCC(C)(C)C)C3)C2)c1Cl] | 194 | (5-Chloro-1-ethyl-3-(5- methylisoxazol-3-yl)-1H- pyrazol-4-yl)(2-(3,3- dimethylbutyl)-2,6- diazaspiro[3.5]nonan-6- yl)methanone | 2.08 | 448.2 | B |
| 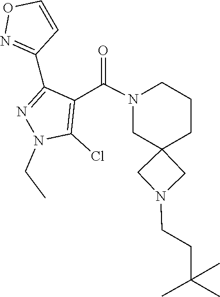 [SMILES: CCn1nc(-c2ccon2)c(C(=O)N2CCCC3(CN(CCC(C)(C)C)C3)C2)c1Cl] | 195 | (5-Chloro-1-ethyl-3- (isoxazol-3-yl)-1H-pyrazol- 4-yl)(2-(3,3-dimethylbutyl)- 2,6-diazaspiro[3.5]nonan- 6-yl)methanone | 1.98 | 434.2 | B |
| 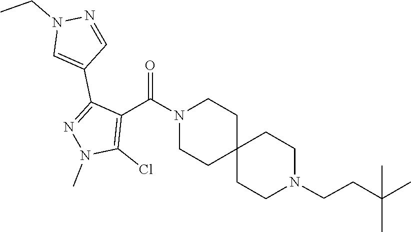 [SMILES: CCn1cc(-c2nn(C)c(Cl)c2C(=O)N2CCC3(CCN(CCC(C)(C)C)CC3)CC2)cn1] | 196 | (5-Chloro-1′-ethyl-1- methyl-1H,1′H-[3,4′- bipyrazol]-4-yl)(9-(3,3- dimethylbutyl)-3,9- diazaspiro[5.5]undecan-3- yl)methanone | 1.67 | 475.3 | B |
| 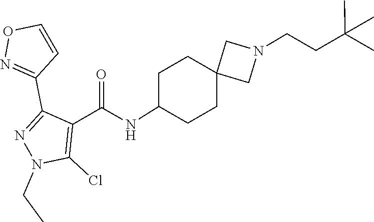 [SMILES: CCn1nc(-c2ccon2)c(C(=O)NC2CCC3(CC2)CN(CCC(C)(C)C)C3)c1Cl] | 197 | 5-Chloro-N-(2-(3,3- dimethylbutyl)-2- azaspiro[3.5]nonan-7-yl)- 1-ethyl-3-(isoxazol-3-yl)- 1H-pyrazole-4- carboxamide | 1.94 | 448.2 | B |
| 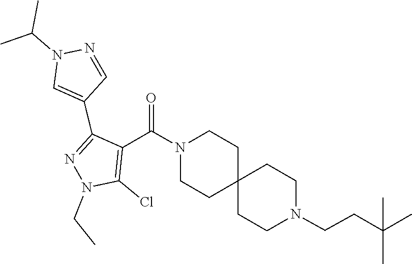 [SMILES: CCn1nc(-c2cnn(C(C)C)c2)c(C(=O)N2CCC3(CCN(CCC(C)(C)C)CC3)CC2)c1Cl] | 198 | (5-Chloro-1-ethyl-1′- isopropyl-1H,1′H-[3,4′- bipyrazol]-4-yl)(9-(3,3- dimethylbutyl)-3,9- diazaspiro[5.5]undecan-3- yl)methanone | 1.97 | 503.3 | B |
| 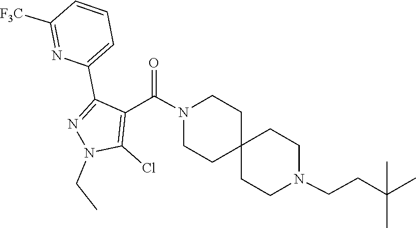 [SMILES: CCn1nc(-c2cccc(C(F)(F)F)n2)c(C(=O)N2CCC3(CCN(CCC(C)(C)C)CC3)CC2)c1Cl] | 199 | (5-Chloro-1-ethyl-3-(6- (trifluoromethyl)pyridin-2- yl)-1H-pyrazol-4-yl)(9-(3,3- dimethylbutyl)-3,9- diazaspiro[5.5]undecan-3- yl)methanone | 2.37 | 540.2 | B |
| 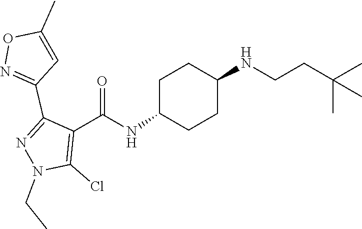 [SMILES: CCn1nc(-c2cc(C)on2)c(C(=O)N[C@H]2CC[C@H](NCCC(C)(C)C)CC2)c1Cl] | 200 | 5-Chloro-N-((1r,4r)-4-((3,3- dimethylbutyl)amino)cyclo hexyl)-1-ethyl-3-(5- methylisoxazol-3-yl)-1H- pyrazole-4-carboxamide | 1.79 | 436.2 | A |
| 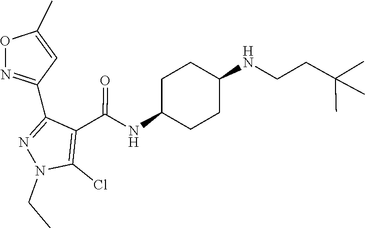 [SMILES: CCn1nc(-c2cc(C)on2)c(C(=O)N[C@H]2CC[C@@H](NCCC(C)(C)C)CC2)c1Cl] | 201 | 5-Chloro-N-((1s,4s)-4- ((3,3- dimethylbutyl)amino)cyclo hexyl)-1-ethyl-3-(5- methylisoxazol-3-yl)-1H- pyrazole-4-carboxamide | 1.78 | 436.2 | A |
| 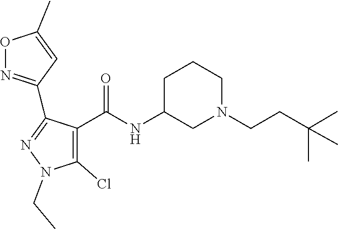 [SMILES: CCn1nc(-c2cc(C)on2)c(C(=O)NC2CCCN(CCC(C)(C)C)C2)c1Cl] | 202 | 5-Chloro-N-(1-(3,3- dimethylbutyl)piperidin-3- yl)-1-ethyl-3-(5- methylisoxazol-3-yl)-1H- pyrazole-4-carboxamide | 2.50 | 422.3 | B |
| 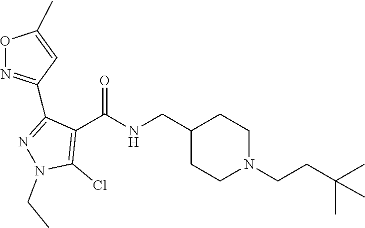 [SMILES: CCn1nc(-c2cc(C)on2)c(C(=O)NCC2CCN(CCC(C)(C)C)CC2)c1Cl] | 203 | 5-Chloro-N-((1-(3,3- dimethylbutyl)piperidin-4- yl)methyl)-1-ethyl-3-(5- methylisoxazol-3-yl)-1H- pyrazole-4-carboxamide | 1.99 | 436.3 | B |
| 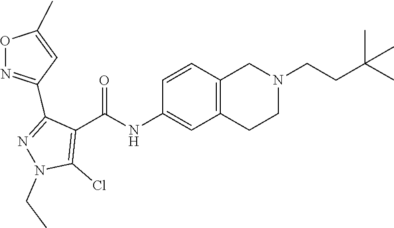 [SMILES: CCn1nc(-c2cc(C)on2)c(C(=O)Nc2ccc3c(c2)CCN(CCC(C)(C)C)C3)c1Cl] | 204 | 5-Chloro-N-(2-(3,3- dimethylbutyl)-1,2,3,4- tetrahydroisoquinolin-6-yl)- 1-ethyl-3-(5- methylisoxazol-3-yl)-1H- pyrazole-4-carboxamide | 2.79 | 470.2 | B |
| 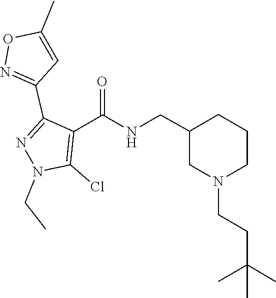 [SMILES: CCn1nc(-c2cc(C)on2)c(C(=O)NCC2CCCN(CCC(C)(C)C)C2)c1Cl] | 205 | 5-Chloro-N-((1-(3,3- dimethylbutyl)piperidin-3- yl)methyl)-1-ethyl-3-(5- methylisoxazol-3-yl)-1H- pyrazole-4-carboxamide | 2.10 | 436.3 | B |
| 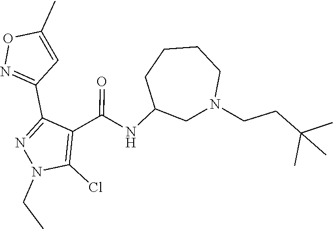 [SMILES: CCn1nc(-c2cc(C)on2)c(C(=O)NC2CCCCN(CCC(C)(C)C)C2)c1Cl] | 206 | 5-Chloro-N-(1-(3,3- dimethylbutyl)azepan-3- yl)-1-ethyl-3-(5- methylisoxazol-3-yl)-1H- pyrazole-4-carboxamide | 2.63 | 436.3 | B |
| 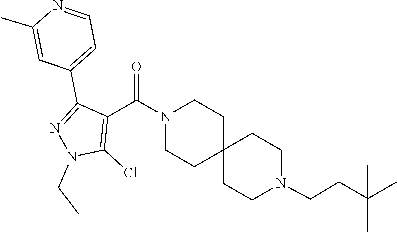 [SMILES: CCn1nc(-c2ccnc(C)c2)c(C(=O)N2CCC3(CCN(CCC(C)(C)C)CC3)CC2)c1Cl] | 207 | (5-Chloro-1-ethyl-3-(2- methylpyridin-4-yl)-1H- pyrazol-4-yl)(9-(3,3- dimethylbutyl)-3,9- diazaspiro[5.5]undecan-3- yl)methanone | 1.84 | 486.0 | B |

### Example 208. 2-(5-Chloro-1-methyl-3-(5-methylisoxazol-3-yl)-1H-pyrazol-4-yl)-1-(9-(3,3-dimethylbutyl)-3,9-diazaspiro[5.5]undecan-3-yl)ethan-1-one

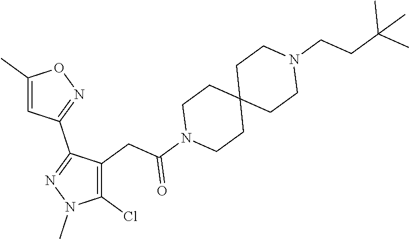 [SMILES: Cc1cc(-c2nn(C)c(Cl)c2CC(=O)N2CCC3(CCN(CCC(C)(C)C)CC3)CC2)no1]

Starting from the product obtained in step 1 of example 120 and intermediate 44 and following the procedure described in step 2 of example 83, the title compound was obtained (31 mg, 33% yield).

HPLC Rt (Method B): 1.99 min; ESI+−MS m/z: 476.3 (M+H)⁺.

This method was used for the preparation of examples 209-213 using suitable starting materials.

| | | | Rt | MS | HPLC |
|---|---|---|---|---|---|
| STRUCTURE | EX | CHEMICAL NAME | (min) | (M + H) | Method |
| 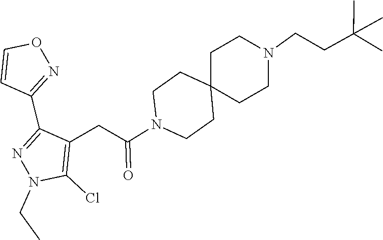 [SMILES: CCn1nc(-c2ccon2)c(CC(=O)N2CCC3(CCN(CCC(C)(C)C)CC3)CC2)c1Cl] | 209 | 2-(5-Chloro-1-ethyl-3- (isoxazol-3-yl)-1H-pyrazol- 4-yl)-1-(9-(3,3- dimethylbutyl)-3,9- diazaspiro[5.5]undecan-3- yl)ethan-1-one | 1.99 | 476.3 | B |
| 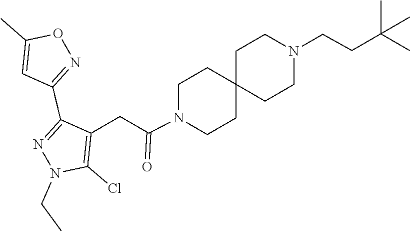 [SMILES: CCn1nc(-c2cc(C)on2)c(CC(=O)N2CCC3(CCN(CCC(C)(C)C)CC3)CC2)c1Cl] | 210 | 2-(5-Chloro-1-ethyl-3-(5- methylisoxazol-3-yl)-1H- pyrazol-4-yl)-1-(9-(3,3- dimethylbutyl)-3,9- diazaspiro[5.5]undecan-3- yl)ethan-1-one | 2.24 | 490.2 | A |
| 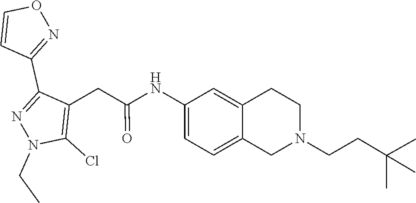 [SMILES: CCn1nc(-c2ccon2)c(CC(=O)Nc2ccc3c(c2)CCN(CCC(C)(C)C)C3)c1Cl] | 211 | 2-(5-Chloro-1-ethyl-3- (isoxazol-3-yl)-1H-pyrazol- 4-yl)-N-(2-(3,3- dimethylbutyl)-1,2,3,4- tetrahydroisoquinolin-6- yl)acetamide | 2.42 | 470.2 | B |
| 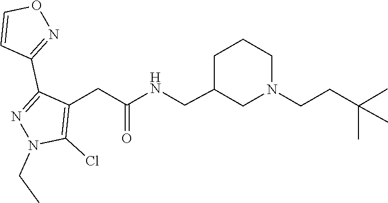 [SMILES: CCn1nc(-c2ccon2)c(CC(=O)NCC2CCCN(CCC(C)(C)C)C2)c1Cl] | 212 | 2-(5-Chloro-1-ethyl-3- (isoxazol-3-yl)-1H-pyrazol- 4-yl)-N-((1-(3,3- dimethylbutyl)piperidin-3- yl)methyl)acetamide | 1.86 | 436.2 | B |
| 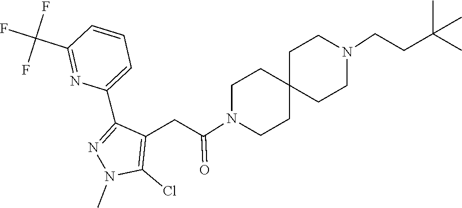 [SMILES: Cn1nc(-c2cccc(C(F)(F)F)n2)c(CC(=O)N2CCC3(CCN(CCC(C)(C)C)CC3)CC2)c1Cl] | 213 | 2-(5-Chloro-1-methyl-3-(6- (trifluoromethyl)pyridin-2- yl)-1H-pyrazol-4-yl)-1-(9- (3,3-dimethylbutyl)-3,9- diazaspiro[5.5]undecan-3- yl)ethan-1-one | 2.34 | 540.0 | B |

### Example 214. 2-(5-Chloro-1-methyl-3-(5-methylisoxazol-3-yl)-1H-pyrazol-4-yl)-1-(9-(3,3-dimethylbutyl)-2,9-diazaspiro[5.5]undecan-2-yl)ethan-1-one

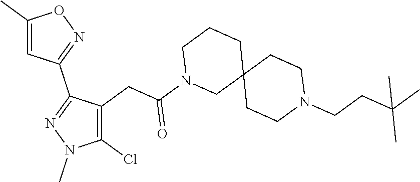 [SMILES: Cc1cc(-c2nn(C)c(Cl)c2CC(=O)N2CCCC3(CCN(CCC(C)(C)C)CC3)C2)no1]

Starting from 2-(5-chloro-1-methyl-3-(5-methyl isoxazol-3-yl)-1H-pyrazol-4-yl)-1-(2,9-diazaspiro[5.5]undecan-2-yl)ethan-1-one, obtained following the procedure described in steps i to 3 of example 120, and following the procedure described in step 3 of example 1, the title compound was obtained (23 mg, 31% yield).

HPLC Rt (Method A): 2.89 min; ESI+−MS m/z: 476.2 (M+H)⁺.

This method was used for the preparation of examples 215-223 using suitable starting materials.

| | | | Rt | MS | HPLC |
|---|---|---|---|---|---|
| STRUCTURE | EX | CHEMICAL NAME | (min) | (M + H) | Method |
| 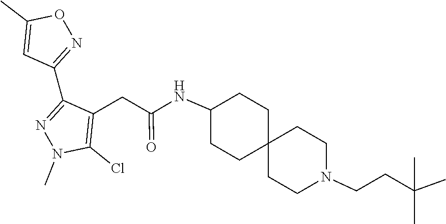 [SMILES: Cc1cc(-c2nn(C)c(Cl)c2CC(=O)NC2CCC3(CC2)CCN(CCC(C)(C)C)CC3)no1] | 215 | 2-(5-Chloro-1-methyl-3-(5- methylisoxazol-3-yl)-1H- pyrazol-4-yl)-N-(3-(3,3- dimethylbutyl)-3- azaspiro[5.5]undecan-9- yl)acetamide | 2.78 | 490.2 | A |
| 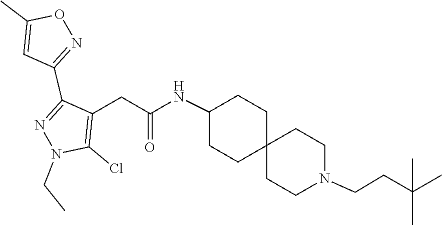 [SMILES: CCn1nc(-c2cc(C)on2)c(CC(=O)NC2CCC3(CC2)CCN(CCC(C)(C)C)CC3)c1Cl] | 216 | 2-(5-Chloro-1-ethyl-3-(5- methylisoxazol-3-yl)-1H- pyrazol-4-yl)-N-(3-(3,3- dimethylbutyl)-3- azaspiro[5.5]undecan-9- yl)acetamide | 2.13 | 504.2 | A |
| 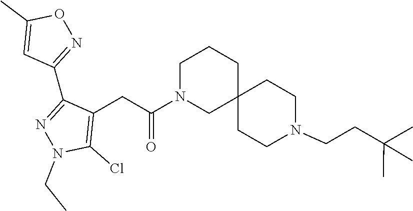 [SMILES: CCn1nc(-c2cc(C)on2)c(CC(=O)N2CCCC3(CCN(CCC(C)(C)C)CC3)C2)c1Cl] | 217 | 2-(5-Chloro-1-ethyl-3-(5- methylisoxazol-3-yl)-1H- pyrazol-4-yl)-1-(9-(3,3- dimethylbutyl)-2,9- diazaspiro[5.5]undecan-2- yl)ethan-1-one | 2.12 | 490.0 | B |
| 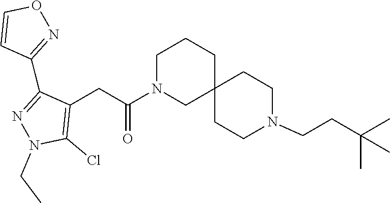 [SMILES: CCn1nc(-c2ccon2)c(CC(=O)N2CCCC3(CCN(CCC(C)(C)C)CC3)C2)c1Cl] | 218 | 2-(5-Chloro-1-ethyl-3- (isoxazol-3-yl)-1H-pyrazol- 4-yl)-1-(9-(3,3- dimethylbutyl)-2,9- diazaspiro[5.5]undecan-2- yl)ethan-1-one | 2.23 | 476.2 | A |
| 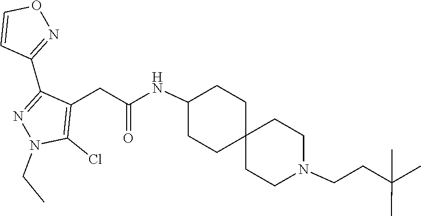 [SMILES: CCn1nc(-c2ccon2)c(CC(=O)NC2CCC3(CC2)CCN(CCC(C)(C)C)CC3)c1Cl] | 219 | 2-(5-Chloro-1-ethyl-3- (isoxazol-3-yl)-1H-pyrazol- 4-yl)-N-(3-(3,3- dimethylbutyl)-3- azaspiro[5.5]undecan-9- yl)acetamide | 2.03 | 490.2 | A |
| 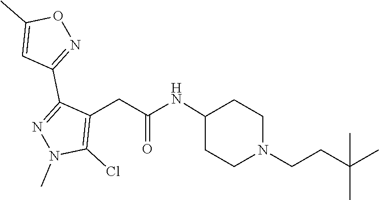 [SMILES: Cc1cc(-c2nn(C)c(Cl)c2CC(=O)NC2CCN(CCC(C)(C)C)CC2)no1] | 220 | 2-(5-Chloro-1-methyl-3-(5- methylisoxazol-3-yl)-1H- pyrazol-4-yl)-N-(1-(3,3- dimethylbutyl)piperidin-4- yl)acetamide | 1.81 | 422.2 | B |
| 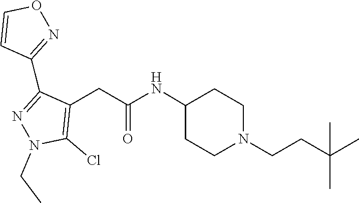 [SMILES: CCn1nc(-c2ccon2)c(CC(=O)NC2CCN(CCC(C)(C)C)CC2)c1Cl] | 221 | 2-(5-Chloro-1-ethyl-3- (isoxazol-3-yl)-1H-pyrazol- 4-yl)-N-(1-(3,3- dimethylbutyl)piperidin-4- yl)acetamide | 1.94 | 422.2 | A |
| 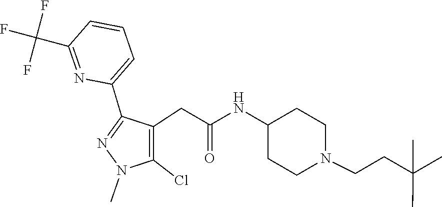 [SMILES: Cn1nc(-c2cccc(C(F)(F)F)n2)c(CC(=O)NC2CCN(CCC(C)(C)C)CC2)c1Cl] | 222 | 2-(5-Chloro-1-methyl-3-(6- (trifluoromethyl)pyridin-2- yl)-1H-pyrazol-4-yl)-N-(1- (3,3- dimethylbutyl)piperidin-4- yl)acetamide | 2.15 | 485.9 | B |
| 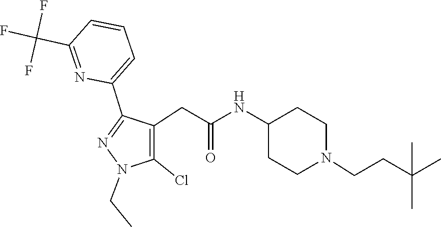 [SMILES: CCn1nc(-c2cccc(C(F)(F)F)n2)c(CC(=O)NC2CCN(CCC(C)(C)C)CC2)c1Cl] | 223 | 2-(5-chloro-1-ethyl-3-(6- (trifluoromethyl)pyridin-2- yl)-1H-pyrazol-4-yl)-N-(1- (3,3- dimethylbutyl)piperidin-4- yl)acetamide | 2.33 | 499.9 | B |
| 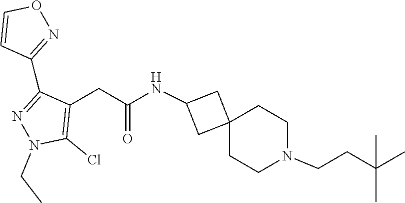 [SMILES: CCn1nc(-c2ccon2)c(CC(=O)NC2CC3(CCN(CCC(C)(C)C)CC3)C2)c1Cl] | 224 | 2-(5-chloro-1-ethyl-3- (isoxazol-3-yl)-1H-pyrazol- 4-yl)-N-(7-(3,3- dimethylbutyl)-7- azaspiro[3.5]nonan-2- yl)acetamide | 1.77 | 462.0 | B |

### Example 225. 2-((5-Chloro-1-ethyl-3-(5-methylisoxazol-3-yl)-1H-pyrazol-4-yl)methyl)-8-(3,3-dimethylbutyl)-2,8-diazaspiro[4.5]decan-3-one

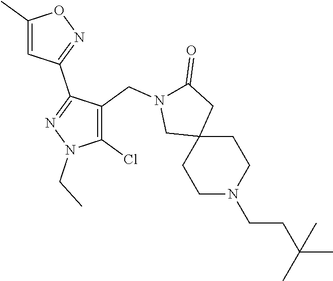 [SMILES: CCn1nc(-c2cc(C)on2)c(CN2CC3(CCN(CCC(C)(C)C)CC3)CC2=O)c1Cl]

### Step 1. 8-(3,3-Dimethylbutyl)-2,8-diazaspiro[4.5]decan-3-one

Starting from 2,8-diazaspiro[4.5]decan-3-one hydrochloride and following the procedure described in step 3 of example 1, the title compound was obtained (715 mg, Quant).

HPLC Rt (Method B): 1.24 min; ESI+−MS m/z: 239.2 (M+H)⁺.

### Step 2. Title Compound

Starting from the compound obtained in step 1 and 3-(5-chloro-4-(chloromethyl)-1-ethyl-1H-pyrazol-3-yl)-5-methylisoxazole (obtained following the procedure described in step 2 of example 114), and following the procedure described in step 3 of example 114, the title compound was obtained (107 mg, 98% yield).

HPLC Rt (Method B): 2.06 min; ESI+−MS m/z: 462.3 (M+H)⁺.

This method was used for the preparation of examples 226-229 using suitable starting materials.

| | | | Rt | MS | HPLC |
|---|---|---|---|---|---|
| STRUCTURE | EX | CHEMICAL NAME | (min) | (M + H) | Method |
| 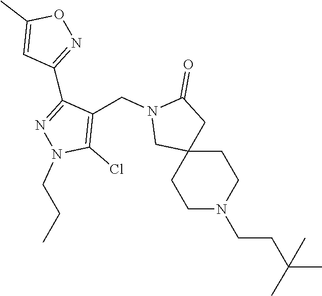 [SMILES: CCCn1nc(-c2cc(C)on2)c(CN2CC3(CCN(CCC(C)(C)C)CC3)CC2=O)c1Cl] | 226 | 2-((5-Chloro-3-(5- methylisoxazol-3-yl)-1- propyl-1H-pyrazol-4- yl)methyl)-8-(3,3- dimethylbutyl)-2,8- diazaspiro[4.5]decan-3- one | 2.23 | 476.3 | B |
| 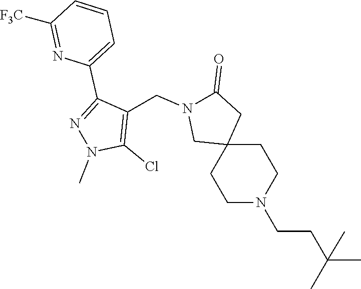 [SMILES: Cn1nc(-c2cccc(C(F)(F)F)n2)c(CN2CC3(CCN(CCC(C)(C)C)CC3)CC2=O)c1Cl] | 227 | 2-((5-Chloro-1-methyl-3- (6-(trifluoromethyl)pyridin- 2-yl)-1H-pyrazol-4- yl)methyl)-8-(3,3- dimethylbutyl)-2,8- diazaspiro[4.5]decan-3- one | 2.25 | 512.2 | B |
| 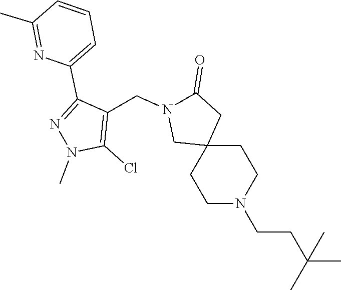 [SMILES: Cc1cccc(-c2nn(C)c(Cl)c2CN2CC3(CCN(CCC(C)(C)C)CC3)CC2=O)n1] | 228 | 2-((5-Chloro-1-methyl-3- (6-methylpyridin-2-yl)-1H- pyrazol-4-yl)methyl)-8- (3,3-dimethylbutyl)-2,8- diazaspiro[4.5]decan-3- one | 2.01 | 458.3 | B |
| 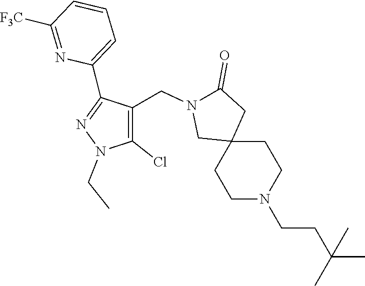 [SMILES: CCn1nc(-c2cccc(C(F)(F)F)n2)c(CN2CC3(CCN(CCC(C)(C)C)CC3)CC2=O)c1Cl] | 229 | 2-((5-Chloro-1-ethyl-3-(6- (trifluoromethyl)pyridin-2- yl)-1H-pyrazol-4- yl)methyl)-8-(3,3- dimethylbutyl)-2,8- diazaspiro[4.5]decan-3- one | 2.43 | 526.2 | B |

### Examples of Biological Activity

### Pharmacological Study

This invention is aimed at providing a series of compounds which show pharmacological activity towards the σ₁ receptor and/or σ₂ receptor and, especially, compounds which have a binding expressed as Kᵢ responding to the following scales:
Kᵢ (σ₁) is preferably <1000 nM, more preferably <500 nM, even more preferably <100 nM; and
Kᵢ (σ₂) is preferably <1000 nM, more preferably <500 nM, even more preferably <100 nM.

### Human σ₁ Receptor Radioligand Assay

Transfected HEK-293 membranes (7 μg) were incubated with 5 nM of [³H](+)-pentazocine in assay buffer containing Tris-HCl 50 mM at pH 8. NBS (non-specific binding) was measured by adding 10 μM haloperidol. The binding of the test compound was measured at either one concentration (% inhibition at 1 or 10 μM) or five different concentrations to determine affinity values (Ki). Plates were incubated at 37° C. for 120 minutes. After the incubation period, the reaction mix was then transferred to MultiScreen HTS, FC plates (Millipore), filtered and plates were washed 3 times with ice-cold 10 mM Tris-HCL (pH7.4). Filters were dried and counted at approximately 40% efficiency in a MicroBeta scintillation counter (Perkin-Elmer) using EcoScint liquid scintillation cocktail.

### Binding Assay to Human σ2/TMEM97 Receptor

Transfected HEK-293 membranes (15 μg) were incubated with 10 nM [³H]-1,3-Di-o-tolylguanidine (DTG) in assay buffer containing Tris-HCl 50 mM at pH 8.0. NSB (non-specific binding) was measured by adding 10 μM haloperidol. The binding of the test compound was measured at either one concentration (% inhibition at 1 or 10 μM) or five different concentrations to determine affinity values (Ki). Plates were incubated at 25° C. for 120 minutes. After the incubation period, the reaction mix was transferred to MultiScreen HTS, FC plates (Millipore), filtered and washed 3 times with ice-cold 10 mM Tris-HCL (pH 8.0). Filters were dried and counted at approximately 40% efficiency in a MicroBeta scintillation counter (Perkin-Elmer) using EcoScint liquid scintillation cocktail.

### Results:

The following scale has been adopted for representing the binding to σ1-receptor expressed as Kᵢ:
+ Kᵢ (σ₁)>1000 nM or inhibition ranges between 1% and 50%
++ 500 nM<=Kᵢ (σ₁)<=1000 nM
+++ 100 nM<=Kᵢ (σ₁)<=500 nM
++++ Kᵢ (σ₁)<100 nM

The following scale has been adopted for representing the binding to 62-receptor expressed as Kᵢ:
+ Kᵢ (σ₂)>1000 nM or inhibition ranges between 1% and 50%
++ 500 nM<=Kᵢ (σ₂)<=1000 nM
+++ 100 nM<=Kᵢ (σ₂)<=500 nM
++++ Kᵢ (σ₂)<100 nM

The results of the compounds showing binding for the 6-1 and/or 6-2 receptor are shown in Table 1:

TABLE 1

| | Binding σ-1 | Binding σ-2 |
|---|---|---|
| Example | Kᵢ (nM) | Kᵢ (nM) |
| 1 | +++ | + |
| 2 | ++ | + |
| 3 | ++ | + |
| 4 | + | ++ |
| 5 | ++ | +++ |
| 6 | +++ | + |
| 7 | +++ | + |
| 8 | +++ | ++ |
| 9 | + | ++ |
| 10 | +++ | +++ |
| 11 | ++ | +++ |
| 12 | +++ | + |
| 13 | +++ | +++ |
| 14 | +++ | + |
| 15 | +++ | + |
| 16 | +++ | + |
| 17 | +++ | + |
| 18 | +++ | + |
| 19 | ++ | + |
| 20 | +++ | +++ |
| 21 | ++ | +++ |
| 22 | +++ | +++ |
| 23 | +++ | ++++ |
| 24 | + | ++++ |
| 25 | ++++ | + |
| 26 | ++ | + |
| 27 | +++ | ++++ |
| 28 | +++ | +++ |
| 29 | ++ | +++ |
| 30 | +++ | + |
| 31 | ++ | + |
| 32 | + | +++ |
| 33 | +++ | +++ |
| 34 | ++ | +++ |
| 35 | ++ | + |
| 36 | ++ | + |
| 37 | +++ | +++ |
| 38 | +++ | +++ |
| 39 | ++ | +++ |
| 40 | +++ | +++ |
| 41 | +++ | +++ |
| 42 | + | +++ |
| 43 | ++ | ++ |
| 44 | ++ | ++ |
| 45 | +++ | +++ |
| 46 | +++ | + |
| 47 | + | + |
| 48 | + | +++ |
| 49 | +++ | + |
| 50 | +++ | ++++ |
| 51 | ++++ | ++++ |
| 52 | ++++ | ++++ |
| 53 | +++ | ++++ |
| 54 | ++++ | ++++ |
| 55 | + | +++ |
| 56 | +++ | +++ |
| 57 | ++ | +++ |
| 58 | +++ | ++++ |
| 59 | ++ | +++ |
| 60 | ++++ | +++ |
| 61 | +++ | ++++ |
| 62 | ++ | +++ |
| 63 | ++++ | +++ |
| 64 | +++ | +++ |
| 65 | +++ | +++ |
| 66 | ++++ | ++++ |
| 67 | ++ | ++++ |
| 68 | +++ | ++++ |
| 69 | ++++ | ++++ |
| 70 | ++++ | ++++ |
| 71 | + | +++ |
| 72 | ++ | +++ |
| 73 | +++ | ++++ |
| 74 | +++ | ++++ |
| 75 | ++ | ++++ |
| 76 | +++ | +++ |
| 77 | +++ | ++++ |
| 78 | +++ | ++++ |
| 79 | + | +++ |
| 80 | +++ | ++ |
| 81 | +++ | +++ |
| 82 | ++ | +++ |
| 83 | ++ | ++++ |
| 84 | + | +++ |
| 85 | + | +++ |
| 86 | + | +++ |
| 87 | ++ | ++++ |
| 88 | ++ | ++++ |
| 89 | + | + |
| 90 | ++ | +++ |
| 91 | ++ | ++++ |
| 92 | ++++ | ++++ |
| 93 | + | +++ |
| 94 | + | +++ |
| 95 | + | + |
| 96 | + | +++ |
| 97 | +++ | ++++ |
| 98 | ++ | ++++ |
| 99 | ++ | ++++ |
| 100 | ++ | ++++ |
| 101 | ++++ | ++++ |
| 102 | +++ | ++++ |
| 103 | ++ | ++++ |
| 104 | +++ | +++ |
| 105 | + | +++ |
| 106 | +++ | ++++ |
| 107 | +++ | ++++ |
| 108 | +++ | + |
| 109 | + | + |
| 110 | ++ | +++ |
| 111 | +++ | ++ |
| 112 | + | +++ |
| 113 | + | +++ |
| 114 | +++ | +++ |
| 115 | +++ | ++++ |
| 116 | +++ | ++++ |
| 117 | +++ | + |
| 118 | + | +++ |
| 119 | ++++ | +++ |
| 120 | +++ | ++++ |
| 121 | +++ | +++ |
| 122 | + | + |
| 123 | + | ++++ |
| 124 | + | +++ |
| 125 | + | ++ |
| 126 | + | + |
| 127 | ++++ | ++++ |
| 128 | +++ | ++++ |
| 129 | +++ | ++++ |
| 130 | ++ | +++ |
| 131 | ++ | ++ |
| 132 | +++ | ++++ |
| 133 | ++ | ++++ |
| 134 | ++++ | ++++ |
| 135 | + | ++++ |
| 136 | ++++ | ++++ |
| 137 | ++++ | ++++ |
| 138 | ++ | ++++ |
| 139 | +++ | ++++ |
| 140 | ++ | ++++ |
| 141 | ++++ | ++++ |
| 142 | ++++ | ++++ |
| 143 | ++++ | ++++ |
| 144 | ++ | +++ |
| 145 | ++ | ++++ |
| 146 | ++ | ++++ |
| 147 | ++++ | ++++ |
| 148 | ++++ | ++++ |
| 149 | +++ | ++++ |
| 150 | +++ | ++++ |
| 151 | ++++ | ++++ |
| 152 | +++ | ++++ |
| 153 | +++ | +++ |
| 154 | +++ | +++ |
| 155 | +++ | ++++ |
| 156 | +++ | ++++ |
| 157 | + | ++++ |
| 158 | +++ | ++++ |
| 159 | ++++ | ++++ |
| 160 | ++++ | ++++ |
| 161 | ++++ | ++++ |
| 162 | ++++ | ++++ |
| 163 | +++ | ++++ |
| 164 | ++++ | +++ |
| 165 | ++++ | ++++ |
| 166 | +++ | ++++ |
| 167 | ++++ | ++++ |
| 168 | +++ | ++++ |
| 169 | + | ++++ |
| 170 | +++ | ++++ |
| 171 | +++ | ++++ |
| 172 | ++ | ++++ |
| 173 | ++ | ++++ |
| 174 | ++ | ++++ |
| 175 | ++ | ++++ |
| 176 | + | ++++ |
| 177 | +++ | ++++ |
| 178 | + | +++ |
| 179 | + | ++++ |
| 180 | +++ | ++++ |
| 181 | + | +++ |
| 182 | +++ | ++++ |
| 183 | ++ | ++++ |
| 184 | +++ | ++++ |
| 185 | +++ | ++++ |
| 186 | +++ | ++++ |
| 187 | ++ | ++++ |
| 188 | ++ | ++++ |
| 189 | ++ | ++++ |
| 190 | +++ | ++++ |
| 191 | +++ | ++++ |
| 192 | +++ | ++++ |
| 193 | ++ | ++++ |
| 194 | ++ | ++++ |
| 195 | ++ | ++++ |
| 196 | ++ | ++++ |
| 197 | +++ | ++++ |
| 198 | ++ | ++++ |
| 199 | +++ | ++++ |
| 200 | ++ | ++++ |
| 201 | +++ | +++ |
| 202 | +++ | +++ |
| 203 | +++ | ++++ |
| 204 | ++++ | ++++ |
| 205 | ++++ | ++++ |
| 206 | ++ | ++ |
| 207 | +++ | ++++ |
| 208 | ++ | ++++ |
| 209 | ++++ | ++++ |
| 210 | ++ | ++++ |
| 211 | ++++ | ++++ |
| 212 | ++++ | +++ |
| 213 | +++ | ++++ |
| 214 | +++ | ++++ |
| 215 | +++ | ++++ |
| 216 | ++++ | ++++ |
| 217 | +++ | ++++ |
| 218 | + | ++++ |
| 219 | +++ | ++++ |
| 220 | +++ | ++++ |
| 221 | ++++ | ++++ |
| 222 | ++++ | ++++ |
| 223 | ++++ | ++++ |
| 224 | +++ | ++++ |
| 225 | +++ | ++++ |
| 226 | +++ | ++++ |
| 227 | ++++ | ++++ |
| 228 | +++ | +++ |
| 229 | ++++ | ++++ |

1. A compound of general formula (I): 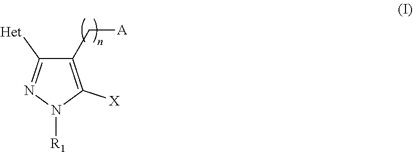
wherein:
X is a halogen atom;
Het is a 5 to 12-membered heterocyclic ring or ring system optionally substituted by one or more R₂ radicals;
A represents —CO—B or —CH₂—B
B is one of the following moieties: 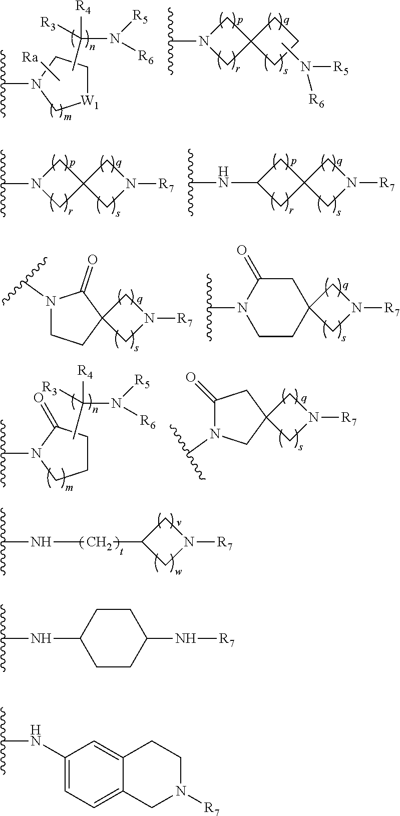
Rₐ is a hydrogen atom or a branched or unbranched C₁₋₆ alkyl radical;
R₁ is a branched or unbranched C₁₋₆ alkyl radical; an aryl radical; or a 5 or 6-membered N-containing heteroaromatic ring;
R₂ is a hydrogen atom, a branched or unbranched C₁₋₆ alkyl radical; a C₃₋₉ cycloalkyl radical, a halogen atom, a C₁₋₆ haloalkyl radical or —OR_{2′} being R_{2′} a hydrogen atom or a C₁₋₆ alkyl radical;
R₃ and R₄ represent a hydrogen atom, a branched or unbranched C₁₋₆ alkyl radical;
or alternatively R₃ and R₄ form together with the atom they are attached to a C₃₋₉ cycloalkyl group;
R₅ is a hydrogen atom or a branched or unbranched C₁₋₆ alkyl radical;
R₆ is a branched or unbranched C₁₋₆ alkyl radical; CH(R_{6′})—(CH₂)ₖ-aryl being R_{6′} a hydrogen atom or a C₁₋₆ alkyl radical; a —(CH₂)ⱼ-heterocyclyl or a —CH₂—CO— heterocyclyl, the heterocyclyl being optionally mono- or bisubstituted;
R₇ is a branched or unbranched C₁₋₆ alkyl radical; —CH(R_{7′})—(CH₂)ₖ-aryl being R_{7′} a hydrogen atom or a C₁₋₆ alkyl radical, the aryl being optionally mono- or bisubstituted;
a —(CH₂)ᵢ-heterocyclyl; or a —CH₂—CO-heterocyclyl, the heterocyclyl being optionally mono- or bisubstituted;
W₁ is —CH₂— or —O—
j is 1 or 2;
k is 0 or 1;
n is 0, 1 or 2;
m is 1, 2 or 3;
p is 1, 2 or 3;
q is 1, 2 or 3;
r is 1, 2 or 3;
s is 1, 2 or 3;
t is 0, 1 or 2;
v is 1, 2, 3 or 4;
w is 1, 2, 3 or 4;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof, or a corresponding solvate thereof.

2. A compound according to claim 1, wherein Het is a group selected from: 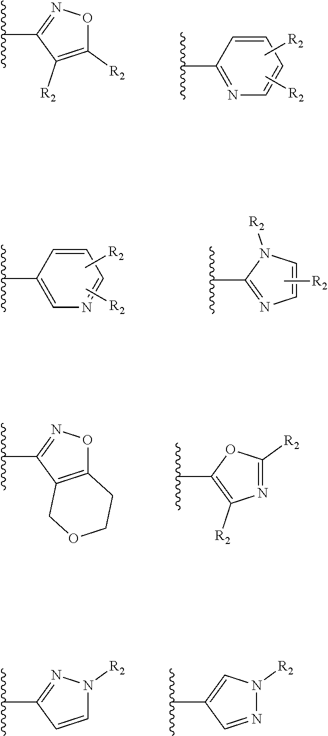
wherein R₂ is as defined in claim 1, preferably, a hydrogen atom, methyl, ethyl, isopropyl, cyclopropyl, trifluromethyl, methoxy or hydroxyl.

3. A compound according to any one of claims 1 to 2 wherein B is: 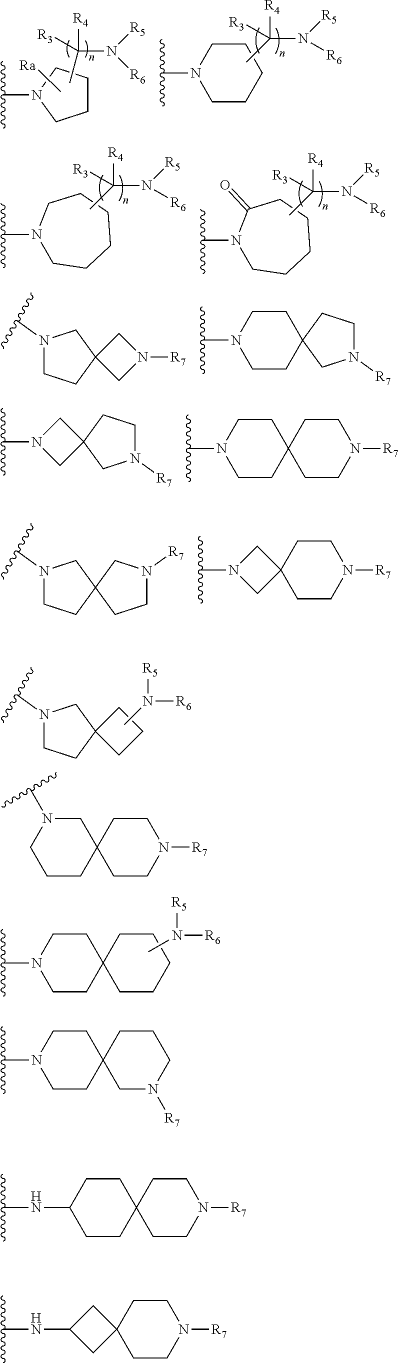 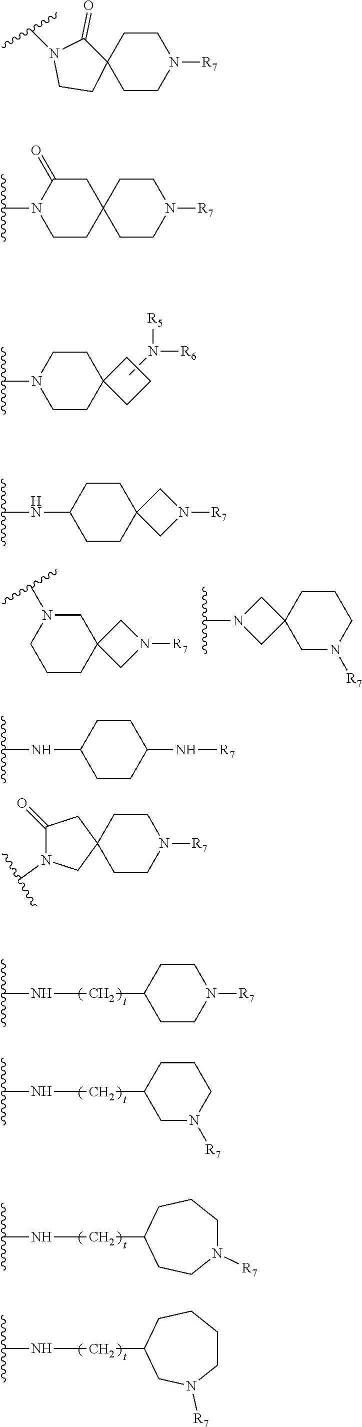 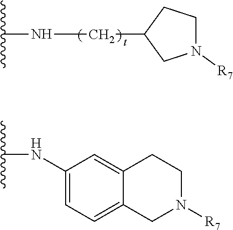
Wherein Rₐ, R₂, R₃, R₄, R₅, R₆, R₇, t and n are as defined in claim 1.

4. A compound according to any one of claims 1 to 3 wherein R₁ is methyl, ethyl, propyl, isopropyl, pyridyl or phenyl.

5. A compound according to any one of claims 1 to 4 wherein R₂ is a hydrogen atom, methyl, ethyl, isopropyl, cyclopropyl, trifluoromethyl, methoxy, ethoxy or hydroxyl.

6. A compound according to any one of claims 1 to 5, wherein R₃ and R₄ represent a hydrogen atom; or alternatively R₃ and R₄ form together with the atom they are attached to a cyclopropane ring.

7. A compound according to any one of claims 1 to 6, wherein R₅ is a hydrogen atom, methyl, ethyl, propyl, isobutyl, isopentyl or dimethylbutyl.

8. A compound according to any one of claims 1 to 7, wherein R₆ is methyl, ethyl, propyl, isobutyl, isopentyl, dimethylbutyl, —(CH₂)ⱼ-phenyl, —(CH₂)ⱼ-tetrahydro-2H-pyranyl or —(CH₂)ⱼ-piperidinyl wherein j is 1 or 2.

9. A compound according to any one of claims 1 to 8, wherein R₇ is methyl, ethyl, propyl, isobutyl, isopentyl, dimethylbutyl, —CH(R_{7′})(CH₂)ₖ-phenyl being R_{7′} a hydrogen atom or a C₁₋₆ alkyl radical and the phenyl being optionally mono- or bisubstituted with —CN or halogen, preferably F; or a —CH₂—CO-piperidine optionally substituted by one or two halogen atoms, preferably F.

10. A compound according to claim 1 with the general formula (Ia), (Ib), (Ic) or (Id): 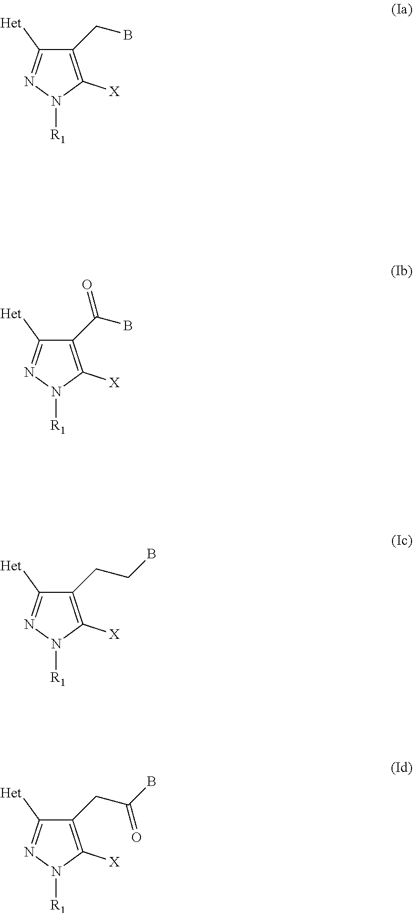
wherein Het, X, B and R₁ are as defined in any one of claims 1 to 9.

11. A compound according to claim 1 with the general formula (Ie), (If), (Ig), (Ih), (Ii), (Ij), (Ik), (Im) or (In): 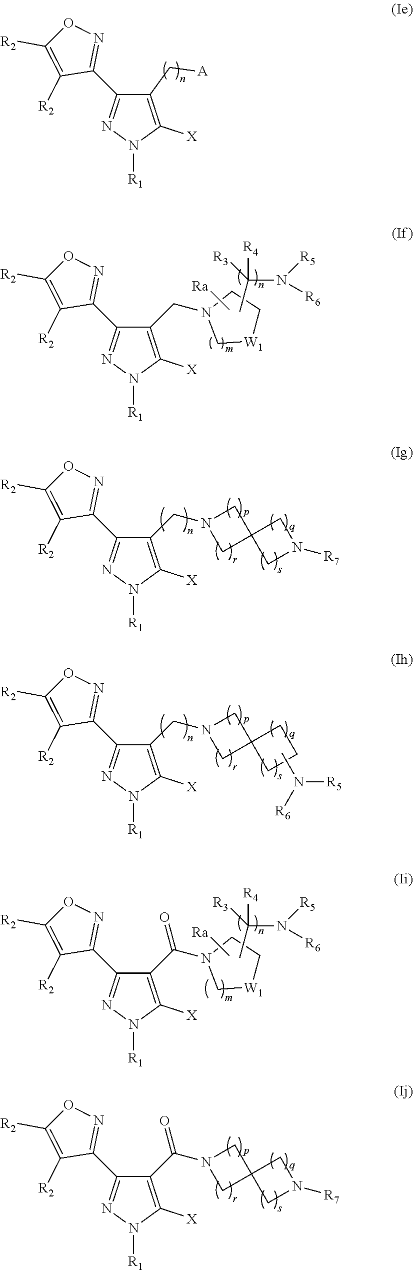 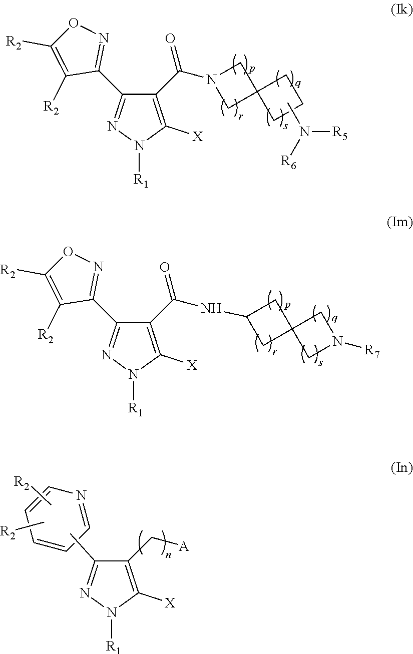
wherein A, X, W₁, Rₐ, R₁, R₂, R₃, R₄, R₅, R₆, R₇, m, n, p, q, r and s are as defined in any one of claims 1 to 6.

12. A compound according to claim 1 selected from:
[1] N-((1-((5-Chloro-1-methyl-3-(5-methylisoxazol-3-yl)-1H-pyrazol-4-yl)methyl)pyrrolidin-3-yl)methyl)-3,3-dimethylbutan-1-amine;
[2] 1-((5-Chloro-1-methyl-3-(5-methylisoxazol-3-yl)-1H-pyrazol-4-yl)methyl)-N-isopentylazepan-3-amine;
[3] 1-((5-Chloro-1-methyl-3-(5-methylisoxazol-3-yl)-1H-pyrazol-4-yl)methyl)-N-phenethylazepan-3-amine;
[4] 1-((5-Chloro-1-methyl-3-(5-methylisoxazol-3-yl)-1H-pyrazol-4-yl)methyl)-N-((tetrahydro-2H-pyran-4-yl)methyl)azepan-3-amine;
[5] (R)-1-((5-Chloro-1-methyl-3-(5-methylisoxazol-3-yl)-1H-pyrazol-4-yl)methyl)-N-isopentylpiperidin-3-amine;
[6] (S)-1-((5-Chloro-1-methyl-3-(5-methylisoxazol-3-yl)-1H-pyrazol-4-yl)methyl)-N-isopentylpiperidin-3-amine;
[7] (S)-1-((5-Chloro-1-methyl-3-(5-methylisoxazol-3-yl)-1H-pyrazol-4-yl)methyl)-N-phenethylpiperidin-3-amine;
[8] 1-((5-Chloro-1-methyl-3-(5-methylisoxazol-3-yl)-1H-pyrazol-4-yl)methyl)-N-(3,3-dimethylbutyl)azepan-3-amine;
[9] N-Butyl-1-((5-chloro-1-methyl-3-(5-methylisoxazol-3-yl)-1H-pyrazol-4-yl)methyl)azepan-3-amine;
[10] (R)-1-((5-Chloro-1-methyl-3-(5-methylisoxazol-3-yl)-1H-pyrazol-4-yl)methyl)-N-isopentylazepan-3-amine;
[11] 1-((5-Chloro-1-methyl-3-(5-methylisoxazol-3-yl)-1H-pyrazol-4-yl)methyl)-N-isopentylpyrrolidin-3-amine;
[12] (S)-1-((5-Chloro-1-methyl-3-(5-methylisoxazol-3-yl)-1H-pyrazol-4-yl)methyl)-N-isopentylazepan-3-amine;
[13] 1-((5-Chloro-1-methyl-3-(5-methylisoxazol-3-yl)-1H-pyrazol-4-yl)methyl)-N-(3,3-dimethylbutyl)pyrrolidin-3-amine;
[14] 1-((5-Chloro-1-ethyl-3-(5-methylisoxazol-3-yl)-1H-pyrazol-4-yl)methyl)-N-isopentylazepan-3-amine;
[15] 1-((5-Chloro-3-(5-isopropylisoxazol-3-yl)-1-methyl-1H-pyrazol-4-yl)methyl)-N-(3,3-dimethylbutyl)azepan-3-amine;
[16] 1-((5-Chloro-3-(5-cyclopropylisoxazol-3-yl)-1-methyl-1H-pyrazol-4-yl)methyl)-N-(3,3-dimethylbutyl)azepan-3-amine;
[17] 1-((5-Chloro-3-(5-isopropylisoxazol-3-yl)-1-methyl-1H-pyrazol-4-yl)methyl)-N-isopentylazepan-3-amine;
[18] 1-((5-Chloro-3-(5-methylisoxazol-3-yl)-1-phenyl-1H-pyrazol-4-yl)methyl)-N-isopentylazepan-3-amine;
[19] 1-((5-Chloro-3-(5-cyclopropylisoxazol-3-yl)-1-methyl-1H-pyrazol-4-yl)methyl)-N-isopentylazepan-3-amine;
[20] 1-((5-Chloro-3-(5-ethylisoxazol-3-yl)-1-methyl-1H-pyrazol-4-yl)methyl)-N-(3,3-dimethylbutyl)azepan-3-amine;
[21] 1-((5-Chloro-3-(5-ethylisoxazol-3-yl)-1-methyl-1H-pyrazol-4-yl)methyl)-N-isopentylazepan-3-amine;
[22] 1-((5-Chloro-1-ethyl-3-(5-methylisoxazol-3-yl)-1H-pyrazol-4-yl)methyl)-N-(3,3-dimethylbutyl)azepan-3-amine;
[23] 1-((5-Chloro-1-methyl-3-(5-methylisoxazol-3-yl)-1H-pyrazol-4-yl)methyl)-N-(3,3-dimethylbutyl)azepan-4-amine;
[24] 1-((5-Chloro-1-methyl-3-(5-methylisoxazol-3-yl)-1H-pyrazol-4-yl)methyl)-N-isopentylazepan-4-amine;
[25] 1-((5-Chloro-1-methyl-3-(pyridin-2-yl)-1H-pyrazol-4-yl)methyl)-N-(3,3-dimethylbutyl)azepan-3-amine;
[26] 1-((5-Chloro-1-methyl-3-(pyridin-2-yl)-1H-pyrazol-4-yl)methyl)-N-isopentylazepan-3-amine;
[27] 1-((5-Chloro-1-methyl-3-(4-methylisoxazol-3-yl)-1H-pyrazol-4-yl)methyl)-N-(3,3-dimethylbutyl)azepan-3-amine;
[28] 1-((5-Chloro-1-methyl-3-(4-methylisoxazol-3-yl)-1H-pyrazol-4-yl)methyl)-N-isopentylazepan-3-amine;
[29] N-((1-((5-Chloro-1-methyl-3-(5-methylisoxazol-3-yl)-1H-pyrazol-4-yl)methyl)pyrrolidin-3-yl)methyl)-3-methylbutan-1-amine;
[30] 1-((5-Chloro-1-methyl-3-(pyridin-3-yl)-1H-pyrazol-4-yl)methyl)-N-(3,3-dimethylbutyl)azepan-3-amine;
[31] 1-((5-Chloro-1-methyl-3-(pyridin-3-yl)-1H-pyrazol-4-yl)methyl)-N-isopentylazepan-3-amine;
[32] 1-((5-Chloro-1-methyl-3-(1-methyl-1H-imidazol-2-yl)-1H-pyrazol-4-yl)methyl)-N-isopentylazepan-3-amine;
[33] 1-((5-Chloro-3-(isoxazol-3-yl)-1-methyl-1H-pyrazol-4-yl)methyl)-N-(3,3-dimethylbutyl)azepan-3-amine;
[34] 1-((5-Chloro-3-(isoxazol-3-yl)-1-methyl-1H-pyrazol-4-yl)methyl)-N-isopentylazepan-3-amine;
[35] 1-((5-Chloro-3-(5-methoxypyridin-2-yl)-1-methyl-1H-pyrazol-4-yl)methyl)-N-(3,3-dimethylbutyl)azepan-3-amine;
[36] 1-((5-Chloro-3-(5-methoxypyridin-2-yl)-1-methyl-1H-pyrazol-4-yl)methyl)-N-isopentylazepan-3-amine;
[37] 1-((5-Chloro-3-(4,5-dimethylisoxazol-3-yl)-1-methyl-1H-pyrazol-4-yl)methyl)-N-isopentylazepan-3-amine;
[38] 1-((5-Chloro-3-(4,5-dimethylisoxazol-3-yl)-1-methyl-1H-pyrazol-4-yl)methyl)-N-(3,3-dimethylbutyl)azepan-3-amine;
[39] 6-(5-Chloro-4-((3-(isopentylamino)azepan-1-yl)methyl)-1-methyl-1H-pyrazol-3-yl)pyridin-2-ol;
[40] (S)-1-((5-Chloro-3-(6,7-dihydro-4H-pyrano[3,4-d]isoxazol-3-yl)-1-methyl-1H-pyrazol-4-yl)methyl)-N-isopentylazepan-3-amine;
[41] N-((1-((5-Chloro-1-methyl-3-(5-methylisoxazol-3-yl)-1H-pyrazol-4-yl)methyl)piperidin-4-yl)methyl)-3-methylbutan-1-amine;
[42] N-((1-((5-Chloro-1-methyl-3-(5-methylisoxazol-3-yl)-1H-pyrazol-4-yl)methyl)pyrrolidin-3-yl)methyl)-N-isobutyl-2-methylpropan-1-amine;
[43] N-((1-((5-Chloro-1-methyl-3-(5-methylisoxazol-3-yl)-1H-pyrazol-4-yl)methyl)piperidin-4-yl)methyl)-2-methylpropan-1-amine;
[44] N-Benzyl-1-(1-((5-chloro-1-methyl-3-(5-methylisoxazol-3-yl)-1H-pyrazol-4-yl)methyl)pyrrolidin-3-yl)methanamine;
[45] N-((1-((5-Chloro-1-methyl-3-(5-methylisoxazol-3-yl)-1H-pyrazol-4-yl)methyl)pyrrolidin-3-yl)methyl)-2-phenylethanamine;
[46] N-((1-((5-Chloro-1-methyl-3-(5-methylisoxazol-3-yl)-1H-pyrazol-4-yl)methyl)pyrrolidin-3-yl)methyl)-N-propylpropan-1-amine;
[47] N-((1-((5-Chloro-1-methyl-3-(5-methylisoxazol-3-yl)-1H-pyrazol-4-yl)methyl)pyrrolidin-3-yl)methyl)propan-1-amine;
[48] N-((1-((5-Chloro-1-methyl-3-(5-methylisoxazol-3-yl)-1H-pyrazol-4-yl)methyl)piperidin-3-yl)methyl)-2-methylpropan-1-amine;
[49] N-((1-((5-Chloro-1-methyl-3-(5-methylisoxazol-3-yl)-1H-pyrazol-4-yl)methyl)pyrrolidin-3-yl)methyl)-2-methylpropan-1-amine;
[50] 3-(5-Chloro-4-((2-(3,3-dimethylbutyl)-2,8-diazaspiro[4.5]decan-8-yl)methyl)-1-methyl-1H-pyrazol-3-yl)-5-methylisoxazole;
[51] 3-(5-Chloro-4-((6-(3,3-dimethylbutyl)-2,6-diazaspiro[3.4]octan-2-yl)methyl)-1-methyl-1H-pyrazol-3-yl)-5-methylisoxazole;
[52] 3-(5-Chloro-4-((2-(3,3-dimethylbutyl)-2,6-diazaspiro[3.4]octan-6-yl)methyl)-1-methyl-1H-pyrazol-3-yl)-5-methylisoxazole;
[53] 3-(5-Chloro-4-((9-(3,3-dimethylbutyl)-3,9-diazaspiro[5.5]undecan-3-yl)methyl)-1-methyl-1H-pyrazol-3-yl)-5-methylisoxazole;
[54] 3-(5-Chloro-4-((7-(3,3-dimethylbutyl)-2,7-diazaspiro[4.4]nonan-2-yl)methyl)-1-methyl-1H-pyrazol-3-yl)-5-methylisoxazole;
[55] N-Benzyl-1-(1-((5-chloro-1-methyl-3-(5-methylisoxazol-3-yl)-1H-pyrazol-4-yl)methyl)piperidin-3-yl)methanamine;
[56] N-Benzyl-1-(1-((5-chloro-1-methyl-3-(5-methylisoxazol-3-yl)-1H-pyrazol-4-yl)methyl)piperidin-4-yl)methanamine;
[57] N-((1-((5-Chloro-1-methyl-3-(5-methylisoxazol-3-yl)-1H-pyrazol-4-yl)methyl)piperidin-3-yl)methyl)-2-phenylethanamine;
[58] N-((1-((5-Chloro-1-methyl-3-(5-methylisoxazol-3-yl)-1H-pyrazol-4-yl)methyl)piperidin-3-yl)methyl)-3-methylbutan-1-amine;
[59] N-((1-((5-Chloro-1-methyl-3-(5-methylisoxazol-3-yl)-1H-pyrazol-4-yl)methyl)azepan-3-yl)methyl)-3-methylbutan-1-amine;
[60] 3-(4-((2-Benzyl-2,6-diazaspiro[3.4]octan-6-yl)methyl)-5-chloro-1-methyl-1H-pyrazol-3-yl)-5-methylisoxazole;
[61] 3-(5-Chloro-1-methyl-4-((2-phenethyl-2,6-diazaspiro[3.4]octan-6-yl)methyl)-1H-pyrazol-3-yl)-5-methylisoxazole;
[62] 1-(1-((5-Chloro-1-methyl-3-(5-methylisoxazol-3-yl)-1H-pyrazol-4-yl)methyl)pyrrolidin-3-yl)-N-isopentylcyclopropanamine;
[63] 1-(1-((5-Chloro-1-methyl-3-(5-methylisoxazol-3-yl)-1H-pyrazol-4-yl)methyl)piperidin-3-yl)-N-isopentylcyclopropanamine;
[64] N-((4-((5-Chloro-1-methyl-3-(5-methylisoxazol-3-yl)-1H-pyrazol-4-yl)methyl)-1,4-oxazepan-2-yl)methyl)-3-methylbutan-1-amine;
[65] 3-(5-Chloro-1-methyl-4-((9-(1-phenylethyl)-3,9-diazaspiro[5.5]undecan-3-yl)methyl)-1H-pyrazol-3-yl)-5-methylisoxazole;
[66] 3-(4-((9-Benzyl-3,9-diazaspiro[5.5]undecan-3-yl)methyl)-5-chloro-1-methyl-1H-pyrazol-3-yl)-5-methylisoxazole;
[67] 3-(5-Chloro-4-((9-isopentyl-3,9-diazaspiro[5.5]undecan-3-yl)methyl)-1-methyl-1H-pyrazol-3-yl)-5-methylisoxazole;
[68] 3-(5-Chloro-1-methyl-4-((9-neopentyl-3,9-diazaspiro[5.5]undecan-3-yl)methyl)-1H-pyrazol-3-yl)-5-methylisoxazole;
[69] (S)-3-(5-Chloro-4-((7-(3,3-dimethylbutyl)-2,7-diazaspiro[4.4]nonan-2-yl)methyl)-1-methyl-1H-pyrazol-3-yl)-5-methylisoxazole;
[70] (R)-3-(5-Chloro-4-((7-(3,3-dimethylbutyl)-2,7-diazaspiro[4.4]nonan-2-yl)methyl)-1-methyl-1H-pyrazol-3-yl)-5-methylisoxazole;
[71] (R)-N-((1-((5-Chloro-1-methyl-3-(5-methylisoxazol-3-yl)-1H-pyrazol-4-yl)methyl)pyrrolidin-3-yl)methyl)-2-phenylethanamine;
[72] (S)-N-((1-((5-chloro-1-methyl-3-(5-methylisoxazol-3-yl)-1H-pyrazol-4-yl)methyl)pyrrolidin-3-yl)methyl)-2-phenylethanamine;
[73] (S)-N-((1-((5-Chloro-1-methyl-3-(5-methylisoxazol-3-yl)-1H-pyrazol-4-yl)methyl)pyrrolidin-3-yl)methyl)-3,3-dimethylbutan-1-amine;
[74] (R)-N-((1-((5-Chloro-1-methyl-3-(5-methylisoxazol-3-yl)-1H-pyrazol-4-yl)methyl)pyrrolidin-3-yl)methyl)-3,3-dimethylbutan-1-amine;
[75] (2s,4r)-6-((5-Chloro-1-methyl-3-(5-methylisoxazol-3-yl)-1H-pyrazol-4-yl)methyl)-N-(3,3-dimethylbutyl)-6-azaspiro[3.4]octan-2-amine;
[76] (2r,4s)-6-((5-Chloro-1-methyl-3-(5-methylisoxazol-3-yl)-1H-pyrazol-4-yl)methyl)-N-(3,3-dimethylbutyl)-6-azaspiro[3.4]octan-2-amine;
[77] N-(((3R,4S)-1-((5-Chloro-1-methyl-3-(5-methylisoxazol-3-yl)-1H-pyrazol-4-yl)methyl)-4-methylpyrrolidin-3-yl)methyl)-3,3-dimethylbutan-1-amine;
[78] N-(((3S,4R)-1-((5-Chloro-1-methyl-3-(5-methylisoxazol-3-yl)-1H-pyrazol-4-yl)methyl)-4-methylpyrrolidin-3-yl)methyl)-3,3-dimethylbutan-1-amine;
[79] 1-((5-Chloro-1-methyl-3-(5-methylisoxazol-3-yl)-1H-pyrazol-4-yl)methyl)-N-(2-(piperidin-1-yl)ethyl)azepan-3-amine;
[80] 1-((5-Chloro-1-methyl-3-(5-methylisoxazol-3-yl)-1H-pyrazol-4-yl)methyl)-N-isopentyl-N-methylazepan-3-amine;
[81] 1-((5-Chloro-1-methyl-3-(5-methylisoxazol-3-yl)-1H-pyrazol-4-yl)methyl)-N-methyl-N-((tetrahydro-2H-pyran-4-yl)methyl)azepan-3-amine;
[82] 1-((5-Chloro-1-methyl-3-(5-methylisoxazol-3-yl)-1H-pyrazol-4-yl)methyl)-N-methyl-N-(2-(piperidin-1-yl)ethyl)azepan-3-amine;
[83] (5-Chloro-1-methyl-3-(5-methylisoxazol-3-yl)-1H-pyrazol-4-yl)(3-(((3,3-dimethylbutyl)amino)methyl)piperidin-1-yl)methanone;
[84] (5-Chloro-1-methyl-3-(5-methylisoxazol-3-yl)-1H-pyrazol-4-yl)(3-(isopentylamino)azepan-1-yl)methanone;
[85] (5-Chloro-1-methyl-3-(5-methylisoxazol-3-yl)-1H-pyrazol-4-yl)(3-(phenethylamino)azepan-1-yl)methanone;
[86] (3-(Benzylamino)azepan-1-yl)(5-chloro-1-methyl-3-(5-methylisoxazol-3-yl)-1H-pyrazol-4-yl)methanone;
[87] (5-Chloro-1-methyl-3-(5-methylisoxazol-3-yl)-1H-pyrazol-4-yl)(2-(3,3-dimethylbutyl)-2,8-diazaspiro[4.5]decan-8-yl)methanone;
[88] (5-Chloro-1-methyl-3-(5-methylisoxazol-3-yl)-1H-pyrazol-4-yl)(9-(3,3-dimethylbutyl)-3,9-diazaspiro[5.5]undecan-3-yl)methanone;
[89] (5-Chloro-1-methyl-3-(5-methylisoxazol-3-yl)-1H-pyrazol-4-yl)(9-isobutyl-3,9-diazaspiro[5.5]undecan-3-yl)methanone;
[90] (5-Chloro-1-methyl-3-(5-methylisoxazol-3-yl)-1H-pyrazol-4-yl)(9-isopentyl-3,9-diazaspiro[5.5]undecan-3-yl)methanone;
[91] (5-Chloro-1-methyl-3-(5-methylisoxazol-3-yl)-1H-pyrazol-4-yl)(2-(3,3-dimethylbutyl)-2,9-diazaspiro[5.5]undecan-9-yl)methanone;
[92] (5-Chloro-1-methyl-3-(5-methylisoxazol-3-yl)-1H-pyrazol-4-yl)(9-(3,3-dimethylbutyl)-2,9-diazaspiro[5.5]undecan-2-yl)methanone;
[93] (5-Chloro-1-methyl-3-(5-methylisoxazol-3-yl)-1H-pyrazol-4-yl)(9-(isopentylamino)-3-azaspiro[5.5]undecan-3-yl)methanone;
[94] (5-Chloro-1-methyl-3-(5-methylisoxazol-3-yl)-1H-pyrazol-4-yl)(8-(3,3-dimethylbutyl)-2,8-diazaspiro[4.5]decan-2-yl)methanone;
[95] (5-Chloro-1-methyl-3-(5-methylisoxazol-3-yl)-1H-pyrazol-4-yl)(3-((isobutylamino)methyl)piperidin-1-yl)methanone;
[96] (5-Chloro-1-methyl-3-(5-methylisoxazol-3-yl)-1H-pyrazol-4-yl)(9-((3,3-dimethylbutyl)amino)-3-azaspiro[5.5]undecan-3-yl)methanone;
[97] (5-Chloro-3-(5-ethylisoxazol-3-yl)-1-methyl-1H-pyrazol-4-yl)(9-(3,3-dimethylbutyl)-3,9-diazaspiro[5.5]undecan-3-yl)methanone;
[98] (9-(3,3-Dimethylbutyl)-3,9-diazaspiro[5.5]undecan-3-yl)(5-fluoro-1-methyl-3-(5-methylisoxazol-3-yl)-1H-pyrazol-4-yl)methanone;
[99] (5-Chloro-1-ethyl-3-(5-methylisoxazol-3-yl)-1H-pyrazol-4-yl)(9-(3,3-dimethylbutyl)-3,9-diazaspiro[5.5]undecan-3-yl)methanone;
[100] (5-Chloro-1-methyl-3-(4-methylisoxazol-3-yl)-1H-pyrazol-4-yl)(9-(3,3-dimethylbutyl)-3,9-diazaspiro[5.5]undecan-3-yl)methanone;
[101] (5-Chloro-3-(5-methylisoxazol-3-yl)-1-phenyl-1H-pyrazol-4-yl)(9-(3,3-dimethylbutyl)-3,9-diazaspiro[5.5]undecan-3-yl)methanone;
[102] (5-Chloro-3-(6-methoxypyridin-2-yl)-1-methyl-1H-pyrazol-4-yl)(9-(3,3-dimethylbutyl)-3,9-diazaspiro[5.5]undecan-3-yl)methanone;
[103] (5-Chloro-1-methyl-3-(4-methylisoxazol-3-yl)-1H-pyrazol-4-yl)(9-(3,3-dimethylbutyl)-2,9-diazaspiro[5.5]undecan-2-yl)methanone;
[104] (5-Chloro-1-methyl-3-(5-methylisoxazol-3-yl)-1H-pyrazol-4-yl)(7-(3,3-dimethylbutyl)-2,7-diazaspiro[3.5]nonan-2-yl)methanone;
[105] (5-Chloro-1-methyl-3-(5-methylisoxazol-3-yl)-1H-pyrazol-4-yl)(2-(3,3-dimethylbutyl)-2,6-diazaspiro[3.4]octan-6-yl)methanone;
[106] 5-Chloro-N-(7-(3,3-dimethylbutyl)-7-azaspiro[3.5]nonan-2-yl)-1-methyl-3-(5-methylisoxazol-3-yl)-1H-pyrazole-4-carboxamide;
[107] 5-Chloro-N-(3-(3,3-dimethylbutyl)-3-azaspiro[5.5]undecan-9-yl)-1-methyl-3-(5-methylisoxazol-3-yl)-1H-pyrazole-4-carboxamide;
[108] (R)-(5-Chloro-1-methyl-3-(5-methylisoxazol-3-yl)-1H-pyrazol-4-yl)(3-(isopentylamino)piperidin-1-yl)methanone;
[109] (S)-(5-Chloro-1-methyl-3-(5-methylisoxazol-3-yl)-1H-pyrazol-4-yl)(3-(isopentylamino)piperidin-1-yl)methanone;
[110] (5-Chloro-1-methyl-3-(5-methylisoxazol-3-yl)-1H-pyrazol-4-yl)(3-(methyl(phenethyl)amino)azepan-1-yl)methanone;
[111] (5-Chloro-1-methyl-3-(5-methylisoxazol-3-yl)-1H-pyrazol-4-yl)(3-(isopentyl(methyl)amino)azepan-1-yl)methanone;
[112] 2-(9-(5-Chloro-1-methyl-3-(5-methylisoxazol-3-yl)-1H-pyrazole-4-carbonyl)-3,9-diazaspiro[5.5]undecan-3-yl)-1-(piperidin-1-yl)ethanone;
[113] 2-(9-(5-Chloro-1-methyl-3-(5-methylisoxazol-3-yl)-1H-pyrazole-4-carbonyl)-3,9-diazaspiro[5.5]undecan-3-yl)-1-(4,4-difluoropiperidin-1-yl)ethanone;
[114] 2-((5-Chloro-1-methyl-3-(5-methylisoxazol-3-yl)-1H-pyrazol-4-yl)methyl)-8-(3,3-dimethylbutyl)-2,8-diazaspiro[4.5]decan-1-one;
[115] 3-((5-Chloro-1-methyl-3-(5-methylisoxazol-3-yl)-1H-pyrazol-4-yl)methyl)-9-(3,3-dimethylbutyl)-3,9-diazaspiro[5.5]undecan-2-one;
[116] 2-((5-Chloro-1-methyl-3-(5-methylisoxazol-3-yl)-1H-pyrazol-4-yl)methyl)-8-(3,3-dimethylbutyl)-2,8-diazaspiro[4.5]decan-3-one;
[117] 1-((5-Chloro-1-methyl-3-(5-methylisoxazol-3-yl)-1H-pyrazol-4-yl)methyl)-3-(isopentylamino)azepan-2-one;
[118] 1-((5-Chloro-1-methyl-3-(5-methylisoxazol-3-yl)-1H-pyrazol-4-yl)methyl)-4-(isopentylamino)azepan-2-one;
[119] (S)-1-(2-(5-Chloro-1-methyl-3-(5-methylisoxazol-3-yl)-1H-pyrazol-4-yl)ethyl)-N-isopentylazepan-3-amine;
[120] (2-(5-Chloro-1-methyl-3-(5-methylisoxazol-3-yl)-1H-pyrazol-4-yl)-1-(9-isopentyl-3,9-diazaspiro[5.5]undecan-3-yl)ethenone;
[121] (5-Chloro-1-methyl-3-(5-methylisoxazol-3-yl)-1H-pyrazol-4-yl)(3-(((3,3-dimethylbutyl)(methyl)amino)methyl)piperidin-1-yl)methanone;
[122] (5-Chloro-1-methyl-3-(5-methylisoxazol-3-yl)-1H-pyrazol-4-yl)(3-((isobutyl(methyl)amino)methyl)piperidin-1-yl)methanone;
[123] (5-Chloro-1-methyl-3-(5-methylisoxazol-3-yl)-1H-pyrazol-4-yl)((2r,4s)-2-((3,3-dimethylbutyl)amino)-6-azaspiro[3.4]octan-6-yl)methanone;
[124] (5-Chloro-3-(6-hydroxypyridin-2-yl)-1-methyl-1H-pyrazol-4-yl)(9-(3,3-dimethylbutyl)-3,9-diazaspiro[5.5]undecan-3-yl)methanone;
[125] (R)-(5-Chloro-1-methyl-3-(5-methylisoxazol-3-yl)-1H-pyrazol-4-yl)(3-((isopentylamino)methyl)piperidin-1-yl)methanone; and
[126] (S)-(5-Chloro-1-methyl-3-(5-methylisoxazol-3-yl)-1H-pyrazol-4-yl)(3-((isopentylamino)methyl)piperidin-1-yl)methanone;
[127] 5-Chloro-N-(7-(3,3-dimethylbutyl)-7-azaspiro[3.5]nonan-2-yl)-1-ethyl-3-(5-methylisoxazol-3-yl)-1H-pyrazole-4-carboxamide;
[128] (5-Chloro-1-methyl-3-(5-methylisoxazol-3-yl)-1H-pyrazol-4-yl)(9-(3,4-difluorobenzyl)-2,9-diazaspiro[5.5]undecan-2-yl)methanone;
[129] (5-Chloro-1-methyl-3-(5-methylisoxazol-3-yl)-1H-pyrazol-4-yl)(9-(4-fluorobenzyl)-3,9-diazaspiro[5.5]undecan-3-yl)methanone;
[130] (5-Chloro-1-methyl-3-(5-methylisoxazol-3-yl)-1H-pyrazol-4-yl)(9-(3,4-difluorobenzyl)-3,9-diazaspiro[5.5]undecan-3-yl)methanone;
[131] 4-((9-(5-Chloro-1-methyl-3-(5-methylisoxazol-3-yl)-1H-pyrazole-4-carbonyl)-3,9-diazaspiro[5.5]undecan-3-yl)methyl)-2-fluorobenzonitrile;
[132] (5-Chloro-3-(5-isopropylisoxazol-3-yl)-1-methyl-1H-pyrazol-4-yl)(9-(3,3-dimethylbutyl)-2,9-diazaspiro[5.5]undecan-2-yl)methanone;
[133] (5-Chloro-3-(2-ethyl-4-methyloxazol-5-yl)-1-methyl-1H-pyrazol-4-yl)(9-(3,3-dimethylbutyl)-2,9-diazaspiro[5.5]undecan-2-yl)methanone;
[134] 5-Chloro-N-(2-(3,3-dimethylbutyl)-2-azaspiro[3.5]nonan-7-yl)-1-ethyl-3-(5-methylisoxazol-3-yl)-1H-pyrazole-4-carboxamide;
[135] (5-Chloro-1,1′-dimethyl-1H,1′H-[3,4′-bipyrazol]-4-yl)(9-(3,3-dimethylbutyl)-2,9-diazaspiro[5.5]undecan-2-yl)methanone;
[136] 5-Chloro-N-(7-(3,3-dimethylbutyl)-7-azaspiro[3.5]nonan-2-yl)-1-ethyl-3-(5-ethylisoxazol-3-yl)-1H-pyrazole-4-carboxamide;
[137] 5-Chloro-N-(2-(3,3-dimethylbutyl)-2-azaspiro[3.5]nonan-7-yl)-1-ethyl-3-(5-ethylisoxazol-3-yl)-1H-pyrazole-4-carboxamide;
[138] (5-Chloro-1′-ethyl-1-methyl-1H,1′H-[3,3′-bipyrazol]-4-yl)(9-(3,3-dimethylbutyl)-2,9-diazaspiro[5.5]undecan-2-yl)methanone;
[139] 5-Chloro-N-(7-(3,3-dimethylbutyl)-7-azaspiro[3.5]nonan-2-yl)-1-ethyl-3-(isoxazol-3-yl)-1H-pyrazole-4-carboxamide;
[140] 5-Chloro-N-(7-(3,3-dimethylbutyl)-7-azaspiro[3.5]nonan-2-yl)-1,1′-dimethyl-1H,1′H-[3,4′-bipyrazole]-4-carboxamide;
[141] 5-Chloro-N-(7-(3,3-dimethylbutyl)-7-azaspiro[3.5]nonan-2-yl)-1-methyl-3-(6-methylpyridin-2-yl)-1H-pyrazole-4-carboxamide;
[142] 5-Chloro-N-(3-(3,3-dimethylbutyl)-3-azaspiro[5.5]undecan-9-yl)-1-ethyl-3-(isoxazol-3-yl)-1H-pyrazole-4-carboxamide;
[143] 5-Chloro-N-(3-(3,3-dimethylbutyl)-3-azaspiro[5.5]undecan-9-yl)-1-ethyl-3-(5-methylisoxazol-3-yl)-1H-pyrazole-4-carboxamide;
[144] (5-Chloro-1-methyl-3-(5-methylisoxazol-3-yl)-1H-pyrazol-4-yl)(6-(3,3-dimethylbutyl)-2,6-diazaspiro[3.5]nonan-2-yl)methanone;
[145] 5-Chloro-N-(7-(3,3-dimethylbutyl)-7-azaspiro[3.5]nonan-2-yl)-1′-ethyl-1-methyl-1H,1′H-[3,4′-bipyrazole]-4-carboxamide;
[146] 5-Chloro-N-(3-(3,3-dimethylbutyl)-3-azaspiro[5.5]undecan-9-yl)-1′-ethyl-1-methyl-1H,1′H-[3,4′-bipyrazole]-4-carboxamide;
[147] 5-Chloro-N-(3-(3,3-dimethylbutyl)-3-azaspiro[5.5]undecan-9-yl)-1-methyl-3-(6-(trifluoromethyl)pyridin-2-yl)-1H-pyrazole-4-carboxamide;
[148] 5-Chloro-N-(3-(3,3-dimethylbutyl)-3-azaspiro[5.5]undecan-9-yl)-1-methyl-3-(6-methylpyridin-2-yl)-1H-pyrazole-4-carboxamide;
[149] 5-Chloro-N-(7-(3,3-dimethylbutyl)-7-azaspiro[3.5]nonan-2-yl)-1-ethyl-1′-isopropyl-1H,1′H-[3,4′-bipyrazole]-4-carboxamide;
[150] (5-Chloro-1-ethyl-3-(5-methylisoxazol-3-yl)-1H-pyrazol-4-yl)(9-(3,3-dimethylbutyl)-2,9-diazaspiro[5.5]undecan-2-yl)methanone;
[151] 5-Chloro-N-(3-(3,3-dimethylbutyl)-3-azaspiro[5.5]undecan-9-yl)-1-ethyl-3-(6-methylpyridin-2-yl)-1H-pyrazole-4-carboxamide;
[152] (5-Chloro-1-ethyl-3-(5-isopropylisoxazol-3-yl)-1H-pyrazol-4-yl)(9-(3,3-dimethylbutyl)-2,9-diazaspiro[5.5]undecan-2-yl)methanone;
N-(7-Benzyl-7-azaspiro[3.5]nonan-2-yl)-5-chloro-1-methyl-3-(5-methylisoxazol-3-yl)-1H-pyrazole-4-carboxamide;
[154] 5-Chloro-1-methyl-3-(5-methylisoxazol-3-yl)-N-(7-neopentyl-7-azaspiro[3.5]nonan-2-yl)-1H-pyrazole-4-carboxamide;
[155] (5-Chloro-1-methyl-3-(6-(trifluoromethyl)pyridin-2-yl)-1H-pyrazol-4-yl)(9-(3,3-dimethylbutyl)-2,9-diazaspiro[5.5]undecan-2-yl)methanone;
[156] (9-Benzyl-2,9-diazaspiro[5.5]undecan-2-yl)(5-chloro-1-methyl-3-(5-methylisoxazol-3-yl)-1H-pyrazol-4-yl)methanone;
[157] (5-Chloro-1-methyl-3-(5-methylisoxazol-3-yl)-1H-pyrazol-4-yl)(9-neopentyl-2,9-diazaspiro[5.5]undecan-2-yl)methanone;
[158] 5-Chloro-N-(2-(1-(3,3-dimethylbutyl)piperidin-4-yl)ethyl)-1-ethyl-3-(5-methylisoxazol-3-yl)-1H-pyrazole-4-carboxamide;
[159] 5-Chloro-N-(1-(3,3-dimethylbutyl)piperidin-4-yl)-1-ethyl-3-(5-methylisoxazol-3-yl)-1H-pyrazole-4-carboxamide;
[160] 5-Chloro-N-(1-(3,3-dimethylbutyl)azepan-4-yl)-1-ethyl-3-(5-methylisoxazol-3-yl)-1H-pyrazole-4-carboxamide;
[161] 5-Chloro-N-(1-(3,3-dimethylbutyl)piperidin-4-yl)-1-ethyl-3-(6-(trifluoromethyl)pyridin-2-yl)-1H-pyrazole-4-carboxamide;
[162] 5-Chloro-N-(1-(3,3-dimethylbutyl)piperidin-4-yl)-1-ethyl-3-(5-isopropylisoxazol-3-yl)-1H-pyrazole-4-carboxamide;
[163] 5-Chloro-N-(1-(3,3-dimethylbutyl)piperidin-4-yl)-1-ethyl-3-(isoxazol-3-yl)-1H-pyrazole-4-carboxamide;
[164] (S)-5-Chloro-N-(1-(3,3-dimethylbutyl)pyrrolidin-3-yl)-1-ethyl-3-(5-methylisoxazol-3-yl)-1H-pyrazole-4-carboxamide;
[165] (R)-5-Chloro-N-(1-(3,3-dimethylbutyl)pyrrolidin-3-yl)-1-ethyl-3-(5-methylisoxazol-3-yl)-1H-pyrazole-4-carboxamide;
[166] 5-Chloro-N-(1-(3,3-dimethylbutyl)piperidin-4-yl)-1-methyl-3-(5-methylisoxazol-3-yl)-1H-pyrazole-4-carboxamide;
[167] 5-Chloro-N-(1-(3,3-dimethylbutyl)piperidin-4-yl)-1-methyl-3-(6-(trifluoromethyl)pyridin-2-yl)-1H-pyrazole-4-carboxamide;
[168] 5-Chloro-N-(1-(3,3-dimethylbutyl)piperidin-4-yl)-1-methyl-3-(6-methylpyridin-2-yl)-1H-pyrazole-4-carboxamide;
[169] (5-Chloro1-methyl-3-(5-methylisoxazol-3-yl)-1H-pyrazol-4-yl)((2s,4r)-2-((3,3-dimethylbutyl)amino)-6-azaspiro[3.4]octan-6-yl)methanone;
[170] (5-Chloro-3-(5-methylisoxazol-3-yl)-1-(pyridin-4-yl)-1H-pyrazol-4-yl)(9-(3,3-dimethylbutyl)-3,9-diazaspiro[5.5]undecan-3-yl)methanone;
[171] (5-Chloro-1-ethyl-3-(isoxazol-3-yl)-1H-pyrazol-4-yl)(9-(3,3-dimethylbutyl)-3,9-diazaspiro[5.5]undecan-3-yl)methanone;
[172] (5-Chloro-3-(5-methylisoxazol-3-yl)-1-(pyridin-2-yl)-1H-pyrazol-4-yl)(9-(3,3-dimethylbutyl)-3,9-diazaspiro[5.5]undecan-3-yl)methanone;
[173] (5-Chloro-1-methyl-3-(pyridin-2-yl)-1H-pyrazol-4-yl)(9-(3,3-dimethylbutyl)-3,9-diazaspiro[5.5]undecan-3-yl)methanone;
[174] (5-Chloro-1-methyl-3-(5-methylisoxazol-3-yl)-1H-pyrazol-4-yl)(2-((3,3-dimethylbutyl)amino)-7-azaspiro[3.5]nonan-7-yl)methanone;
[175] (9-Benzyl-3,9-diazaspiro[5.5]undecan-3-yl)(5-chloro-1-methyl-3-(5-methylisoxazol-3-yl)-1H-pyrazol-4-yl)methanone;
[176] (5-Chloro-3-(2-ethyl-4-methyloxazol-5-yl)-1-methyl-1H-pyrazol-4-yl)(9-(3,3-dimethylbutyl)-3,9-diazaspiro[5.5]undecan-3-yl)methanone;
[177] (5-Chloro-3-(5-isopropylisoxazol-3-yl)-1-methyl-1H-pyrazol-4-yl)(9-(3,3-dimethylbutyl)-3,9-diazaspiro[5.5]undecan-3-yl)methanone;
[178] (5-Chloro-1,1′-dimethyl-1H,1′H-[3,4′-bipyrazol]-4-yl)(9-(3,3-dimethylbutyl)-3,9-diazaspiro[5.5]undecan-3-yl)methanone;
[179] (5-Chloro-1′-ethyl-1-methyl-1H,1′H-[3,3′-bipyrazol]-4-yl)(9-(3,3-dimethylbutyl)-3,9-diazaspiro[5.5]undecan-3-yl)methanone;
[180] (5-Chloro-1-methyl-3-(6-(trifluoromethyl)144ndecane-2-yl)-1H-pyrazol-4-yl)(9-(3,3-dimethylbutyl)-3,9-diazaspiro[5.5]144ndecane-3-yl)methanone;
[181] (5-Chloro-1,1′-dimethyl-1H,1′H-[3,3′-bipyrazol]-4-yl)(9-(3,3-dimethylbutyl)-3,9-diazaspiro[5.5]undecan-3-yl)methanone;
[182] (5-Chloro-3-(5-methylisoxazol-3-yl)-1-propyl-1H-pyrazol-4-yl)(9-(3,3-dimethylbutyl)-3,9-diazaspiro[5.5]undecan-3-yl)methanone;
[183] (5-Chloro-1-isopropyl-3-(5-methylisoxazol-3-yl)-1H-pyrazol-4-yl)(9-(3,3-dimethylbutyl)-3,9-diazaspiro[5.5]undecan-3-yl)methanone;
[184] (5-Chloro-1-ethyl-3-(5-methylisoxazol-3-yl)-1H-pyrazol-4-yl)(7-(3,3-dimethylbutyl)-2,7-diazaspiro[3.5]nonan-2-yl)methanone;
[185] (5-Chloro-3-(5-methylisoxazol-3-yl)-1-propyl-1H-pyrazol-4-yl)(7-(3,3-dimethylbutyl)-2,7-diazaspiro[3.5]nonan-2-yl)methanone;
[186] (5-Chloro-3-(6-ethoxypyridin-2-yl)-1-methyl-1H-pyrazol-4-yl)(9-(3,3-dimethylbutyl)-3,9-diazaspiro[5.5]undecan-3-yl)methanone;
[187] (5-Chloro-1-ethyl-3-(isoxazol-3-yl)-1H-pyrazol-4-yl)(7-(3,3-dimethylbutyl)-2,7-diazaspiro[3.5]nonan-2-yl)methanone;
[188] (5-Chloro-1-isopropyl-3-(5-methylisoxazol-3-yl)-1H-pyrazol-4-yl)(7-(3,3-dimethylbutyl)-2,7-diazaspiro[3.5]nonan-2-yl)methanone;
[189] (5-Chloro-1-methyl-3-(6-methylpyridin-2-yl)-1H-pyrazol-4-yl)(9-(3,3-dimethylbutyl)-3,9-diazaspiro[5.5]undecan-3-yl)methanone;
[190] (5-Chloro-3-(5-cyclopropylisoxazol-3-yl)-1-ethyl-1H-pyrazol-4-yl)(9-(3,3-dimethylbutyl)-3,9-diazaspiro[5.5]undecan-3-yl)methanone;
[191] (5-Chloro-3-(5-cyclopropylisoxazol-3-yl)-1-methyl-1H-pyrazol-4-yl)(9-(3,3-dimethylbutyl)-3,9-diazaspiro[5.5]undecan-3-yl)methanone;
[192] (5-Chloro-1-ethyl-3-(5-isopropylisoxazol-3-yl)-1H-pyrazol-4-yl)(9-(3,3-dimethylbutyl)-3,9-diazaspiro[5.5]undecan-3-yl)methanone;
[193] (5-Chloro-1-methyl-3-(5-methylisoxazol-3-yl)-1H-pyrazol-4-yl)(2-(3,3-dimethylbutyl)-2,6-diazaspiro[3.5]nonan-6-yl)methanone;
[194] (5-Chloro-1-ethyl-3-(5-methylisoxazol-3-yl)-1H-pyrazol-4-yl)(2-(3,3-dimethylbutyl)-2,6-diazaspiro[3.5]nonan-6-yl)methanone;
[195] (5-Chloro-1-ethyl-3-(isoxazol-3-yl)-1H-pyrazol-4-yl)(2-(3,3-dimethylbutyl)-2,6-diazaspiro[3.5]nonan-6-yl)methanone;
[196] (5-Chloro-1′-ethyl-1-methyl-1H,1′H-[3,4′-bipyrazol]-4-yl)(9-(3,3-dimethylbutyl)-3,9-diazaspiro[5.5]undecan-3-yl)methanone;
[197] 5-Chloro-N-(2-(3,3-dimethylbutyl)-2-azaspiro[3.5]nonan-7-yl)-1-ethyl-3-(isoxazol-3-yl)-1H-pyrazole-4-carboxamide;
[198] (5-Chloro-1-ethyl-1′-isopropyl-1H,1′H-[3,4′-bipyrazol]-4-yl)(9-(3,3-dimethylbutyl)-3,9-diazaspiro[5.5]undecan-3-yl)methanone;
[199] (5-Chloro-1-ethyl-3-(6-(trifluoromethyl)pyridin-2-yl)-1H-pyrazol-4-yl)(9-(3,3-dimethylbutyl)-3,9-diazaspiro[5.5]undecan-3-yl)methanone;
[200] 5-Chloro-N-((1r,4r)-4-((3,3-dimethylbutyl)amino)cyclohexyl)-1-ethyl-3-(5-methylisoxazol-3-yl)-1H-pyrazole-4-carboxamide;
[201] 5-Chloro-N-((1s,4s)-4-((3,3-dimethylbutyl)amino)cyclohexyl)-1-ethyl-3-(5-methylisoxazol-3-yl)-1H-pyrazole-4-carboxamide;
[202] 5-Chloro-N-(1-(3,3-dimethylbutyl)piperidin-3-yl)-1-ethyl-3-(5-methylisoxazol-3-yl)-1H-pyrazole-4-carboxamide;
[203] 5-Chloro-N-((1-(3,3-dimethylbutyl)piperidin-4-yl)methyl)-1-ethyl-3-(5-methylisoxazol-3-yl)-1H-pyrazole-4-carboxamide;
[204] 5-Chloro-N-(2-(3,3-dimethylbutyl)-1,2,3,4-tetrahydroisoquinolin-6-yl)-1-ethyl-3-(5-methylisoxazol-3-yl)-1H-pyrazole-4-carboxamide;
[205] 5-Chloro-N-((1-(3,3-dimethylbutyl)piperidin-3-yl)methyl)-1-ethyl-3-(5-methylisoxazol-3-yl)-1H-pyrazole-4-carboxamide;
[206] 5-Chloro-N-(1-(3,3-dimethylbutyl)azepan-3-yl)-1-ethyl-3-(5-methylisoxazol-3-yl)-1H-pyrazole-4-carboxamide;
[207] (5-Chloro-1-ethyl-3-(2-methylpyridin-4-yl)-1H-pyrazol-4-yl)(9-(3,3-dimethylbutyl)-3,9-diazaspiro[5.5]undecan-3-yl)methanone;
[208] 2-(5-Chloro-1-methyl-3-(5-methylisoxazol-3-yl)-1H-pyrazol-4-yl)-1-(9-(3,3-dimethylbutyl)-3,9-diazaspiro[5.5]undecan-3-yl)ethan-1-one
[209] 2-(5-Chloro-1-ethyl-3-(isoxazol-3-yl)-1H-pyrazol-4-yl)-1-(9-(3,3-dimethylbutyl)-3,9-diazaspiro[5.5]undecan-3-yl)ethan-1-one;
[210] 2-(5-Chloro-1-ethyl-3-(5-methylisoxazol-3-yl)-1H-pyrazol-4-yl)-1-(9-(3,3-dimethylbutyl)-3,9-diazaspiro[5.5]undecan-3-yl)ethan-1-one;
[211] 2-(5-Chloro-1-ethyl-3-(isoxazol-3-yl)-1H-pyrazol-4-yl)-N-(2-(3,3-dimethylbutyl)-1,2,3,4-tetrahydroisoquinolin-6-yl)acetamide;
[212] 2-(5-Chloro-1-ethyl-3-(isoxazol-3-yl)-1H-pyrazol-4-yl)-N-((1-(3,3-dimethylbutyl)piperidin-3-yl)methyl)acetamide;
[213] 2-(5-Chloro-1-methyl-3-(6-(trifluoromethyl)pyridin-2-yl)-1H-pyrazol-4-yl)-1-(9-(3,3-dimethylbutyl)-3,9-diazaspiro[5.5]undecan-3-yl)ethan-1-one;
[214] 2-(5-Chloro-1-methyl-3-(5-methylisoxazol-3-yl)-1H-pyrazol-4-yl)-1-(9-(3,3-dimethylbutyl)-2,9-diazaspiro[5.5]undecan-2-yl)ethan-1-one;
[215] 2-(5-Chloro-1-methyl-3-(5-methylisoxazol-3-yl)-1H-pyrazol-4-yl)-N-(3-(3,3-dimethylbutyl)-3-azaspiro[5.5]undecan-9-yl)acetamide;
[216] 2-(5-Chloro-1-ethyl-3-(5-methylisoxazol-3-yl)-1H-pyrazol-4-yl)-N-(3-(3,3-dimethylbutyl)-3-azaspiro[5.5]undecan-9-yl)acetamide;
[217] 2-(5-Chloro-1-ethyl-3-(5-methylisoxazol-3-yl)-1H-pyrazol-4-yl)-1-(9-(3,3-dimethylbutyl)-2,9-diazaspiro[5.5]undecan-2-yl)ethan-1-one;
[218] 2-(5-Chloro-1-ethyl-3-(isoxazol-3-yl)-1H-pyrazol-4-yl)-1-(9-(3,3-dimethylbutyl)-2,9-diazaspiro[5.5]undecan-2-yl)ethan-1-one;
[219] 2-(5-Chloro-1-ethyl-3-(isoxazol-3-yl)-1H-pyrazol-4-yl)-N-(3-(3,3-dimethylbutyl)-3-azaspiro[5.5]undecan-9-yl)acetamide;
[220] 2-(5-Chloro-1-methyl-3-(5-methylisoxazol-3-yl)-1H-pyrazol-4-yl)-N-(1-(3,3-dimethylbutyl)piperidin-4-yl)acetamide;
[221] 2-(5-Chloro-1-ethyl-3-(isoxazol-3-yl)-1H-pyrazol-4-yl)-N-(1-(3,3-dimethylbutyl)piperidin-4-yl)acetamide;
[222] 2-(5-Chloro-1-methyl-3-(6-(trifluoromethyl)pyridin-2-yl)-1H-pyrazol-4-yl)-N-(1-(3,3-dimethylbutyl)piperidin-4-yl)acetamide;
[223] 2-(5-chloro-1-ethyl-3-(6-(trifluoromethyl)pyridin-2-yl)-1H-pyrazol-4-yl)-N-(1-(3,3-dimethylbutyl)piperidin-4-yl)acetamide;
[224] 2-(5-Chloro-1-ethyl-3-(isoxazol-3-yl)-1H-pyrazol-4-yl)-N-(7-(3,3-dimethylbutyl)-7-azaspiro[3.5]nonan-2-yl)acetamide
[225] 2-((5-Chloro-1-ethyl-3-(5-methylisoxazol-3-yl)-1H-pyrazol-4-yl)methyl)-8-(3,3-dimethylbutyl)-2,8-diazaspiro[4.5]decan-3-one;
[226] 2-((5-Chloro-3-(5-methylisoxazol-3-yl)-1-propyl-1H-pyrazol-4-yl)methyl)-8-(3,3-dimethylbutyl)-2,8-diazaspiro[4.5]decan-3-one;
[227] 2-((5-Chloro-1-methyl-3-(6-(trifluoromethyl)pyridin-2-yl)-1H-pyrazol-4-yl)methyl)-8-(3,3-dimethylbutyl)-2,8-diazaspiro[4.5]decan-3-one;
[228] 2-((5-Chloro-1-methyl-3-(6-methylpyridin-2-yl)-1H-pyrazol-4-yl)methyl)-8-(3,3-dimethylbutyl)-2,8-diazaspiro[4.5]decan-3-one and
[229] 2-((5-Chloro-1-ethyl-3-(6-(trifluoromethyl)pyridin-2-yl)-1H-pyrazol-4-yl)methyl)-8-(3,3-dimethylbutyl)-2,8-diazaspiro[4.5]decan-3-one.
or a pharmaceutical acceptable salt, stereoisomer, co-crystal, prodrug or solvate thereof.

13. Process for the preparation of a compound of general formula (Ia): 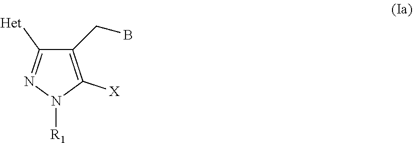
which comprises reacting a compound of formula (VI): 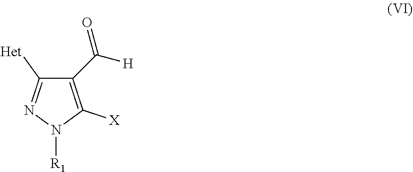
with a compound of formula (VII):
HB (VII)
wherein Het, X, B and R₁ are as defined in any one of claims 1 to 9.

14. Process for the preparation of a compound of general formula (Ib): 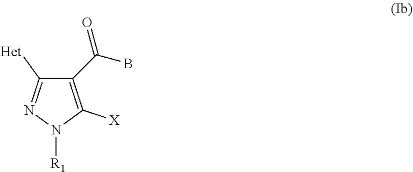
comprising the reaction between a compound of general formula (VIII): 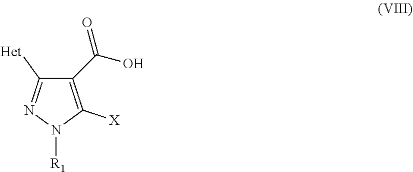
with a compound of formula (VII):
HB (VII)
wherein Het, X, B and R₁ are as defined in any one of claims 1 to 9.

15. Process for the preparation of a compound of general formula (Ic): 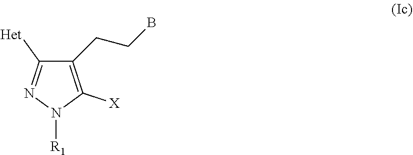
which comprises reacting a compound of formula (XII): 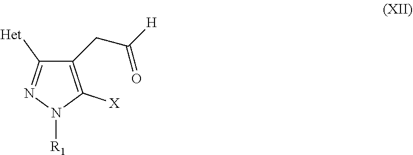
with a compound of formula (VII):
HB (VII)
wherein Het, X, B and R₁ are as defined in any one of claims 1 to 9.

16. Process for the preparation of a compound of general formula (Id): 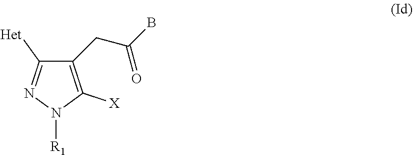
comprising the reaction between a compound of general formula (XIII): 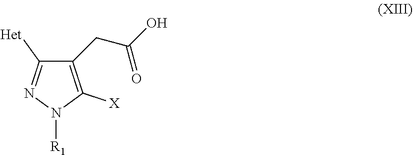
with a compound of formula (VII):
HB (VII)
wherein Het, X, B and R₁ are as defined in any one of claims 1 to 9.

17. A compound according to any one of claims 1 to 12 for use as a medicament.

18. A compound according to any one of claims 1 to 12 for use in the treatment and/or prophylaxis of diseases and/or disorders mediated by a sigma receptor.

19. A compound for use according to claim 18 wherein said sigma receptor is sigma-1 receptor and/or a sigma-2 receptor.

20. A compound for use according to claim 18 wherein the disease or disorder is pain, selected from neuropathic pain, inflammatory pain, chronic pain or any other pain conditions involving allodynia and/or hyperalgesia; or a CNS disorder or disease, selected from the group consisting of addiction to drugs and chemical substances including cocaine, amphetamine, ethanol and nicotine, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia or paranoia.

21. A pharmaceutical composition comprising a compound according to any one of claims 1 to 12 or a pharmaceutically acceptable salt, isomer, co-crystal, prodrug or solvate thereof, and at least a pharmaceutically acceptable carrier, additive, adjuvant or vehicle.
